SEQUENCE LISTING (109 sequences)

SEQ ID NO: 1 - protein, 6 aa, Artificial Sequence
  Synthesized Construct
KKKFKF

SEQ ID NO: 2 - protein, 6 aa, Artificial Sequence
  Synthesized Construct
KKKWKW

SEQ ID NO: 3 - protein, 6 aa, Artificial Sequence
  Synthesized Construct
KKKLKL

SEQ ID NO: 4 - protein, 6 aa, Artificial Sequence
  Synthesized Construct
KKKKKK

SEQ ID NO: 5 - protein, 12 aa, Artificial Sequence
  Synthesized Construct
KKKKKKKKKKKK

SEQ ID NO: 6 - protein, 12 aa, Artificial Sequence
  Synthesized Construct
KFYLYAIDTHRM

SEQ ID NO: 7 - protein, 12 aa, Artificial Sequence
  Synthesized Construct
KIIKWGIFWMQG

SEQ ID NO: 8 - protein, 12 aa, Artificial Sequence
  Synthesized Construct
NFFKKFRFTFTM

SEQ ID NO: 9 - protein, 6 aa, Artificial Sequence
  Synthesized Construct
XXXXXX

SEQ ID NO: 10 - protein, 6 aa, Artificial Sequence
  Synthesized Construct
XXXXXX

SEQ ID NO: 11 - protein, 6 aa, Artificial Sequence
  Synthesized Construct
XXXXXX

SEQ ID NO: 12 - protein, 6 aa, Artificial Sequence
  Synthesized Construct
XXXXXX

SEQ ID NO: 13 - protein, 12 aa, Artificial Sequence
  Synthesized Construct
XXXXXXXXXXXX

SEQ ID NO: 14 - protein, 18 aa, Artificial Sequence
  Synthesized Construct
XXXXXXXXXXXXXXXXXX

SEQ ID NO: 15 - protein, 6 aa, Artificial Sequence
  Synthesized Construct
XXXXXX

SEQ ID NO: 16 - protein, 6 aa, Artificial Sequence
  Synthesized Construct
XXXXXX

SEQ ID NO: 17 - protein, 7 aa, Artificial Sequence
  Synthesized Construct
XXXXXXX

SEQ ID NO: 18 - protein, 7 aa, Artificial Sequence
  Synthesized Construct
XXXXXXX

SEQ ID NO: 19 - protein, 7 aa, Artificial Sequence
  Synthesized Construct
XXXXXXX

SEQ ID NO: 20 - protein, 7 aa, Artificial Sequence
  Synthesized Construct
XXXXXXX

SEQ ID NO: 21 - protein, 6 aa, Artificial Sequence
  Synthesized Construct
XXXXXX

SEQ ID NO: 22 - protein, 7 aa, Artificial Sequence
  Synthesized Construct
XXXXXXX

SEQ ID NO: 23 - protein, 7 aa, Artificial Sequence
  Synthesized Construct
XXXXXXX

SEQ ID NO: 24 - protein, 7 aa, Artificial Sequence
  Synthesized Construct
XXXXXXX

SEQ ID NO: 25 - protein, 7 aa, Artificial Sequence
  Synthesized Construct
XXXXXXX

SEQ ID NO: 26 - protein, 7 aa, Artificial Sequence
  Synthesized Construct
XXXXXXX

SEQ ID NO: 27 - protein, 7 aa, Artificial Sequence
  Synthesized Construct
XXXXXXX

SEQ ID NO: 28 - protein, 7 aa, Artificial Sequence
  Synthesized Construct
XXXXXXX

SEQ ID NO: 29 - protein, 6 aa, Artificial Sequence
  Synthesized Construct
XXXXXX

SEQ ID NO: 30 - protein, 6 aa, Artificial Sequence
  Synthesized Construct
DDDDDD

SEQ ID NO: 31 - protein, 6 aa, Artificial Sequence
  Synthesized Construct
HHHHHH

SEQ ID NO: 32 - protein, 6 aa, Artificial Sequence
  Synthesized Construct
AAKKAA

SEQ ID NO: 33 - protein, 6 aa, Artificial Sequence
  Synthesized Construct
AAKKKA

SEQ ID NO: 34 - protein, 6 aa, Artificial Sequence
  Synthesized Construct
AKKKKA

SEQ ID NO: 35 - protein, 6 aa, Artificial Sequence
  Synthesized Construct
AKKKKK

SEQ ID NO: 36 - protein, 6 aa, Artificial Sequence
  Synthesized Construct
FKFKKK

SEQ ID NO: 37 - protein, 6 aa, Artificial Sequence
  Synthesized Construct
XXXXXX

SEQ ID NO: 38 - protein, 8 aa, Artificial Sequence
  Synthesized Construct
FKFSLFSG

SEQ ID NO: 39 - protein, 8 aa, Artificial Sequence
  Synthesized Construct
DFKLNFKF

SEQ ID NO: 40 - protein, 8 aa, Artificial Sequence
  Synthesized Construct
FKFNLFSG

SEQ ID NO: 41 - protein, 6 aa, Artificial Sequence
  Synthesized Construct
YKYKKK

SEQ ID NO: 42 - protein, 6 aa, Artificial Sequence
  Synthesized Construct
KKFKKF

SEQ ID NO: 43 - protein, 6 aa, Artificial Sequence
  Synthesized Construct
KFKKKF

SEQ ID NO: 44 - protein, 6 aa, Artificial Sequence
  Synthesized Construct
KIGVVR

SEQ ID NO: 45 - protein, 6 aa, Artificial Sequence
  Synthesized Construct
AKVKKK

SEQ ID NO: 46 - protein, 6 aa, Artificial Sequence
  Synthesized Construct
AKFKKK

SEQ ID NO: 47 - protein, 10 aa, Artificial Sequence
  Synthesized Construct
RGRERFEMFR

SEQ ID NO: 48 - protein, 11 aa, Artificial Sequence
  Synthesized Construct
YGRKKRRQRRR

SEQ ID NO: 49 - protein, 8 aa, Artificial Sequence
  Synthesized Construct
FFFKFKKK

SEQ ID NO: 50 - protein, 10 aa, Artificial Sequence
  Synthesized Construct
FFFFFKFKKK

SEQ ID NO: 51 - protein, 6 aa, Artificial Sequence
  Synthesized Construct
FFFKKK

SEQ ID NO: 52 - protein, 6 aa, Artificial Sequence
  Synthesized Construct
FFFFKK

SEQ ID NO: 53 - protein, 6 aa, Artificial Sequence
  Synthesized Construct
FXFXXX

SEQ ID NO: 54 - protein, 6 aa, Artificial Sequence
  Synthesized Construct
FXFXXX

SEQ ID NO: 55 - protein, 6 aa, Artificial Sequence
  Synthesized Construct
XKKFKF

SEQ ID NO: 56 - protein, 6 aa, Artificial Sequence
  Synthesized Construct
XKKFKF

SEQ ID NO: 57 - protein, 6 aa, Artificial Sequence
  Synthesized Construct
XKKFKF

SEQ ID NO: 58 - protein, 6 aa, Artificial Sequence
  Synthesized Construct
AXXFXA

SEQ ID NO: 59 - protein, 6 aa, Artificial Sequence
  Synthesized Construct
XKXKKK

SEQ ID NO: 60 - protein, 6 aa, Artificial Sequence
  Synthesized Construct
KKKXKX

SEQ ID NO: 61 - protein, 6 aa, Artificial Sequence
  Synthesized Construct
KKKXKX

SEQ ID NO: 62 - protein, 6 aa, Artificial Sequence
  Synthesized Construct
XKXKKK

SEQ ID NO: 63 - protein, 6 aa, Artificial Sequence
  Synthesized Construct
XKXKKK

SEQ ID NO: 64 - protein, 6 aa, Artificial Sequence
  Synthesized Construct
XKXKKK

SEQ ID NO: 65 - protein, 6 aa, Artificial Sequence
  Synthesized Construct
XKXKKK

SEQ ID NO: 66 - protein, 6 aa, Artificial Sequence
  Synthesized Construct
XKXKKK

SEQ ID NO: 67 - protein, 12 aa, Artificial Sequence
  Synthesized Construct
MKFMKMHNKKRY

SEQ ID NO: 68 - protein, 12 aa, Artificial Sequence
  Synthesized Construct
LTAVKKVKAPTR

SEQ ID NO: 69 - protein, 12 aa, Artificial Sequence
  Synthesized Construct
LIPIRKKYFFKL

SEQ ID NO: 70 - protein, 12 aa, Artificial Sequence
  Synthesized Construct
KLSLIWLHTHWH

SEQ ID NO: 71 - protein, 12 aa, Artificial Sequence
  Synthesized Construct
IRYVTHQYILWP

SEQ ID NO: 72 - protein, 12 aa, Artificial Sequence
  Synthesized Construct
YNKIGVVRLFSE

SEQ ID NO: 73 - protein, 12 aa, Artificial Sequence
  Synthesized Construct
YRHRWEVMLWWP

SEQ ID NO: 74 - protein, 12 aa, Artificial Sequence
  Synthesized Construct
WAVKLFTFFMFH

SEQ ID NO: 75 - protein, 12 aa, Artificial Sequence
  Synthesized Construct
YQSWWFFYFKLA

SEQ ID NO: 76 - protein, 12 aa, Artificial Sequence
  Synthesized Construct
WWYKLVATHLYG

SEQ ID NO: 77 - protein, 12 aa, Artificial Sequence
  Synthesized Construct
QTLSLHFQTRPP

SEQ ID NO: 78 - protein, 12 aa, Artificial Sequence
  Synthesized Construct
TRLAMQYVGYFW

SEQ ID NO: 79 - protein, 12 aa, Artificial Sequence
  Synthesized Construct
RYWYRHWSQHDN

SEQ ID NO: 80 - protein, 12 aa, Artificial Sequence
  Synthesized Construct
AQYIMFKVFYLS

SEQ ID NO: 81 - protein, 12 aa, Artificial Sequence
  Synthesized Construct
TGIRIYSWKMWL

SEQ ID NO: 82 - protein, 12 aa, Artificial Sequence
  Synthesized Construct
SRYLMYVNIIYI

SEQ ID NO: 83 - protein, 12 aa, Artificial Sequence
  Synthesized Construct
RYWMNAFYSPMW

SEQ ID NO: 84 - protein, 12 aa, Artificial Sequence
  Synthesized Construct
NFYTYKLAYMQM

SEQ ID NO: 85 - protein, 12 aa, Artificial Sequence
  Synthesized Construct
MGYSSGYWSRQV

SEQ ID NO: 86 - protein, 12 aa, Artificial Sequence
  Synthesized Construct
YFYMKLLWTKER

SEQ ID NO: 87 - protein, 12 aa, Artificial Sequence
  Synthesized Construct
RIMYLYHRLQHT

SEQ ID NO: 88 - protein, 12 aa, Artificial Sequence
  Synthesized Construct
RWRHSSFYPIWF

SEQ ID NO: 89 - protein, 12 aa, Artificial Sequence
  Synthesized Construct
QVRIFTNVEFKH

SEQ ID NO: 90 - protein, 12 aa, Artificial Sequence
  Synthesized Construct
RYLHWYAVAVKV

SEQ ID NO: 91 - protein, 6 aa, Artificial Sequence
  Synthesized Construct
XXXXXX

SEQ ID NO: 92 - protein, 5 aa, Artificial Sequence
  Synthesized Construct
XXXXX

SEQ ID NO: 93 - protein, 6 aa, Artificial Sequence
  Synthesized Construct
XXXXXX

SEQ ID NO: 94 - protein, 6 aa, Artificial Sequence
  Synthesized Construct
XXXXXX

SEQ ID NO: 95 - protein, 6 aa, Artificial Sequence
  Synthesized Construct
XXXXXX

SEQ ID NO: 96 - protein, 12 aa, Artificial Sequence
  Synthesized Construct
XXXXXXXXXXXX

SEQ ID NO: 97 - protein, 6 aa, Artificial Sequence
  Synthesized Construct
XXXXXX

SEQ ID NO: 98 - protein, 9 aa, Artificial Sequence
  Synthesized Construct
XKFKKKXXX

SEQ ID NO: 99 - protein, 6 aa, Artificial Sequence
  Synthesized Construct
XKFKKK

SEQ ID NO: 100 - protein, 6 aa, Artificial Sequence
  Synthesized Construct
DDDDDD

SEQ ID NO: 101 - protein, 6 aa, Artificial Sequence
  Synthesized Construct
HHHHHH

SEQ ID NO: 102 - protein, 6 aa, Artificial Sequence
  Synthesized Construct
AAAKAA

SEQ ID NO: 103 - protein, 6 aa, Artificial Sequence
  Synthesized Construct
XKKFKF

SEQ ID NO: 104 - protein, 6 aa, Artificial Sequence
  Synthesized Construct
XKFKKK

SEQ ID NO: 105 - protein, 9 aa, Artificial Sequence
  Synthesized Construct
XKKFKFXXX

SEQ ID NO: 106 - protein, 6 aa, Artificial Sequence
  Synthesized Construct
XXXXXX

SEQ ID NO: 107 - protein, 8 aa, Artificial Sequence
  Synthesized Construct
ANFFAHSS

SEQ ID NO: 108 - protein, 6 aa, Artificial Sequence
  Synthesized Construct
XKKFKF

SEQ ID NO: 109 - protein, 4 aa, Artificial Sequence
  Synthesized Construct
XKXX

(19) United States
(12) Patent Application Publication
(10) Pub. No.: US 2013/0109581 A1
(43) Pub. Date: May 2, 2013

(54) POSITIVELY CHARGED SPECIES AS BINDING REAGENTS IN THE SEPARATION OF PROTEIN AGGREGATES FROM MONOMERS

(71) Applicants: David Peretz, Emeryville, CA (US); Alice Yam, Emeryville, CA (US); Xuemei Wang, Emeryville, CA (US); Man Gao, Emeryville, CA (US)

(72) Inventors: Cleo Salisbury, Emeryville, CA (US); David Peretz, Emeryville, CA (US); Alice Yam, Emeryville, CA (US); Xuemei Wang, Emeryville, CA (US); Man Gao, Emeryville, CA (US)

(21) Appl. No.: 13/508,037

(22) PCT Filed: Nov. 4, 2010
(86) PCT No.: PCT/US10/55528

§ 371 Date: Nov. 20, 2012

Related U.S. Application Data
(60) Provisional application No. 61/258,188, filed on Nov. 4, 2009.
(60) Provisional application No. 61/265,340, filed on Nov. 30, 2009.

(51) Int. Cl. G01N 33/68

(52) U.S. Cl. CPC G01N 33/6896
USPC 506/9; 435/7.94; 435/7.92; 436/501; 435/7.1

(57) ABSTRACT

The invention provides methods for detecting the presence of an aggregate in a sample by contacting the sample suspected of containing an aggregate with an aggregate-specific binding reagent under conditions that allow the binding of the reagent to the aggregate, if present; and detecting the presence of the aggregate, if any, in the sample by its binding to the reagent; where the aggregate-specific binding reagent typically has a net charge of at least about positive one at the pH at which the sample is contacted with the ASB reagent, is attached to a solid support at a charge density of at least about 60 nmol net charge per square meter, and binds preferentially with aggregates over monomers when attached to the solid support. Methods for detecting the presence of oligomer are also provided. Compositions for use in the methods are provided.

### BACKGROUND

Protein misfolding is a normal occurrence in cells. However, misfolded proteins tend to self-associate, which results in protein aggregates of various sizes and structures. As persistent misfolded proteins can lead to toxic aggregates, the cell contains pathways and machinery to reduce the amount of misfolded proteins in the cell. Misfolded proteins intermediates are recognized by molecular chaperones, which assist in the correct folding of the intermediate. If misfolded proteins escape correction by chaperones, the ubiquitin-proteosome pathway generally degrades them.

The accumulation of misfolded proteins is associated with a variety of diseases. Protein conformational diseases include a variety of clinically unrelated diseases, such as transmissible spongiform encephalopathies, Alzheimer's disease, ALS, and diabetes, which arise from an aberrant conformational transition of a normal protein into a pathogenic conformer. This transition, in turn, can lead to self-association of the pathogenic conformer into smaller aggregates such as oligomers or larger aggregates such as fibrils with consequent tissue deposition and is hypothesized to lead to damage of the surrounding tissue.

Detection of the aggregates of conformational disease proteins in living subjects and samples obtained from living subjects has proven difficult. The current techniques for confirming the presence of aggregates in living patients are crude and invasive. For example, histopathological examination would require biopsies that are risky to the subject. Histopathology is inherently prone to sampling error as lesions and deposits of aggregated pathogenic conformer can be missed depending on the area where the biopsy is taken. Thus, definitive diagnosis and palliative treatments for these conditions before death of the subject remains a substantially unmet challenge.

Deposition of amyloid-beta protein (Aβ) aggregates, mainly Aβ1-40 (Aβ40) and 1-42 (Aβ42), has been exhaustively linked to Alzheimer's disease (AD) and is considered to be the gold-standard marker for the disease. However, the only definitive test for AD is immunohistochemical staining of plaques of fibrillar Aβ aggregate from post-mortem brain samples. Currently, there are no FDA-approved ante-mortem diagnostic tests for AD. Plasma or CSF samples could be used for ante-mortem tests. Some ante-mortem AD tests have focused on the cerebrospinal fluid (CSF) and attempt to quantitate soluble monomeric Aβ42. However, this biomarker only serves as an indirect measurement of AD.

Recent literature has suggested that small, soluble, non-fibrillar oligomeric species of Aβ are likely to be the neurotoxic agents directly contributing to the Alzheimer's disease phenotype (Hoshi et al., PNAS, 2003, 100, 6370; Lambert et al., PNAS, 1998, 95, 6448). Furthermore, using antibodies raised against Aβ42, elevated levels of Aβ oligomeric species were found in cerebrospinal fluid (CSF) taken from patients with Alzheimer's disease compared to CSF taken from healthy control subjects (Georganopoulou et al. PNAS, 2005, 102, 2273). However, to date, no small molecule that is capable of binding oligomer has been reported.

Thus, a test that can specifically detect aggregated Aβ directly from the CSF or other body fluids such as plasma would have a great advantage. Early detection of aggregates such as soluble Aβ oligomers will allow faster and more efficient diagnosis and evaluation of potential therapies for Alzheimer's disease.

Tests that can detect pathogenic aggregates of other conformational disease proteins directly from samples of body fluid are also desired, as they would also allow faster and earlier diagnosis and evaluation of potential therapies for these conformational diseases.

In addition, quality control of manufactured polypeptides would also benefit from the use of reagents that bind specifically to aggregates. Because polypeptides, such as recombinant insulin or therapeutic antibodies, are generally produced at high levels, aggregates tend to form. Thus, there is a need for reagents which can specifically bind to aggregates for their removal from preparations of desired polypeptides.

### BRIEF SUMMARY OF PREFERRED EMBODIMENTS

The invention described herein meets these needs by providing methods for detecting the presence of aggregates in a sample with an aggregate-specific binding reagent. In preferred embodiments, the methods detect the presence of oligomers.

Thus, one aspect includes methods for detecting the presence of aggregate in a sample including the steps of contacting a sample suspected of containing aggregate with an aggregate-specific binding reagent under conditions that allow binding of said reagent to said aggregate, if present, to form a complex; and detecting the presence of aggregate, if any, in said sample by its binding to said aggregate-specific binding reagent, wherein said aggregate-specific binding reagent has a net charge of at least about positive one at the pH at which said sample is contacted with said aggregate-specific binding reagent, is attached to a solid support at a charge density of at least about 60 nmol net charge per square meter, and binds preferentially to aggregate over monomer when attached to said solid support.

Another aspect includes methods for detecting the presence of aggregate in a sample including the steps of contacting a sample suspected of containing aggregate with an aggregate-specific binding reagent under conditions that allow binding of said reagent to said aggregate, if present, to form a complex; contacting said complex with a conformational protein-specific binding reagent under conditions that allow binding; and detecting the presence of aggregate, if any, in said sample by its binding to said conformational protein-specific binding reagent, wherein said aggregate-specific binding reagent has a net charge of at least about positive one at the pH at which said sample is contacted with said aggregate-specific binding reagent, is attached to a solid support at a charge density of at least about 60 nmol net charge per square meter, and binds preferentially to aggregate over monomer when attached to said solid support. In certain embodiments, the methods further include removing unbound sample after forming said complex. In certain embodiments, the conformational protein-specific binding reagent is an antibody. In preferred embodiments, the aggregate contains Aβ protein and said conformational protein-specific binding reagent is an anti-Aβ antibody.

Yet another aspect includes methods for detecting the presence of aggregate in a sample including the steps of contacting a sample suspected of containing aggregate with an aggregate-specific binding reagent under conditions that allow binding of said reagent to said aggregate, if present, to form a first complex; removing unbound sample; dissociating said aggregate from said first complex thereby providing dissociated aggregate; contacting said dissociated aggregate with a first conformational protein-specific binding reagent under conditions that allow binding to form a second complex; and detecting the presence of aggregate, if any, in said sample by detecting the formation of said second complex; wherein said aggregate-specific binding reagent has a net charge of at least about positive one at the pH at which said sample is contacted with said aggregate-specific binding reagent, is attached to a solid support at a charge density of at least about 60 nmol net charge per square meter, and binds preferentially to aggregate over monomer when attached to said solid support. In certain embodiments, the formation of said second complex is detected using a detectably labeled second conformational protein-specific binding reagent. In certain embodiments, the first conformational protein-specific binding reagent is coupled to a solid support. In certain embodiments, the aggregate is dissociated from said first complex by exposing said first complex to guanidine thiocyanate or by exposing said complex to high pH or low pH. In preferred embodiments, the aggregate includes Aβ protein and said conformational protein-specific binding reagent is an anti-Aβ antibody.

Another aspect provides methods for detecting the presence of aggregate in a sample including the steps of contacting a sample suspected of containing aggregate with a conformational protein-specific binding reagent under conditions that allow binding of said reagent to said aggregate, if present, to form a complex; removing unbound sample; contacting said complex with an aggregate-specific binding reagent under conditions that allow the binding of said reagent to said aggregate, wherein said reagent includes a detectable label; and detecting the presence of aggregate, if any, in said sample by its binding to said aggregate-specific binding reagent; wherein said aggregate-specific binding reagent has a net charge of at least about positive one at the pH at which said sample is contacted with said aggregate-specific binding reagent, is attached to a solid support at a charge density of at least about 60 nmol net charge per square meter, and binds preferentially to aggregate over monomer when attached to said solid support. In certain embodiments, the conformational protein-specific protein is coupled to a solid support.

Yet another aspect provides methods for detecting the presence of aggregate in a sample including the steps of providing a solid support containing an aggregate-specific binding reagent; combining said solid support with a detectably labeled ligand, wherein said aggregate-specific binding reagent's binding avidity to said detectably labeled ligand is weaker than said reagent's binding avidity to said aggregate; combining a sample suspected of containing aggregate with said solid support under conditions which allow said aggregate, when present in said sample, to bind to said reagent and replace said ligand; and detecting complexes formed between said aggregate and said aggregate-specific binding reagent; wherein said aggregate-specific binding reagent has a net charge of at least about positive one at the pH at which said sample is contacted with said aggregate-specific binding reagent, is attached to a solid support at a charge density of at least about 60 nmol net charge per square meter, and binds preferentially to aggregate over monomer when attached to said solid support.

Another aspect provides methods for reducing the amount of aggregate in a polypeptide sample including the steps of: contacting a polypeptide sample suspected of containing aggregate with an aggregate-specific binding reagent under conditions that allow binding of said reagent to said aggregate, if present, to form a complex; and recovering unbound polypeptide sample; wherein said aggregate-specific binding reagent has a net charge of at least about positive one at the pH at which said sample is contacted with said aggregate-specific binding reagent, is attached to a solid support at a charge density of at least about 60 nmol net charge per square meter, and binds preferentially to aggregate over monomer when attached to said solid support.

Yet another aspect provides methods for discriminating between aggregate and monomer in a sample including the steps of: contacting a sample suspected of containing aggregate with an aggregate-specific binding reagent under conditions that allow binding of said reagent to said aggregate, if present, to form a complex; and discriminating between aggregate and monomer, if any, in said sample by binding of aggregate to said aggregate-specific binding reagent; wherein said aggregate-specific binding reagent has a net charge of at least about positive one at the pH at which said sample is contacted with said aggregate-specific binding reagent, is attached to a solid support at a charge density of at least about 60 nmol net charge per square meter, and binds preferentially to aggregate over monomer when attached to said solid support when attached to said solid support.

Another aspect provides methods for assessing whether there is an increased probability of conformational disease for a subject including the steps of: contacting a biological sample from a subject suspected of having an conformational disease with an aggregate-specific binding reagent under conditions that allow binding of said reagent to pathogenic aggregate, if present, to form a complex; detecting the presence of pathogenic aggregate, if any, in said biological sample by its binding to said aggregate-specific binding reagent; and determining that there is an increased probability that said subject has conformational disease if the amount of pathogenic aggregate in said biological sample is higher than the amount of aggregate in a sample from a subject without conformational disease; wherein said aggregate-specific binding reagent has a net charge of at least about positive one at the pH at which said sample is contacted with said aggregate-specific binding reagent, is attached to a solid support at a charge density of at least about 60 nmol net charge per square meter, and binds preferentially to aggregate over monomer when attached to said solid support.

Another aspect provides methods for assessing the effectiveness of treatment for conformational disease including the steps of: contacting a biological sample from a patient having undergone treatment for conformational disease with an aggregate-specific binding reagent under conditions that allow binding of said reagent to pathogenic aggregate, if present, to form a complex; detecting the presence of pathogenic aggregate, if any, in said sample by its binding to said aggregate-specific binding reagent; and determining that said treatment is effective if the amount of pathogenic aggregate in said biological sample is lower than the amount of pathogenic aggregate in a control, wherein said control is the amount of pathogenic aggregate in a biological sample from said patient prior to treatment for conformational disease, wherein said aggregate-specific binding reagent has a net charge of at least about positive one at the pH at which said sample is contacted with said aggregate-specific binding reagent, is attached to a solid support at a charge density of at least about 60 nmol net charge per square meter, and binds preferentially to aggregate over monomer when attached to said solid support.

Yet another aspect includes method for detecting the presence of oligomer in a sample including the steps of: providing a sample suspected of containing oligomer, wherein said sample lacks aggregates other than oligomers; contacting said sample with an aggregate-specific binding reagent under conditions that allow binding of said reagent to said oligomer, if present, to form a complex; and detecting the presence of oligomer, if any, in said sample by its binding to said aggregate-specific binding reagent; wherein said aggregate-specific binding reagent has a net charge of at least about positive one at the pH at which said sample is contacted with said aggregate-specific binding reagent, is attached to a solid support at a charge density of at least about 2000 nmol net charge per square meter, and binds preferentially to aggregate over monomer when attached to said solid support.

Another aspect includes methods for detecting the presence of oligomer in a sample including the steps of: providing a sample suspected of containing oligomer; removing aggregate other than oligomer from said sample; contacting said sample with an aggregate-specific binding reagent under conditions that allow binding of said reagent to said oligomer, if present, to form a complex; and detecting the presence of oligomer, if any, in said sample by its binding to said aggregate-specific binding reagent; wherein said aggregate-specific binding reagent has a net charge of at least about positive one at the pH at which said sample is contacted with said aggregate-specific binding reagent, is attached to a solid support at a charge density of at least about 2000 nmol net charge per square meter, and binds preferentially to aggregate over monomer when attached to said solid support. In certain embodiments the aggregate removing is by centrifugation.

Yet another aspect provides methods for detecting the presence of oligomer in a sample including the steps of: contacting a sample suspected of containing oligomer with an aggregate-specific binding reagent under conditions that allow binding of said reagent to said oligomer, if present, to form a complex; contacting said complex with a second reagent, wherein said reagent binds preferentially to either oligomer or aggregates other than oligomer; detecting the presence of oligomer, if any, in said sample by its binding or lack of binding to said second reagent; wherein said aggregate-specific binding reagent has a net charge of at least about positive one at the pH at which said sample is contacted with said aggregate-specific binding reagent, is attached to a solid support at a charge density of at least about 2000 nmol net charge per square meter, and binds preferentially to aggregate over monomer when attached to said solid support. In certain embodiments of the aspects including detecting the presence of oligomers, the aggregate other than oligomer includes fibrils.

In certain embodiments of the aspects described above, the aggregate, pathogenic aggregate, or oligomer of interest (e.g., to be detected, reduced, or discriminated) is soluble.

In certain embodiments of the aspects described above, the method further includes a step of treating the complex formed between said aggregate-specific binding reagent and said aggregate or oligomer with a detergent. In certain embodiments, the step of treating is performed after the step of contacting. In certain embodiments, the detergent is a neutral detergent. In certain embodiments, the comprises both positive and negative charges. In certain preferred embodiments, the detergent comprises a long carbon chain. In some preferred embodiments, the detergent is selected from the group consisting of polyethylene glycol sorbitan monolaurate, n-tetradecyl-N,N-dimethyl-3-ammonio-1-propanesulfonate, n-hexadecyl-N,N-dimethyl-3-ammonio-1-propanesulfonate, n-dodecyl-N,N-dimethyl-3-ammonio-1-propanesulfonate, amidosulfobetaine-14, 3-[N,N-dimethyl(3-myristoylaminopropyl)ammonio]propanesulfonate, amidosulfobetain-16, 3[N,N-dimethyl-N-(3-palmitamidopropyl)ammonio]propane-1-sulfonate, 4-n-octylbenzoylamido-propyl-dimethylammonio sulfobetaine, and N,N-dimethyl-N-dodecylglycine betaine.

In certain embodiments of the aspects described above, the solid support is selected from the group consisting of: nitrocellulose, polystyrene latex, polyvinyl fluoride, diazotized paper, nylon membrane, activated bead, magnetically responsive bead, titanium oxide, silicon oxide, polysaccharide bead, polysaccharide membrane, agarose, glass, polyacrylic acid, polyethyleneglycol, polyethyleneglycol-polystyrene hybrid, controlled pore glass, glass slide, gold bead, and cellulose. In certain emaggregate-specific binding reagent is detectably labeled. In certain embodiments, the sample is a biological sample including bodily tissues or fluid. In certain embodiments, the biological sample includes whole blood, blood fractions, blood components, plasma, platelets, serum, cerebrospinal fluid (CSF), bone marrow, urine, tears, milk, lymph fluid, organ tissue, brain tissue, nervous system tissue, muscle tissue, non-nervous system tissue, biopsy, necropsy, fat biopsy, cells, feces, placenta, spleen tissue, lymph tissue, pancreatic tissue, bronchoalveolar lavage, or synovial fluid. In preferred embodiments, the sample includes cerebrospinal fluid (CSF). In certain embodiments, the sample includes polypeptide.

In certain embodiments, the aggregate-specific binding reagent has a net charge of at least about positive two, at least about positive three, at least about positive four, at least about positive five, at least about positive six, and at least about positive seven at the pH at which the sample is contacted with said aggregate-specific binding reagent. In certain embodiments, the aggregate-specific binding reagent is attached to a solid support at a charge density of at least about 90 nmol net charge per square meter, at least about 120 nmol net charge per square meter, at least about 500 nmol net charge per square meter, at least about 1000 nmol net charge per square meter, at least about 2000 nmol net charge per square meter, at least about 3000 nmol net charge per square meter, at least about 4000 nmol net charge per square meter, or at least about 5000 nmol net charge per square meter. In preferred embodiments, the aggregate-specific binding reagent is attached to a solid support at a charge density of at least about 6000 nmol net charge per square meter. In certain embodiments, the aggregate-specific binding reagent has a binding affinity and/or avidity for aggregate that is at least about two times higher than the binding affinity and/or avidity for monomer. In certain embodiments, the aggregate-specific binding reagent includes at least one positively charged functional group having a pKa at least about 1 pH unit higher than the pH at which the sample is contacted with said aggregate-specific binding reagent. In certain embodiments, the at least one positively charged functional group in the aggregate-specific binding reagent is closest to the solid support among all functional groups of the aggregate-specific binding reagent. In certain embodiments, the aggregate-specific binding reagent includes a hydrophobic functional group. In some embodiments, the hydrophobic functional group is an aromatic hydrophobic functional group. In other embodiments, hydrophobic functional group is an aliphatic hydrophobic functional group. In certain embodiments, the aggregate-specific binding reagent includes only one positively charged functional group and at least one hydrophobic functional group. In certain embodiments, the aggregate-specific binding reagent includes at least one positively charged functional group and only one hydrophobic functional group. In certain embodiments, the aggregate-specific binding reagent includes only one positively charged functional group and only one hydrophobic functional group. In some embodiments, the aggregate-specific binding reagent includes at least one amino acid that is an L-isomer. In some embodiments, the aggregate-specific binding reagent includes at least one amino acid that is a D-isomer.

In certain embodiments, the aggregate is non-pathogenic. In certain embodiments, the non-pathogenic aggregate is yeast prion protein sup35 or hormone. In certain embodiments, the non-pathogenic aggregate is an aggregate of polypeptide. In other embodiments, the aggregate is pathogenic. In certain embodiments, the pathogenic aggregate is an aggregate associated with preeclampsia, tauopathy, TDP-43 proteinopathy, or serpinopathy. In certain embodiments, the pathogenic aggregate is an aggregate associated with an amyloid disease. In certain embodiments, the amyloid disease is selected from the group consisting of systemic amyloidosis, AA amyloidosis, synucleinopathy, Alzheimer's disease, prion disease, ALS, immunoglobulin-related diseases, serum amyloid A-related diseases, Huntington's disease, Parkinson's disease, diabetes type II, dialysis amyloidosis, and cerebral amyloid angiopathy. In preferred embodiments, the pathogenic aggregate is an aggregate associated with Alzheimer's disease. In certain other preferred embodiments, the pathogenic aggregate is an aggregate associated with cerebral amyloid angiopathy. In certain embodiments, the aggregate associated with Alzheimer's disease or cerebral amyloid angiopathy includes amyloid-beta (Aβ) protein. In some embodiments, the Aβ protein is Aβ40. In other embodiments, the Aβ protein is Aβ42. In certain embodiments, the aggregate associated with Alzheimer's disease includes tau protein. In certain embodiments, the pathogenic aggregate includes amylin. In certain embodiments, the pathogenic aggregate includes Amyloid A protein. In certain embodiments, the pathogenic aggregate includes alpha-synuclein.

In certain embodiments, the aggregate-specific binding reagent includes at least one amino acid with at least one net positive charge at the pH at which said sample is contacted with said aggregate-specific binding reagent. In certain embodiments, the at least one amino acid is positively charged at physiological pH. In certain embodiments, the at least one amino acid is a natural amino acid selected from the group consisting of lysine and arginine. In certain embodiments, the at least one amino acid is an unnatural amino acid selected from the group consisting of ornithine, methyllysine, diaminobutyric acid, homoarginine, and 4-aminomethylphenylalanine. In certain embodiments, the aggregate-specific binding reagent includes a hydrophobic amino acid. In certain embodiments, the hydrophobic amino acid is an aromatic hydrophobic amino acid. In certain embodiments, the hydrophobic amino acid is an aliphatic hydrophobic amino acid. In certain embodiments, the hydrophobic amino acid is selected from the group consisting of tryptophan, phenylalanine, valine, leucine, isoleucine, methionine, tyrosine, homophenylalanine, phenylglycine, 4-chlorophenylalanine, norleucine, norvaline, thienylalanine, 4-nitrophenylalanine, 4-aminophenylalanine, pentafluorophenylalanine, 2-naphthylalanine, p-biphenylalanine, styrylalanine, substituted phenylalanines, halogenated phenylalanines, aminoisobutyric acid, allyl glycine, cyclohexylalanine, cyclohexylglycine, 1-napthylalanine, pyridylalanine, and 1,2,3,4-tetrahydroisoquinoline-3-carboxylic acid. In preferred embodiments, the aggregate-specific binding reagent includes a peptide selected from the group consisting of KKKFKF (SEQ ID NO: 1), KKKWKW (SEQ ID NO: 2), KKKLKL (SEQ ID NO: 3), KKKKKK (SEQ ID NO: 4), KKKKKKKKKKKK (SEQ ID NO: 5), AAKKAA (SEQ ID NO: 32), AAKKKA (SEQ ID NO: 33), AKKKKA (SEQ ID NO: 34), AKKKKK (SEQ ID NO: 35), FKFKKK (SEQ ID NO: 36), kkkfkf (SEQ ID NO: 37), FKFSLFSG (SEQ ID NO: 38), DFKLNFKF (SEQ ID NO: 39), FKFNLFSG (SEQ ID NO: 40), YKYKKK (SEQ ID NO: 41), KKFKKF (SEQ ID NO: 42), KFKKKF (SEQ ID NO: 43), KIGVVR (SEQ ID NO: 44), AKVKKK (SEQ ID NO: 45), AKFKKK (SEQ ID NO: 46), RGRERFEMFR (SEQ ID NO: 47), YGRKKRRQRRR (SEQ ID NO: 48), FFFKFKKK (SEQ ID NO: 49), FFFFFKFKKK (SEQ ID NO: 50), FFFKKK (SEQ ID NO: 51), and FFFFKK (SEQ ID NO: 52). In some preferred embodiments, the aggregate-specific binding reagent includes a peptide selected from the group consisting of F-fdb-F-fdb-fdb-fdb (SEQ ID NO: 53), FoFooo (SEQ ID NO: 54), monoBoc-ethylenediamine+BrCH2CO-KKFKF (SEQ ID NO: 55), triethylamine+BrCH2CO-KKFKF (SEQ ID NO: 56), tetramethylethylenediamine+BrCH2CO-KKFKF (SEQ ID NO: 57) and SEQ ID NOs: 58-66. In some preferred embodiments, the aggregate-specific binding reagent includes a peptide selected from the group consisting of KFYLYAIDTHRM (SEQ ID NO: 6), KIIKWGIFWMQG (SEQ ID NO: 7), NFFKKFRFTFTM (SEQ ID NO: 8), MKFMKMHNKKRY (SEQ ID NO: 67), LTAVKKVKAPTR (SEQ ID NO: 68), LIPIRKKYFFKL (SEQ ID NO: 69), KLSLIWLHTHWH (SEQ ID NO: 70), IRYVTHQYILWP (SEQ ID NO: 71), YNKIGVVRLFSE (SEQ ID NO: 72), YRHRWEVMLWWP (SEQ ID NO: 73), WAVKLFTFFMFH (SEQ ID NO: 74), YQSWWFFYFKLA (SEQ ID NO: 75), WWYKLVATHLYG (SEQ ID NO: 76), QTLSLHFQTRPP (SEQ ID NO: 77), TRLAMQYVGYFW (SEQ ID NO: 78), RYWYRHWSQHDN (SEQ ID NO: 79), AQYIMFKVFYLS (SEQ ID NO: 80), TGIRIYSWKMWL (SEQ ID NO: 81), SRYLMYVNIIYI (SEQ ID NO: 82), RYWMNAFYSPMW (SEQ ID NO: 83), NFYTYKLAYMQM (SEQ ID NO: 84), MGYSSGYWSRQV (SEQ ID NO: 85), YFYMKLLWTKER (SEQ ID NO: 86), RIMYLYHRLQHT (SEQ ID NO: 87), RWRHSSFYPIWF (SEQ ID NO: 88), QVRIFTNVEFKH (SEQ ID NO: 89), and RYLHWYAVAVKV (SEQ ID NO: 90). In some preferred embodiments, the aggregate-specific binding reagent includes a peptoid selected from the group consisting of SEQ ID NOs: 9-14 and 91-96. In preferred embodiments, the aggregate-specific binding reagent includes a peptoid selected from the group consisting of

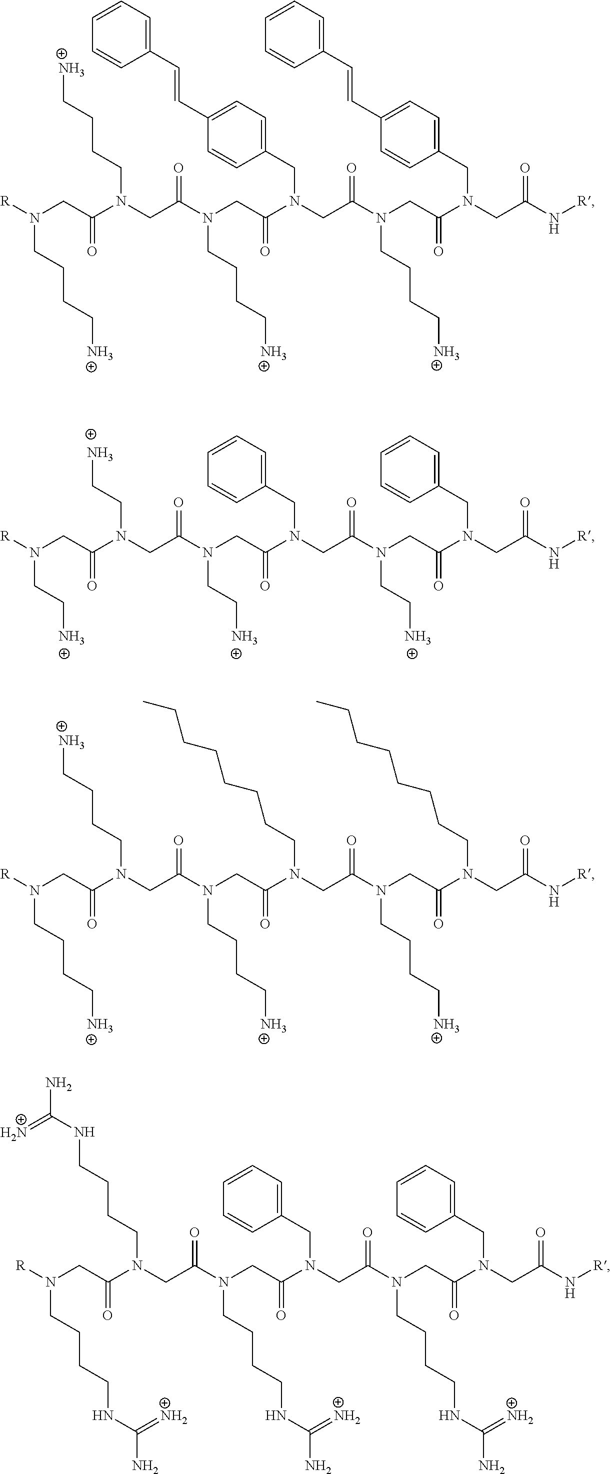

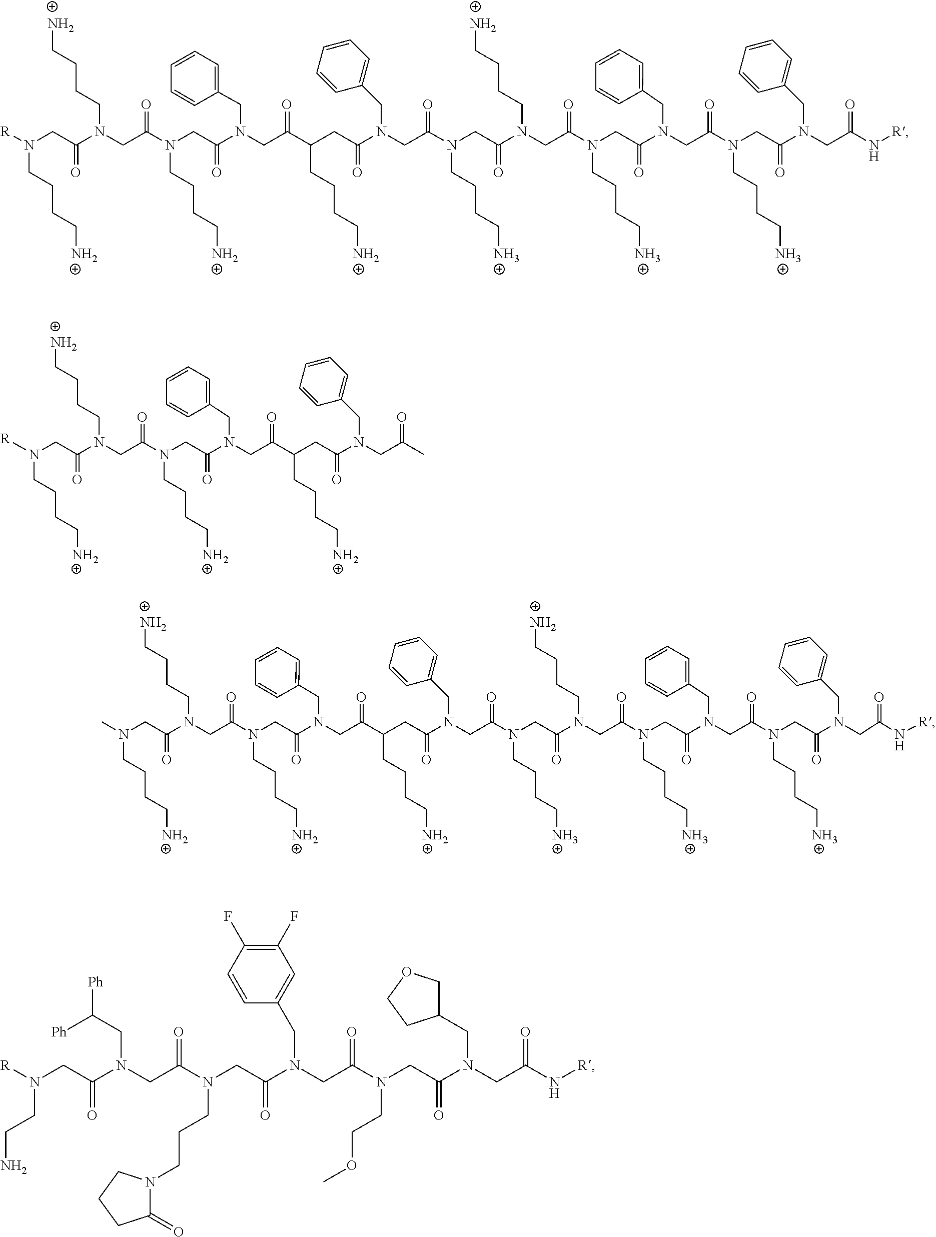

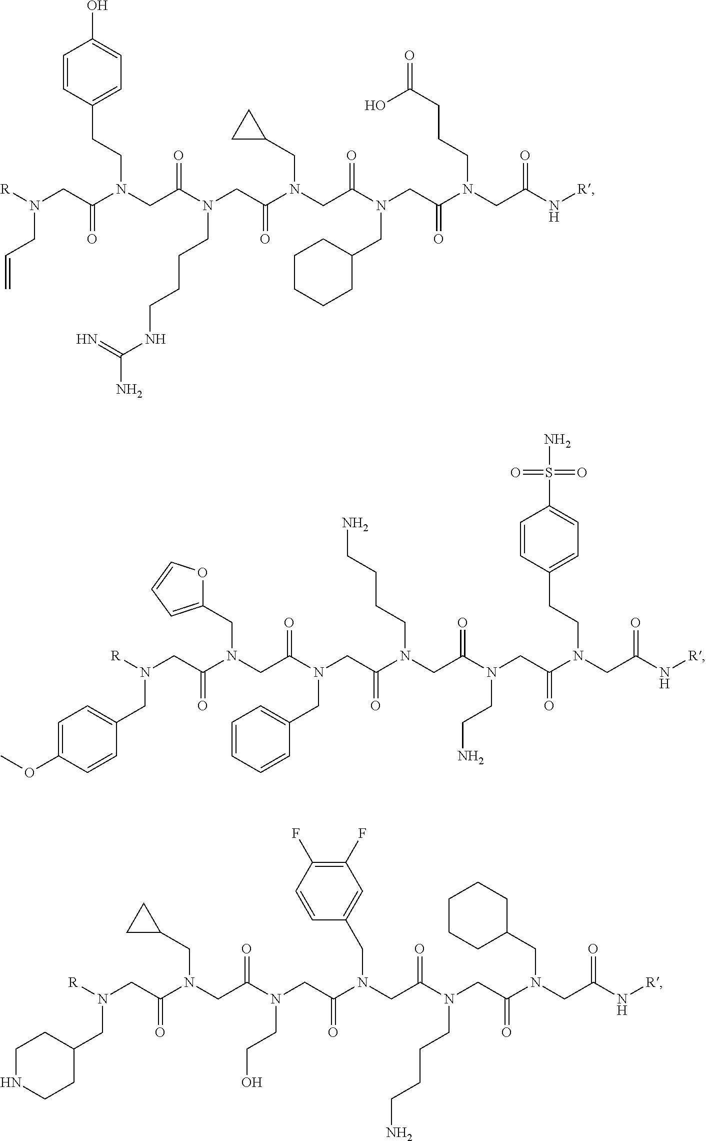

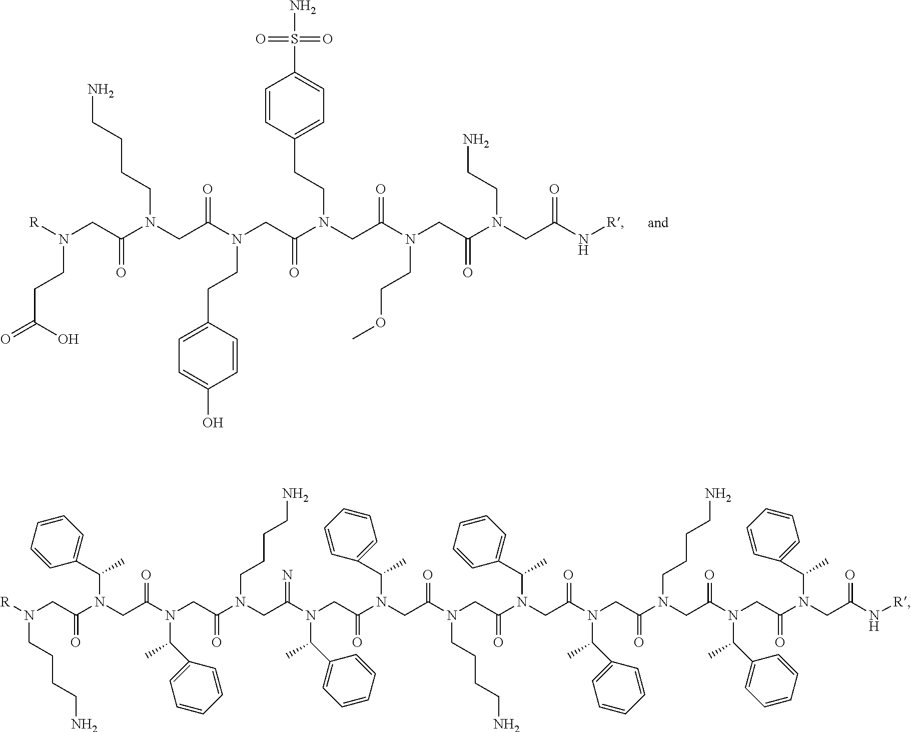

wherein R and R′ is any group. In certain embodiments, the aggregate-specific binding reagent includes

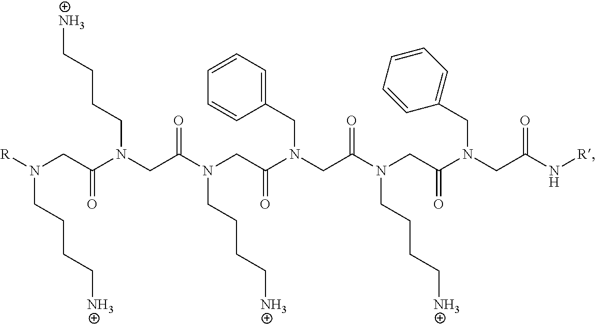

wherein R and R′ is any group. In certain embodiments, the aggregate-specific binding reagent includes the dendron

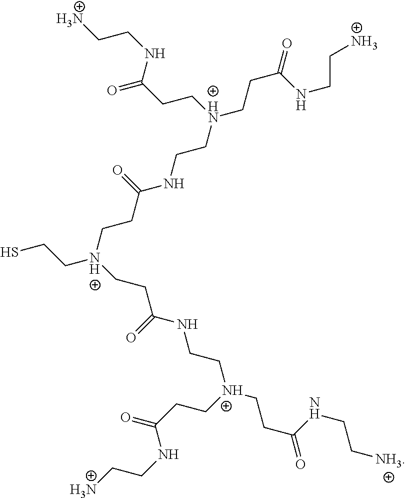

In certain embodiments, the aggregate-specific binding reagent includes a functional group selected from the group consisting of amines, alkyl groups, heterocycles, cycloalkanes, guanidine, ether, allyl, and aromatics. In certain embodiments, the aggregate-specific binding reagent includes an aromatic functional group selected from the group consisting of naphtyl, phenol, aniline, phenyl, substituted phenyl, nitrophenyl, halogenenated phenyl, biphenyl, styryl, diphenyl, benzyl sulfonamide, aminomethylphenyl, thiophene, indolyl, naphthyl, furan, and imidazole. In certain embodiments, the halogenenated phenyl is chlorophenyl or fluorophenyl. In certain embodiments, the aggregate-specific binding reagent includes an amine functional group selected from the group consisting of primary, secondary, tertiary, and quaternary amines. In certain embodiments, the aggregate-specific binding reagent includes an alkyl functional group selected from the group consisting of isobutyl, isopropyl, sec-butyl, and methyl and octyl. In certain embodiments, the aggregate-specific binding reagent includes. a heterocycle functional group selected from the group consisting of tetrohydrofuran, pyrrolidine, and piperidine. In certain embodiments, the aggregate-specific binding reagent includes a cycloalkane functional group selected from the group consisting of cyclopropyl and cyclohexyl. In certain embodiments, the aggregate-specific binding reagent includes repeating motifs. In certain embodiments, the aggregate-specific binding reagent includes positively charged groups with the same spacing as that of the negatively charged groups of the aggregate.

In certain embodiments, the aggregate-specific binding reagent comprises SEQ ID NO: 1 or SEQ ID NO: 15, In certain embodiments, the aggregate comprises amylin, wherein said aggregate-specific binding reagent comprises SEQ ID NO: 15, and wherein said aggregate-specific binding reagent is attached to a solid support at a charge density of at least about 8000 nmol to about 15000 nmol net charge per square meter. In certain embodiments, the aggregate comprises alpha-synuclein, wherein said aggregate-specific binding reagent comprises SEQ ID NO: 15, and wherein said aggregate-specific binding reagent is attached to a solid support at a charge density of at least about 8000 nmol to about 15000 nmol net charge per square meter. In certain embodiments, the aggregate comprises Amyloid A protein, wherein said aggregate-specific binding reagent comprises SEQ ID NO: 15, and wherein said aggregate-specific binding reagent is attached to a solid support at a charge density of at least about 8000 nmol to about 15000 nmol net charge per square meter. In certain embodiments, the further step of detergent treatment is included, and the detergent is n-tetradecyl-N,N-dimethyl-3-ammonio-1-propanesulfonate, wherein said aggregate is a pathogenic aggregate that comprises Aβ40 protein, wherein said aggregate-specific binding reagent comprises SEQ ID NO: 15, and wherein said aggregate-specific binding reagent is attached to a solid support at a charge density of about 8000 nmol to about 15000 nmol net charge per square meter. In certain embodiments, the sample comprises cerebrospinal fluid (CSF).

Another aspect includes peptide aggregate-specific binding reagents, wherein said reagent includes an amino acid sequence selected from the group consisting of: KKKFKF (SEQ ID NO: 1), KKKWKW (SEQ ID NO: 2), KKKLKL (SEQ ID NO: 3), KKKKKKKKKKKK (SEQ ID NO: 5), AAKKAA (SEQ ID NO: 32), AAKKKA (SEQ ID NO: 33), AKKKKA (SEQ ID NO: 34), AKKKKK (SEQ ID NO: 35), FKFKKK (SEQ ID NO: 36), kkkfkf (SEQ ID NO: 37), FKFSLFSG (SEQ ID NO: 38), DFKLNFKF (SEQ ID NO: 39), FKFNLFSG (SEQ ID NO: 40), YKYKKK (SEQ ID NO: 41), KKFKKF (SEQ ID NO: 42), KFKKKF (SEQ ID NO: 43), KIGVVR (SEQ ID NO: 44), AKVKKK (SEQ ID NO: 45), AKFKKK (SEQ ID NO: 46), RGRERFEMFR (SEQ ID NO: 47), FFFKFKKK (SEQ ID NO: 49), FFFFFKFKKK (SEQ ID NO: 50), FFFKKK (SEQ ID NO: 51), and FFFFKK (SEQ ID NO: 52). Yet another aspect includes peptide aggregate-specific binding reagents, wherein said reagent includes a peptide consisting of the amino acid sequence of KKKKKK. Another aspect includes peptide aggregate-specific binding reagents, wherein said reagent includes an amino acid sequence selected from the group consisting of: F-fdb-F-fdb-fdb-fdb (SEQ ID NO: 53), FoFooo (SEQ ID NO: 54), monoBoc-ethylenediamine+BrCH2CO-KKFKF (SEQ ID NO: 55), triethylamine+BrCH2CO-KKFKF (SEQ ID NO: 56), tetramethylethylenediamine+BrCH2CO-KKFKF (SEQ ID NO: 57) and SEQ ID NOs: 58-66. Another aspect includes a peptoid aggregate-specific binding reagent, wherein said reagent comprises a peptoid selected from the group consisting of SEQ ID NOs: 9-14 and 91-95. Another aspect includes peptoid aggregate-specific binding reagents, wherein said reagent includes a peptoid selected from the group consisting of:

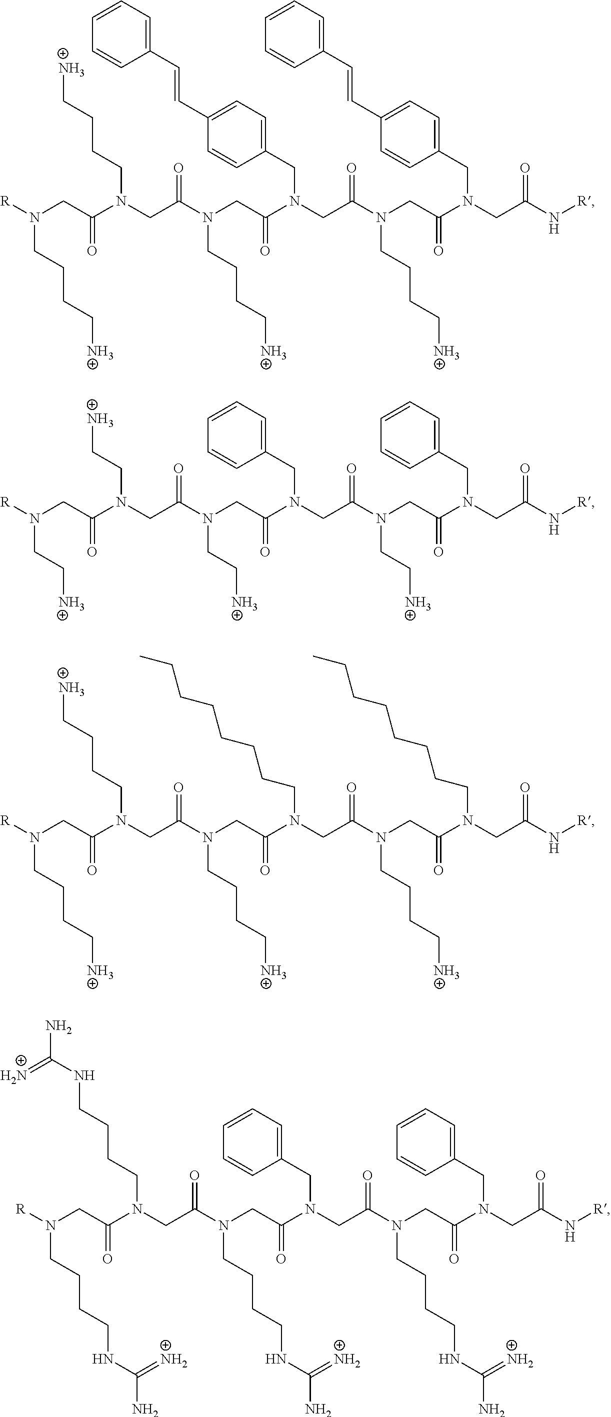

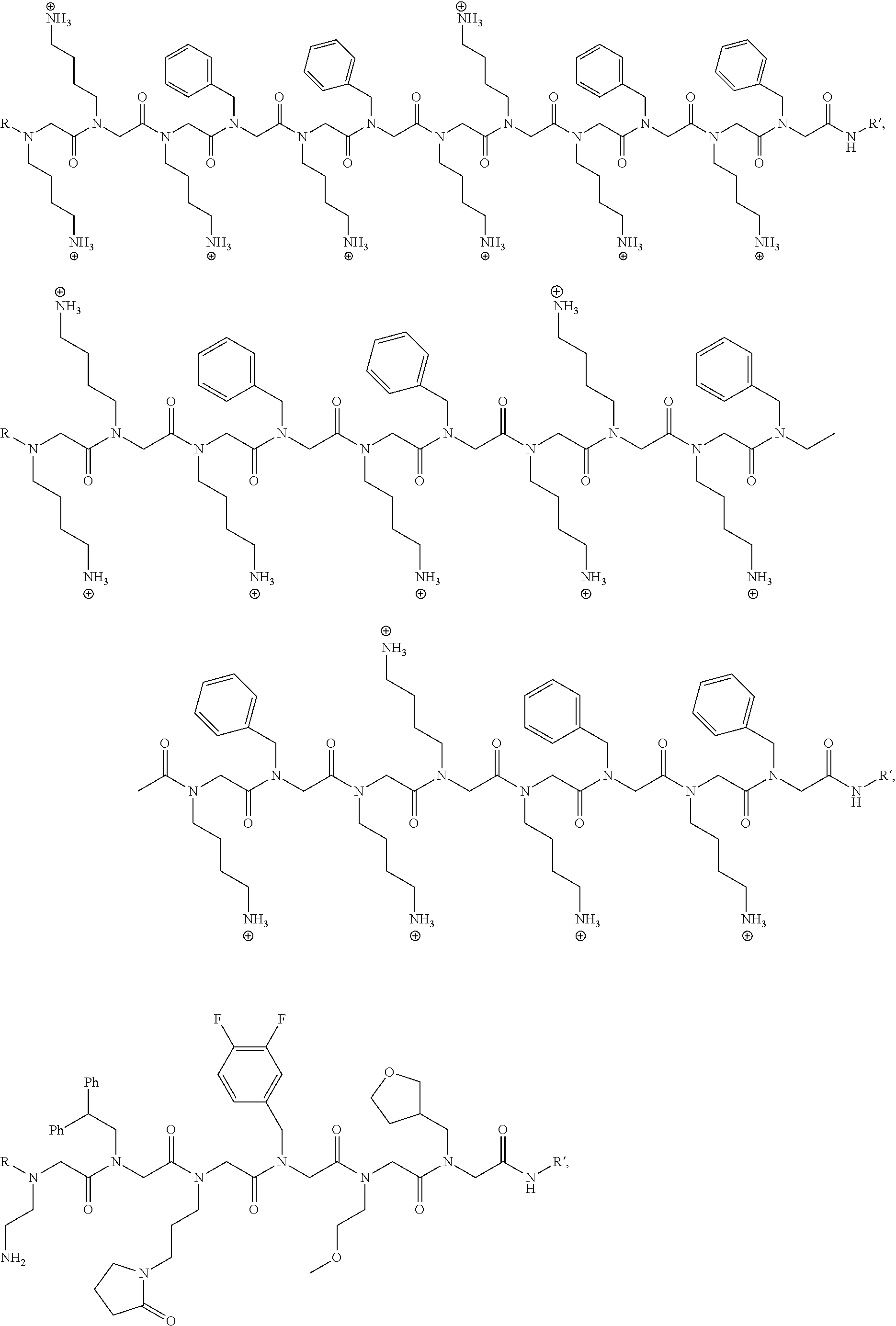

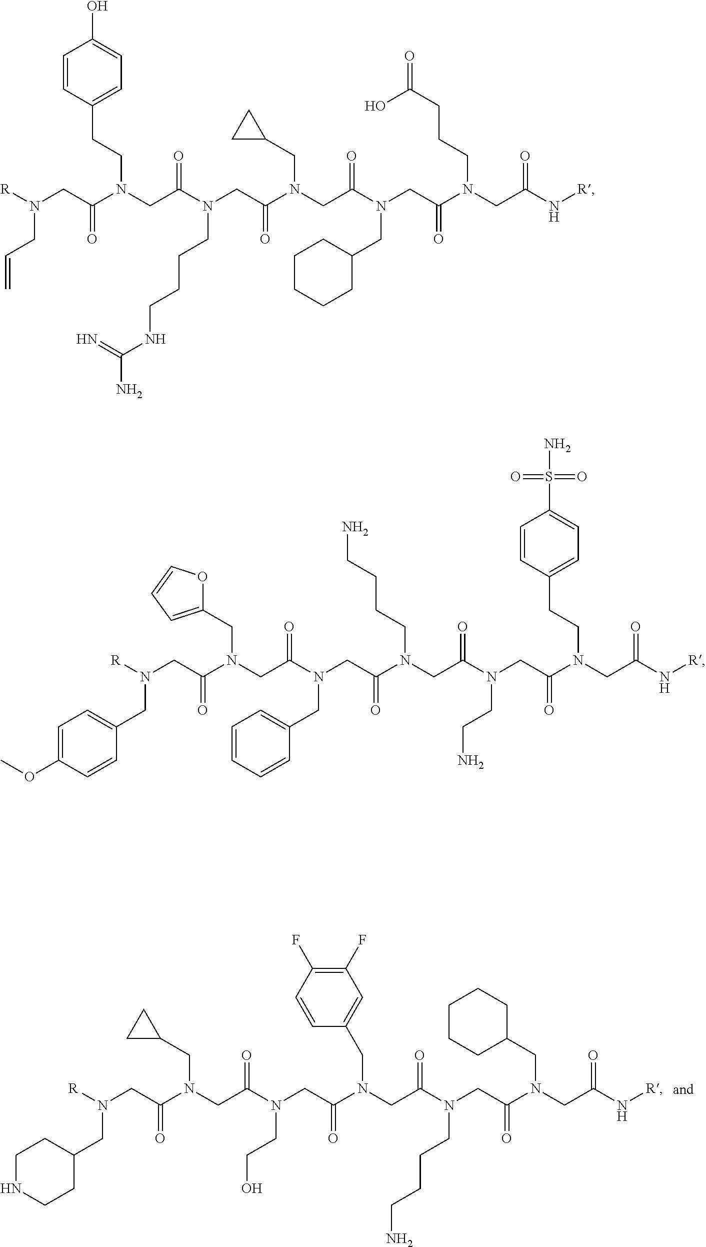

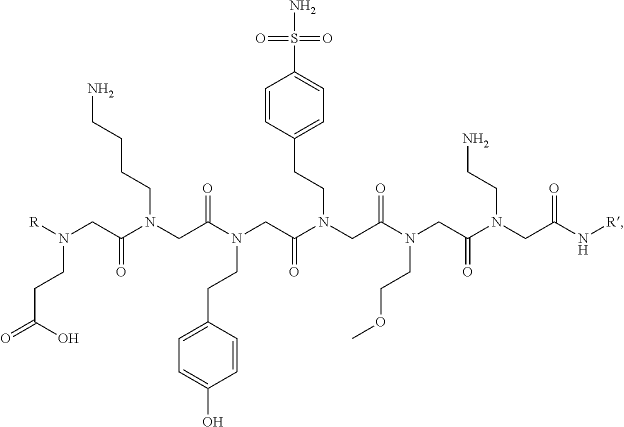

wherein R and R′ is any group. Another aspect includes dendron aggregate-specific binding reagents, wherein said reagent includes

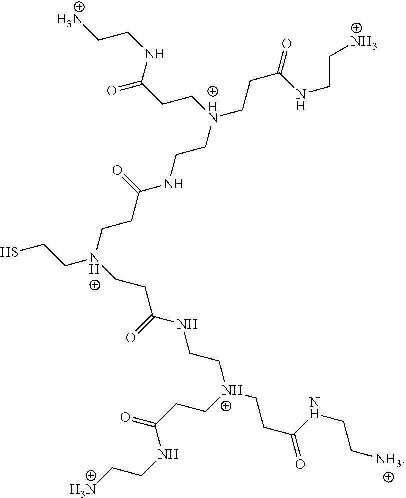

In certain embodiments, the reagent includes a hydrophobic functional group. In certain embodiments, the hydrophobic functional group is an aromatic hydrophobic functional group. In certain embodiments, the hydrophobic functional group is an aliphatic hydrophobic functional group. In certain embodiments, the reagent includes a functional group selected from the group consisting of amines, alkyl groups, heterocycles, cycloalkanes, guanidine, ether, allyl, and aromatics. In certain embodiments, the aggregate-specific binding reagent includes an aromatic functional group selected from the group consisting of naphtyl, phenol, aniline, phenyl, substituted phenyl, nitrophenyl, halogenenated phenyl, biphenyl, styryl, diphenyl, benzyl sulfonamide, aminomethylphenyl, thiophene, indolyl, naphthyl, furan, and imidazole. In certain embodiments, the halogenenated phenyl is chlorophenyl or fluorophenyl. In certain embodiments, the aggregate-specific binding reagent includes an amine functional group selected from the group consisting of primary, secondary, tertiary, and quaternary amines. In certain embodiments, the aggregate-specific binding reagent includes an alkyl functional group selected from the group consisting of isobutyl, isopropyl, sec-butyl, and methyl and octyl. In certain embodiments, the aggregate-specific binding reagent includes. a heterocycle functional group selected from the group consisting of tetrohydrofuran, pyrrolidine, and piperidine. In certain embodiments, the aggregate-specific binding reagent includes a cycloalkane functional group selected from the group consisting of cyclopropyl and cyclohexyl. In certain embodiments, the reagent is detectably labeled.

Another aspect includes compositions including a solid support and an aggregate-specific binding reagent of above described aspects. In certain embodiments, the aggregate-specific binding reagent is attached at a charge density of at least about 60 nmol net charge per square meter, and wherein said composition binds preferentially to aggregate over monomer. In certain embodiments, the aggregate-specific binding reagent is attached to a solid support at a charge density of at least about 90 nmol net charge per square meter, at least about 120 nmol net charge per square meter, at least about 500 nmol net charge per square meter, at least about 1000 nmol net charge per square meter, at least about 2000 nmol net charge per square meter, at least about 3000 nmol net charge per square meter, at least about 4000 nmol net charge per square meter, or at least about 5000 nmol net charge per square meter, and wherein said composition binds preferentially to aggregate over monomer. In certain embodiments, the aggregate-specific binding reagent is attached to a solid support at a charge density of at least about 6000 nmol, at least about 7000 nmol net charge per square meter, at least about 8000 nmol net charge per square meter, or at least about 9000 nmol net charge per square meter, and wherein said composition binds preferentially to aggregate over monomer. In certain embodiments, the solid support is selected from the group consisting of: nitrocellulose, polystyrene latex, polyvinyl fluoride, diazotized paper, nylon membrane, activated head, magnetically responsive bead, titanium oxide, silicon oxide, polysaccharide head, polysaccharide membrane, agarose, glass, polyacrylic acid, polyethyleneglycol, polyethyleneglycol-polystyrene hybrid, controlled pore glass, glass slide, gold bead, and cellulose.

Another aspect includes compositions including A composition comprising a solid support and an aggregate-specific binding reagent, wherein said aggregate-specific binding reagent comprises

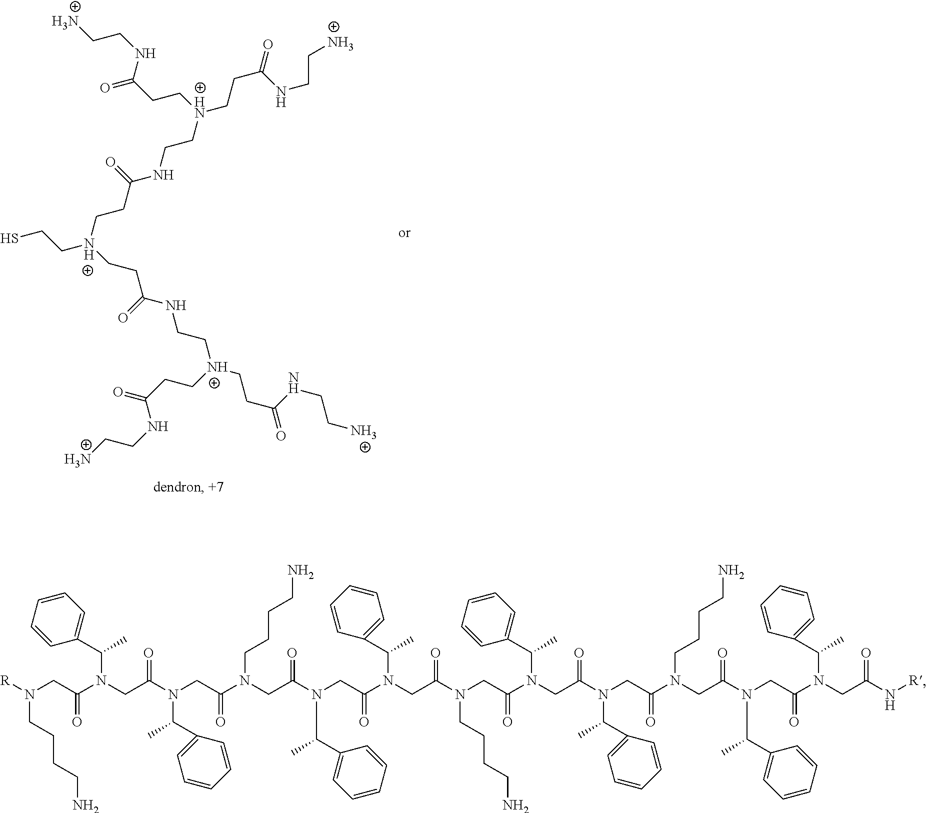

further wherein said solid support comprises a bead

Another aspect includes a composition comprising a solid support and a peptide aggregate-specific binding reagent, wherein said reagent comprises an amino acid sequence selected from the group consisting of: KFYLYAIDTHRM (SEQ ID NO: 6), KIIKWGIFWMQG (SEQ ID NO: 7), MKFMKMHNKKRY (SEQ ID NO: 67), LTAVKKVKAPTR (SEQ ID NO: 68), LIPIRKKYFFKL (SEQ ID NO: 69), KLSLIWLHTHWH (SEQ ID NO: 70), IRYVTHQYILWP (SEQ ID NO: 71), YNKIGVVRLFSE (SEQ ID NO: 72), YRHRWEVMLWWP (SEQ ID NO: 73), WAVKLFTFFMFH (SEQ ID NO: 74), YQSWWFFYFKLA (SEQ ID NO: 75), further wherein said solid support comprises a bead. In certain embodiments, the aggregate-specific binding reagent is attached at a charge density of at least about 60 nmol net charge per square meter, and wherein said composition binds preferentially to aggregate over monomer. In certain embodiments, the aggregate-specific binding reagent is attached to a solid support at a charge density of at least about 90 nmol net charge per square meter, at least about 120 nmol net charge per square meter, at least about 500 nmol net charge per square meter, at least about 1000 nmol net charge per square meter, at least about 2000 nmol net charge per square meter, at least about 3000 nmol net charge per square meter, at least about 4000 nmol net charge per square meter, or at least about 5000 nmol net charge per square meter, and wherein said composition binds preferentially to aggregate over monomer.

Another aspect includes kits containing the above-described compositions. In certain embodiments, the kit further comprises an instruction of using said kit to detect aggregates.

Another aspect includes a kit comprising: a solid support; an aggregate-specific binding reagent, wherein said aggregate-specific binding reagent comprises an amino acid sequence selected from the group consisting of YGRKKRRQRRR, KFYLYAIDTHRM (SEQ ID NO: 6), KIIKWGIFWMQG (SEQ ID NO: 7), NFFKKFRFTFTM (SEQ ID NO: 8), MKFMKMHNKKRY (SEQ ID NO: 67), LTAVKKVKAPTR (SEQ ID NO: 68), LIPIRKKYFFKL (SEQ ID NO: 69), KLSLIWLHTHWH (SEQ ID NO: 70), IRYVTHQYILWP (SEQ ID NO: 71), YNKIGVVRLFSE (SEQ ID NO: 72), YRHRWEVMLWWP (SEQ ID NO: 73), WAVKLFTFFMFH (SEQ ID NO: 74), YQSWWFFYFKLA (SEQ ID NO: 75), WWYKLVATHLYG (SEQ ID NO: 76), QTLSLHFQTRPP (SEQ ID NO: 77), TRLAMQYVGYFW (SEQ ID NO: 78), RYWYRHWSQHDN (SEQ ID NO: 79), AQYIMFKVFYLS (SEQ ID NO: 80), TGIRIYSWKMWL (SEQ ID NO: 81), SRYLMYVNIIYI (SEQ ID NO: 82), RYWMNAFYSPMW (SEQ ID NO: 83), NFYTYKLAYMQM (SEQ ID NO: 84), MGYSSGYWSRQV (SEQ ID NO: 85), YFYMKLLWTKER (SEQ ID NO: 86), RIMYLYHRLQHT (SEQ ID NO: 87), RWRHSSFYPIWF (SEQ ID NO: 88), QVRIFTNVEFKH (SEQ ID NO: 89), and RYLHWYAVAVKV (SEQ ID NO: 90), wherein said aggregate-specific binding reagent is attached to said solid support at a charge density of at least about 60 nmol net charge per square meter, and wherein said aggregate-specific binding reagent binds preferentially to aggregate over monomer when attached to said solid support; and an instruction of using said kit to detect aggregates.

Another aspect includes a kit comprising: a solid support; an aggregate-specific binding reagent, wherein said aggregate-specific binding reagent comprises

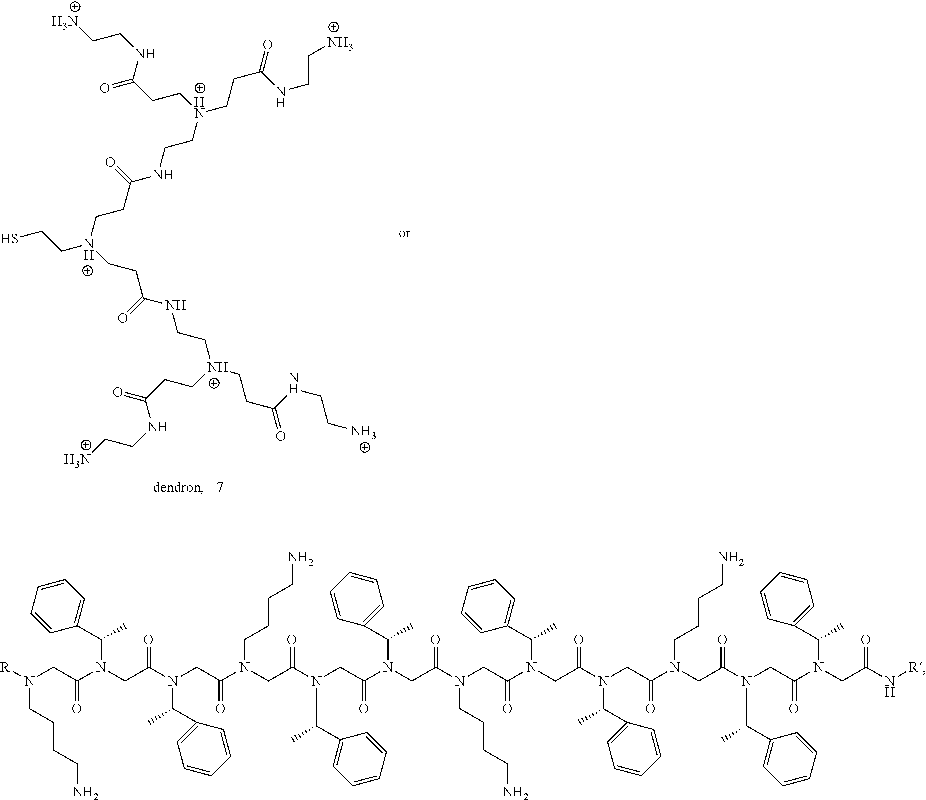

wherein said aggregate-specific binding reagent is attached to said solid support at a charge density of at least about 60 nmol net charge per square meter, and wherein said aggregate-specific binding reagent binds preferentially to aggregate over monomer when attached to said solid support; and an instruction of using said kit to detect aggregates.

In certain embodiments of the compositions or the kits, the aggregate-specific binding reagent is attached at a charge density of at least about 60 nmol net charge per square meter, and wherein said composition binds preferentially to aggregate over monomer. In certain embodiments, the aggregate-specific binding reagent is attached to a solid support at a charge density of at least about 90 nmol net charge per square meter, at least about 120 nmol net charge per square meter, at least about 500 nmol net charge per square meter, at least about 1000 nmol net charge per square meter, at least about 2000 nmol net charge per square meter, at least about 3000 nmol net charge per square meter, at least about 4000 nmol net charge per square meter, or at least about 5000 nmol net charge per square meter, and wherein said composition binds preferentially to aggregate over monomer.

A preferred aspect provides methods for detecting the presence of aggregate includes Aβ in a sample including the steps of: contacting a sample suspected of containing aggregate including Aβ with an aggregate-specific binding reagent under conditions that allow binding of said reagent to said aggregate, if present, to form a first complex; removing unbound sample; dissociating said aggregate from said first complex thereby providing dissociated aggregate; contacting said dissociated aggregate with a first anti-Aβ antibody coupled to a solid support under conditions that allow binding to form a second complex; and detecting the presence of aggregate, if any, in said sample by detecting the formation of said second complex using a detectably labeled second anti-Aβ antibody; wherein said aggregate-specific binding reagent has a net charge of at least about positive one at the pH at which said sample is contacted with said aggregate-specific binding reagent, is attached to a solid support at a charge density of at least about 60 nmol net charge per square meter, and binds preferentially to aggregate over monomer when attached to said solid support. In certain embodiments, the aggregate-specific binding reagent includes a peptoid selected from the group consisting of:

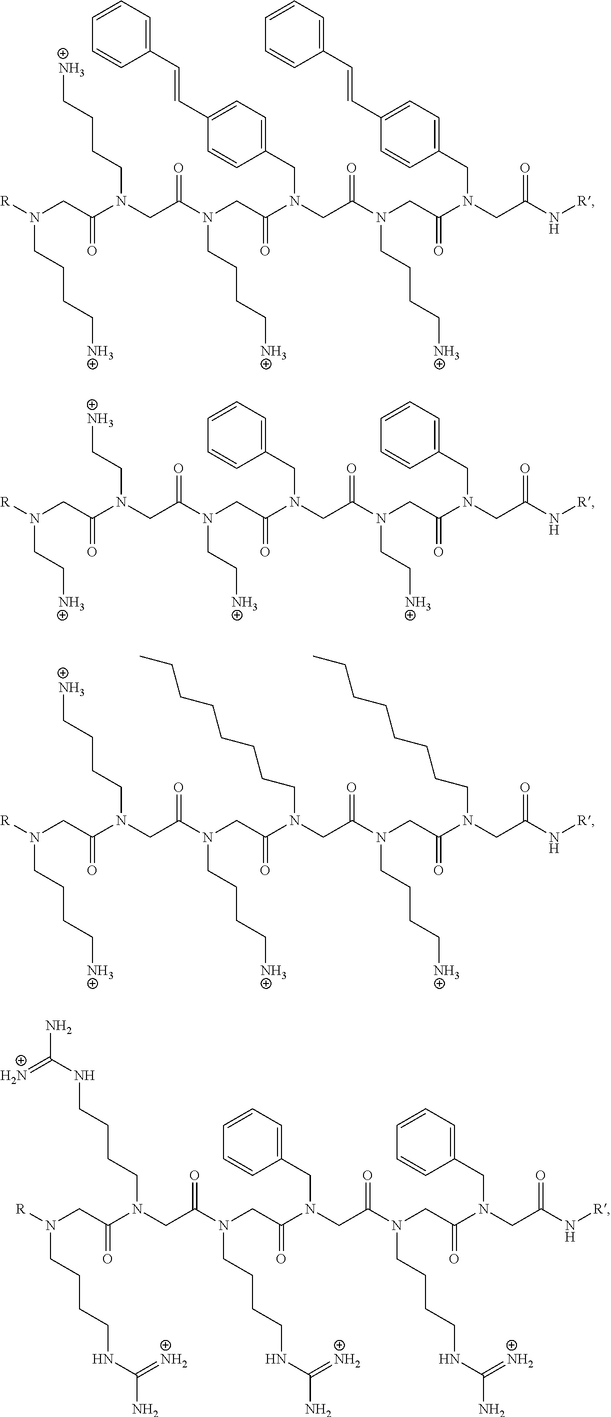

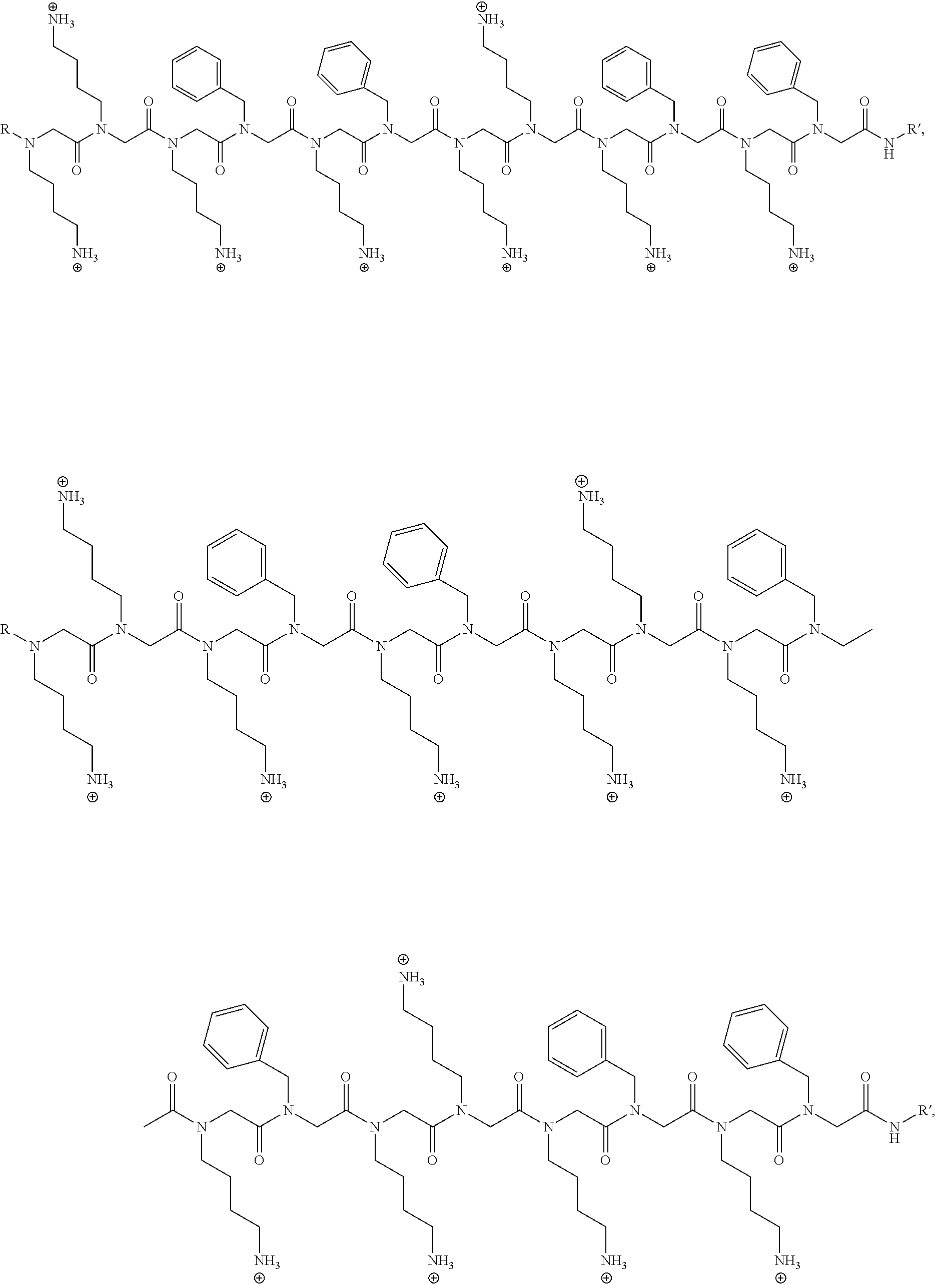

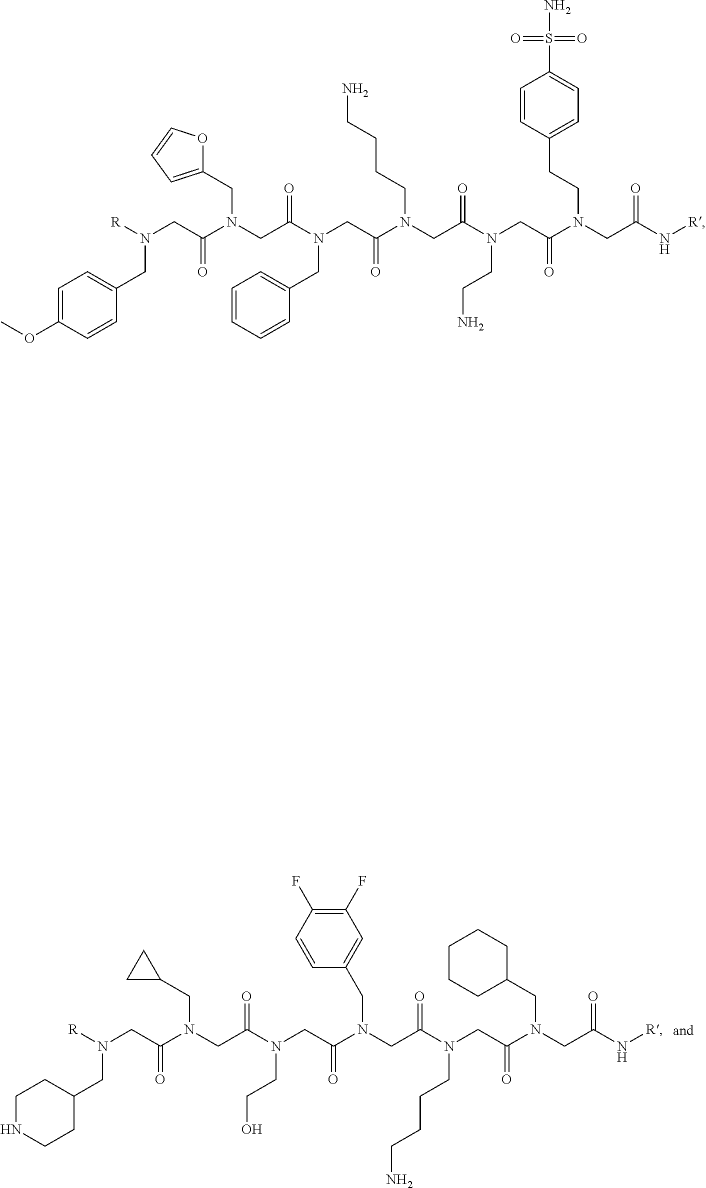

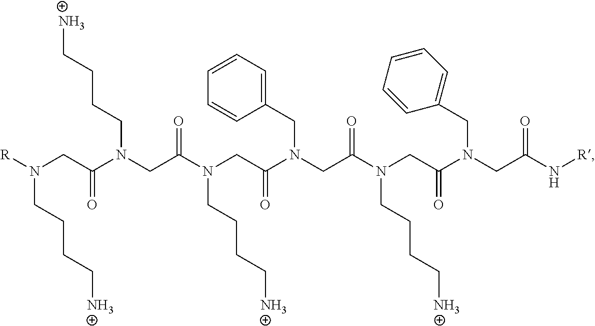

wherein R and R′ is any group.
In certain embodiments, the aggregate-specific binding reagent includes a a peptide selected from the group consisting of: KKKFKF (SEQ ID NO: 1), KKKWKW (SEQ ID NO: 2), KKKLKL (SEQ ID NO: 3), FKFKKK (SEQ ID NO: 36), FFFKFKKK (SEQ ID NO: 49), FFFFFKFKKK (SEQ ID NO: 50), FFFKKK (SEQ ID NO: 51), FFFFKK (SEQ ID NO: 52), KKFKKF (SEQ ID NO: 42), KFKKKF (SEQ ID NO: 43), kkkfkf (SEQ ID NO: 37), KIGVVR (SEQ ID NO: 44), MKFMKMHNKKRY (SEQ ID NO: 67), LIPIRKKYFFKL (SEQ ID NO: 69), RGRERFEMFR (SEQ ID NO: 47), and SEQ ID NOs 53, 55, 56 and 58-66.

### BRIEF DESCRIPTION OF THE DRAWINGS

FIG. 1 shows the potential aggregate-specific binding reagents that were tested. The sequence/name of each aggregate-specific binding reagent is indicated along with the presumed net charge of each molecule (based on functional group pKa at pH 7). The structure of the “R” group can be seen in FIG. 2.

FIG. 2 shows the reaction by which maleimide-displaying beads were conjugated to thiolated peptides by a Michael addition reaction.

FIG. 3 shows the steps of the Misfolded Protein Assay (MPA).

FIG. 4 shows the results of testing the ability of aggregate-specific binding reagents of various charge, scaffold, and hydrophobicity to capture oligomers. Part A shows fully negative, fully positive, and neutral peptides as well as peptides containing hydrophobic or aliphatic residues, and a peptoid and a dendron. Part B shows peptides with various combinations of charge and hydrophobicity, as well as a peptoid. The y-axis indicates relative light units from the Abeta ELISA.

FIG. 5 demonstrates that capture of oligomers increases non-linearly with ligand density. Part A shows loading density versus capture efficiency when 3 microliters of beads were added to each sample, and part B shows loading density versus capture efficiency when 15 microliters of beads were added to each sample.

FIG. 6 shows the comparison of two positively charged peptides, KKKKKK and KKKKKKKKKKKK, in an oligomer capture assay.

FIG. 7 shows the analysis of E22G globulomer structures. Part A shows an SDS-PAGE analyis of E22G and wild-type globulomers. Part B shows size exclusion chromatography of the two globulomers.

FIG. 8 shows a binding assay testing the capture of E22G and wild-type globulomers by PSR1 and a glutathione negative control.

FIG. 9 shows SDS-PAGE analysis of the E22K globulomer. Part A shows analysis of E22K and wild-type globulomers without crosslinking. Part B shows the analysis with cross-linked globulomers.

FIG. 10 shows a binding assay testing the capture of E22K, E22G, and wild-type globulomers by PSR1 and a glutathione negative control.

FIG. 11 shows additional peptoid aggregate-specific binding reagents tested for their ability to capture oligomers.

FIG. 12 shows capture of globulomers by various peptoid aggregate-specific binding reagents.

FIG. 13 shows capture of globulomers with PSR1 directly conjugated to beads (Part A) and with biotin-PSR1 bound to streptavidin-coated beads (Part B).

FIG. 14 shows the reaction for binding biotinylated derivatives to streptavidin-derivatized magnetic beads.

FIG. 15A shows Batch 1, the control peptoids prepared in this study (PSR1 analog, negatively charged PSR1 analog, all positive control). FIG. 15B shows Batch 2, the peptoids prepared to examine requirement for charge as well as pattern of charges.

FIG. 16 shows prion aggregate capture by Batch 1 peptoids. Data is shown in triplicate.

FIG. 17 shows prion aggregate capture by Batch 2 peptoids. Data is shown in triplicate.

FIG. 18A shows Abeta (1-42) aggregates from an Alzheimer's brain homogenate (ADBH) captured by the peptoids shown in FIG. 15. FIG. 18B shows Abeta (1-42) aggregates from ADBH captured by the positively charged peptoids shown in FIG. 15.

FIG. 19 shows the limit of detection analysis for PSR1 and all positively charged species capturing Abeta aggregates from AD BH.

FIG. 20 shows total Tau signal as captured by PSR1 beads, glutathione control beads, 7+ and 7− peptoids beads.

FIG. 21 shows that the fold change in MPA signal does not change linearly with PSR1 coating concentrations.

FIG. 22A shows Abeta (1-40) aggregates from ADBH captured by the peptoids shown in FIG. 15. FIG. 22B shows Abeta (1-40) aggregates from ADBH captured by the positively charged peptoids shown in FIG. 15.

FIG. 23 shows charge density experiment for peptide aggregate-specific binding reagents KKKFKF and KKKLKL and peptoid aggregate-specific binding reagent PSR1. The results for PSR1 are shown for PSR1 conjugated to beads and for PSR1 conjugated to cellulose.

FIG. 24 shows the ability of the Misfolded Protein Assay (MPA) to differentiate brain homogenates from control and diseased patients. Part A shows prion aggregate capture in 5 normal (N) and 11 vCJD patient samples. ANOVA shows that these 16 samples do not come from a single population. Part B shows Tau and Abeta 1-42 aggregate capture in 4 normal (N) and 10 AD patient samples. ANOVA shows that these 14 samples do not come from a single population. The y-axis in both graphs is the relative light units detected in the target marker ELISA assay.

FIG. 25 shows the results of a study looking at the impact of charge on oligomer capture. 0 or 1 ng/mL Abeta42 oligomers were spiked into CSF and captured with heads hearing potential hexapeptide aggregate-specific binding reagents. Capture of the oligomers is shown by the black bars, and background capture of monomeric Abeta40 and 42 from CSF is shown in striped and white bars. The x axis shows the hexapeptide sequence (PSR1, SEQ ID NO:15, shown for reference). The y axis shows relative light units from the Abeta immunoassay.

FIG. 26 shows a comparison of charge vs. oligomer capture signal for the hexapeptide reagents in FIG. 25. Charge is calculated based on the pKa of the individual functional groups relative to the pH of the assay buffer.

FIG. 27 shows potential aggregate specific binding reagents with different orientations and monomer chirality.

FIG. 28 shows the results of the orientation and chirality study for the reagents shown in FIG. 27. Abeta42 oligomers were spiked into CSF and captured with beads bearing the potential aggregate-specific binding reagents from FIG. 27. Capture of the oligomers is shown by the black bars, and background capture of monomeric Abeta40 and 42 from CSF is shown in striped and white bars. The x axis shows the reagent sequence. The y axis shows relative light units from the Abeta immunoassay.

FIG. 29 shows the results of a study looking at the impact of hydrophobic residues on oligomer capture. Abeta42 oligomers were spiked into CSF and captured with beads bearing potential hexapeptide aggregate-specific binding reagents. Capture of the oligomers is shown by the light bars, and background capture of monomeric Abeta42 from CSF is shown in darker bars. The x axis shows the hexapeptide sequence. The y axis shows relative light units from the Abeta immunoassay.

FIGS. 30A-C show the results of a study looking at the impact of aromatic residues on oligomer capture. Abeta42 oligomers were spiked into CSF and captured with beads bearing potential hexapeptide aggregate-specific binding reagents. FIG. 30A shows peptides of the format XKXKKK, where X is the residue indicated on the x axis (PSR1 shown for reference). The y axis shows relative light units from the Abeta immunoassay. Capture of the oligomers is shown by the black bars, and background capture of monomeric Abeta42 from CSF is shown in white bars. FIG. 30B shows peptides of the format KKKXKX, where X is the residue indicated on the x axis PSR1 shown for reference). The y axis shows relative light units from the Abeta immunoassay. Capture of the oligomers is shown by the horizontal striped bars, and background capture of monomeric Abeta42 and 40 from CSF is shown in black, white, and stippled bars. FIG. 30C shows peptides of the format XKXKKK, where X is the residue indicated on the x axis. The y axis shows relative light units from the Abeta immunoassay. Capture of the oligomers is shown by the light bars, and background capture of monomeric Abeta42 from CSF is shown in dark bars.

FIG. 31 shows the results of a study looking at the impact of different types of aromatic residues on oligomer capture. Abeta42 oligomers were spiked into CSF and captured with beads bearing potential aggregate-specific binding reagents with thiophene rings, charged aromatics, and PSR1. Capture of the oligomers is shown by the horizontally striped bars, and background capture of monomeric Abeta42 and 40 from CSF is shown in black, white, and stippled bars. The x axis shows the binding reagent. The y axis shows relative light units from the Abeta immunoassay.

FIG. 32 shows the results of a study looking at the nature of the charged residues on oligomer capture. Abeta42 oligomers were spiked into CSF and captured with beads bearing potential aggregate-specific binding reagents with diaminobutanoic acid (fdb), ornithane (Orn, with the side chain incorporated into the peptide backbone), and PSR1. Capture of the oligomers is shown by the first bars, and background capture of monomeric Abeta42 and 40 from CSF is shown in 2nd-4th bars. The x axis shows the reagent. The y axis shows relative light units from the Abeta immunoassay.

FIGS. 33A-C shows the results of testing additional positively charged aggregate specific binding reagents. Abeta42 oligomers were spiked into CSF and captured with beads bearing the potential aggregate-specific binding reagents. For FIGS. 33A and 3313, capture of the oligomers is shown by the diagonal striped bars, and background capture of monomeric Abeta42 from CSF is shown in solid bars. The x axis shows the reagent. The y axis shows relative light units from the Abeta immunoassay. For FIG. 33C, capture of 0.5 ng/mL oligomer (first bar), 0.05 ng/mL oligomer (second bar), and 0 ng/mL oligomer (third bar) spiked into CSF was tested. The x axis shows the reagent (see Tables 13 and 14 for code and structure). The y axis shows relative light units from the Abeta immunoassay.

FIG. 34 shows two identical peptide arrays (˜1120 12mer peptides in each) that were incubated with 3 ng/mL monomeric or oligomeric Abeta1-42 for the purpose of identifying peptides that would preferentially bind to oligomeric Abeta1-42. Bound Abeta1-42 was detected by western blot using anti-Abeta antibodies (6E10) that recognize the N-terminus of the peptide. A significant number of peptides binding to oligomers, but not monomers, were detected. Only a few peptides (circled) recognized both monomeric and oligomeric Abeta without much selectivity. Signals associated with Abeta peptide capture were quantified using Kodak image station software, and the peptides were ranked from highest to lowest net intensity. The top 5-10% of peptides were considered to be top binders.

FIG. 35 shows the NMPA background reduction by 1% TW20 or 1% ZW 3-14 washing in NMPA. Different matrixes (TBSTT and CSF) were incubated with ASR1. With or without 1% TW20 or 1% ZW 3-14 was used to wash the pulldown beads after the incubation. The x axis shows the pulldown matrix in NMPA and the detergent used for after pulldown washing. The y axis shows relative light units from the Abeta immunoassay.

FIG. 36 shows NMPA background reduction and sensitivity improvement with detergent washing. Abeta42 oligomers were spiked into TBSTT or CSF and incubated with ASR1. Pulldown beads were washed with or without 1% detergent after the incubation. The x axis of top and bottom graph shows spiked oligomer levels. The y axis of top graph shows relative light units from the Abeta immunoassay. The y axis of bottom graph shows S/N ratio of Abeta 42 from the Abeta immunoassay.

FIG. 37 shows the detergent structures and names.

FIG. 38 shows native gel analysis of various Aβ42 aggregates.

FIG. 39 shows the capture of Aβ40 aggregates in AD CSF by PSR1 and Ac-FKFKKK.

FIG. 40 depicts the amount of Aβ40 oligomer detected by the Misfolded Protein Assay in the supernatant and pellet of Alzheimer's Disease CSF and normal CSF centrifuged at 16,000 g for 10 minutes or 134,000 g for 1 hour. Legend: Small checks: total amount Aβ40; Large checks: 17,000 g supernatant; Horizontal line: 16,000 pellet; Vertical line: 134,000 g supernatant, Diagonal line: 134,000 g pellet.

FIG. 41 shows a histological evaluation of AA amyloidosis in spleen. Typical examples of different degrees of splenic amyloid deposits stained with Congo red dye are depicted. Amyloid exhibit green birefringence when studied under polarised light. 1+, very thin focal deposits at follicles (A), 2+, more pronounced perifollicular amyloid deposits in limited area of the spleen (B and C), 3+, moderate amyloid deposits around most or all follicles (1)), 4+, extensive amyloid deposit localized around follicles but often forming continuous infiltration (E and F). (×25)

FIG. 42 demonstrates that PSR1-coated beads can capture AA-related moieties. (A-C) Immunoblotting using a monoclonal anti-mouse SAA antibody on PSR1-depleted input (A), eluate (B) and beads (C) fractions. This Misfolded Protein Assay (MPA) was performed with 3 or 9 uL of PSR1-coated beads using 1, 4 or 8 uL of 10% w/v spleen homogenate from a mouse with splenic AA (AA) and a control untreated mouse (Ctrl) as inputs. (1)) Detection of SAA-related species by sandwich ELISA. Values under the detection limit are represented as 0 ug/mL.

FIG. 43 shows the optimization of the AA MPA assay. An immunoblot using a polyclonal anti-mouse SAA/AA antibody (“AA138”) on input, PSR1-depleted input beads and eluate fractions is depicted. MPA was performed with 6 ul of PSR1-coated beads and 10% w/v spleen homogenate corresponding to 50 ug of total protein from a mouse with splenic AA (AA+), control mouse that was challenged by single AgNO₃ injection (AgNO₃ primed) and a control untreated mouse (untreated) as inputs. Actin was used as a loading control.

FIG. 44 demonstrates that denaturation of AA aggregates prevents the detection of AA-related moieties. Detection of SAA-related species by sandwich ELISA on eluate fractions is depicted. Denaturation was achieved by mixing 9 uL of 10% w/v spleen homogenate from a mouse with splenic AA with 13.5 uL of denaturing buffer and incubating for 10 or 30 minutes at room temperature or 37° C. at 750 rpm and was followed by neutralization with 5.4 uL of neutralizing buffer (denat-AA). A buffered control (buff-AA) was prepared mixing 9 uL of 10% w/v spleen homogenate from a mouse with splenic AA with premixed 13.5 uL of denaturating buffer and 5.4 uL of neutralizing buffer. MPA was performed using the above-described four denaturated samples, as well as the buffered AA sample, an undenaturated AA-containing sample (undenat-AA), an undenaturated spleen homogenate sample from a control AgNO3-treated mouse (undenat-AgNO3) and an undenaturated spleen homogenate sample from a control untreated mouse (undenat-BL6).

FIG. 45 shows that PSR1 beads bind preferentially to in vitro-synthesized amylin fibrils over amylin monomers in both buffer (A) and plasma (B).

FIG. 46 depicts amylin aggregates in pancreatic tissue from Type II diabetes patients can not be detected by ELISA (native) unless they are treated with a denaturant (denatured). There are only low levels of amylin found in pancreatic tissue from a normal non-diseased patient. Legend: circle: normal, native; square: Type II diabetes, native; triangle; normal, denatured; inverted triangle: Type II diabetes, denatured

FIG. 47 demonstrates that PSR1 preferentially detects amylin fibrils over monomers from pancreatic tissue. Legend: circle: normal, native; square: Type II diabetes, native; triangle; normal, denatured; inverted triangle: Type II diabetes, denatured

FIG. 48 demonstrates that amylin fibrils in Type II diabetes pancreatic tissue bind to PSR-1, but not to glutathione or 5 L (negative version of PSR1) control beads in plasma. Legend: circle: 5 L bead; square: glutathione bead; triangle: PSR1 bead.

FIG. 49 shows that alpha-synuclein (aSyn) fibrils are not detected by ELISA. Legend: closed circle, denatured fibril; open circle, native

FIG. 50 shows that PSR1 beads but not control beads can capture alpha synuclein fibrils spiked into CSF or plasma. Legend: closed square: PSR1-aSyn fibril in CSF; open square: CTRL-aSyn fibril in CSF; closed triangle: PSR1—aSyn fibril in plasma; inverted open triangle: aSyn fibril in plasma.

FIG. 51 shows that PSR1 binds preferentially to alpha-synuclein fibrils over monomers in CSF and plasma. Legend: closed square: aSyn fibril in CSF; open square: denatured aSyn in CSF; closed triangle: native aSyn fibril in plasma; open triangle: denatured aSyn in plasma.

FIG. 52 depicts the amount of alpha synuclein eluted from PSR1 beads under different conditions. Legend: light bars: GdnSCN; dark bars: NaOH

FIG. 53 depicts Kaplan-Meier survival plots of Tg(SHaPrP) mice. (A) Tg(SHaPrP) mice were inoculated with serial 10-fold dilutions of a 10% (wt/vol) 263K hamster brain homogenate ranging from 10⁻² to 10⁻¹² for the estimation of the prion titre. (B) Bioassay of Tg(SHaPrP) mice that were i.c. inoculated with PSR1 beads that were incubated with pooled infectious prion plasma from 263K prion symptomatic hamsters. Hamsters were bled and sacrificed after the indicated days post inoculation with 263K prions. Mice were either inoculated with 5.25 or 10.5 μl beads in PBS or TBSTT as indicated in the Figure.

FIG. 54 depicts the pathology of brain sections from Tg(SHaPrP) mice. Mice inoculated with 263K prion-infected hamster brain homogenate (B), inoculated with PSR1 beads incubated with plasma from pool 2 (117-118 dpi) (C) and from pool 1 (143-154 dpi) (1)) show vacuoles as shown by hematoxylin and eosin staining, PrP^{Sc} depositions as visualized by the PrP antibody SAF84 and astrocytic gliosis as evidenced by an antibody directed against GFAP. Non-inoculated mice (A) showed no signs of vacuolation, PrP^{Sc} depositions or gliosis. Histoblot analysis was used to show PrP^{Sc} deposition after proteinase K digestion and staining with POM1.

FIG. 55 depicts Western blot analysis of proteinase K digested brain homogenates from Tg(SHaPrP) mice. (A-C) Proteinase K resistant material is present in Tg(SHaPrP) i.e. inoculated with PSR1 beads incubated with plasma from pool 1 (143-154 dpi; Mice #1-9) and 2 (117-118 dpi; Mice #1-3). Control samples are labeled with no: brain homogenate from healthy mice and 263: brain homogenate from mice inoculated with 263K prion. The molecular weight standard is shown in kilodaltons. Mouse #1 was inoculated with 10.5 μl beads in PBS, mice #2-4 with 5.25 μl beads in PBS, mice #5 and 6 with 10.5 μl beads in TBSTT, mice #7 and 8 with 5.25 μl beads in TBSTT, and mice #1-3 (117-118 dpi) with 10.5 μl beads in TBSTT.

### BRIEF DESCRIPTION OF TABLES

Table 1 lists exemplary conformational diseases and the associated conformational proteins.

Table 2 lists exemplary peptide sequences for making ASB reagents.

Table 3 lists exemplary peptoid regions suitable for making ASB reagents.

Table 4 provides a key to the abbreviations used in Table 3.

Table 5 provides the relevant structures for the peptoid sequences listed in Table 3.

Table 6 provides characterization information for peptoids tested in Example 3.

Table 7 shows the total prion signal as captured by Streptavidin magnetic beads conjugated with increasing density of PSR1 (+++A+A).

### BRIEF DESCRIPTION OF SEQUENCE LISTING

SEQ ID NOs: 1 to 8 provide the amino acid sequences of exemplary peptides for use in making ASB reagents.

SEQ ID NOs: 9-29 provide the modified amino acid sequences of exemplary peptoids for use in making ASB reagents.

### DETAILED DESCRIPTION OF THE INVENTION

This invention relates to the discovery of reagents which bind preferentially to aggregates over monomers when attached to a solid support at certain charge densities. These aggregates may be associated with conformational diseases such as Alzheimer's disease, diabetes, systemic amyloidoses, etc.

The discovery of reagents which preferentially bind to aggregates over monomers allows the development of detection assays, diagnostic assays and purification or isolation methods utilizing these reagents for conformational diseases or other uses.

While not wishing to be held to any theory, it is believed that the ability of these ASB reagents to preferentially bind and detect aggregates is due to the repepating nature of the monomeric units within the aggregate.

Many aggregates share similar physical properties. For example, PrP^{Sc}, the aggregate of the prion protein, exhibits the following characteristics: increased β-sheet content (˜3% in PrP^{C} to >40% in PrP^{Sc}) and PrP^{Sc} fibers are composed of β-sheets that are oriented perpendicularly along the fiber axis. Aggregates of Aβ peptides share similar β-sheet structure (Luhrs, et al., 2005, PNAS102: 17342). Applicants believe that binding to these repeating protein surfaces is the mechanism by which the aggregate-specific reagents of the invention bind preferentially to aggregates over monomers when attached to the solid support.

The ASB reagents of the invention have a net charge of at least about positive one and are attached to a solid support at a charge density of at least about 60 nmol net charge per square meter. While not wanting to be held to any particular theory, Applicants believe that the postive charge of the ASB reagents allows them to bind to aggregates via ionic interactions between the positive charges of the ASB reagent and negative charges on the aggregate. These negative charges may be provided by exposed negatively-charged residues of misfolded conformers in the aggregate or by negative charges on salts, lipids, or other species contained in the aggregate. Although, ionic interactions are critical, structure and size of the aggregates also play a role in binding as ASB reagents are capable of preferentially binding to aggregates having exposed positive charges.

Furthermore, ASB reagents display increased preference for aggregates over monomers as the charge density of the ASB reagent on a solid support is increased. While not wanting to be held to any particular theory, Applicants believe that increased charge density allows for the ASB reagents to bind with more avidity to aggregates containing ordered structures which have repeated patterns of exposed negative charges.

These ASB reagents need not be part of a larger structure or other type of scaffold molecule in order to exhibit this preferential binding to aggregate. It will be apparent to one of ordinary skill in the art that, while the exemplified ASB reagents provide a starting point (in terms of size or sequence characteristics, for example) for ASB reagents useful in methods of this invention that many modifications can be made to produce ASB reagents with more desirable attributes (e.g, higher affinity, greater stability, greater solubility, less protease sensitivity, greater specificity, easier to synthesize, etc.).

In general, the ASB reagents described herein are able to bind preferentially to aggregates over monomers when attached to a solid support at certain charge densities. Thus, these reagents allow for ready detection of the presence of aggregates in virtually any sample, biological or non-biological, including living or dead brain, spinal cord, cerebrospinal fluid, or other nervous system tissue as well as blood and spleen. The ASB reagents are therefore useful in a wide range of isolation, purification, detection, diagnostic and therapeutic applications.

The practice of the present invention will employ, unless otherwise indicated, conventional methods of chemistry, biochemistry, molecular biology, immunology and pharmacology, within the skill of the art. Such techniques are explained fully in the literature. See, e.g., Remington's Pharmaceutical Sciences, 18th Edition (Easton, Pa.: Mack Publishing Company, 1990); Methods In Enzymology (S. Colowick and N. Kaplan, eds., Academic Press, Inc.); and Handbook of Experimental Immunology, Vols. I-IV (D. M. Weir and C. C. Blackwell, eds., 1986, Blackwell Scientific Publications); Sambrook, et al., Molecular Cloning: A Laboratory Manual (2nd Edition, 1989); Handbook of Surface and Colloidal Chemistry (Birdi, K. S. ed., CRC Press, 1997); Short Protocols in Molecular Biology, 4th ed. (Ausubel et al. eds., 1999, John Wiley & Sons); Molecular Biology Techniques: An Intensive Laboratory Course, (Ream et al., eds., 1998, Academic Press); PCR (Introduction to Biotechniques Series), 2nd ed. (Newton & Graham eds., 1997, Springer Verlag); Peters and Dalrymple, Fields Virology (2d ed), Fields et al. (eds.), B. N. Raven Press, New York, N.Y.

It is understood that the reagents and methods of this invention are not limited to particular formulations or process parameters as such may, of course, vary. It is also to be understood that the terminology used herein is for the purpose of describing particular embodiments of the invention only, and is not intended to be limiting.

### I. DEFINITIONS

In order to facilitate an understanding of the invention, selected terms used in the application will be discussed below.

Proteins may exist in more than one conformation as a result of protein misfolding. As used herein, the term “conformer” refers to a protein monomer of a certain conformation. For example, in vivo, the majority of proteins are present as correctly folded conformers. As used in this disclosure with respect to conformers, the terms “native” or “cellular” refer to the correctly folded conformer of a protein. Proteins may also exist as misfolded conformers. In many cases, these misfolded conformers are pathogenic. As used herein, the term “pathogenic” may mean that the protein or conformer actually causes a disease or it may simply mean that the protein or conformer is associated with a disease and therefore is present when the disease is present. Examples of proteins for which a pathogenic conformer exists are listed in the right-hand column of Table 1. Thus, a pathogenic protein or conformer as used in connection with this disclosure is not necessarily a protein that is the specific causative agent of a disease and therefore may or may not be infectious. The term “non-pathogenic” when used with respect to conformers refers to the native conformer of a protein whose presence is not associated with disease. A pathogenic conformer associated with a particular disease, for example, Alzheimer's disease, may be described as a “pathogenic Alzheimer's disease conformer”.

In some cases, non-native conformers of a protein are not associated with disease. For example, yeast prions, such as Sup35p, may exist in a yeast cell as non-native conformers but have no effect on the vigor or viability of the yeast cells. Other examples of proteins that form non-native conformers that are not associated with disease are curlin (E. coli), chaplins (Streptomyces coelicolor), prion Het-s (Podospora anserina), malarial coat protein, spider silk in some spiders, Melanocyte protein Pmel 17, tissue-type plasminogen activator (tPA), and hormones, such as ACTH, beta endorphin, prolactin, and growth hormone.

In contrast to the non-native conformers disussed above, some proteins may not exist as non-native conformers in vivo but are capable of forming non-native conformers in vitro. Some examples of these proteins capable of forming non-native conformers are myoglobin, SH3 domain of the p85α subunit of phosphatidylinositol 3-kinase, acylphosphatase, and HypF-N (E. coli).

As used herein, the term “aggregate” refers to a complex containing more than one copy of a non-native conformer of a protein that arises from non-native interactions among the conformers. Aggregates may contain multiple copies of the same protein, multiple copies of more than one protein, and additional components including, without limitation, glycoproteins, lipoproteins, lipids, glycans, nucleic acids, and salts. Aggregates may exist in structures such as inclusion bodies, plaques, or aggresomes. Some examples of aggregates are amorphous aggregates, oligomers, and fibrils. Amorphous aggregates are typically disordered and insoluble. An “oligomer” as used herein contains more than one copy of a non-native conformer of a protein. Typically, they contain at least 2 monomers, but no more than 1000 monomers, or in some cases, no more than 10⁶ monomers. Oligomers include small micellar aggregates and protofibrils. Small micellar aggregates are typically soluble, ordered, and spherical in structure. Protofibrils are also typically soluble, ordered aggregates with beta-sheet structure. Protofibrils are typically curvilinear in structure and contain at least 10, or in some cases, at least 20 monomers. Fibrils are typically insoluble and highly ordered aggregates. Fibrils typically contain hundreds to thousands of monomers. Fibrils include, for example, amyloids, which exhibit cross-beta sheet structure and can be identified by apple-green birefringence when stained with Congo Red and seen under polarized light. When contained in a single sample, aggregates such as amorphous aggregates, oligomers, and fibrils may be separated by centrifugation. For example, centrifugation at 14,000×g for 10 minutes will typically remove only very large aggregates, such as large fibrils and amorphous aggregates (10-1000 MDa), and centrifugation at 100,000×g for one hour will typically remove aggregates larger than 1 MDa, such as smaller fibrils and amorphous aggregates. Size and solubility of aggregates will affect the sedimentation velocity required for separation.

Aggregates of the invention may contain any of the proteins discussed above that exist or are capable of existing as non-native conformers. In many cases, the aggregates are associated with disease. Examples of such diseases and their associated conformational proteins are listed in Table 1. In other cases, aggregates are associated with high yield manufacture of proteins for pharmaceutical or other industrial use. For example, proteins such as recombinant insulin or therapeutic antibodies, tend to aggregate when produced at high levels. Aggregates may also be found as a form of natural storage in secretory granules (Science, 2009, 325: 328).

The term “aggregate-specific binding reagent” or “ASB reagent” refers to any type of reagent, including but not limited to peptides and peptoids, which binds preferentially to an aggregate compared to monomer when attached to a solid support at certain charge densities. The binding may be due to increased affinity, avidity, or specificity. For example, in certain embodiments, the aggregate-specific binding reagents described herein bind preferentially to aggregates but, nonetheless, may also be capable of binding monomers at a weak, yet detectable, level. Typically, weak binding, or background binding, is readily discernible from the preferential interaction with the aggregate of interest, e.g., by use of appropriate controls. In general, aggregate-specific binding reagents used in methods of the invention bind aggregates in the presence of an excess of monomers. Preferably, ASB reagents bind aggregates with an affinity/avidity that is at least about two times higher than the binding affinity/avidity for monomer.

“PSR1” is one example of an ASB reagent. PSR1 contains the sequence of SEQ ID NO: 15. The structure of SEQ ID NO: 15 is shown in Table 5.

An aggregate-specific binding reagent is said to “bind” with another peptide or protein if it binds specifically, non-specifically or in some combination of specific and non-specific binding. A reagent is said to “bind preferentially” to an aggregate if it binds with greater affinity, avidity, and/or greater specificity to the aggregate than to monomer. The terms “bind preferentially,” “preferentially bind,” “bind selectively,” “selectively bind,” and “selectively capture” are use interchangeably herein.

“Conformational protein” refers to the native and misfolded conformers of a protein.

Many conformational proteins are conformational disease proteins. “Conformational disease protein” refers to the native and pathogenic misfolded conformers of a protein associated with a conformational disease where the structure of the protein has changed (e.g., misfolded) such that it results in the formation of aggregates such as unwanted soluble oligomers or amyloid fibrils. Examples of conformational disease proteins include, without limitation, Alzheimer's disease proteins, such as Aβ and tau; prion proteins such as PrP^{Sc} and PrP^{C}, Parkinson's disease proteins such as alpha-synuclein, AA amyloidosis proteins such as Amyloid A protein, and the diabetes protein amylin. A non-limiting list of diseases with associated proteins that assume two or more different conformations is shown below.

TABLE 1

| | Conformational |
|---|---|
| Disease | Disease Protein(s) |
| Prion diseases | PrP^{Sc} |
| (e.g., Creutzfeldt-Jakob disease, scrapie, | |
| bovine spongiform encephalopathy) | |
| Alzheimer's Disease | Aβ peptides, Tau |
| | non-Aβ component |
| ALS | SOD1, tau |
| Pick's disease | Pick body (tau) |
| Parkinson's disease | Lewy body (tau, |
| | alpha-synuclein) |
| Diabetes Type II | Amylin |
| Multiple myeloma-plasma cell dyscrasias | IgG light chain |
| | IgG heavy chain |
| Familial amyloidotic polyneuropathy | Transthyretin |
| Medullary carcinoma of thyroid | Procalcitonin |
| Chronic Renal failure | beta2-microglobulin |
| Congestive heart failure | atrial natriuretic factor |
| senile cardiac and systemic amyloidosis | Transthyretin |
| Familial Amyloid Polyneuropathy | |
| Chronic inflammation | Serum amyloid A |
| (e.g., Rheumatoid arthritis) | |
| Atherosclerosis | ApoA1 |
| Familial amyloidosis (Finnish) | Gelsolin |
| All tauopathies, including argyrophilic | Tau |
| grain dementia, corticobasal degeneration, | |
| dementia pugilistica, Hallervorden-Spatz | |
| disease, myotonic dystrophy, etc. | |
| Synucleinopathies, including Gaucher's | Alpha-synuclein |
| disease, multisystem atrophy, Lewy body | |
| dementia, etc. | |
| Corneal dystrophy, gelatinous drop-like | Lactoferrin |
| Aortic amyloidosis in the elderly | Medin |
| Cutaneous amyloidosis | Keratin |
| Heriditary cerebral hemorrhage (Icelandic) | Cystatin C |
| Huntington's Disease | Huntingtin |
| Hereditary non-neuropathic systemic | Lysozyme |
| amyloidosis | |
| Lattice corneal dystrophy | Keratoepithelin |
| Cerebral amyloid angiopathy | Beta amyloid |
| Sporadic Inclusion Body Myositis | Beta amyloid, Tau |
| Cerebral Beta-amyloid angiopathy | Beta amyloid |
| Retinal ganglion cell degeneration (FTLD) | TDP-43 |
| (Ubi+, Tau−) | |
| Amyotrophic lateral sclerosis (ALS) | Superoxide dismutase, TDP-43 |
| Familial British Dementia | ABri |
| Familial Danish Demetia | ADan |
| CADASIL | Notch3 |
| Alexander Disease | Glial fibrillary acidic protein |
| Seipinopathies | Seipins |
| (e.g., Silver Syndrome, Spastic Paraplegia, | |
| dHMN-V, Charcot-Marie-Tooth Disease | |
| Type 2) | |
| Serpinopathies | Serpins |
| (e.g., liver cirrhosis, dementia of familial | |
| encephalopathy) | |
| AL (light chain) amyloidosis | Immuoglobulin light chains |
| AH (heavy chain) amyloidosis | Immunoglobulin heavy chains |
| AA (secondary) amyloidosis | Amyloid A protein |
| Heavy Chain Deposition disease | Immunoglobulin heavy chains |
| ApoAI amyloidosis | Apolipoprotein AI |
| ApoAII amyloidosis | Apolipoprotein AII |
| ApoAIV amyloidosis | Apolipoprotein AIV |
| Fibrinogen amyloidosis | Fibrinogen |
| Includion body myositis/myopathy | amyloid beta peptide |
| Cataracts | Crystallins |
| Pituitary prolactinoma | Prolactin |
| Pulmonary alveolar proteinosis | surfectant protein C (SP-C) |
| Odontogenic (Pindborg) tumor amyloid | Odontogenic ameloblast- |
| | associated protein |
| Seminal vesicle amyloid | Semenogelin I |
| Cystic Fibrosis | CFTR protein |
| Sickle Cell Disease | Hemoglobin |
| Critical illness myopathy (CIM) | hyperproteolytic state of |
| | myosin ubiquitination |
| Preeclampsia | Anti-trypsin |
| (Am J Obstet Gynecol, 2008 November, | |
| 199(5): 551.e1-16) | |

A “conformational disease protein” as used herein is not limited to polypeptides having the exact sequence as those described herein. It is readily apparent that the terms encompass conformational disease proteins from any of the identified or unidentified species or diseases (e.g., Alzheimer's, Parkinson's, etc.).

“Conformational protein-specific binding reagent” or “CPSB reagent” refers to any type of reagent which interacts with more than one conformer of a specific protein. Preferably, conformational protein-specific binding reagents bind to both native and misfolded conformers of a conformational protein. In some instances the conformational protein-specific binding reagent may bind to both monomers and aggregates of the protein. In certain cases, CPSB reagents recognize aggregate structure regardless of protein sequence. An example of such a CPSB reagent is the A11 antibody, which recognizes aggregates of Abeta, PrP, and alpha-synuclein (Kayed et al. 2003, Science 300: 486). In other cases, CPSB reagents only recognize Abeta aggregates. However, in many cases the CPSB reagent will only bind to monomers of a protein. In methods of the invention where the CPSB reagent is used as the capture reagent, the CPSB must bind to aggregates. In methods of the invention where the CPSB reagent is used to detect aggregate, the CPSB is not required to bind aggregates. If it does not bind aggregates, it will be necessary to denature the aggregate in order for it to be detected. Typically, CPSB reagents are monoclonal or polyclonal antibodies.

The terms “prion”, “prion protein”, “PrP protein” and “PrP” are used interchangeably herein to refer to both the aggregate (variously referred to as scrapie protein, pathogenic protein form, pathogenic isoform, pathogenic prion and PrP^{Sc}) and the non-aggregate (variously referred to as cellular protein form, cellular isoform, non-pathogenic isoform, non-pathogenic prion protein, and PrP^{C}), as well as the denatured form and various recombinant forms of the prion protein which may not have either the pathogenic conformation or the normal cellular conformation. The aggregate is associated with disease state (spongiform encephalopathies) in humans and animals. The non-aggregate is normally present in animal cells and may, under appropriate conditions, be converted to the pathogenic PrP^{Sc} conformation. Prions are naturally produced in a wide variety of mammalian species, including human, sheep, cattle, and mice.

The term “Alzheimer's disease (AD) protein” or “AD protein” are used interchangeably herein to refer to both the aggregate (variously referred to as pathogenic protein form, pathogenic isoform, pathogenic Alzheimer's disease protein, and Alzheimer's disease conformer) and the non-aggregate (variously referred to as normal cellular form, non-pathogenic isoform, non-pathogenic Alzheimer's disease protein), as well as the denatured form and various recombinant forms of the Alzheimer's disease protein which may not have either the pathogenic conformation or the normal cellular conformation. Exemplary Alzheimer's disease proteins include Aβ and the tau protein.

The terms “amyloid-beta,” “amyloid-β,” “Abeta”, “Aβ,” “Aβ42”, “Aβ40,” “Aβx-42,” “Aβx-40,” and “Aβ40/42” as used herein all refer to amyloid-β peptides, which are a family of up to 43 amino acids in length found extracellularly after the cleavage of the amyloid precursor protein (APP). The term Aβ is used to refer generally to the amyloid-β peptides in any form. The term “Aβ40” refers to “Aβx-40.” The term “Aβ42” refers to “Aβx-42.” The term “Aβ1-42” refers to a fragment corresponding to amino acids 1 to 42 of APP. The term “Aβ1-40” refers to a fragment corresponding to amino acids 1 to 40 of APP. The term Aβ40/42 is used to refer to both the Aβ40 and Aβ42 isoforms. “Globulomer” refers to a soluble oligomer formed by Aβ42 (Barghorn et al., Journal of Neurochemistry, 2005).

The term “diabetes protein” is used herein to refer to both the aggregate (variously referred to as pathogenic protein form, pathogenic isoform, pathogenic diabetes disease protein) and the non-aggregate (variously referred to as normal cellular form, non-pathogenic isoform, non-pathogenic diabetes disease protein), as well as the denatured form and various recombinant forms of the diabetes disease protein which may not have either the pathogenic conformation or the normal cellular conformation. An exemplary Type II diabetes protein is amylin, which is also known as Islet Amyloid Polypeptide (IAPP).

By “isolated” is meant, when referring to a polynucleotide or a polypeptide, that the indicated molecule is separate and discrete from the whole organism with which the molecule is found in nature or, when the polynucleotide or polypeptide is not found in nature, is sufficiently free of other biological macromolecules so that the polynucleotide or polypeptide can be used for its intended purpose.

“Peptoid” is used generally to refer to a peptide mimic that contains at least one, preferably two or more, amino acid substitutes, preferably N-substituted glycines. Peptoids are described in, inter alia, U.S. Pat. No. 5,811,387. As used herein, a “peptoid reagent” is a molecule having an amino-terminal region, a carboxy-terminal region, and at least one “peptoid region” between the amino-terminal region and the carboxy-terminal region. The amino-terminal region refers to a region on the amino-terminal side of the reagent that typically does not contain any N-substituted glycines. The amino-terminal region can be H, alkyl, substituted alkyl, acyl, an amino protecting group, an amino acid, a peptide, or the like. The carboxy-terminal region refers to a region on the carboxy-terminal end of the peptoid that does not contain any N-substituted glycines. The carboxy-terminal region can include H, alkyl, alkoxy, amino, alkylamino, dialkylamino, a carboxy protecting group, an amino acid, a peptide, or the like.

The “peptoid region” is the region starting with and including the N-substituted glycine closest to the amino-terminus and ending with and including the N-substituted glycine closest to the carboxy-terminus. The peptoid region generally refers to a portion of a reagent in which at least three of the amino acids therein are replaced by N-substituted glycines.

“Physiological pH” refers to a pH of about 5.5 to about 8.5; or about 6.0 to about 8.0; or usually about 6.5 to about 7.5.

“Aliphatic” refers to a straight-chained or branched hydrocarbon moiety. Aliphatic groups can include heteroatoms and carbonyl moieties.

“Amino acid” refers to any of the twenty naturally occurring and genetically encoded α-amino acids or protected derivatives thereof, and any unnatural or non-alpha amino acids. Protected derivatives of amino acids can contain one or more protecting groups on the amino moiety, carboxy moiety, or side chain moiety. Examples of amino-protecting groups include formyl, trityl, phthalimido, trichloroacetyl, chloroacetyl, bromoacetyl, iodoacetyl, and urethane-type blocking groups such as benzyloxycarbonyl, 4-phenylbenzyloxycarbonyl, 2-methylbenzyloxycarbonyl, 4-methoxybenzyloxycarbonyl, 4-fluorobenzyloxycarbonyl, 4-chlorobenzyloxycarbonyl, 3-chlorobenzyloxycarbonyl, 2-chlorobenzyloxycarbonyl, 2,4-dichlorobenzyloxycarbonyl, 4-bromobenzyloxycarbonyl, 3-bromobenzyloxycarbonyl, 4-nitrobenzyloxycarbonyl, 4-cyanobenzyloxycarbonyl, t-butoxycarbonyl, 2-(4-xenyl)-isopropoxycarbonyl, 1,1-diphenyleth-1-yloxycarbonyl, 1,1-diphenylprop-1-yloxycarbonyl, 2-phenylprop-2-yloxycarbonyl, 2-(p-toluoyl)-prop-2-yloxycarbonyl, cyclopentanyloxy-carbonyl, 1-methylcyclopentanyloxycarbonyl, cyclohexanyloxycarbonyl, 1-methylcyclohexanyloxycarbonyl, 2-methylcyclohexanyloxycarbonyl, 2-(4-toluoylsulfonyl)-ethoxycarbonyl, 2-(methylsulfonyl)ethoxycarbonyl, 2-(triphenylphosphino)-ethoxycarbonyl, fluorenylmethoxycarbonyl (“FMOC”), 2-(trimethylsilyl)ethoxycarbonyl, allyloxycarbonyl, I-(trimethylsilylmethyl)prop-1-enyloxycarbonyl, 5-benzisoxalylmethoxycarbonyl, 4-acetoxybenzyloxycarbonyl, 2,2,2-trichloroethoxycarbonyl, 2-ethynyl-2-propoxycarbonyl, cyclopropylmethoxycarbonyl, 4-(decycloxy)benzyloxycarbonyl, isobornyloxycarbonyl, 1-piperidyloxycarbonlyl and the like; benzoylmethylsulfonyl group, 2-nitrophenylsulfenyl, diphenylphosphine oxide and like amino-protecting groups. Examples of carboxy-protecting groups include methyl, p-nitrobenzyl, p-methylbenzyl, p-methoxybenzyl, 3,4-dimethoxybenzyl, 2,4-dimethoxybenzyl, 2,4,6-trimethoxybenzyl, 2,4,6-trimethylbenzyl, pentamethylbenzyl, 3,4-methylenedioxybenzyl, benzhydryl, 4,4′-dimethoxybenzhydryl, 2,2′,4,4′-tetramethoxybenzhydryl, t-butyl, t-amyl, trityl, 4-methoxytrityl, 4,4′-dimethoxytrityl, 4,4′,4″-trimethoxytrityl, 2-phenylprop-2-yl, trimethylsilyl, t-butyldimethylsilyl, phenacyl, 2,2,2-trichloroethyl, .beta.-(di(n-butyl)methylsilyl)ethyl, p-toluenesulfonylethyl, 4-nitrobenzylsulfonylethyl, allyl, cinnamyl, 1-(trimethylsilylmethyl)prop-1-en-3-yl and like moieties. The species of protecting group employed is not critical so long as the derivatized protecting group can be selectively removed at the appropriate point without disrupting the remainder of the molecule. Further examples of protecting groups are found in E. Haslam, Protecting Groups in Organic Chemistry, (J. G. W. McOmie, ed., 1973), at Chapter 2; and T. W. Greene and P. G. M. Wuts, Protective Groups in Organic Synthesis, (1991), at Chapter 7, the disclosures of each of which are incorporated herein by reference in their entireties.

“N-Substituted glycine” refers to a residue of the formula —(NR—CH₂—CO—)— where each R is a non-hydrogen moiety.

Also included are salts, esters, and protected forms (e.g., N-protected with Fmoc or Boc, etc.) of the N-substituted glycines.

Methods for making amino acid substitutes, including N-substituted glycines, are disclosed, inter alia, in U.S. Pat. No. 5,811,387, which is incorporated herein by reference in its entirety.

“Subunit” refers to a molecule that can be linked to other subunits to form a chain, e.g., a peptide. Amino acids and N-substituted glycines are example subunits. When linked with other subunits, a subunit can be referred to as a “residue.”

### II. REAGENTS TO BE USED IN METHODS OF THIS INVENTION

Aggregate-specific binding reagents (“ASB reagents”) to be used in this invention are those reagents which bind preferentially to aggregates over monomers when attached to a solid support at certain charge densities.

Typically, ASB reagents have a net charge of at least about positive one at the pH at which a sample is contacted with the ASB reagent and are attached to a solid support at a charge density of at least about 60 nmol net charge per square meter. Preferably such ASB reagents are either peptides or modified peptides, including those commonly known as peptoids.

In certain embodiments, such ASB reagents are polycationic. Most preferably, the ASB reagents have a net charge of at least about positive two, at least about positive three, at least about positive four, at least about positive five, at least about positive six, at least about positive seven, at least about positive eight, at least about positive nine, at least about positive ten, at least about positive eleven, or at least about positive twelve at the pH at which a sample is contacted with the ASB reagent. The ASB reagants may have any net charge above about positive one. In general, as the net charge of the ASB reagent increases, the reagent will bind to aggregates over monomers with increased preference.

Preferably, the ASB reagents are attached to a solid support at a charge density of at least about 60 nmol net charge per square meter, at least about 90 nmol net charge per square meter, at least about 120 nmol net charge per square meter, at least about 500 nmol net charge per square meter, at least about 1000 nmol net charge per square meter, at least about 2000 nmol net charge per square meter, at least about 3000 nmol net charge per square meter, at least about 4000 nmol net charge per square meter, at least about 5000 nmol net charge per square meter, at least about 6000 nmol net charge per square meter, at least about 7000 nmol net charge per square meter, at least about 8000 nmol net charge per square meter, at least about 9000 nmol net charge per square meter, at least about 10,000 nmol net charge per square meter, at least about 12,000 nmol net charge per square meter, at least about 13,000 nmol net charge per square meter, at least about 14,000 nmol net charge per square meter, at least about 15,000 nmol net charge per square meter, at least about 16,000 nmol net charge per square meter, at least about 18,000 nmol net charge per square meter, at least about 20,000 nmol net charge per square meter, at least about 40,000 nmol net charge per square, at least about 60,000 nmol net charge per square meter, at least about 80,000 nmol net charge per square meter, at least about 100,000 nmol net charge per square meter, at least about 500,000 nmol net charge per square meter, at least about 1,000,000 nmol net charge per square meter, at least about 2,000,000 nmol net charge per square meter, at least about 2,400,000 nmol net charge per square meter, at least about 2,800,000 nmol net charge per square meter, at least about 3,000,000 nmol net charge per square meter, at least about 4,000,000 nmol net charge per square meter, at least about 5,000,000 nmol net charge per square meter, at least about 5,400,000 nmol net charge per square meter, at least about 6,000,000 nmol net charge per square meter, at least about 6,600,000 nmol net charge per square meter, or at least about 7,000,000 nmol net charge per square meter.

In certain embodiments, the ASB reagents are attached to a solid support at a charge density of at least about 10 nmol net charge per square meter, at least about 12 nmol net charge per square meter, at least about 20 nmol net charge per square meter, at least about 30 nmol net charge per square meter, at least about 40 nmol net charge per square meter, at least about 50 nmol net charge per square meter, at least about 60 nmol net charge per square meter, at least about 70 nmol net charge per square meter, at least about 80 nmol net charge per square meter, at least about 90 nmol net charge per square meter, at least about 100 nmol net charge per square meter, at least about 110 nmol net charge per square meter, at least about 120 nmol net charge per square meter, at least about 150 nmol net charge per square meter, at least about 200 nmol net charge per square meter, at least about 250 nmol net charge per square meter, at least about 300 nmol net charge per square meter, at least about 350 nmol net charge per square meter, at least about 400 nmol net charge per square meter, or at least about 450 nmol net charge per square meter. Applicants believe that ASB reagents attached to a solid support at this lower range of charge densities are likely only to bind preferentially to fibrils over monomers instead of to smaller aggregates over monomers.

In preferred embodiments, the ASB reagents have a binding affinity and/or avidity for aggregate that is at least about two times higher, at least about 2.5 times higher, at least about 3 times higher, at least about 3.5 times higher, at least about 4 times higher, at least about 4.5 times higher, at least about 5 times higher, at least about 5.5 times higher, at least about 6 times higher, at least about 6.5 times higher, at least about 7 times higher, at least about 7.5 times higher, at least about 8 times higher, at least about 8.5 times higher, at least about 9 times higher, at least about 9.5 times higher, at least about 10 times higher, or at least about 20 times higher than the binding affinity and/or avidity for monomer.

In preferred embodiments, the ASB reagents contain at least one positively-charged functional group having a pKa of at least 1 pH unit, of at least about 2 pH units, of at least about 3 pH units, or at least about 4 pH units higher then the pH at which a sample is contacted with the ASB reagent. Typically, a sample will be contacted with the ASB reagent at physiological pH. In certain embodiments, however, the pH may be lower or higher than physiological pH without it being detrimental to the sample. In such embodiments, the sample may be contacted with the ASB reagent at a pH of around 1, at a pH of around 2, at a pH of around 3, at a pH of around 4, at a pH of around 5, at a pH of around 6, at a pH of around 7, at a pH of around 8, at a pH of around 9, or at a pH of around 10.

In some embodiments, the ASB reagents also contain a hydrophobic functional group. The hydrophobic functional group may be, for example, an aromatic or an aliphatic hydrophobic functional group.

In certain embodiments, the ASB reagents may contain functional groups such as amines, alkyl groups, heterocycles, cycloalkanes, guanidine, ether, allyl, and aromatics. In certain embodiments, the aggregate-specific binding reagent includes an aromatic functional group selected from the group consisting of naphtyl, phenol, aniline, phenyl, substituted phenyl, nitrophenyl, halogenenated phenyl, biphenyl, styryl, diphenyl, benzyl sulfonamide, aminomethylphenyl, thiophene, indolyl, naphthyl, furan, and imidazole. In certain embodiments, the halogenenated phenyl is chlorophenyl or fluorophenyl. In certain embodiments, the aggregate-specific binding reagent includes an amine functional group selected from the group consisting of primary, secondary, tertiary, and quaternary amines. In certain embodiments, the aggregate-specific binding reagent includes an alkyl functional group selected from the group consisting of isobutyl, isopropyl, sec-butyl, and methyl and octyl. In certain embodiments, the aggregate-specific binding reagent includes. a heterocycle functional group selected from the group consisting of tetrohydrofuran, pyrrolidine, and piperidine. In certain embodiments, the aggregate-specific binding reagent includes a cycloalkane functional group selected from the group consisting of cyclopropyl and cyclohexyl. Such aromatic functional groups include naphtyl, phenol, and aniline. In further embodiments, the ASB reagents contain repeating motifs. In other embodiments, the ASB reagents contain positively charged groups with the same spacing as that of the negatively charged groups of an aggregate.

### A. ASB Peptide Reagents

In preferred embodiments, ASB reagents are peptides. Typically, ASB peptide reagents contain at least one net positive charge, at least two net positive charges, at least three net positive charges, at least four net positive charges, at least five net positive charges, at least six net positive charges, at least seven net positive charges, at least eight net positive charges, at least nine net positive charges, at least ten net positive charges, at least eleven net positive charges, or at least twelve net positive charges at the pH at which a sample is contacted with the ASB reagent. In preferred embodiments, the at least one amino acid is also positively charged at physiological pH. In preferred embodiments, the at least one amino acid is a natural amino acid such as lysine or arginine. In other embodiments, the at least one amino acid is an unnatural amino acid such as ornithine, methyllysine, diaminobutyric acid, homoarginine, or 4-aminomethylphenylalanine. In preferred embodiments, the ASB reagents contain a hydrophobic amino acid. The hydrophobic amino acid may be an aliphatic hydrophobic amino acid. In preferred embodiments, the hydrophobic amino acid is tryptophan, phenylalanine, valine, leucine, isoleucine, methionine, tyrosine, homophenylalanine, phenylglycine, 4-chlorophenylalanine, norleucine, norvaline, thienylalanine, 4-nitrophenylalanine, 4-aminophenylalanine, pentafluorophenylalanine, 2-naphthylalanine, p-biphenylalanine, styrylalanine, substituted phenylalanines, halogenated phenylalanines, aminoisobutyric acid, allyl glycine, cyclohexylalanine, cyclohexylglycine, 1-napthylalanine, pyridylalanine, or 1,2,3,4-tetrahydroisoquinoline-3-carboxylic acid.

ASB peptide reagents can include modifications to the specific ASB peptide reagents listed herein, such as deletions, additions and substitutions (generally conservative in nature), so long as the peptide maintains the desired characteristics. In certain embodiments, conservative amino acid replacements are preferred. Conservative amino acid replacements are those that take place within a family of amino acids that are related in their side chains. Genetically encoded amino acids are generally divided into four families: (1) acidic=aspartate, glutamate; (2) basic=lysine, arginine, histidine; (3) non-polar=alanine, valine, leucine, isoleucine, proline, phenylalanine, methionine, tryptophan; and (4) uncharged polar=glycine, asparagine, glutamine, cysteine, serine, threonine, tyrosine. Phenylalanine, tryptophan, and tyrosine are sometimes classified jointly as aromatic amino acids. For example, it is reasonably predictable that an isolated replacement of a leucine with an isoleucine or valine, an aspartate with a glutamate, a threonine with a serine, or a similar conservative replacement of an amino acid with a structurally related amino acid will not have a major effect on the biological activity. These modifications may be deliberate, as through site-directed mutagenesis, or may be accidental, such as through mutations of hosts that produce the proteins or errors due to PCR amplification. Furthermore, modifications may be made that have one or more of the following effects: increasing affinity, avidity, and/or specificity for aggregates; and increasing stability and resistance to proteases.

ASB peptide reagents may contain one or more analogs of an amino acid (including, for example, unnatural amino acids, etc.), peptides with substituted linkages, as well as other modifications known in the art, both naturally occurring and non-naturally occurring (e.g., synthetic). Thus, synthetic peptides, dimers, multimers (e.g., tandem repeats, multiple antigenic peptide (MAP) forms, linearly-linked peptides), cyclized, branched molecules and the like are considered to be peptides. This also includes molecules containing one or more N-substituted glycine residues (a “peptoid”) and other synthetic amino acids or peptides. (See, e.g., U.S. Pat. Nos. 5,831,005; 5,877,278; and 5,977,301; Nguyen et al. (2000) Chem. Biol. 7(7):463-473; and Simon et al. (1992) Proc. Natl. Acad. Sci. USA 89(20):9367-9371 for descriptions of peptoids).

For a general review of these and other amino acid analogs and peptidomimetics see, Nguyen et al. (2000) Chem. Biol. 7(7):463-473; Spatola, A. F., in Chemistry and Biochemistry of Amino Acids, Peptides and Proteins, B. Weinstein, eds., Marcel Dekker, New York, p. 267 (1983). See also, Spatola, A. F., Peptide Backbone Modifications (general review), Vega Data, Vol. 1, Issue 3, (March 1983); Morley, Trends Pharm Sci (general review), pp. 463-468 (1980); Hudson, D. et al., Int J Pept Prot Res, 14:177-185 (1979) (—CH₂NH—, CH₂CH₂—); Spatola et al., Life Sci, 38:1243-1249 (1986) (—CH₂—S); Hann J. Chem. Soc. Perkin Trans. I, 307-314 (1982) (—CH—CH—, cis and trans); Almquist et al., J Med Chem, 23:1392-1398 (1980) (—COCH₂—); Jennings-White et al., Tetrahedron Lett, 23:2533 (1982) (—COCH₂—); Szelke et al., European Appln. EP 45665 CA: 97:39405 (1982) (—CH(OH)CH₂—); Holladay et al., Tetrahedron Lett, 24:4401-4404 (1983) (—C(OH)CH₂—); and Hruby, Life Sci, 31:189-199 (1982) (—CH₂—S—).

It will also be apparent that any combination of the natural amino acids and non-natural amino acid analogs can be used to make the ASB reagents described herein. Commonly encountered amino acid analogs that are not gene-encoded include, but are not limited to, ornithine (Orn); aminoisobutyric acid (Aib); benzothiophenylalanine (BtPhe); albizziin (Abz); t-butylglycine (Tle); phenylglycine (PhG); cyclohexylalanine (Cha); norleucine (Nle); 2-naphthylalanine (2-Nal); 1-naphthylalanine (1-Nal); 2-thienylalanine (2-Thi); 1,2,3,4-tetrahydroisoquinoline-3-carboxylic acid (Tic); N-methylisoleucine (N-MeIle); homoarginine (Har); Nα-methylarginine (N-MeArg); phosphotyrosine (pTyr or pY); pipecolinic acid (Pip); 4-chlorophenylalanine (4-ClPhe); 4-fluorophenylalanine (4-FPhe); 1-aminocyclopropanecarboxylic acid (1-NCPC); 4-aminomethylphenylalanine (AmF); and sarcosine (Sar). Any of the amino acids used in the ASB reagents may be either the D- or, more typically, L-isomer.

Other non-naturally occurring analogs of amino acids that may be used to form the ASB reagents described herein include peptoids and/or peptidomimetic compounds such as the sulfonic and boronic acid analogs of amino acids that are biologically functional equivalents are also useful in the compounds of the present invention and include compounds having one or more amide linkages optionally replaced by an isostere. In the context of the present invention, for example, —CONH— may be replaced by —CH₂NH—, —NHCO—, —SO₂NH—, CH₂O—, —CH₂CH₂—, CH₂S—, CH₂SO—, —CH—CH— (cis or trans), —COCH₂—, —CH(OH)CH₂— and 1,5-disubstituted tetrazole such that the radicals linked by these isosteres would be held in similar orientations to radicals linked by —CONH—. One or more residues in the ASB reagents described herein may include N-substituted glycine residues.

Thus, the reagents also may include one or more N-substituted glycine residues (peptides having one or more N-substituted glycine residues may be referred to as “peptoids”). For example, in certain embodiments, one or more proline residues of any of the ASB reagents described herein are replaced with N-substituted glycine residues. Particular N-substituted glycines that are suitable in this regard include, but are not limited to, N—(S)-(1-phenylethyl)glycine; N-(4-hydroxyphenyl)glycine; N-(cyclopropylmethyl)glycine; N-(isopropyl)glycine; N-(3,5-dimethoxybenzyl)glycine; and N-butylglycine. Other N-substituted glycines may also be suitable to replace one or more amino acid residues in the ASB reagent sequences described herein.

The ASB reagents described herein may be monomers, multimers, cyclized molecules, branched molecules, linkers and the like. Multimers (i.e., dimers, trimers and the like) of any of the sequences described herein or biologically functional equivalents thereof are also contemplated. The multimer can be a homomultimer, i.e., composed of identical monomers, e.g., each monomer is the same peptide sequence. Alternatively, the multimer can be a heteromultimer, by which is meant that not all the monomers making up the multimer are identical.

Multimers can be formed by the direct attachment of the monomers to each other or to substrate, including, for example, multiple antigenic peptides (MAPS) (e.g., symmetric MAPS), peptides attached to polymer scaffolds, e.g., a PEG scaffold and/or peptides linked in tandem with or without spacer units.

Alternatively, linking groups can be added to the monomeric sequences to join the monomers together and form a multimer. Non-limiting examples of multimers using linking groups include tandem repeats using glycine linkers; MAPS attached via a linker to a substrate and/or linearly linked peptides attached via linkers to a scaffold. Linking groups may involve using bifunctional spacer units (either homobifunctional or heterobifunctional) as are known to one of skill in the art. By way of example and not limitation, many methods for incorporating such spacer units in linking peptides together using reagents such as succinimidyl-4-(p-maleimidomethyl)cyclohexane-1-carboxylate (SMCC), succinimidyl-4-(p-maleimidophenyl)butyrate and the like are described in the Pierce Immunotechnology Handbook (Pierce Chemical Co., now Thermo Fisher, Rockville, Ill.) and are also available from Sigma Chemical Co. (St. Louis, Mo.) and Aldrich Chemical Co. (Milwaukee, Wis.) (now Sigma-Aldrich, St. Louis, Mo.) and described in “Comprehensive Organic Transformations”, VCK-Verlagsgesellschaft, Weinheim/Germany (1989). One example of a linking group which may be used to link the monomeric sequences together is —Y₁—F—Y₂ where Y₁ and Y₂ are identical or different and are alkylene groups of 0-20, preferably 0-8, more preferably 0-3 carbon atoms, and F is one or more functional groups such as —O—, —S—S—, —C(O)—O—, —NR—, —C(O)—NR—, —NR—C(O)—O—, —NR—C(O)—NR—, —NR—C(S)—NR—, —NR—C(S)—O—. Y₁ and Y₂ may be optionally substituted with hydroxy, alkoxy, hydroxyalkyl, alkoxyalkyl, amino, carboxyl, carboxyalkyl and the like. It will be understood that any appropriate atom of the monomer can be attached to the linking group.

Further, the ASB reagents described herein may be linear, branched or cyclized. Monomer units can be cyclized or may be linked together to provide the multimers in a linear or branched fashion, in the form of a ring (for example, a macrocycle), in the form of a star (dendrimers) or in the form of a ball (e.g., fullerenes). Skilled artisans will readily recognize a multitude of polymers that can be formed from the monomeric sequences disclosed herein. In certain embodiments, the multimer is a cyclic dimer. Using the same terminology as above, the dimer can be a homodimer or a heterodimer.

Cyclic forms, whether monomer or multimer, can be made by any of the linkages described above, such as but not limited to, for example: (1) cyclizing the N-terminal amine with the C-terminal carboxylic acid either via direct amide bond formation between the nitrogen and the C-terminal carbonyl, or via the intermediacy of spacer group such as for example by condensation with an epsilon-amino carboxylic acid; (2) cyclizing via the formation of a bond between the side chains of two residues, e.g., by forming a amide bond between an aspartate or glutamate side chain and a lysine side chain, or by disulfide bond formation between two cysteine side chains or between a penicillamine and cysteine side chain or between two penicillamine side chains; (3) cyclizing via formation of an amide bond between a side chain (e.g., aspartate or lysine) and either the N-terminal amine or the C-terminal carboxyl respectively; and/or (4) linking two side chains via the intermediacy of a short carbon spacer group.

Furthermore, the ASB reagents described herein may also include additional peptide or non-peptide components. Non-limiting examples of additional peptide components include spacer residues, for example two or more glycine (natural or derivatized) residues or aminohexanoic acid linkers on one or both ends or residues that may aid in solubilizing the peptide reagents, for example acidic residues such as aspartic acid (Asp or D). In certain embodiments, for example, the peptide reagents are synthesized as multiple antigenic peptides (MAPs). Typically, multiple copies of the peptide reagents (e.g., 2-10 copies) are synthesized directly onto a MAP carrier such as a branched lysine or other MAP carrier core. See, e.g., Wu et al. (2001) J Am Chem. Soc. 2001 123(28):6778-84; Spetzler et al. (1995) Int J Pept Protein Res. 45(1):78-85.

Non-limiting examples of non-peptide components (e.g., chemical moieties) that may be included in the ASB reagents described herein include, one or more detectable labels, tags (e.g., biotin, His-Tags, oligonucleotides), dyes, members of a binding pair, and the like, at either terminus or internal to the peptide reagent. The non-peptide components may also be attached (e.g., via covalent attachment of one or more labels), directly or through a spacer (e.g., an amide group), to position(s) on the compound that are predicted by quantitative structure-activity data and/or molecular modeling to be non-interfering. ASB Reagents as described herein may also include chemical moieties such as amyloid-specific dyes (e.g., Congo Red, Thioflavin, etc.). Derivatization (e.g., labeling, cyclizing, attachment of chemical moieties, etc.) of compounds should not substantially interfere with (and may even enhance) the binding properties, biological function and/or pharmacological activity of the reagent.

The above described peptides can be prepared using standard methods known to those of skill in the art, including but not limited to expression from recombinant constructs and peptide synthesis.

### B. Examples of Preferred Peptides to be Used as Basis for ASB Reagents

Non-limiting examples of peptides useful in making the aggregate-specific binding reagents of the invention preferably derived from sequences shown in Table 2. The peptides in the table are represented by conventional one letter amino acid codes and are depicted with their amino-terminus at the left and carboxy-terminus at the right.

Any of the sequences in the table may optionally include Gly linkers (Gn where n=1, 2, 3, or 4) at the amino- and/or carboxy-terminus. Typically, aminohexanoic acid (Ahx) is used as a linker. Any of the sequences in the table may also optionally include a capping group at the amino- and/or carboxy-terminus. One example of such a capping group is an acetyl group. It is preferred that the capping group is not negatively charged.

TABLE 2
Peptide sequences for making ASB reagents

| Peptide sequence | SEQ ID NO |
|---|---|
| KKKFKF | 1 |
| KKKWKW | 2 |
| KKKLKL | 3 |
| KKKKKK | 4 |
| KKKKKKKKKKKK | 5 |
| KFYLYAIDTHRM | 6 |
| KIIKWGIFWMQG | 7 |
| NFFKKFRFTFTM | 8 |
| AAKKAA | 32 |
| AAKKKA | 33 |
| AKKKKA | 34 |
| AKKKKK | 35 |
| FKFKKK | 36 |
| kkkfkf | 37 |
| FKFSLFSG | 38 |
| DFKLNFKF | 39 |
| FKFNLFSG | 40 |
| YKYKKK | 41 |
| KKFKKF | 42 |
| KFKKKF | 43 |
| KIGVVR | 44 |
| AKVKKK | 45 |
| AKFKKK | 46 |
| RGRERFEMFR | 47 |
| YGRKKRRQRRR | 48 |
| FFFKFKKK | 49 |
| FFFFKFKKK | 50 |
| FFFKKK | 51 |
| FFFFKK | 52 |
| F-fdb-F-fdb-fdb-fdb | 53 |
| FoFooo | 54 |
| monoBoc-ethylenediamine + BrCH2CO-KKFKF | 55 |
| triethylamine + BrCH2CO-KKFKF | 56 |
| tetramethylethylenediamine + BrCH2CO-KKFKF | 57 |
| Ala-AmF-AmF-Phe-AmF-Ala | 58 |
| XKXKKK | 59 |
| X = Thi, thienylalanine | |
| KKKXKX | 60 |
| X = 4-Cl Phe, 4-chlorophenylalanine | |
| KKKXKX | 61 |
| X = 4-NO2, 4-nitrophenylalanine | |
| XKXKKK | 62 |
| X = F5Phe, pentafluorophenylalanine | |
| XKXKKK | 63 |
| X = Nap, 2-naphthylalanine | |
| XKXKKK | 64 |
| X = Bip, p-biphenylalanine | |
| XKXKKK | 65 |
| X = Sty, styrylalanine | |
| XKXKKK | 66 |
| X = Tic, 1,2,3,4- | |
| tetrahydroisoquinoline-3-carboxylic acid | |
| MKFMKMHNKKRY | 67 |
| LTAVKKVKAPTR | 68 |
| LIPIRKKYFFKL | 69 |
| KLSLIWLHTHWH | 70 |
| IRYVTHQYILWP | 71 |
| YNKIGVVRLFSE | 72 |
| YRHRWEVMLWWP | 73 |
| WAVKLFTFFMFH | 74 |
| YQSWWFFYFKLA | 75 |
| WWYKLVATHLYG | 76 |
| QTLSLHFQTRPP | 77 |
| TRLAMQYVGYFW | 78 |
| RYWYRHWSQHDN | 79 |
| AQYIMFKVFYLS | 80 |
| TGIRIYSWKMWL | 81 |
| SRYLMYVNIIYI | 82 |
| RYWMNAFYSPMW | 83 |
| NFYTYKLAYMQM | 84 |
| MGYSSGYWSRQV | 85 |
| YFYMKLLWTKER | 86 |
| RIMYLYHRLQHT | 87 |
| RWRHSSFYPIWF | 88 |
| QVRIFTNVEFKH | 89 |
| RYLHWYAVAVKV | 90 |

Unnatural Amino Acids

| Symbol | Description |
|---|---|
| Dab | the gamma amino acid 2,4-diaminobutanoic acid |
| O | ornithane |
| o | the delta amino acid 2, 5-diaminopentanoic acid |
| 5FPhe | pentafluorophenylalanine |
| Nap | 2-naphthylalanine |
| Peptide sequence | SEQ ID NO |
| Bip | p-biphenylalanine |
| Sty | styrylalanine |
| Tic | 1,2,3,4-tetrahydroisoquinoline-3-carboxylic acid |
| Fdb | the alpha amino acid 2,4-diaminobutanoic acid |
| Thi | thienylalanine |
| AmF | 4-aminomethylphenylalanine |
| 4Cl-F | 4-chlorophenylalanine |
| 4NO2-F | 4-nitrophenylalanine |

### C. Peptoid ASB Reagents

In particularly preferred embodiments, the ASB reagents are peptoids. Methods for making peptoids are disclosed in U.S. Pat. Nos. 5,811,387 and 5,831,005, as well as methods disclosed herein. Preferred peptoids are described below. ASB peptoid reagents can include modifications to the specific ASB peptoid reagents listed herein, such as deletions, additions and substitutions (generally conservative in nature), so long as the peptoid maintains the desired characteristics.

### Preferred Peptoid Sequences

Table 3 lists example peptoid regions (amino to carboxy directed) suitable for preparing ASB reagents to be used in this invention. Table 4 provides a key to the abbreviations used in Table 3. Table 5 provides the relevant structures of each of the sequences. Preparations of the specific ASB reagents are described herein below.

TABLE 3
Representative peptoid reagents for ASB reagents

| | SEQ |
|---|---|
| | ID |
| Peptoid Region Sequence | NO: |
| Nab-Nab-Nab-Nst-Nab-Nst | 9 |
| Nae-Nae-Nae-Nbn-Nae-Nbn | 10 |
| Nab-Nab-Nab-Noc-Nab-Noc | 11 |
| Ngb-Ngb-Ngb-Nbn-Ngb-Nbn | 12 |
| Nab-Nab-Nab-Nbn-Nab-Nbn-Nab-Nab-Nab- | 13 |
| Nbn-Nab-Nbn | |
| Nab-Nab-Nab-Nbn-Nab-Nbn-Nab-Nab-Nab- | 14 |
| Nbn-Nab-Nbn-Nab-Nab-Nab-Nbn-Nab-Nbn | |
| Nab-Nab-Nab-Nbn-Nab-Nbn | 15 |
| Nab-Nab-Nab-Nab-Nab-Nab | 16 |
| Nab-Nab-Nab-Nab-Nab-Nab-Nab | 17 |
| Nab-Nab-Nbn-Nab-Nbn-Nab-Nbn | 18 |
| Nab-Nbn-Nab-Nbn-Nab-Nbn-Nab | 19 |
| Nab-Nab-Nab-Nbn-Nbn-Nbn-Nab | 20 |
| Nea-Ndpc-Napp-Nffb-Nme-Nthf | 91 |
| Nall-Nhpe-Ncpm-Nchm-Ngab | 92 |
| Nmba-Nfur-Nbn-Nlys-Nea-Nbsa | 93 |
| Namp-Ncpm-Nhye-Nffb-Nlys-Nchm | 94 |
| Nglu-Nlys-Nhpe-Nbsa-Nme-Nea | 95 |
| (Nlys-Nspe-Nspe)4 | 96 |

TABLE 4
Abbreviations key to Table 3.

| Peptoid Residue | |
|---|---|
| Abbreviation | Amino Acid Substitute |
| Nab | N-(4-aminobutyl)glycine |
| Nae = Nea | N-(4-aminoethyl)glycine |
| Nall | N-allylglycine |
| Namp | N-(piperidin-4-ylmethyl)glycine |
| Napp | 3-(2-oxopyrrolidin-1-yl)propyl)glycine |
| Nbn | N-benzylglycine |
| Nbsa | N-(4-sulfamoylphenethyl)glycine |
| Nbzp | 2-(4-benzoylbenzyl)glycine |
| NChm | N-(cyclohexylmethyl)glycine |
| Ncpm | N-(cyclopropylmethyl)glycine |
| Ncpm | N-(cyclopropylmethyl)glycine |
| Ndmb | N-(3,5-dimethoxybenzyl)glycine |
| Ndpc | N-(2,2-diphenylethyl)glycine |
| Nffb | N-(3,4-difluorobenzyl)glycine |
| Nfur | N-(3-furylmethyl)glycine |
| Ngab | N-(4-carboxyethyl)glycine |
| Ngb | N-(4-guanidinobutyl)glycine |
| Nglu | N-(2-carboxyethyl)glycine |
| Nglu | N-(2-carboxyethyl)glycine |
| Nhpe = Ntyr | N-(2-(4-hydroxyphenyl)ethyl)glycine |
| Nhph | N-(4-hydroxyphenyl)glycine |
| Nhrg = Ngb | N-(4-guanidinobutyl)glycine |
| Nhye | N-(2-hydroxyethyl)glycine |
| Nip | N-isopropylglycine |
| Nlys | N-(4-aminobutyl)glycine |
| Nmba | N-(4-methoxybenzyl)glycine |
| Nme | N-(2-methoxyethyl)glycine |
| Nmpe | N-(2-(4-methoxyphenyl)ethyl)glycine |
| Nnm | N-((8'-naphthyl)methyl)glycine |
| Noc | N-(octyl)glycine |
| Noct | N-octylglycine |
| Nspe | (S)-N-(1-phenylethyl)glycine |
| Nst | N-(methylstilbene)glycine |
| Nstl | N-(methylstilbene)glycine |
| Nthf | N-tetrahydrofufurylglycine |
| Ntrp | N-(2-3'-indolylethyl)glycine |
| Ntyr | N-(2-(4-hydroxyphenyl)ethyl)glycine |

TABLE 5
Relevant structures of peptoid regions of Table 3.

| SEQ | |
|---|---|
| ID | |
| NO: | Structure |
| 9 | 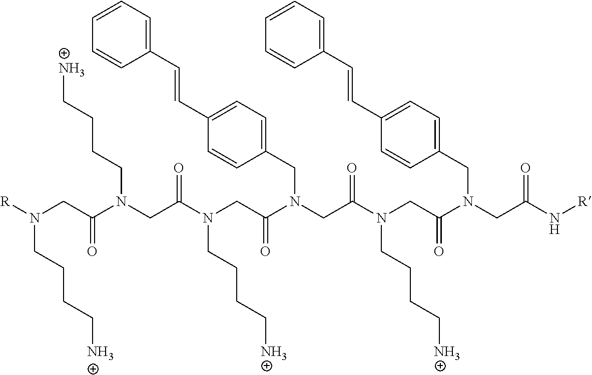 |
| 10 | 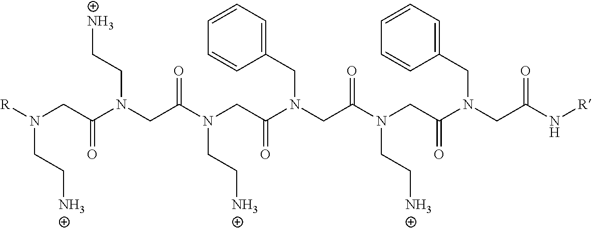 |
| 11 | 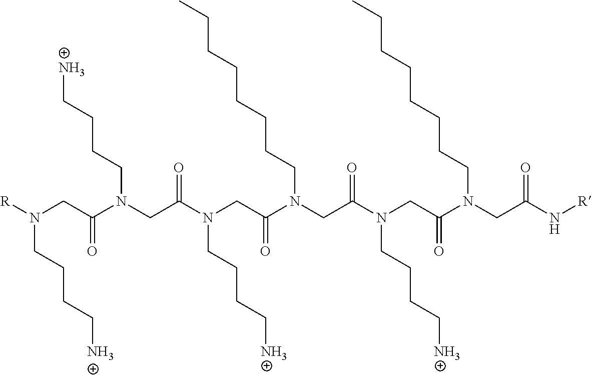 |
| 12 | 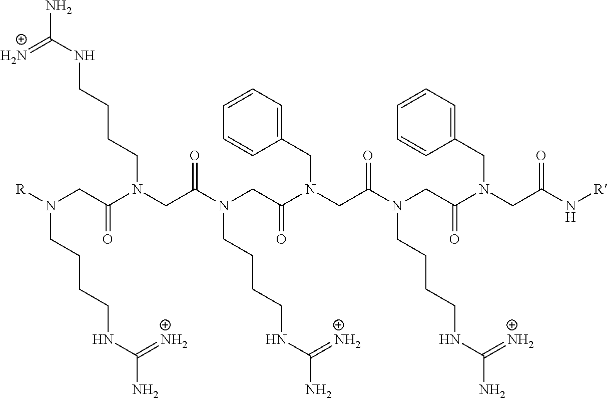 |
| 13 | 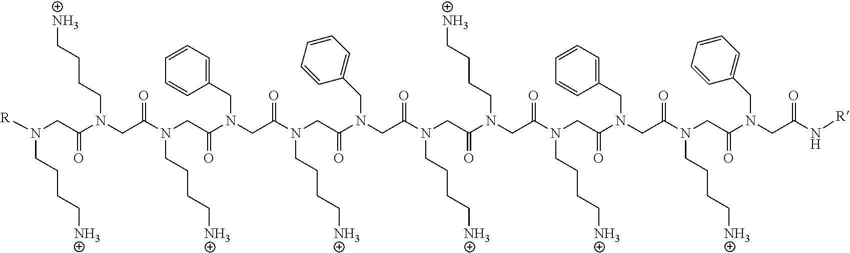 |
| 14 | 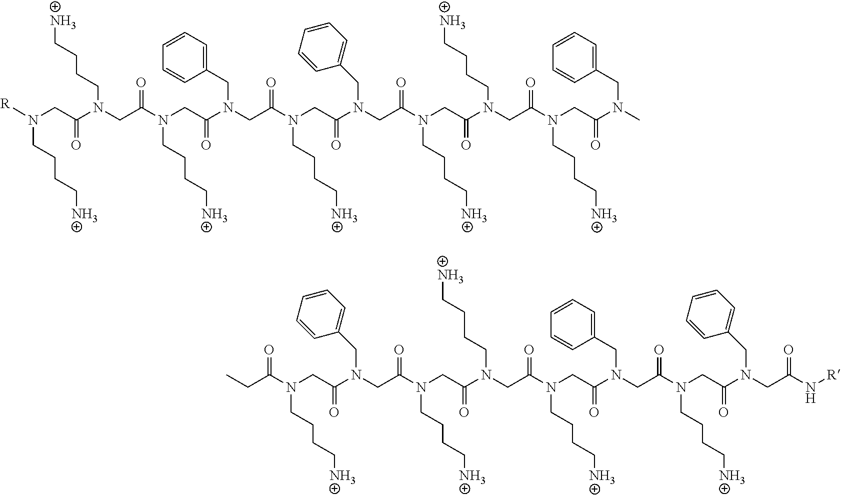 |
| 15 | 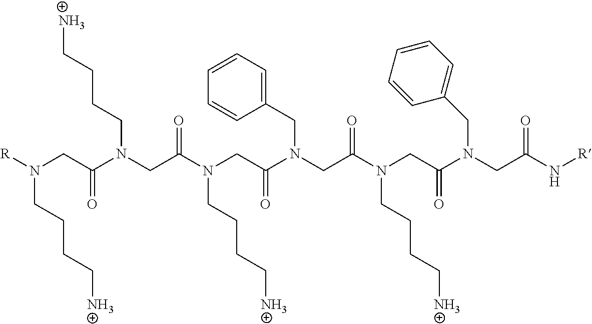 |
| 16 | 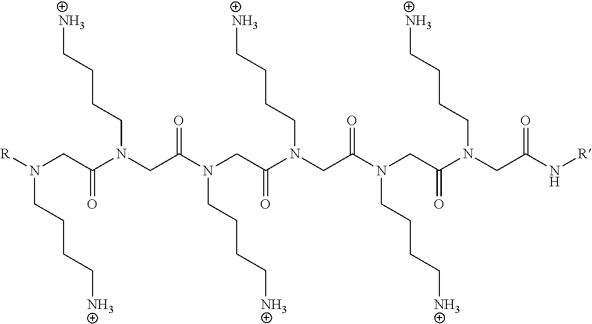 |
| 17 | 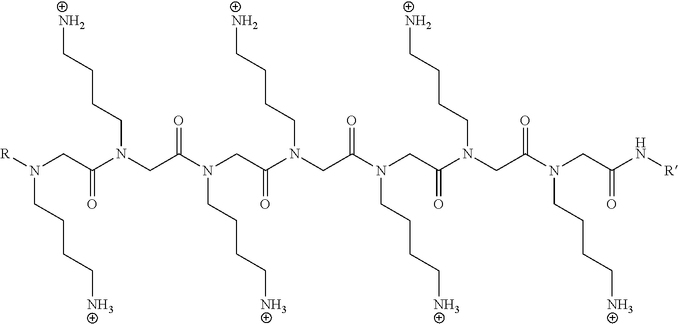 |
| 18 | 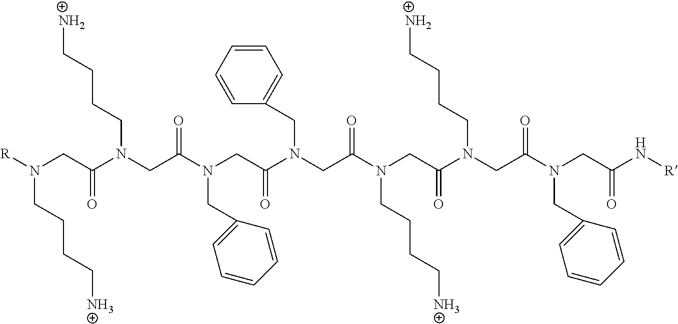 |
| 19 | 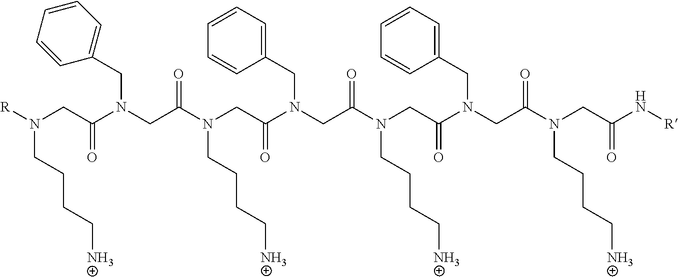 |
| 20 | 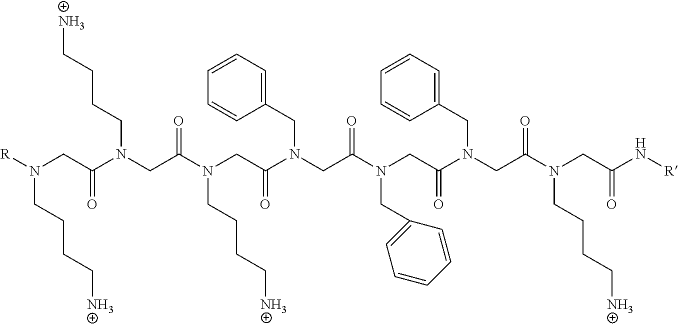 |
| 91 | 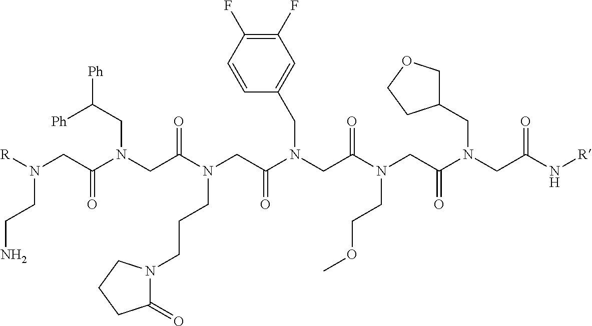 |
| 92 | 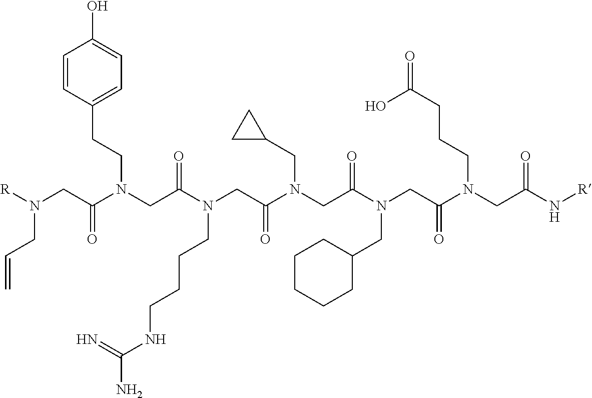 |
| 93 | 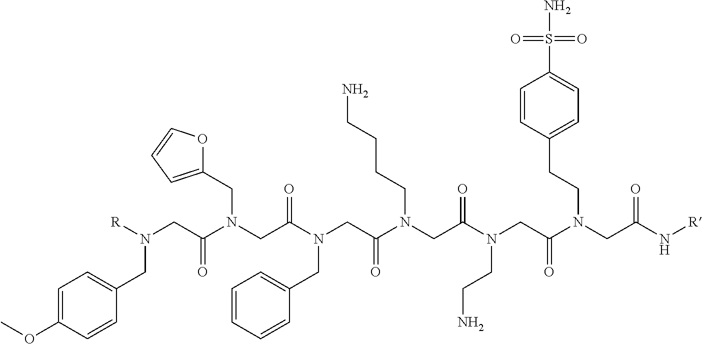 |
| 94 | 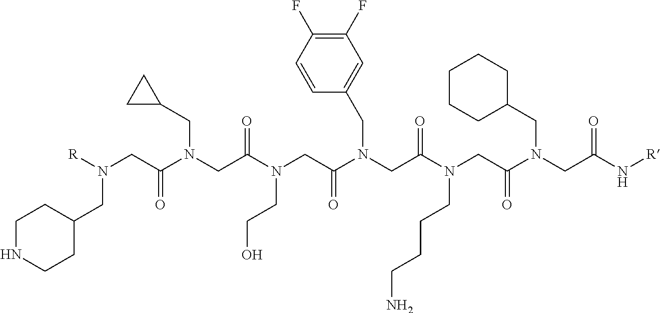 |
| 95 | 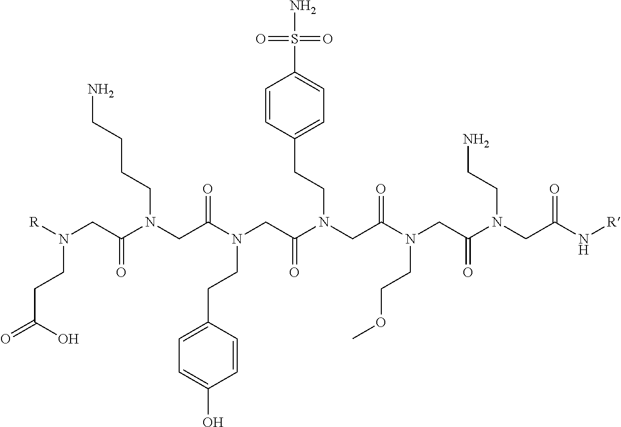 |
| 96 | 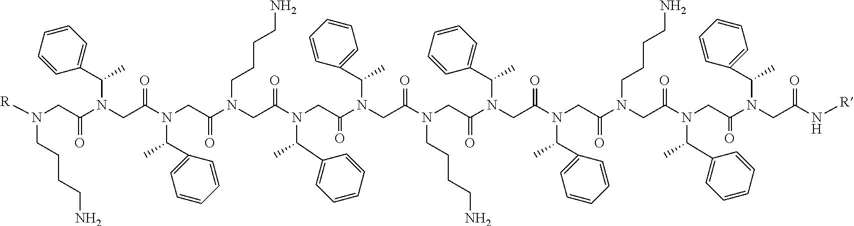 |

In a particularly preferred embodiment, the ASB reagent contains the structure of PSR1:

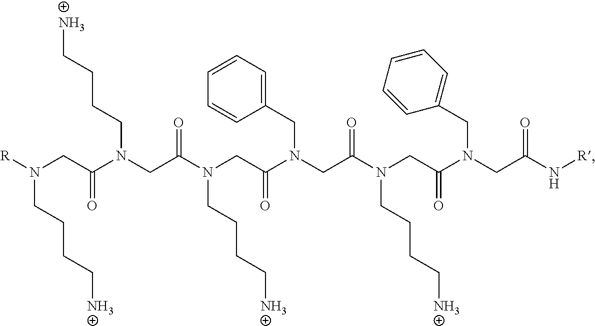

where R and R′ can be any group.
D. ASB Reagents from Other Scaffolds

In certain embodiments of the invention, the ASB reagents include positively charged organic molecule scaffolds other than peptides and peptoids. In preferred embodiments, the ASB reagents are dendrons. In a particularly preferred embodiment, the ASB reagent includes the structure of

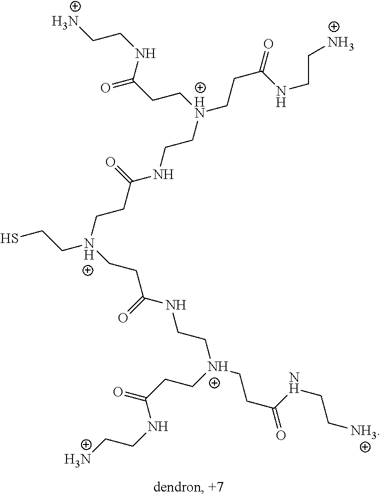

E. Identifying ASB Reagents to be Used in Methods of this Invention

The ASB reagents to be used in methods of this invention bind preferentially to aggregates over monomers when attached to a solid support at certain charge densities. This property can be tested using any known binding assay, for example standard immunoassays such as ELISAs, Western blots and the like; labeled peptides; ELISA-like assays; and/or cell-based assays, in particular those assays described in the below section regarding “Detection of Aggregates by Binding of Aggregate to ASB Reagent”.

One convenient method of testing the specificity of the ASB reagents used in methods of the present invention is to select a sample containing both aggregates and monomers. Typically such samples include tissue from diseased animals. ASB reagents as described herein that are known to bind specifically to aggregates are attached to a solid support (by methods well-known in the art and as further described below) and used to separate (“pull down”) aggregate from the other sample components and obtain a quantitative value directly related to the number of reagent-protein binding interactions on the solid support. This result can be compared to that of an ASB reagent with unknown binding specificity to determine whether such reagent can bind preferentially to aggregates.

### III. DETECTION OF AGGREGATES BY BINDING OF AGGREGATE TO ASB REAGENT

The described ASB reagents can be used in a variety of assays to screen samples (e.g., biological samples such as blood, brain, spinal cord, CSF or organ samples), for example, to detect the presence or absence of aggregates in these samples. Unlike many current reagents, the ASB reagents described herein will allow for detection in virtually any type of biological, including blood sample, blood products, CSF, or biopsy samples, or non-biological sample.

The detection methods can be used, for example, in methods for diagnosing a disease associated with an aggregate and any other situation where knowledge of the presence or absence of the aggregate is important.

Use of Aggregate-Specific Binding Reagents as either Capture or Detection Reagents

The ASB reagents to be used in methods of the invention typically have a net charge of at least about positive one at the pH at which a sample is contacted with the ASB reagent, are attached to a solid support at a charge density of at least about 60 nmol net charge per square meter, and bind preferentially with aggregates over monomers when attached to the solid support. For samples expected to contain aggregates of more than one conformational protein or where it is critical for purposes of the method to determine which type of aggregate is present, aggregate-specific binding reagents should be used for detection in combination with CPSB reagents which have different binding specificities and/or affinities for different types of conformational proteins. For example, if the aggregate-specific binding reagent is used as a capture reagent, a conformational protein-specific binding reagent should be used as a detection reagent or vice versa. If, however, the particular sample to be assayed is expected to only contain a single type of aggregate or if it is not critical for the purposes of the method to determine which aggregate is present, then the ASB reagent can be used as both a capture and detection reagent.

### Methods Using Aggregate-Specific Binding Reagents as Capture Agents

In preferred embodiments, the invention provides methods for detecting the presence of an aggregate in a sample by contacting a sample suspected of containing an aggregate with an aggregate-specific binding reagent under conditions that allow binding of the reagent to the aggregate, if present; and detecting the presence of the aggregate, if any, in the sample by its binding to the reagent; where the aggregate-specific binding reagent has a net charge of at least about positive one at the pH at which the sample is contacted with the ASB reagent, is attached to a solid support at a charge density of at least about 60 nmol net charge per square meter, and binds preferentially with aggregates over monomers when attached to the solid support.

For use in methods of the invention, the sample can be anything known to, or suspected of, containing an aggregate. The sample can be a biological sample (that is, a sample prepared from a living or once-living organism) or a non-biological sample. Typically, a biological sample contains bodily tissues or fluid. Suitable biological samples include, but are not limited to whole blood, blood fractions, blood components, plasma, platelets, serum, cerebrospinal fluid (CSF), bone marrow, urine, tears, milk, lymph fluid, organ tissue, brain tissue, nervous system tissue, muscle tissue, non-nervous system tissue, biopsy, necropsy, fat biopsy, cells, feces, placenta, spleen tissue, lymph tissue, pancreatic tissue, bronchoalveolar lavage, or synovial fluid. Preferred biological samples include plasma and CSF. In certain embodiments, the sample contains polypeptide.

The sample is contacted with one or more ASB reagents described herein under conditions that allow the binding of the ASB reagent(s) to the aggregate if it is present in the sample. It is well within the competence of one of ordinary skill in the art to determine the particular conditions based on the disclosure herein. Typically, the sample and the ASB reagent(s) are incubated together in a suitable buffer at physiological pH at a suitable temperature (e.g., about 4-37° C.), for a suitable time period (e.g., about 1 hour to overnight) to allow the binding to occur.

In these embodiments of the method, the aggregate-specific binding reagent is a capture reagent and the presence of aggregate in the sample is detected by its binding to the aggregate-specific binding reagent. After capture, the presence of the aggregate may be detected by the very same aggregate-specific binding reagent serving simultaneously as a capture and detection reagent. Alternatively, there can be a distinct detection reagent, which can be either a different aggregate-specific binding reagent or, preferably, one or more conformational protein-specific binding reagents. In preferred embodiments, the CPSB reagent is a labeled antibody. In preferred embodiments, after the capture step, the unbound sample is removed, the aggregate is dissociated from the complex it forms with the ASB reagent to provide a dissociated aggregate. The dissociated aggregate is contacted with a first CPSB reagent to allow formation of a second complex, and the presence of aggregate in the sample is detected by detecting the formation of the second complex. In preferred embodiments, the formation of the second complex is detected using a detectably labeled second CPSB reagent. The first CPSB reagent is preferably coupled to a solid support. In particularly preferred embodiments, the aggregate contains an Abeta protein and the CPSB reagent is an anti-Abeta antibody.

### Methods Using Aggregate-Specific Binding Reagents as Detection Agents

In other embodiments, the invention provides methods for detecting the presence of an aggregate in a sample by contacting a sample suspected of containing an aggregate with a conformational protein-specific binding reagent which binds to both monomers and aggregates of the conformational protein under conditions that allow the binding of the CPSB reagent to the aggregate, if present, to form a first complex; contacting the first complex with an ASB reagent under conditions that allow binding, and detecting the presence of the aggregate, if any, in the sample by its binding to the ASB reagent, where the ASB reagent has a net charge of at least about positive one at the pH at which the sample is contacted with the ASB reagent, is attached to a solid support at a charge density of at least about 60 nmol net charge per square meter, and binds preferentially with aggregates over monomers when attached to the solid support. Typically, after the capture step the unbound sample is removed. The CPSB reagent is preferably couple to a solid support.

### A. Reagents to Capture Aggregates

In preferred embodiments, the capture reagent is an aggregate-specific binding reagent which has a net charge of at least about positive one at the pH at which the sample is contacted with the ASB reagent, is attached to a solid support at a charge density of at least about 60 nmol net charge per square meter, and binds preferentially with aggregates over monomers when attached to the solid support. In other embodiments, the capture reagent is a conformational protein-specific binding reagent which binds to both monomers and aggregates of the conformational protein.

Capture reagents are contacted with samples under conditions that allow any aggregates in the sample to bind to the reagent and form a complex. Such binding conditions are readily determined by one of ordinary skill in the art and are further described herein. Typically, the method is carried out in the wells of a microtiter plate or in small volume plastic tubes, but any convenient container will be suitable. The sample is generally a liquid sample or suspension and may be added to the reaction container before or after the capture reagent.

If the capture reagent is an aggregate-specific binding reagent described above, it is coupled to a solid support of preferably at least about 60 nmol net charge per square meter.

If the capture reagent is instead a CPSB reagent, it is preferably coupled to a solid support, which is described in further detail in the following section. In some embodiments, the solid support is attached prior to application of the sample. A solid support (e.g., magnetic beads) is first reacted with a capture reagent as described herein such that the capture reagent is sufficiently immobilized to the support. The solid support with attached capture reagent is then contacted with a sample suspected of containing aggregates under conditions that allow the capture reagent to bind to aggregates.

Alternatively, if the capture reagent is a CPSB reagent, it may be first contacted with the sample suspected of containing aggregates before being attached to the solid support, followed by attachment of the capture reagent to the solid support (for example, the reagent can be biotinylated and the solid support includes avidin or streptavidin linked to a solid support).

In certain embodiments, after a complex between the capture reagent and aggregate is established, unbound sample material (that is, any components of the sample that have not bound to the capture reagent, including any unbound aggregates) can be removed. For example, if the capture reagent is coupled to a solid support, unbound materials can be reduced by separating the solid support from the reaction solution (containing the unbound sample materials) for example, by centrifugation, precipitation, filtration, magnetic force, etc. The solid support with the complex may optionally be subjected to one or more washing steps to remove any residual sample materials before carrying out the next steps of the method.

In some embodiments, following the removal of unbound sample materials and any optional washes, the bound aggregates are dissociated from the complex and detected using any known detection method. Alternatively, the bound aggregates in the complex are detected without dissociation from the capture reagent.

### B. Dissociation and Denaturation of Aggregate

After being bound to the capture reagent to form a complex, the aggregate may be treated to facilitate detection of the aggregate.

In some embodiments, the unbound material is removed and the aggregate is then dissociated from the complex. “Dissociation” refers to the physical separation of the aggregate from the capture reagent such that the aggregate can be detected separately from the capture reagent. Dissociation of the aggregate from the complex can be accomplished, for example using low concentration (e.g., 0.4 to 1.0 M) of guanidinium hydrochloride or guanidinium isothiocyanate.

When the CPSB reagent used in the method is only capable of detecting denatured protein, the dissociated aggregate is also denatured. “Denaturation” refers to disrupting the native conformation of a polypeptide. Denaturation without dissociation from the reagent can be accomplished, for example, if the reagent contains an activatable reactive group (e.g., a photoreactive group) that covalently links the reagent and the aggregate.

In preferred embodiments, the aggregate is simultaneously dissociated and denatured.

Aggregates may be simultaneously dissociated and denatured using high concentrations of salt or chaotropic agent, e.g., between about 3M to about 6M of a guanidinium salt such as guanidinium thiocyanate (GdnSCN), or guanidinium HCl (GdnHCl). Preferably, the chaotropic agent is removed or diluted before detection is carried out because they may interfere with binding of the detection reagent.

In other embodiments, the aggregate is simultaneously dissociated from the complex with the capture reagent and denatured by altering pH, e.g., by either raising the pH to 12 or above (“high pH”) or lowering the pH to 2 or below (“low pH”). Exposure of the complex to high pH is preferred. A pH of between 12.0 and 13.0 is generally sufficient; preferably, a pH of between 12.5 and 13.0, of between 12.7 to 12.9, or of 12.9 is used. Alternatively, exposure of the complex to a low pH can be used to dissociate and denature the pathogenic protein from the reagent. For this alternative, a pH of between 1.0 and 2.0 is sufficient. In some embodiments, the aggregate is treated with pH 12.5-13.2 for a suitable amount of time, e.g., 90° C. for 10 minutes.

Exposure of the first complex to either a high pH or a low pH is generally carried out for only a short time e.g. 60 minutes, preferably for no more than 15 minutes, more preferably for no more than 10 minutes. In some embodiments, the exposure is carried out above room temperature, for example, at about 60° C., 70° C., 80° C., or 90° C. After exposure for sufficient time to dissociate the aggregate, the pH can be readily readjusted to neutral (that is, pH of between about 7.0 and 7.5) by addition of either an acidic reagent (if high pH dissociation conditions are used) or a basic reagent (if low pH dissociation conditions are used). One of ordinary skill in the art can readily determine appropriate protocols and examples are described herein.

In general, to affect a high pH dissociation condition, addition of NaOH to a concentration of about 0.05 N to about 0.2 N is sufficient. Preferably, NaOH is added to a concentration of between about 0.05 N to about 0.15 N; more preferably, about 0.1 N NaOH is used. Once the dissociation is accomplished, the pH can be readjusted to neutral (that is, between about 7.0 and 7.5) by addition of suitable amounts of an acidic solution, e.g., phosphoric acid, sodium phosphate monobasic.

In general, to affect a low pH dissociation condition, addition of H₃PO₄ to a concentration of about 0.2 M to about 0.7 M is sufficient. Preferably, H₃PO₄ is added to a concentration of between 0.3 M and 0.6 M; more preferably, 0.5 M H₃PO₄ is used. Once the dissociation is accomplished, the pH can be readjusted to neutral (that is, between about 7.0 and 7.5) by addition of suitable amounts of a basic solution, e.g., NaOH or KOH.

If desirable, dissociation of the aggregate from the complex can also be accomplished without denaturing the protein, for example using low concentration (e.g., 0.4 to 1.0 M) of guanidinium hydrochloride or guanidinium isothiocyanate. See, WO2006076497 (International Application PCT/US2006/001090) for additional conditions for dissociating the aggregate from the complex without denaturing the protein. Alternatively, the captured aggregates can be also denatured without dissociation from the reagent if, for example, the reagent is modified to contain an activatable reactive group (e.g., a photoreactive group) that can be used to covalently link the reagent and the aggregate.

After dissociation, the aggregate is then separated from the capture reagent. This separation can be accomplished in similar fashion to the removal of the unbound sample materials described above except that the portion containing the unbound materials (now the dissociated aggregate) is retained and the portion containing the capture reagent is discarded.

### C. Detection of Captured Aggregate

Detection of aggregates may be accomplished using a conformational protein-specific binding reagent. In preferred embodiments, the CPSB reagent is an antibody (monoclonal or polyclonal) that recognizes an epitope on the conformational protein.

Detection of the captured aggregates in the sample may also be accomplished by using an ASB reagent. Such a reagent may be used in embodiments where the capture reagent is either the same or a different aggregate-specific binding reagent or a conformational protein-specific binding agent.

When the method utilizes a first aggregate-specific binding reagent and a second aggregate-specific binding reagent, the first and second reagents can be the same or different. By “the same” is meant that the first and second reagents differ only in the inclusion of a detectable label in the second reagent. The first and second reagents are “different,” for example, if they have a different structure or are derived from fragments from a different region of a prion protein.

### General Detection Methods

Any suitable means of detection can then be used to identify binding between the capture reagent and aggregates.

Analytical methods suitable for use to detect binding include methods such as fluorescence, electron microscopy, atomic force microscopy, UV/Visible spectroscopy, FTIR, nuclear magnetic resonance spectroscopy, Raman spectroscopy, mass spectrometry, HPLC, capillary electrophoresis, surface plasmon resonance spectroscopy, Micro-Electro-Mechanical Systems (MEMS), or any other method known in the art.

Binding may also be detected through the use of labeled reagents or antibodies, often in the form of an ELISA. Detectable labels suitable for use in the invention include any molecule capable of detection, including, but not limited to, radioactive isotopes, fluorescers, chemiluminescers, chromophores, fluorescent semiconductor nanocrystals, enzymes, enzyme substrates, enzyme cofactors, enzyme inhibitors, chromophores, dyes, metal ions, metal sols, ligands (e.g., biotin, strepavidin or haptens) and the like. Additional labels include, but are not limited to, those that use fluorescence, including those substances or portions thereof that are capable of exhibiting fluorescence in the detectable range. Particular examples of labels that may be used in the invention include, but are not limited to, horseradish peroxidase (HRP), fluorescein, FITC, rhodamine, dansyl, umbelliferone, dimethyl acridinium ester (DMAE), Texas red, luminol, NADPH and β-galactosidase. Additionally, the detectable label may include an oligonucleotide tag, which can be detected by a method of nucleic acid detection including, e.g., polymerase chain reaction (PCR), transcription-mediated amplification (TMA), branched DNA (b-DNA), nucleic acid sequence-based amplification (NASBA), and the like. Preferred detectable labels include enzymes, especially alkaline phosphatase (AP), horseradish peroxidase (HRP), and fluorescent compounds. As is well known in the art, the enzymes are utilized in combination with a detectable substrate, e.g., a chromogenic substrate or a fluorogenic substrate, to generate a detectable signal.

In addition to the use of labeled detection reagents (described above), immunoprecipitation may be used to separate out reagents that are bound to the aggregate. Preferably, the immunoprecipitation is facilitated by the addition of a precipitating enhancing agent. A precipitation-enhancing agent includes moieties that can enhance or increase the precipitation of the reagents that are bound to proteins. Such precipitation enhancing agents include polyethylene glycol (PEG), protein G, protein A and the like. Where protein G or protein A are used as precipitation enhancing agents, the protein can optionally be attached to a bead, preferably a magnetic bead. Precipitation can be further enhanced by use of centrifugation or with the use of magnetic force. Use of such precipitating enhancing agents is known in the art.

Western blots, for example, typically employ a tagged primary antibody that detects denatured protein from an SDS-PAGE gel, on samples obtained from a “pull-down” assay (as described herein), that has been electroblotted onto nitrocellulose or PVDF. The primary antibody is then detected (and/or amplified) with a probe for the tag (e.g., streptavidin-conjugated alkaline phosphatase, horseradish peroxidase, ECL reagent, and/or amplifiable oligonucleotides). Binding can also be evaluated using detection reagents such as a peptide with an affinity tag (e.g., biotin) that is labeled and amplified with a probe for the affinity tag (e.g., streptavidin-conjugated alkaline phosphatase, horseradish peroxidase, ECL reagent, or amplifiable oligonucleotides).

Cell based assays can also be employed, for example, where the aggregate is detected directly on individual cells (e.g., using a fluorescently labeled reagent that enables fluorescence based cell sorting, counting, or detection of the specifically labeled cells).

Assays that amplify the signals from the detection reagent are also known. Examples of which are assays that utilize biotin and avidin, and enzyme-labeled and mediated immunoassays, such as ELISA assays. Further examples include the use of branched DNA for signal amplification (see, e.g., U.S. Pat. Nos. 5,681,697; 5,424,413; 5,451,503; 5,4547,025; and 6,235,483); applying target amplification techniques like PCR, rolling circle amplification, Third Wave's invader (Arruda et al. 2002 Expert. Rev. Mol. Diagn. 2:487; U.S. Pat. Nos. 6,090,606, 5,843,669, 5,985,557, 6,090,543, 5,846,717), NASBA, TMA etc. (U.S. Pat. No. 6,511,809; EP 0544212A1); and/or immuno-PCR techniques (see, e.g., U.S. Pat. No. 5,665,539; International Publications WO 98/23962; WO 00/75663; and WO 01/31056).

In addition, microtitre plate procedures similar to sandwich ELISA may be used, for example, a aggregate-specific binding reagent or a conformational protein-specific binding reagent as described herein is used to immobilize protein(s) on a solid support (e.g., well of a microtiter plate, bead, etc.) and an additional detection reagent which could include, but is not limited to, another aggregate-specific binding reagent or a conformational protein-specific binding reagent with an affinity and/or detection label such as a conjugated alkaline phosphatase, horseradish peroxidase, ECL reagent, or amplifiable oligonucleotides is used to detect the aggregate.

### Preferred Methods for Detecting Dissociated Captured Aggregate

If the capture reagent and bound aggregate are dissociated prior to detection, the dissociated aggregates can be detected in an ELISA type assay, either as a direct ELISA or an antibody Sandwich ELISA type assay, which are described more fully below. Although the term “ELISA” is used to describe the detection with antibodies, the assay is not limited to ones in which the antibodies are “enzyme-linked.” The detection antibodies can be labeled with any of the detectable labels described herein and well-known in the immunoassay art. ELISAs such as described in Lau et al. PNAS USA 104(28): 11551-11556 (2007) can be performed to quantify the amount of aggregate dissociated from the capture reagent.

The dissociated aggregate can be prepared for a standard ELISA by passively coating it onto the surface of a solid support. Methods for such passive coating are well known and typically are carried out in 100 mM NaHCO₃ at pH 8 for several hours at about 37° C. or overnight at 4° C. Other coating buffers are well-known (e.g, 50 mM carbonate pH 9.6, 10 mM Tris pH 8, or 10 mM PBS pH 7.2) The solid support can be any of the solid supports described herein or well-known in the art but preferably the solid support is a microtiter plate, e.g., a 96-well polystyrene plate. Where the dissociation has been carried out using a high concentration of chaotropic agent, the concentration of the chaotropic agent will be reduced by dilution by at least about 2-fold prior to coating on the solid support. Where the dissociation has been carried out using a high or low pH, followed by neutralization, the dissociated aggregate can be used for coating without any further dilution. The plate(s) can be washed to remove unbound material.

If a standard ELISA is to be performed, then a detectably labeled binding molecule, such as a conformational protein-specific binding reagent or an aggregate-specific binding reagent attached to a solid support (either the same one used for capture or a different one) is added. This detectably labeled binding molecule is allowed to react with any captured aggregate, the plate washed and the presence of the labeled molecule detected using methods well known in the art. The detection molecule need not be specific for the aggregate but can bind to both aggregate and monomer, as long as the capture reagent is specific for the aggregate. In preferred embodiments, the detectably labeled binding molecule is an antibody. Such antibodies include ones that are well known as well as antibodies that are generated by well known methods which are specific for both the native and misfolded conformers of a conformational protein.

In an alternative embodiment, the dissociated aggregates are detected using an antibody sandwich type ELISA. In this embodiment, the dissociated aggregate is “recaptured” on a solid support having a first antibody specific for the aggregate or the conformational protein. The solid support with the recaptured aggregate is optionally washed to remove any unbound materials, and then contacted with a second antibody specific for the conformational protein or aggregate under conditions that allow the second antibody to bind to the recaptured aggregate.

The first and second antibodies will typically be different antibodies and will preferably recognize different epitopes on the conformational protein. For example, the first antibody will recognize an epitope at the N-terminal end of the conformational protein and the second antibody will recognize an epitope at other than the N-terminal, or vice versa. Other combinations of first and second antibody can be readily selected. In this embodiment, the second antibody, but not the first antibody, will be detectably labeled.

When the dissociation of the aggregate from the reagent is carried out using a chaotropic agent, the chaotropic agent should be removed or diluted by at least 15-fold prior to carrying out the detection assay. When the dissociation is effected using a high or low pH and neutralization, the dissociated aggregate can be used without further dilution. When the dissociated aggregate is denatured prior to carrying out the detection, the first and second antibodies will both bind to the denatured conformer.

### Preferred Methods for Detecting Undissociated Captured Aggregate

In other exemplary assays, the capture reagent and bound aggregate are not dissociated prior to detection. When the capture reagent is an ASB coupled to a solid support, a sample containing or suspected of containing aggregate can be added to the solid support. After a period of incubation sufficient to allow any aggregates to bind to the reagent, the solid support can be washed to remove unbound moieties and a detectably labeled secondary binding molecule as described above, such as a conformational protein-specific binding reagent or a second same or different aggregate-specific binding reagent attached to a solid support, is added. Alternatively, a conformational protein-specific binding reagent coupled to a solid support (e.g., coated onto the wells of a microtiter plate) is used as a capture reagent and detection can be accomplished using an aggregate-specific binding reagent attached to a solid support.

### D. Solid Supports Used in Assays

The ASB reagents are provided on a solid support. In certain embodiments, CPSB reagents are provided on a solid support. The ASB reagents or CPSB reagents are provided on a solid support prior to contacting the sample or, in the case of a CPSB reagent, the reagent can be adapted for binding to the solid support after contacting the sample and binding to any aggregate therein (e.g., by using a biotinylated reagent and a solid support including an avidin or streptavidin).

A solid support, for purposes of the invention, can be any material that is an insoluble matrix and can have a rigid or semi-rigid surface to which a molecule of interest (e.g., reagents of the invention, conformational proteins, antibodies, etc) can be linked or attached. Exemplary solid supports include, but are not limited to, substrates such as nitrocellulose, polyvinylchloride; polypropylene, polystyrene, latex, polycarbonate, nylon, dextran, chitin, sand, silica, pumice, agarose, cellulose, glass, metal, polyacrylamide, silicon, rubber, polysaccharides, polyvinyl fluoride, diazotized paper, activated beads, magnetically responsive beads, and any materials commonly used for solid phase synthesis, affinity separations, purifications, hybridization reactions, immunoassays and other such applications. The support can be particulate or can be in the form of a continuous surface and includes membranes, mesh, plates, pellets, slides, disks, capillaries, hollow fibers, needles, pins, chips, solid fibers, gels (e.g. silica gels) and beads, (e.g., pore-glass beads, silica gels, polystyrene beads optionally cross-linked with divinylbenzene, grafted co-poly beads, polyacrylamide beads, latex beads, dimethylacrylamide beads optionally crosslinked with N—N′-bis-acryloylethylenediamine, iron oxide magnetic beads, and glass particles coated with a hydrophobic polymer.

ASB reagents or CPSB reagents as described herein can be readily coupled to the solid support using standard techniques which attach the ASB reagent or CPSB reagent, for example covalently, by absorption, coupling or through the use of binding pairs.

Immobilization to the support may be enhanced by first coupling the ASB reagent or CPSB reagent to a protein (e.g., when the protein has better solid phase-binding properties). Suitable coupling proteins include, but are not limited to, macromolecules such as serum albumins including bovine serum albumin (BSA), keyhole limpet hemocyanin, immunoglobulin molecules, thyroglobuline, ovalbumin, and other proteins well known to those skilled in the art. Other reagents that can be used to bind molecules to the support include polysaccharides, polylactic acids, polyglycolic acids, polymeric amino acids, amino acid copolymers, and the like. Such molecules and methods of coupling these molecules to proteins, are well known to those of ordinary skill in the art. See, e.g., Brinkley, M. A., (1992) Bioconjugate Chem., 3:2-13; Hashida et al. (1984) J. Appl. Biochem., 6:56-63; and Anjaneyulu and Staros (1987) International J. of Peptide and Protein Res. 30:117-124.

If desired, the ASB reagents or CPSB reagents to be added to the solid support can readily be functionalized to create styrene or acrylate moieties, thus enabling the incorporation of the molecules into polystyrene, polyacrylate or other polymers such as polyimide, polyacrylamide, polyethylene, polyvinyl, polydiacetylene, polyphenylene-vinylene, polypeptide, polysaccharide, polysulfone, polypyrrole, polyimidazole, polythiophene, polyether, epoxies, silica glass, silica gel, siloxane, polyphosphate, hydrogel, agarose, cellulose and the like. In preferred embodiments, the solid support is a magnetic bead, more preferably a polystyrene/iron oxide bead.

The ASB reagents or CPSB reagents can be attached to the solid support through the interaction of a binding pair of molecules. Such binding pairs are well known and examples are described elsewhere herein. One member of the binding pair is coupled by techniques described above to the solid support and the other member of the binding pair is attached to the reagent (before, during, or after synthesis). The ASB reagent or CPSB reagent thus modified can be contacted with the sample and interaction with the aggregate, if present, can occur in solution, after which the solid support can be contacted with the reagent (or reagent-protein complex). Preferred binding pairs for this embodiment include biotin and avidin, and biotin and streptavidin. In addition to biotin-avidin and biotin-streptavidin, other suitable binding pairs for this embodiment include, for example, antigen-antibody, hapten-antibody, mimetope-antibody, receptor-hormone, receptor-ligand, agonist-antagonist, lectin-carbohydrate, Protein A-antibody Fc. Such binding pairs are well known (see, e.g., U.S. Pat. Nos. 6,551,843 and 6,586,193) and one of ordinary skill in the art would be competent to select suitable binding pairs and adapt them for use with the present invention. When the capture reagent is adapted for attachment to the support as described above, the sample can be contacted with the capture reagent before or after the capture reagent is attached to the support.

Alternatively, the ASB reagents or CPSB reagents can be covalently attached to the solid support using conjugation chemistries that are well known in the art. For example, thiol containing ASB or CPSB reagents can be directly attached to solid supports, e.g., carboxylated magnetic beads, using standard methods known in the art (See, e.g., Chrisey, L. A., Lee, G. U. and O'Ferrall, C. E. (1996). Covalent attachment of synthetic DNA to self-assembled monolayer films. Nucleic Acids Research 24(15), 3031-3039; Kitagawa, I., Shimozono, T., Aikawa, T., Yoshida, T. and Nishimura, H. (1980). Preparation and characterization of hetero-bifunctional cross-linking reagents for protein modifications. Chem. Pharm. Bull. 29(4), 1130-1135). Carboxylated magnetic beads are first coupled to a heterobifunctional cross-linker that contains a maleimide functionality (BMPH from Pierce Biotechnology Inc.) using carbodiimide chemistry. The thiolated ASB or CPSB reagent is then covalently coupled to the maleimide functionality of the BMPH coated beads. When used in the embodiments of the detection methods of the invention, the solid support aids in the separation of the complex including the reagent and the aggregate from the unbound sample. Particularly convenient magnetic beads for thiol coupling are Dynabeads™ M-270 Carboxylic Acid from Dynal (now Invitrogen Corporation, Carlsbad, Calif.). The ASB or CPSB reagent may also include a linker, for example, one or more aminohexanoic acid moieties.

### E. Preferred Detection Methods for Aggregates

Preferred embodiments are described below.

In preferred embodiments, the methods of the invention capture and detect the aggregate using an ASB reagent, which has a net charge of at least about positive one at the pH at which the sample is contacted with the ASB reagent, is attached to a solid support at a charge density of at least about 60 nmol net charge per square meter, and binds preferentially with aggregates over monomers when attached to the solid support, said method includes contacting a sample suspected of containing the aggregate with an ASB reagent under conditions that allow binding of the ASB reagent to the aggregate, if present, to form a complex; and detecting the aggregate, if any, in the sample by its binding to the ASB reagent. Binding of the aggregate can be detected, for example, by dissociating the complex and detecting aggregate with a CPSB reagent.

In one embodiment, the aggregate to be captured is an aggregate associated with Alzheimer's disease, such as Aβ40, Aβ42, or tau. In such a case, the sample is preferably plasma or cerebrospinal fluid. The ASB reagent is preferably derived from SEQ ID NOs: 1-8, and includes peptoid reagents such as

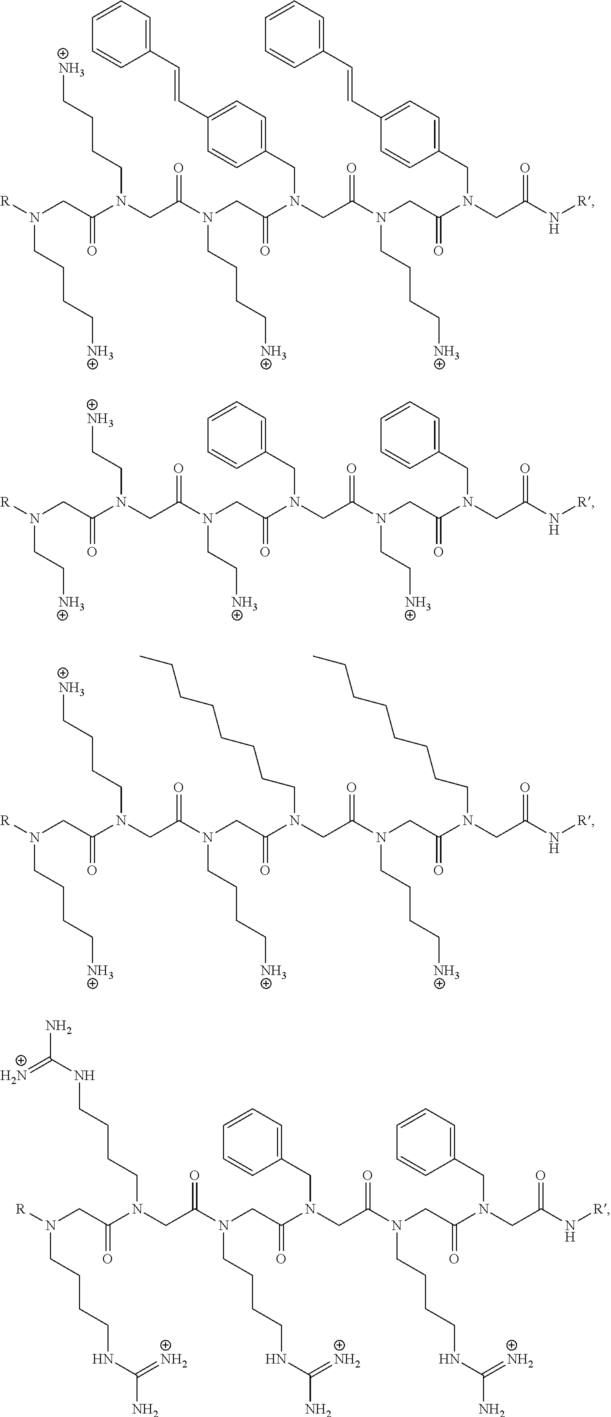

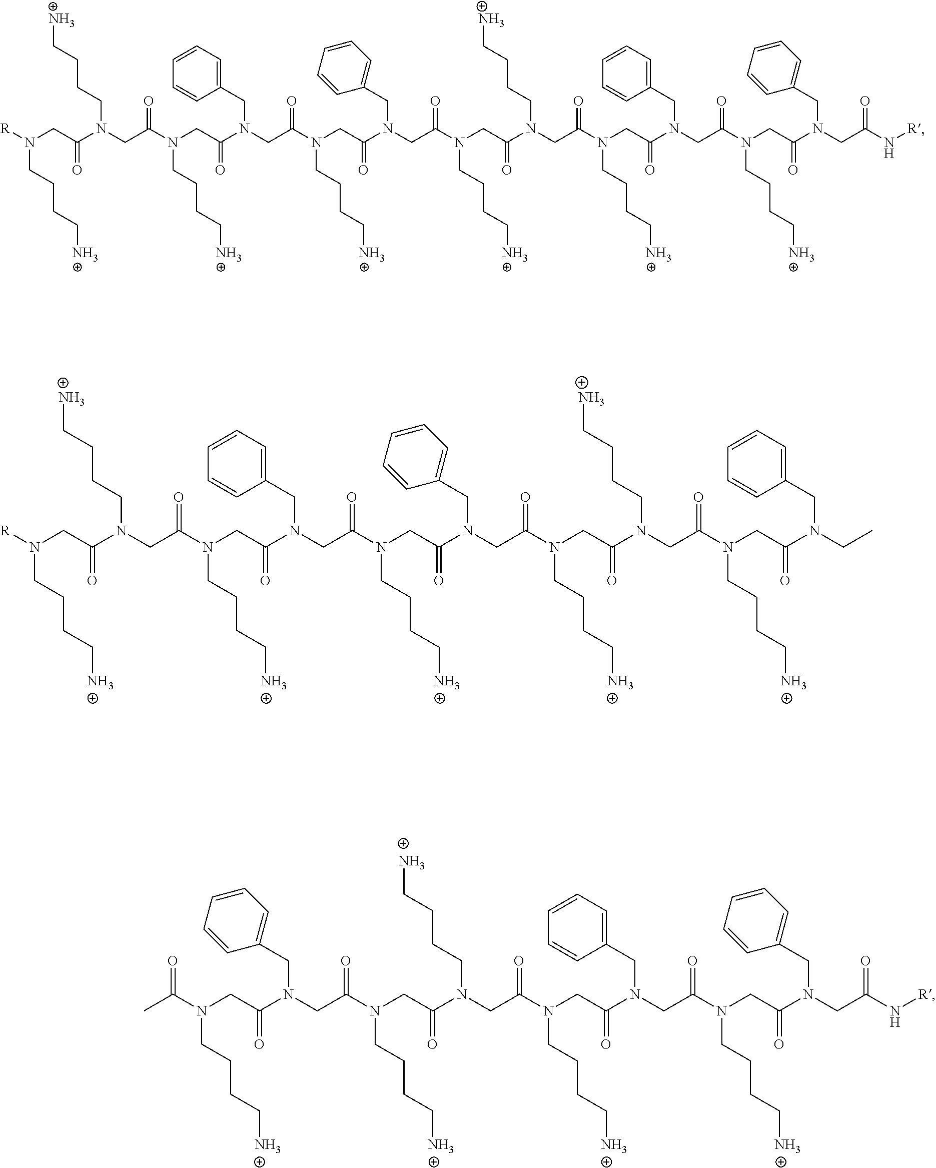

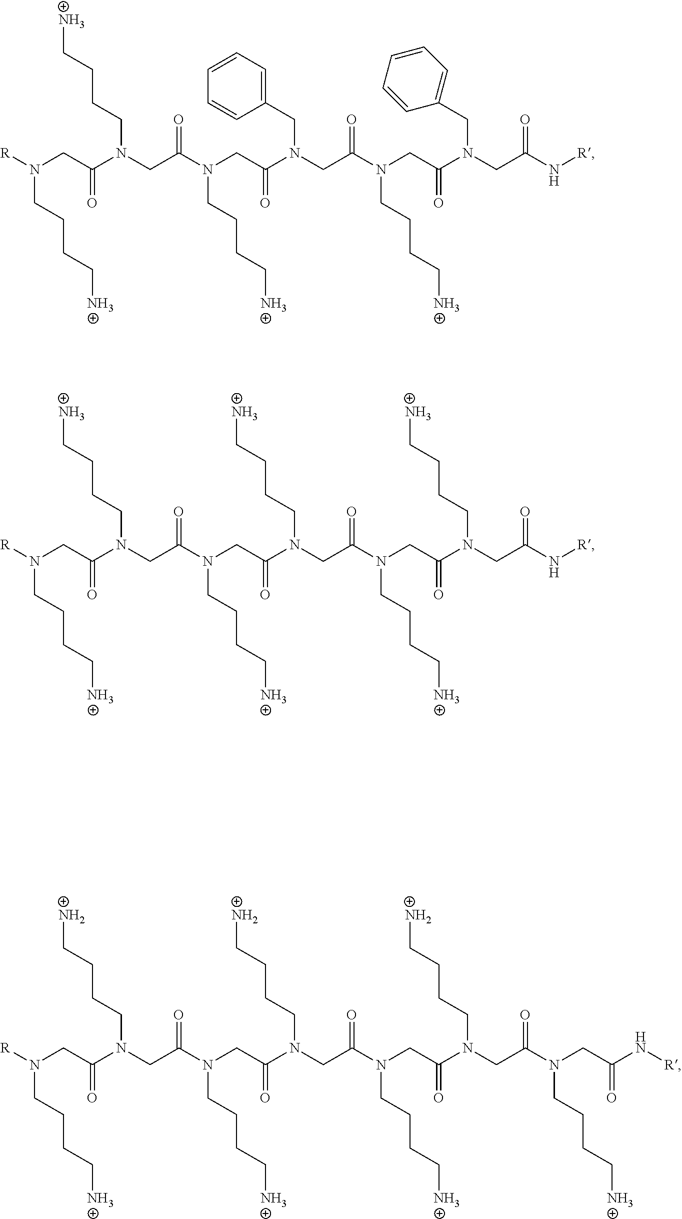

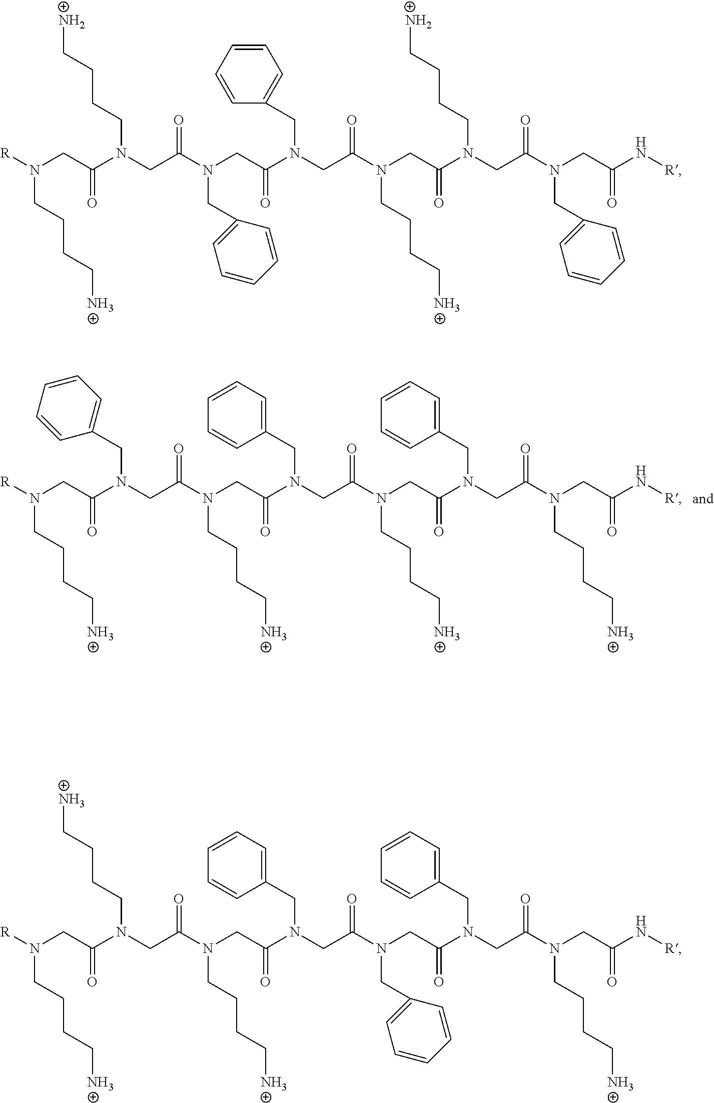

where R and R′ can be any group.

In other preferred embodiments, the methods of the invention capture the aggregate using a ASB reagent which has a net charge of at least about positive one at the pH at which the sample is contacted with the ASB reagent, is attached to a solid support at a charge density of at least about 60 nmol net charge per square meter, and binds preferentially with aggregates over monomers when attached to the solid support, and detect the aggregate using a CPSB reagent. The method includes contacting a sample suspected of containing the aggregate with an ASB reagent under conditions that allow the binding of the reagent to the aggregate, if present, to form a first complex; contacting the first complex with a CPSB reagent under conditions that allow binding; and detecting the presence of the aggregate, if any, in the sample by its binding to the CPSB binding reagent. Typically, unbound sample is removed after forming the first complex and before contacting the first complex with the CPSB reagent. The CPSB binding reagent can be a labeled anti-conformational protein antibody.

In still yet another preferred embodiment, the methods of the invention capture and detect the presence of an aggregate using a ASB reagent which has a net charge of at least about positive one at the pH at which the sample is contacted with the ASB reagent, is attached to a solid support at a charge density of at least about 60 nmol net charge per square meter, and binds preferentially with aggregates over monomers when attached to the solid support. The method includes contacting a sample suspected of containing the aggregate with a ASB reagent under conditions that allow the binding of the ASB reagent to the aggregate, if present, to form a first complex; removing unbound sample materials; dissociating the aggregate from the first complex thereby providing dissociated aggregate; contacting the dissociated aggregate with a first CPSB reagent under conditions that allow binding to form a second complex; and detecting the presence of the aggregate, if any, in the sample by detecting the formation of the second complex. The formation of the second complex is preferably detected using a detectably labeled second CPSB reagent, and the first CPSB reagent is preferably coupled to a solid support.

In an alternative, the invention provides a method for capturing the aggregate using a first ASB reagent which has a net charge of at least about positive one at the pH at which the sample is contacted with the ASB reagent, is attached to a solid support at a charge density of at least about 60 nmol net charge per square meter, and binds preferentially with aggregates over monomers when attached to the solid support, and detecting the aggregate using a second ASB reagent as described herein. The method involves contacting a sample suspected of containing the aggregate with the first ASB reagent under conditions that allow binding of the first reagent to the aggregate, if present, to form a first complex; contacting the sample suspected of containing the aggregate with a second ASB reagent under conditions that allow binding of the second reagent to the aggregate in the first complex, wherein the second reagent has a detectable label; and detecting the aggregate, if any, in a sample by its binding to the second reagent.

In yet another alternative, the invention provides a method for capturing the aggregate using a CPSB reagent and detecting the aggregate using a ASB reagent which has a net charge of at least about positive one at the pH at which the sample is contacted with the ASB reagent, is attached to a solid support at a charge density of at least about 60 nmol net charge per square meter, and binds preferentially with aggregates over monomers when attached to the solid support. The method involves (a) contacting a sample suspected of containing the aggregate with a CPSB reagent under conditions that allow binding of the reagent to the aggregate, if present, to form a complex; (b) removing unbound sample materials; (c) contacting the complex with a ASB reagent under conditions that allow the binding of the ASB reagent to the aggregate, wherein the ASB reagent includes a detectable label; and detecting the aggregate, if any, in the sample by its binding to the ASB reagent; wherein the ASB reagent has a net charge of at least about positive one at the pH at which the sample is contacted with the ASB reagent, is attached to a solid support at a charge density of at least about 60 nmol net charge per square meter, and binds preferentially with aggregates over monomers when attached to the solid support.

In all of the above methods “unbound sample” refers to those components within the sample that are not captured in the contacting steps. The unbound sample may be removed by methods that are well known in the art, for example, by washing, centrifugation, filtration, magnetic separation and combinations of these techniques. Preferably, in the methods of the invention, unbound samples are removed by washing the complexes with buffer and/or magnetic separation.

In preferred embodiments, methods of the invention are used for detection of conformational diseases, including systemic amyloidoses, tauopathies, synucleinopathies, and preeclampsia.

### F. Methods for Detecting Oligomers

The invention described herein provides methods for detecting oligomers. In preferred embodiments, the invention provides methods for detecting the presence of oligomer in a sample by providing a sample suspected of containing oligomer which lacks aggregates other than oligomers, contacting the sample with an ASB reagent under conditions that allow binding of the reagent to the oligomer, if present, to form a complex, and detecting the presence of oligomer, if any, in the sample by its binding to the ASB reagent, where the ASB reagent has a net charge of at least about positive one at the pH at which the sample is contacted with the ASB reagent, is attached to a solid support at a charge density of at least about 2000 nmol net charge per square meter, and binds preferentially with aggregates over monomers when attached to the solid support.

For use in methods of detecting oligomers, the sample can be anything known to, or suspected of, containing an aggregate. The sample can be a biological sample (that is, a sample prepared from a living or once-living organism) or a non-biological sample. Typically, a biological sample contains bodily tissues or fluid. Suitable biological samples include, but are not limited to whole blood, blood fractions, blood components, plasma, platelets, serum, cerebrospinal fluid (CSF), bone marrow, urine, tears, milk, lymph fluid, organ tissue, brain tissue, nervous system tissue, muscle tissue, non-nervous system tissue, biopsy, necropsy, fat biopsy, cells, feces, placenta, spleen tissue, lymph tissue, pancreatic tissue, bronchoalveolar lavage, or synovial fluid. Preferred biological samples include plasma and CSF. In certain embodiments, the sample contains polypeptide.

In an alternative embodiments, the invention provides methods for detecting the presence of oligomer in a sample by providing a sample suspected of containing oligomer, removing aggregate other than oligomer from the sample, contacting the sample with an ASB reagent under conditions that allow binding of the reagent to the oligomer, if present, to form a complex, and detecting the presence of oligomer, if any, in the sample by its binding to the ASB reagent, where the ASB reagent has a net charge of at least about positive one at the pH at which the sample is contacted with the ASB reagent, is attached to a solid support at a charge density of at least about 2000 nmol net charge per square meter, and binds preferentially with aggregates over monomers when attached to the solid support. In preferred embodiments, aggregate other than oligomer is removed from the sample by centrifugation.

In yet another embodiment, the invention provides methods for detecting the presence of oligomer in a sample by contacting a sample suspected of containing oligomer with an ASB reagent under conditions that allow binding of the reagent to the oligomer, if present, to form a complex, contacting the complex with a second reagent, where the reagent binds preferentially to either oligomer or aggregates other than oligomer, and detecting the presence of oligomer, if any, in the sample by its binding or lack of binding to the second reagent, where the ASB reagent has a net charge of at least about positive one at the pH at which the sample is contacted with the ASB reagent, is attached to a solid support at a charge density of at least about 2000 nmol net charge per square meter, and binds preferentially with aggregates over monomers when attached to the solid support. In preferred embodiments, the second reagent is A11 antibody, which recognizes oligomers but not fibrils.

In preferred embodiments of methods for detecting the presence of oligomers, aggregates other than oligomers include fibrils.

Methods for Removing Non-Oligomer Aggregate from a Sample

Non-oligomer aggregates may be removed from a sample by any methods known in the art. Typically, non-oligomer aggregates such as amorphous aggregates and fibrils may be removed from a sample by centrifugation. Preferred centrifugation conditions used by practitioners in the art are varied (Philo, AAPS J, 2006, 8 (3) Art. 65). However, centrifugation at 14,000×g for 10 minutes will typically remove only very large aggregates, including large fibrils and some amorphous aggregates (10-1000 MDa), and centrifugation at 100,000×g for one hour will typically remove aggregates larger than 1 MDa, including smaller fibrils and amorphous aggregates. The size, solubility, and ionic strength of aggregates and the concentration, temperature, and pH of the sample will all affect the centrifugation acceleration and speed required for separation (Sipe, J. (ed.), 2005, Amyloid Proteins: The Beta Sheet Conformation and Disease, 410-425, Wiley-VCH; Stine, et al, JBC, 2003, 278, 11612-22).

### G. Detection Methods for Conformational Diseases

### Conformational Diseases

This invention relates to methods to detect aggregates of non-native conformers using an aggregate-specific binding reagent, to assess whether there is an increased probability of aggregate-mediated disease, and to assess the effectiveness of treatment for an aggregate-mediated disease. Conformational disease proteins and their corresponding diseases include those listed in Table 1.

Conformational diseases of this invention include any disease associated with proteins which form two or more different conformations. Those of particular interest herein include amyloid diseases, all which display a cross-beta sheet signature, such as Alzheimer's disease, systemic amyloidoses, tauopathies, and synucleinopathies. Other diseases of interest are diabetes and poly-glutamine diseases, along with non-amyloid proteinopathies like serpinopathies.

In certain embodiments, the methods of the invention also include use of a conformational protein-specific binding reagent (“CPSB reagent”) to either capture or detect both monomers and aggregates. The particular CPSB reagent used will depend on the protein being detected. For example, if the conformational disease to be diagnosed is Alzheimer's disease, then the CPSB reagent may be an antibody which recognizes both the monomer and aggregates of the Alzheimer's disease protein Aβ.

### Methods for Detection of Pathogenic Alzheimer's Disease Aggregates

Methods for detection of pathogenic Alzheimer's disease aggregates containing misfolded conformers such as Aβ40, Aβ42, or tau are provided.

In particularly preferred embodiments, these methods capture the pathogenic Alzheimer's disease aggregate with an ASB reagent which has a net charge of at least about positive one at the pH at which the sample is contacted with the ASB reagent, is attached to a solid support at a charge density of at least about 60 nmol net charge per square meter, and binds preferentially with aggregates over monomers when attached to the solid support, and detect the captured aggregate with a CPSB reagent.

In particular, the methods include contacting a sample suspected of containing the pathogenic Alzheimer's disease aggregate with an ASB reagent under conditions that allow the binding of the ASB reagent to the pathogenic Alzheimer's disease aggregate, if present, to form a first complex; removing unbound sample materials; dissociating the pathogenic Alzheimer's disease aggregate from the first complex thereby providing dissociated pathogenic Alzheimer's disease aggregater; contacting the dissociated pathogenic Alzheimer's disease aggregate with a CPSB reagent under conditions that allow binding to form a second complex; and detecting the presence of the pathogenic Alzheimer's disease aggregate, if any, in the sample by detecting the formation of the second complex. The pathogenic Alzheimer's disease aggregate in the first complex is preferably dissociated and denatured with about 0.05N NaOH or about 0.1N NaOH, at about 90° C. or about 80° C. before contacting the CPSB reagent. When the pathogenic Alzheimer's disease aggregate contains Aβ40 or Aβ42, it is preferably dissociated and denatured at about 0.1 N NaOH at about 80° C. for about 30 minutes. Preferably, a sandwich ELISA is used.

Dissociation and/or denaturation can be accomplished using the methods described in Section IV(B). Typically, the pathogenic Alzheimer's disease aggregate is simultaneously dissociated and denatured by altering the pH from low to high or high to low pH.

In preferred embodiments, the ASB reagent is derived from SEQ ID NOs: 1-8, or peptoids including

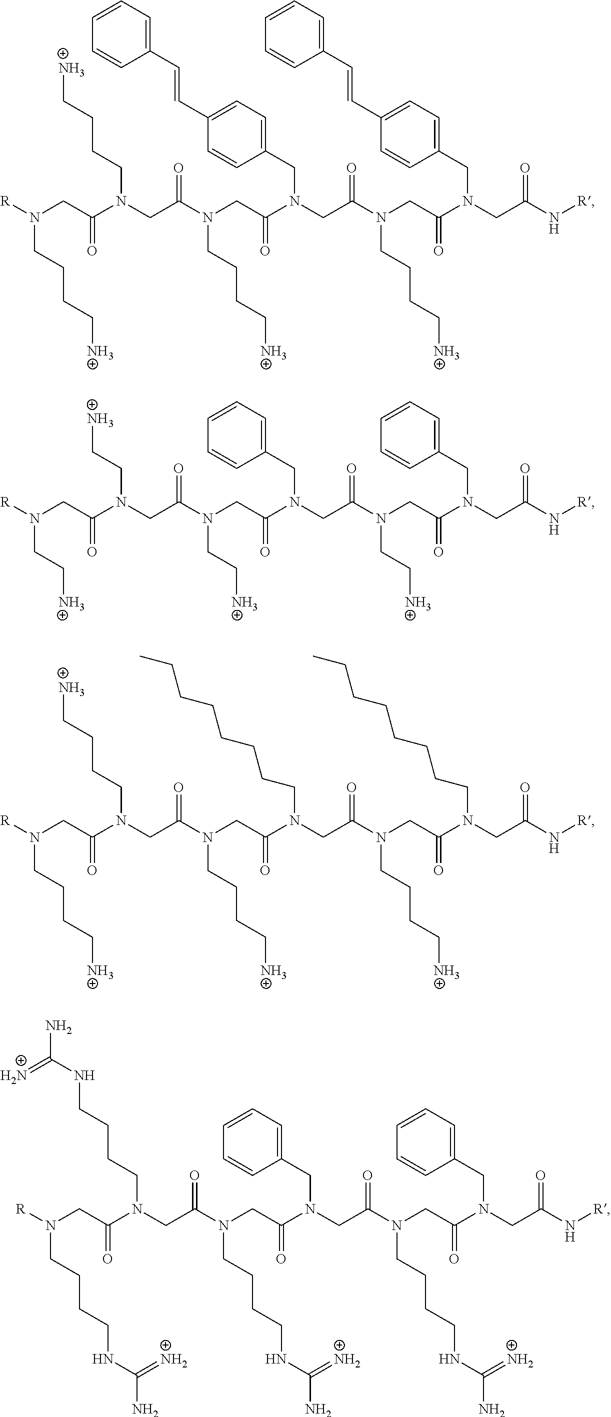

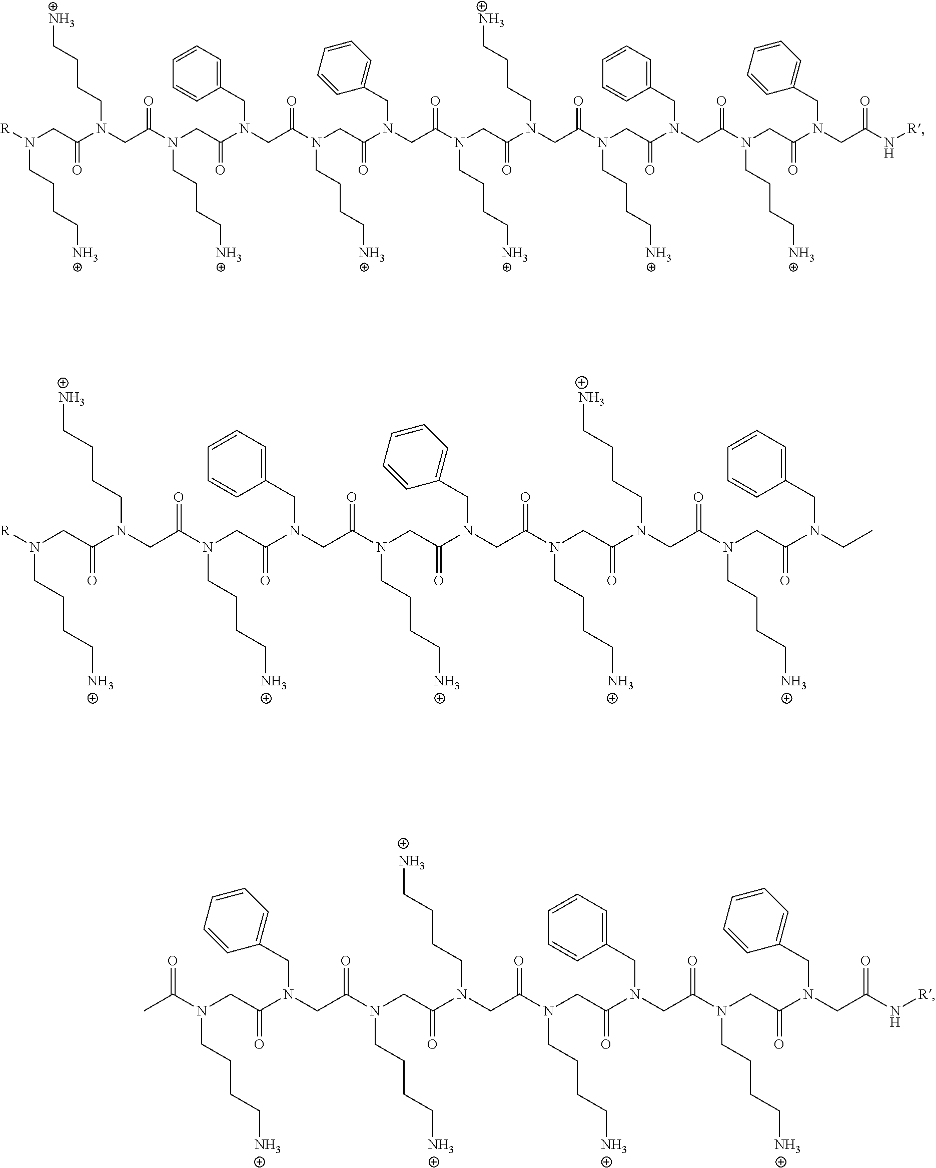

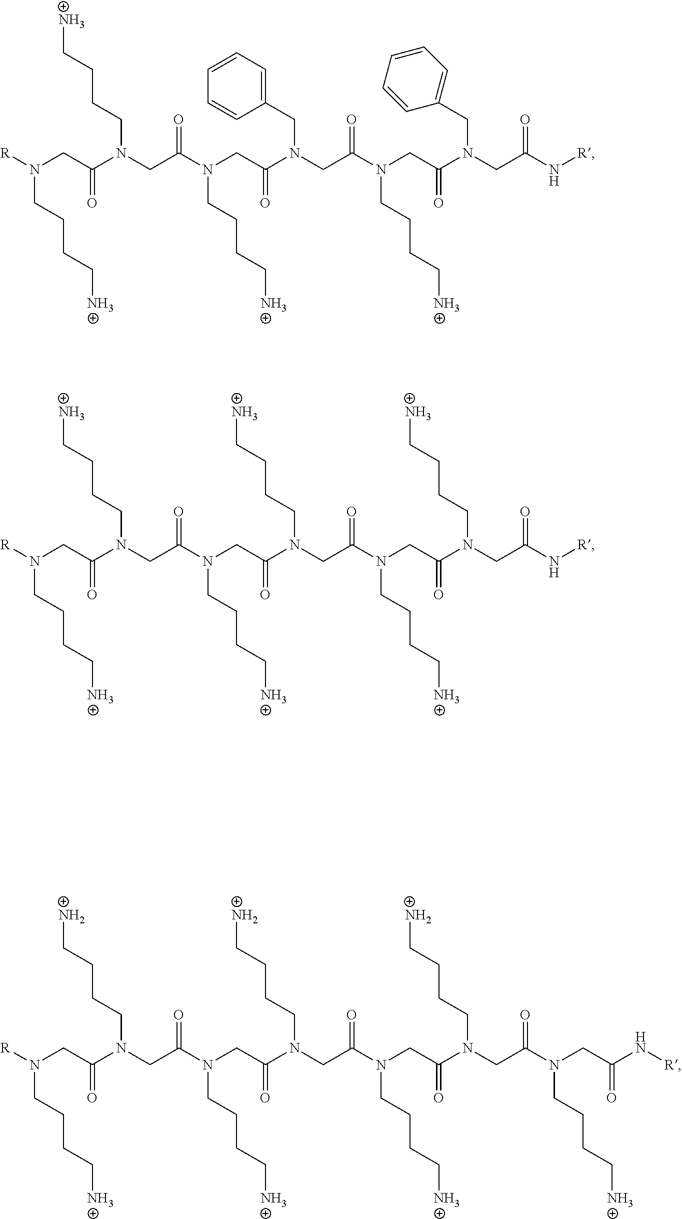

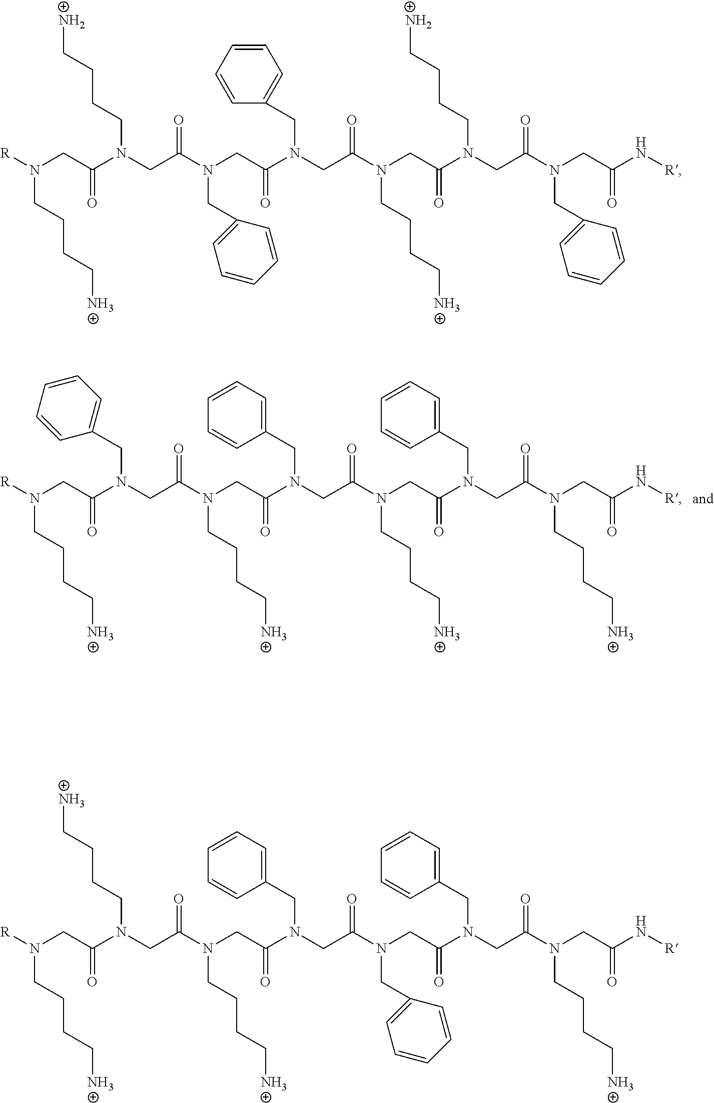

where R and R′ can be any group, and the reagent is coupled to a solid support, such as a magnetic bead.

The CPSB reagent is preferably an anti-Alzheimer's disease protein antibody coupled to a solid support such as a microtiter plate and formation of the second complex is preferably detected using a second detectably labeled CPSB reagent. When the pathogenic Alzheimer's disease aggregate contains Aβ40 or Aβ42, preferred anti-Alzheimer's disease protein antibodies include 11A50-B10 (Covance), a antibody specific for C-terminus of Aβ40; 12F4 (Covance), a antibody specific for C-terminus of Aβ42; 4G8, specific for Aβ amino acids 18-22; 20.1, specific for Aβ amino acids 1-10; and 6E10, specific for Aβ amino acids 3-8. In particularly preferred embodiments, 12F4 or 11A50-B10 are the capture antibodies on an ELISA plate and 14G8 is used as the second detectably labeled CPSB reagent. The sample is preferably plasma or cerebrospinal fluid (CSF).

Thus, in particularly preferred embodiments, methods for detecting the presence of a pathogenic Alzheimer's disease aggregate include, but are not limited to, the steps of: contacting a sample of plasma or CSF suspected of containing pathogenic Alzheimer's disease aggregate with PSR1 coupled to a magnetic bead under conditions that allow the binding of PSR1 to a pathogenic Alzheimer's disease aggregate, if present, to form a first complex; removing unbound sample materials; dissociating and/or denaturing the pathogenic Alzheimer's disease aggregate from the first complex by altering pH, thereby providing a dissociated pathogenic Alzheimer's disease aggregate; contacting the dissociated pathogenic Alzheimer's disease aggregate with an anti-Alzheimer's disease protein antibody bound to a solid support under conditions that allow binding to form a second complex; and detecting the formation of the second complex by incubating with a second labeled anti-Alzheimer's disease protein antibody.

### H. Competition Assays

In some aspects, the methods of this invention detect aggregates via competitive binding. Means of detection can be used to determine when a ligand which weakly binds to the ASB binding reagent is displaced by aggregate. The ASB reagent adsorbed onto a solid support is combined with a detectably labeled ligand that binds to the ASB reagent with a binding avidity weaker than that with which the aggregate binds to the ASB reagent. The ligand-ASB reagent complexes are detected. Sample is then added. Since the binding avidity of the detectably labeled ligand is weaker than the binding avidity of the aggregate for the ASB reagent, the aggregate will replace the labeled ligand and the decrease in detected amounts of the labeled ligand bound to the ASB reagent indicate complexes formed between the ASB reagent and aggregates in the sample.

Thus, in certain embodiments, the presence of an aggregate is detected by providing a solid support including an ASB reagent; combining the solid support with a detectably labeled ligand, wherein the ASB reagent's binding avidity to the detectably labeled ligand is weaker than the ASB reagent's binding avidity to the aggregate; combining a sample suspected of containing an aggregate with the solid support under conditions which allow the aggregate, when present in the sample, to bind to the ASB reagent and replace the ligand; and detecting complexes formed between the ASB reagent and the aggregate from the sample; wherein the ASB reagent has a net charge of at least about positive one at the pH at which the sample is contacted with the ASB reagent, is attached to a solid support at a charge density of at least about 60 nmol net charge per square meter, and hinds preferentially to aggregates over monomers when attached to the solid support.

### IV. OTHER METHODS

In general, the ASB reagents described herein are able to bind preferentially to aggregates of conformational proteins when the ASB reagent attached to a solid support at certain charge densities. Thus, these reagents allow for ready detection of the presence of aggregates in virtually any sample, biological or non-biological, including living or dead brain, spinal cord, or other nervous system tissue as well as blood. Samples may contain polypeptides, recombinant or synthetic. The reagents are thus useful in a wide range of isolation, purification, detection, diagnostic and therapeutic applications.

For example, ASB reagents attached to an affinity support may be used to isolate aggregates. ASB reagents can be affixed to a solid support by, for example, adsorption, covalent linkage, etc., so that the reagents retain their aggregate-selective binding activity. Optionally, spacer groups may be included, for example so that the binding site of the ASB reagent remains accessible. The immobilized ASB reagents can then be used to bind the aggregate from a biological sample, such as blood, plasma, brain, spinal cord, and other tissues. The bound reagents or complexes are recovered from the support by, for example, a change in pH or the aggregate may be dissociated from the complex.

Thus, in certain embodiments, the invention provides methods for reducing the amount of aggregates from a polypeptide sample by contacting a polypeptide sample suspected of containing aggregate with an ASB reagent under conditions that allow binding of the reagent to the aggregate, if present, to form a complex, and recovering unbound polypeptide sample, where the ASB reagent has a net charge of at least about positive one at the pH at which the sample is contacted with the ASB reagent, is attached to a solid support at a charge density of at least about 60 nmol net charge per square meter, and binds preferentially with aggregates over monomers when attached to the solid support. In certain embodiments, the method will further include detecting the presence of the complex to determine whether a sample contains aggregates. Detection of the complex may be achieved by allowing a second aggregate-specific binding reagent having a detectable label or a conformational protein-specific binding reagent having a detectable label to bind to the aggregate. Recombinant or synthetic protein production is critical for many industries such as pharmaceuticals, biofuels, and medical and other life science research. Such polypeptide samples may contain, for example, proteins manufactured for pharmaceutical use, such as recombinant insulin and therapeutic antibodies. These polypeptides may be produced at high levels, such that aggregates of the polypeptide tend to form at a relatively high rate. Methods provided by the invention for reducing the amount of aggregate from a polypeptide sample will be useful in quality control of these proteins generated for pharmaceutical use and in quality control of proteins produced for other industries.

In other embodiments, the invention provides a method for discriminating between aggregate and monomer in a sample by contacting a sample suspected of containing aggregate with an ASB reagent under conditions that allow binding of the reagent to the aggregate, if present, to form a complex; and discriminating between aggregate and monomer by binding of the aggregate to the reagent; where the ASB reagent has a net charge of at least about positive one at the pH at which the sample is contacted with the ASB reagent, is attached to a solid support at a charge density of at least about 60 nmol net charge per square meter, and binds preferentially with aggregates over monomers when attached to the solid support. In preferred embodiments, binding of the aggregate to the reagent will be detected by allowing a second aggregate-specific binding reagent having a detectable label or a conformational protein-specific binding reagent having a detectable label to bind to the aggregate. The unbound sample may be removed after formation of the complex before detecting the aggregate with a labeled reagent. Alternatively, the complex may be dissociated to provide dissociated aggregate, and then the dissociated aggregate may be allowed to bind a first CPBS reagent to form a second complex, and the formation of the second complex detected. In certain embodiments, the second complex may be detected using a detectably labeled second CPSB reagent. In certain embodiments, the first CPSB will be coupled to a solid support.

In certain embodiments, the invention provides a method for assessing whether there is an increased probability of conformational disease for a subject by contacting a biological sample suspected of having a conformational disease with an ASB reagent under conditions that allow binding of the reagent to the pathogenic aggregate, if present, to form a complex; detecting the presence of the pathogenic aggregate, if any, in the sample by its binding to the reagent; and determining that there is an increased probability that the subject has the conformational disease if the amount of pathogenic aggregate in the biological sample is higher than the amount of aggregate in a sample from a subject without the conformational disease; wherein the ASB reagent has a net charge of at least about positive one at the pH at which the sample is contacted with the ASB reagent, is attached to a solid support at a charge density of at least about 60 nmol net charge per square meter, and binds preferentially with aggregates over monomers when attached to the solid support. In preferred embodiments, binding of the aggregate to the reagent will be detected by allowing a second aggregate-specific binding reagent having a detectable label or a conformational protein-specific binding reagent having a detectable label to bind to the aggregate. The unbound sample may be removed after formation of the complex before detecting the aggregate with a labeled reagent. Alternatively, the complex may be dissociated to provide dissociated aggregate, and then the dissociated aggregate may be allowed to bind a first CPBS reagent to form a second complex, and the formation of the second complex detected. In certain embodiments, the second complex may be detected using a detectably labeled second CPSB reagent. In certain embodiments, the first CPSB will be coupled to a solid support.

In other embodiments, the invention provides a method for assessing the effectiveness of treatment for a conformational disease by contacting a biological sample from a patient having undergone treatment for the conformational disease with an ASB reagent under conditions that allow binding of the reagent to the pathogenic aggregate, if present, to form a complex; detecting the presence of the pathogenic aggregate, if any, in the sample by its binding to the reagent; and determining that the treatment is effective if the amount of pathogenic aggregate in the biological sample is lower than the amount of pathogenic aggregate in a biological sample taken from the patient prior to treatment for the conformational disease; wherein the ASB reagent has a net charge of at least about positive one at the pH at which the sample is contacted with the ASB reagent, is attached to a solid support at a charge density of at least about 60 nmol net charge per square meter, and binds preferentially with aggregates over monomers when attached to the solid support. In preferred embodiments, binding of the aggregate to the reagent will be detected by allowing a second aggregate-specific binding reagent having a detectable label or a conformational protein-specific binding reagent having a detectable label to bind to the aggregate. The unbound sample may be removed after formation of the complex before detecting the aggregate with a labeled reagent. Alternatively, the complex may be dissociated to provide dissociated aggregate, and then the dissociated aggregate may be allowed to bind a first CPBS reagent to form a second complex, and the formation of the second complex detected. In certain embodiments, the second complex may be detected using a detectably labeled second CPSB reagent. In certain embodiments, the first CPSB will be coupled to a solid support.

Several variations and combinations using the reagents described herein may be applied in the methods of the invention.

### V. COMPOSITIONS AND KITS

The invention provides compositions including aggregate-specific binding reagents and soild supports. Thus, in preferred embodiments, the invention provides peptide aggregate-specific binding reagents where the reagents contain the amino acid sequences of KKKFKF, KKKWKW, KKKLKL, or KKKKKKKKKKKK. In certain embodiments, the invention provides peptide aggregate-specific binding reagents where the reagents contain a peptide consisting of KKKKKK.

In preferred embodiments, the invention provides peptoid aggregate-specific binding reagents, where the reagents include

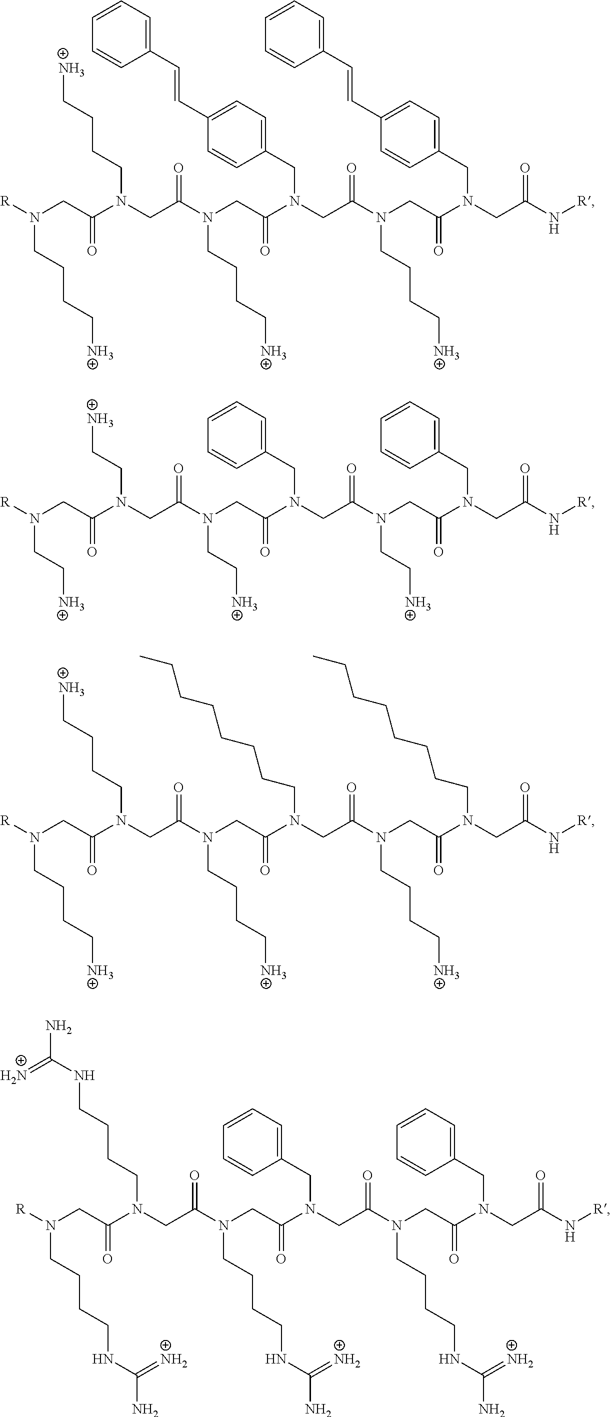

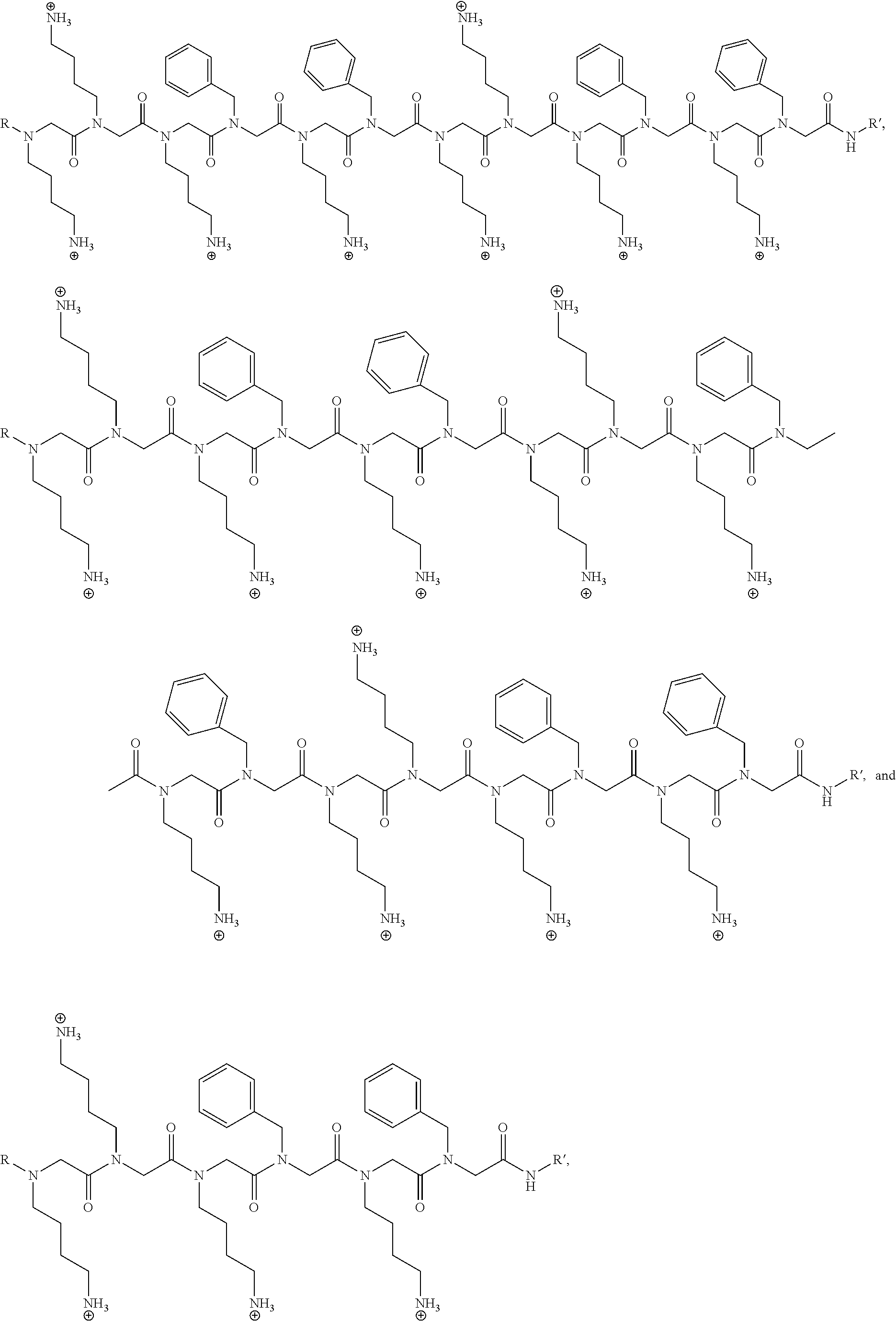

wherein R and R′ is any group.

In certain embodiments, the invention provides dendron aggregate-specific binding reagents that bind preferentially to aggregate over monomer when attached to the solid support, where the reagents include

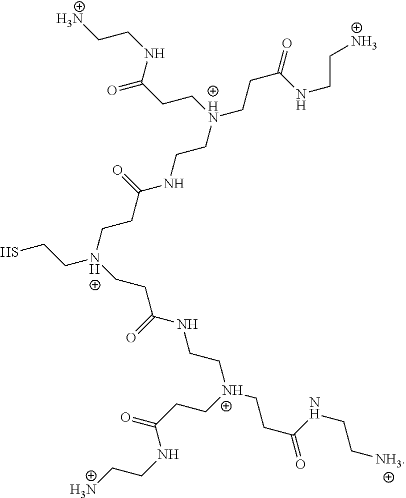

The aggregate-specific binding reagents of the compositions of the invention may also contain a hydrophobic functional group. The hydrophobic functional group may be, for example, an aromatic or an aliphatic hydrophobic functional group. In certain embodiments, the ASB reagents may contain functional groups such as amines, alkyl groups, heterocycles, cycloalkanes, guanidine, ether, allyl, and aromatics. Such aromatic functional groups include naphtyl, phenol, aniline, phenyl, substituted phenyl, nitrophenyl, halogenenated phenyl, biphenyl, styryl, diphenyl, benzyl sulfonamide, aminomethylphenyl, thiophene, indolyl, naphthyl, furan, and imidazole. In further embodiments, the ASB reagents contain repeating motifs. In other embodiments, the ASB reagents are detectably labeled.

The invention also provides compositions including solid supports and the aggregate-specific binding reagents described above. In preferred embodiments, the peptide, peptoid, or dendron aggregate-specific binding reagent is attached to the solid support at a charge density of at least about 60 nmol net charge per square meter, at least about 90 nmol net charge per square meter, at least about 120 nmol net charge per square meter, at least about 500 nmol net charge per square meter, at least about 1000 nmol net charge per square meter, at least about 2000 nmol net charge per square meter, at least about 3000 nmol net charge per square meter, at least about 4000 nmol net charge per square meter, at least about 5000 nmol net charge per square meter, or at least about 6000 nmol net charge per square meter and the composition binds preferentially to aggregate over monomer when attached to the solid support.

The solid support can be any material that is an insoluble matrix and can have a rigid or semi-rigid surface to which a molecule of interest (e.g., reagents of the invention, conformational proteins, antibodies, etc) can be linked or attached. Exemplary solid supports include, but are not limited to, substrates such as nitrocellulose, polyvinylchloride; polypropylene, polystyrene, latex, polycarbonate, nylon, dextran, chitin, sand, silica, pumice, agarose, cellulose, glass, metal, polyacrylamide, silicon, rubber, polysaccharides, polyvinyl fluoride, diazotized paper, activated beads, magnetically responsive beads, and any materials commonly used for solid phase synthesis, affinity separations, purifications, hybridization reactions, immunoassays and other such applications. The support can be particulate or can be in the form of a continuous surface and includes membranes, mesh, plates, pellets, slides, disks, capillaries, hollow fibers, needles, pins, chips, solid fibers, gels (e.g. silica gels) and beads, (e.g., pore-glass beads, silica gels, polystyrene beads optionally cross-linked with divinylbenzene, grafted co-poly beads, polyacrylamide beads, latex beads, dimethylacrylamide beads optionally crosslinked with N—N′-bis-acryloylethylenediamine, iron oxide magnetic beads, and glass particles coated with a hydrophobic polymer.

The invention further provides kits for performing the methods of the invention. Typically, the kits contain the compositions described in the previous two paragraphs.

### EXAMPLES

The following non-limiting examples are described for illustration.

### Example 1

### Assay for Testing Ability of Reagents to Capture Aggregates

This Example describes an assay designed to test the ability of reagents to bind aggregates.

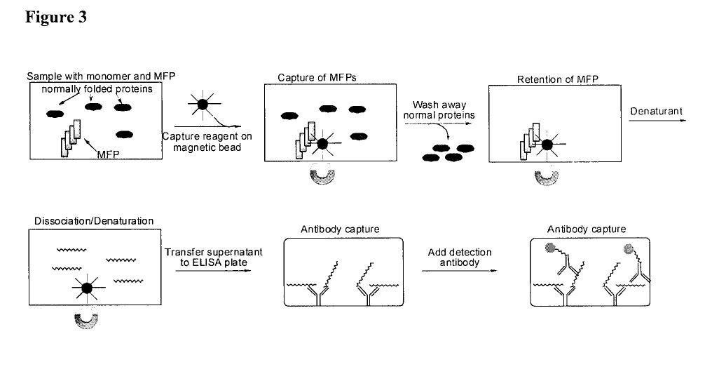

To assess the ability of these reagents to capture protein aggregates, the previously described Misfolded Protein Assay or MPA (Lau et al., 2007, PNAS, 104: 11551) was used (FIG. 3). In this assay, the capture reagent of interest is attached to beads which are incubated with a sample of interest containing a mixture of normal monomeric and aggregated proteins to allow for capture and then washed to remove unbound material. After this enrichment step, an elution buffer is used to dissociate the captured material from the beads as well as to denature any aggregates. The eluted material is then applied to a sandwich ELISA that is specific for the protein of interest. Strong aggregate-binding reagents acting as effective capture reagents show a high signal from the sample containing a mixture of monomers and aggregates, but not from the control sample, which contains only physiologic levels of monomeric protein.

Example 2 describes the use of this assay to test the ability of reagents with various properties to bind preferentially to oligomeric beta amyloid 1-42 (termed a “globulomer” by Barghorn et al., Journal of Neurochemistry, 2005) over monomeric beta amyloid 1-42 in CSF spiked with globulomer. Example 3 describes the use of this assay to test the abilities of various peptoid reagents to bind preferentially to disease-associated aggregates in buffer, CSF, or plasma spiked with diseased brain homogenates. Example 4 described the use of this assay to test the ability of a peptoid reagent to bind preferentially to various disease-associated aggregates over their normal counterpart monomers in brain homogenate from patients with the disease.

### Example 2

### Evaluating Binding Ability of Reagents

This Example demonstrates the effects of different capture reagent properties on their ability to bind preferentially to oligomers over monomers. Reagents having an overall positive charge and a high charge density on a solid support showed an increased ability to bind preferentially to oligomers. Furthermore, the addition of hydrophobic residues to the reagents improved preferential binding, whereas the specific scaffold of the reagent was not important as long as it was positively charged.

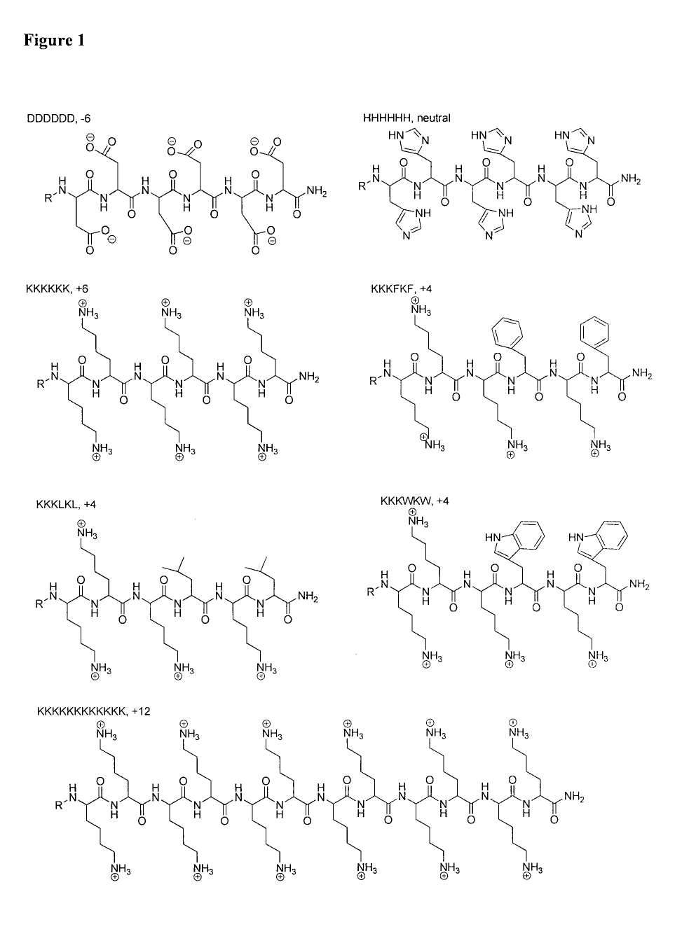

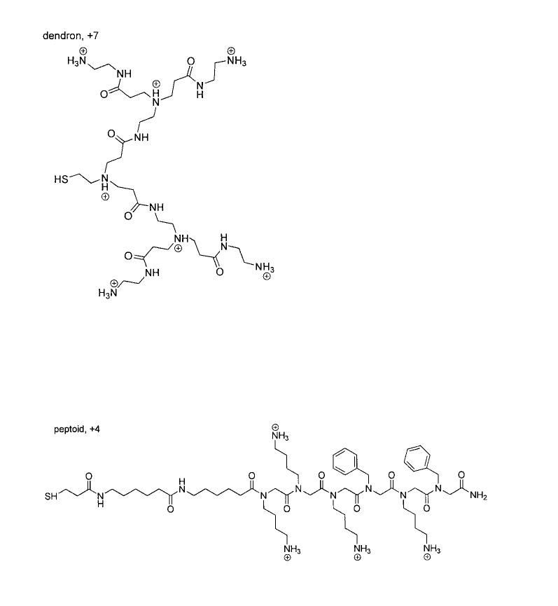

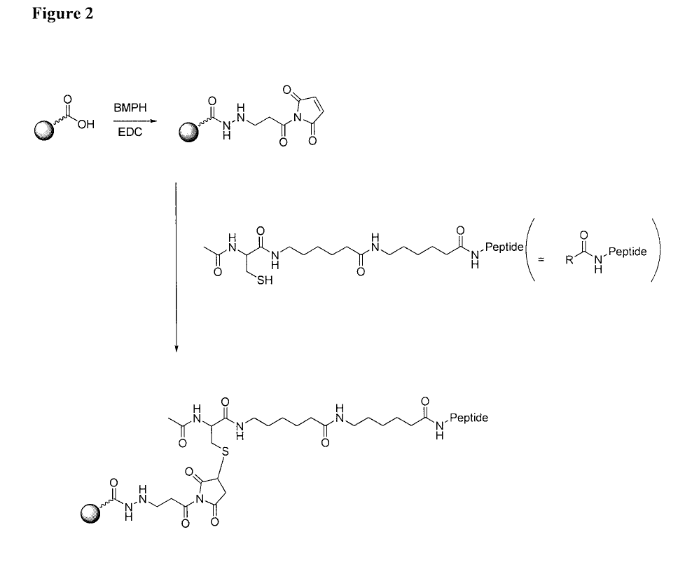

Protein aggregates can bind to a capture reagent through a variety of mechanisms such as ionic bonding, hydrogen bonding, and hydrophobic interactions. A series of potential aggregate-specific binding reagents with widely varying charge, hydrophobicity, and scaffolds (dendrimer, peptide, peptoid) were designed to test these possible modes of binding (FIG. 1). The reagents were conjugated onto magnetic Dynal M270 beads (FIG. 2) by the following methods.

Beads displaying carboxylic acids were treated with EDC and BMPH to create maleimide-displaying beads, to which thiolated peptides (or other thiolated organic molecules) were added through a Michael addition reaction. Dynal M270 magnetized beads (30 mg/mL bead) displaying carboxylic acids were vortexed and placed into a 15 ml falcon tube. The tube was placed into a magnet, and the supernatant removed. The beads were washed 2 times with 0.1 M MES buffer, pH 5, and then the washing buffer was removed. The coupling solution (33 mM BMPH, 130 mM EDC in MES buffer) was added, and the tube was rocked for 30 minutes at room temperature. After washing in 1×MES, 1× Tris, pH 7.5, the beads were quenched with Tris buffer (50 mM Tris buffer, pH 7.5) for 15 minutes. The beads were then washed 2 times in phosphate buffer and added to 5 mM thiolated ligand in degassed phosphate. The beads were rotated for 21 hours, then washed in 0.1 M phosphate buffer, pH 7, 1×PBS, and stored.

To prepare the globulomer, beta amyloid (1-42) was monomerized with incubation in hexafluoroisopropanol. The hexafluoroisopropanol was removed by vacuum centrifugation. DMSO, PBS, and 2% SDS were then added to the sample. The sample was vortexed and sonicated and then incubated at 37° C. After 6 hours, the sample was diluted with water, vortexed, and incubated at 37° C. for an additional 19 hours. The sample was ultracentrifuged at 135000×g for 1 hour at 4° C., and the supernatant retained. The globulomers were spiked into CSF for the assay.

Aliquots of beads conjugated to various reagents were added to wells of a 96-well plate. Globulomer-spiked CSF in a Tris buffer was added to each well, and the plates were incubated for 1 hour at 37° C. with shaking. For the negative control, normal CSF was used, which is considered to contain only monomers of beta amyloid. The beads were washed with TBST wash buffer, and bound materials were eluted with a denaturing solution (typically 0.1-0.15 N NaOH). A reconditioning buffer was added to the eluate prior to beta amyloid detection via a beta amyloid (1-42) specific sandwich ELISA.

### Charge

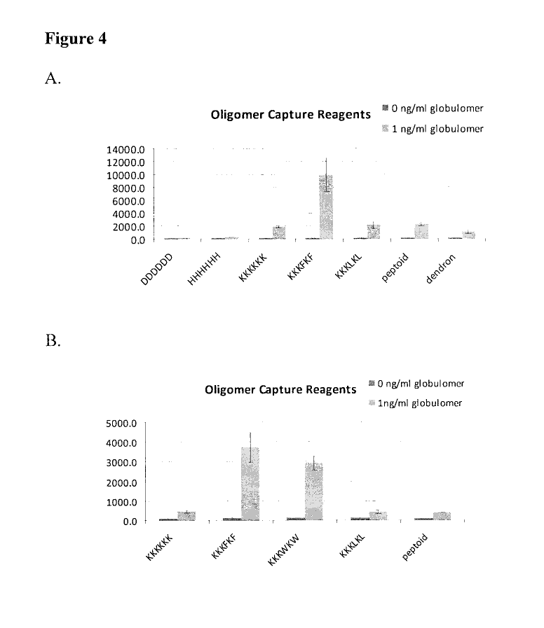

First, it was determined whether charge-based interactions allow for oligomer capture. Peptides containing negatively charged residues (e.g. aspartic acid, D), positively charged residues (e.g. lysine, K), and neutral residues (e.g. histidine, H) were tested. Representative results are shown in FIG. 4A. Negatively charged (DDDDDD) and neutral (HHHHHH) peptides provided little enrichment of the oligomeric species, whereas positively charged (KKKKKK) peptides provided significant capture. It was postulated that negatively charged residues on the oligomer (or salts, lipids, or other species bound to the oligomer) interact with the positively charged capture peptide.

### Hydrophobic Interactions

Although positive charge alone was sufficient for enrichment of oligomers, hydrophobic interactions were tested to determine whether they provided additional capture efficiency. The all positively charged peptide KKKKKK was compared with peptides containing aromatic hydrophobic residues (e.g. tryptophan, W, or phenylalanine, F) and aliphatic residues (e.g. leucine, L). The peptides KKKFKF and KKKWKW provided increased capture efficiency relative to the corresponding peptides with aliphatic hydrophobic residues or no hydrophobic residues (FIG. 4B), demonstrating that addition of aromatic hydrophobic residues improved the capture.

### Alternative Scaffolds

In order to assess whether the enrichment method was limited to peptidic scaffolds or whether other positively charged organic molecule scaffolds could also enrich oligomers, two additional scaffolds, peptoids and dendrons, were tested. Peptoids are linear polymers of N-substituted glycines, and therefore retain spacing similar to that of peptides, but are achiral and tend to have different conformations in solution than peptides. Dendrons are branched polymers with little structural similarity to peptides. In the NMPA assay, the positively-charged peptoid and dendron shown in FIG. 1 were both capable of enriching oligomers (FIG. 4A), demonstrating that a peptidic scaffold is not critical for capture.

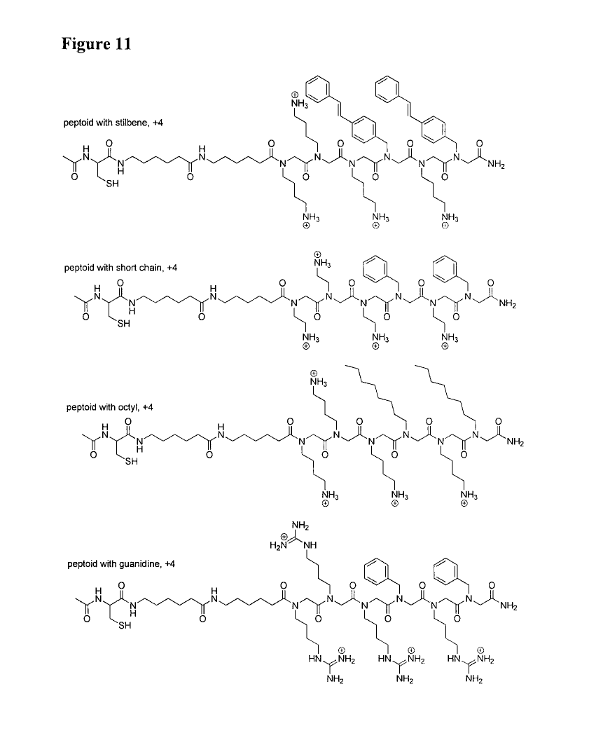

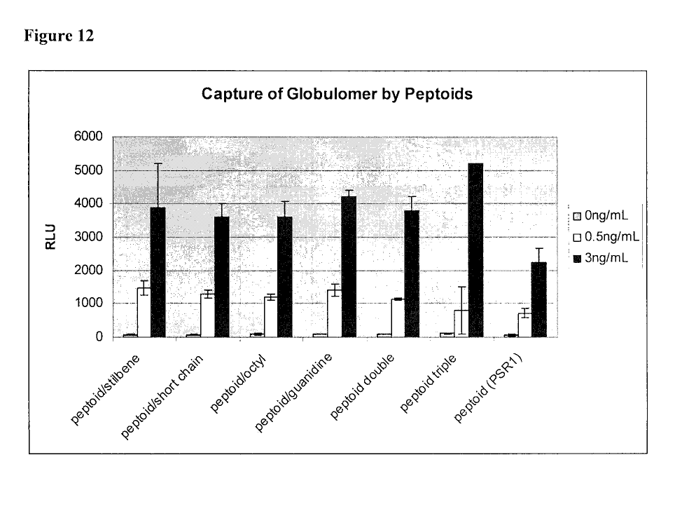

To investigate the effect of different hydrophobicities and charge on the ability of peptoid scaffolds to capture oligomers, an additional set of peptoids was tested with globulomer-spiked CSF. FIG. 11 shows the structures and charges of the additional peptoids tested. The stilbene and octyl peptoids have a different hydrophobic monomer compared with the original PSR1 peptoid (replacement of the benzyl group with a larger aromatic stilbene or an aliphatic octyl chain). The short chain and guanidine peptoids have different cationic groups than the original peptoid. The short chain peptoid has an ethyl rather than a butyl spacer between the side chain amine and the peptoid backbone, and the guanidine has a more basic side chain than the original peptoid. The double and triple peptoids have increased length relative to PSR1, and therefore more charges per ligand. FIG. 12 shows the results of MPA assays with these peptoid reagents. All of the additional peptoids captured globulomer similarly to PSR1.

### Avidity

The results described above showed that a net positive charge was important for efficacious capture, whereas the specific scaffold was less important. Therefore, it was postulated that one major binding modality was through ionic interactions. Individual ionic interactions are relatively weak, thus the interaction between oligomer and capture reagent may have an avidity component in which capture efficiency is based on the combined strength of multiple ligands. In such a case, the density of ligands on a surface is important.

To assess this possibility, a series of beads attached to different amounts of the positively charged peptoid were prepared, such that each bead had a different charge density display. In order to determine the amount of ligand loading, amine quanitation was used. Aliquots of beads were placed in a magnet, and the supernatant removed. 80% phenol in ethanol, 0.2 mM KCN in pyridine/water, and 6% ninhydrin in ethanol was added to each tube. The aliquots were vortexed and heated for 7 minutes at 100° C. After cooling to room temperature, 60% ethanol was added. The tubes were placed in a magnet, and the absorbance of the supernatant at 570 nm was determined. The loading of the beads was determined according to Beer's law, using the extinction coefficient of 15000 M-1 cm-1.

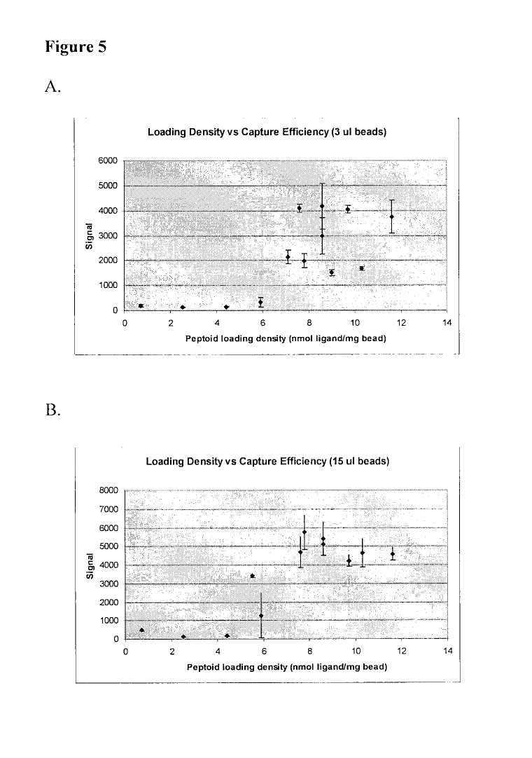

Beads (3 ul or 15 ul) were added to samples containing 0.5 ng/mL globulomer spiked into CSF. As can be seen in FIG. 5, capture increased non-linearly with ligand density, such that there was a minimum density required for oligomer capture. For PSR1, this limit was ˜5 nmol ligand/mg bead for binding preferentially to globulomer. Given the approximate 2-5 m² of surface area per gram bead, this value was approximately 1500 nmol ligand/m², or roughly 6000 nmol positive charges/m², assuming that all amines were protonated at the pH 7.4 assay conditions.

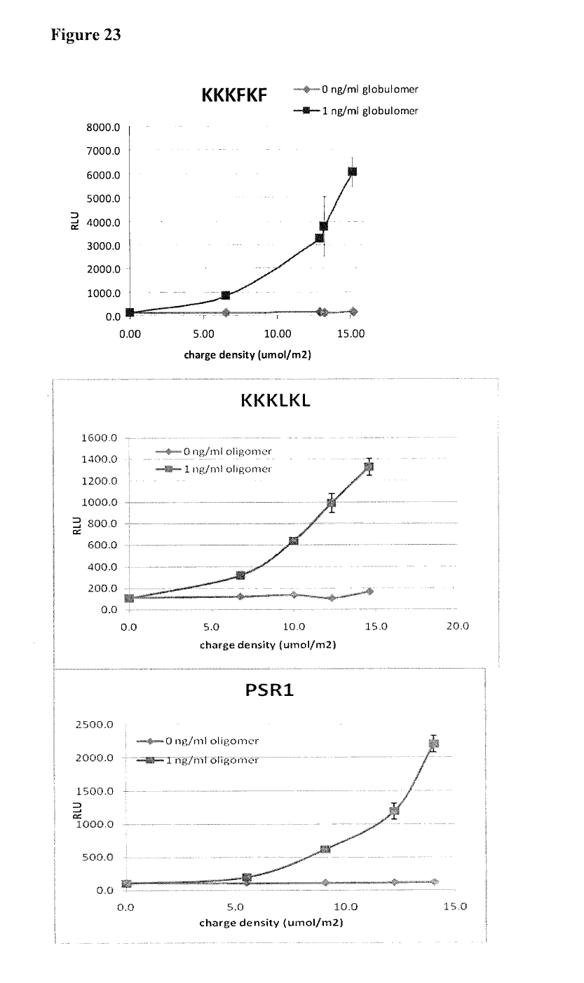

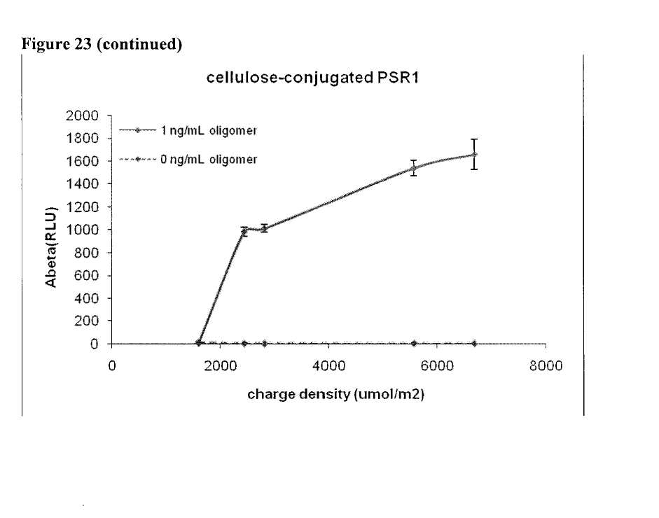

Additional experiments were carried out to assess the relationship between binding efficiency of the capture reagent and minimum charge required for specific capture of oligomers. A series of beads bearing a capture reagent (the peptide KKKFKF, the peptide KKKLKL, or the peptoid PSR1) was prepared in loading densities ranging from ˜6000 nmol/m2 to ˜15000 nmol/m2. Methods were the same as described in the two paragraphs above, but 1 ng/mL globulomer was used and 3 μl beads were added. Similar to the initial experiments on charge density described above, capture of oligomers increased exponentially with charge density (FIG. 23). At the lowest loading densities tested (˜6000 nmol/m2), it was still possible to distinguish between background and captured oligomer. For highly efficient reagents such as the peptide KKKFKF, it was estimated that as little as 500 nmol/m2 ligand, or 2000 nmol positive charges/m², would be sufficient for selective capture of oligomers.

It is possible to observe avidity-based capture with the reagent conjugated to other solid supports. PSR1 was conjugated to a cellulose membrane using a protocol shown in the following paragraph, on which much higher levels of loading can be reached. An increase in PSR1 loading of approximately 100× higher than what was loaded on beads continued to increase the ability of PSR1 to bind preferentially to oligomers in solution (FIG. 23).

For conjugating peptoids/peptides directly on the membrane: A cellulose membrane (Whatman 50) was immersed in 10:1:90 solution of epibromohydrin:perchloric acid:dioxane and allowed to incubate 1-3 h rt. After washing with methanol and drying, the membrane was aminated by incubation in neat trioxadecanediamine at 70 C for 1 h. After washing, the membrane was quenched (in 3M NaOMe), washed and dried again. Spots were demarcated by spotting 1 ul of a 0.4M solution of FmocGly preactivated with 11013T and DIC in NMP and incubating for 20 min. The coupling was repeated and the membrane capped with 2% acetic anhydride in DMF, followed by 2% acetic anhydride/2% DIEA in DMF. The membrane was washed with DMF, deprotected with 4% DBU in DMF (2×10-20 min), washed with DMF and methanol, and then dried. Activated maleimidoproprionic acid (0.4 M with HOBt and DIC) was added to the spots of the membrane, the coupling repeated, and the membrane washed with NMP, water, and methanol. Aliquots (2 ul) of 10 mM thiolated peptoid in DMF/phosphate buffer were added to the membrane. The thiolated peptoid addition was repeated, the membrane quenched (with BME) and washed (water, methanol, DMF, and methnaol), and finally dried before use

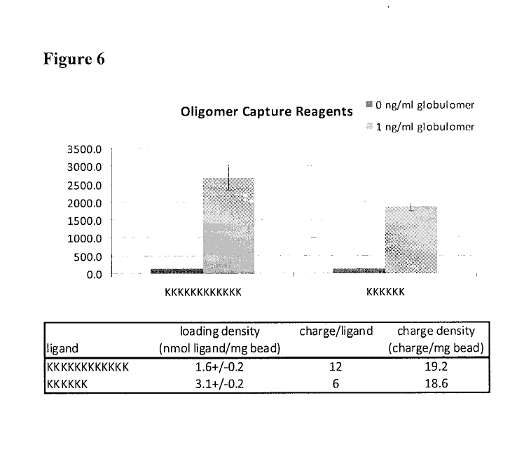

In order to further probe this charge density effect, the charge on a single ligand was increased to determine whether the increase in charge/ligand could compensate for decreased surface density. Two positively charged peptides, KKKKKK (loading level: 3.1 nmol/mg bead) and KKKKKKKKKKKK (loading level: 1.6 nmol/mg bead) were compared (FIG. 6). Doubling the number of charges per ligand (from 6 to 12) did not necessarily double the capture efficiency if there was a concomitant decrease in loading of ligand onto the bead.

### Avidity and Choice of Solid Support

The role of avidity in capture of oligomers was also examined by comparing two different solid supports for the PSR1 peptoid reagent. When PSR1 is directly conjugated to magnetic beads, the density of the peptoid ligand is ˜3.5 μmol/m², and therefore the charge density is ˜14 μmmol charge/m². In contrast, the density of biotin-PSR1 bound to streptavidin-coated magnetic beads is ˜0.033 μmol/m², and therefore the charge density is ˜0.12 μmol charge/m². The oligomer capture abilities of these two PSR1 beads were compared to evaluate the effect of beads with different levels of charge density.

Equivalent amounts of PSR1 and two different input levels, 3 or 30 μl of beads directly conjugated to PSR1 (30 mg/ml, “PSR1 beads”) or 10 or 100 μl of streptavidin beads bound to biotin-PSR1 (10 mg/ml, “b-PSR1 beads”), were used in MPA assays with a mixture of 80 μl of globulomer-spiked CSF and 20 μl of 5×TBSTT. Globulomers were added in their native conformation (“native glob”) or as monomers (“denatured glob”) for the negative control. Globulomers were denatured in 5M GdnSCN at room temperature for 30 minutes. Although the equivalent amounts of beads were used, the charge density of the PSR1 beads was approximately 100 times greater than that of the b-PSR1 beads.

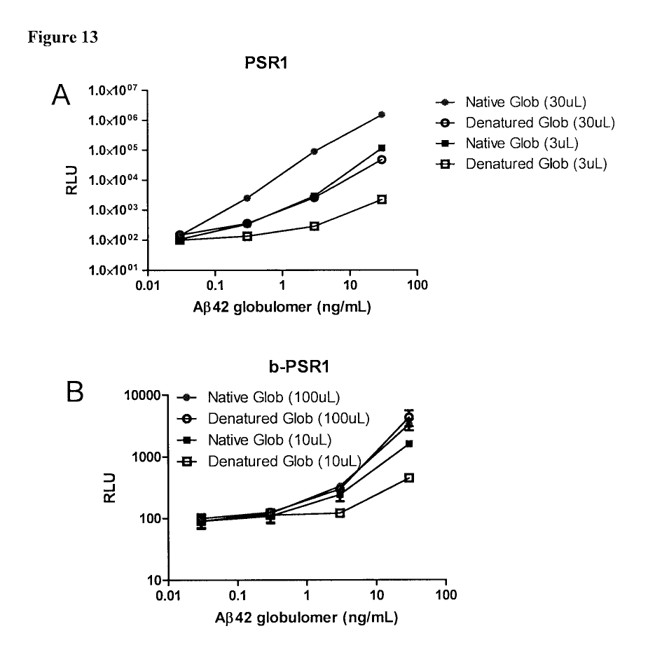

PSR1 beads showed higher sensitivity and specificity for globulomers while the low-density b-PSR1 beads showed limited specificity and sensitivity (FIGS. 13 A and B), which further indicated that charge density is critical for capture of oligomers.

### Example 3

### Detection of Fibrils with Various Peptoid Aggregate-Specific Binding Reagents with Varying Charges and at Varying Charge Densities

This Example describes the capture of fibril aggregates with peptoid reagents. Similar to the conclusions made in Example 2, an overall positive charge and a high charge density on a solid support were critical for increased preferential binding of peptoid reagents to fibril aggregates over monomers.

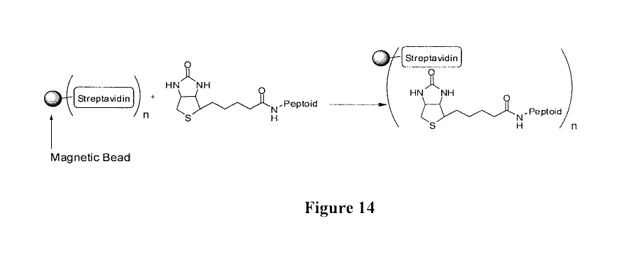

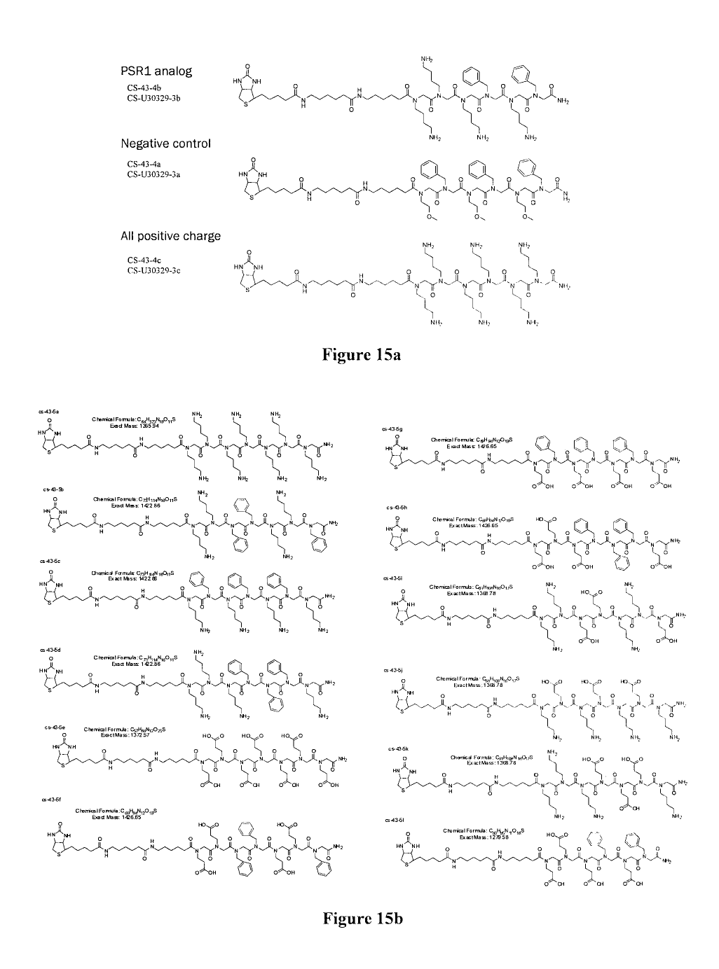

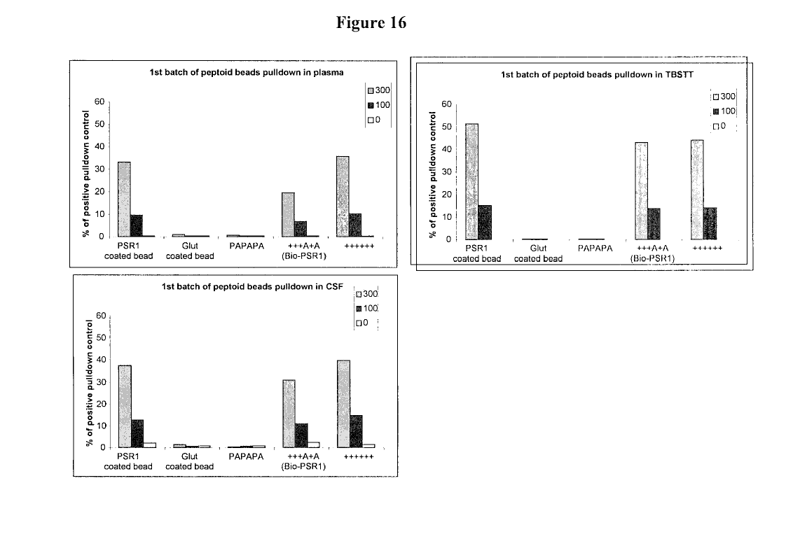

Compounds were prepared as biotinylated derivatives which can be bound to streptavidin-derivatized magnetic beads for testing (see FIG. 14). The peptoids were prepared using the submonomer method, essentially as described previously by Zuckermann, et al. (J. Am. Chem. Soc. (1992) 114:10646-10647; J. Am. Chem. Soc. (2003) 125:8841-8845; J. Pept. Prot. Res. (1992) 40:498) and purified by HPLC (FIG. 15, Table 6).

Peptoids are abbreviated to describe the order and identity of their submonomers. The peptoid submonomers are denoted: “+” indicating a submonomer that would be positively charged at pH 7; “−” indicating a submonomer that would be negatively charged at pH 7; “A” indicating an aromatic submonomer; and “P” indicating a polar uncharged submonomer. The sequence is noted N->C and the biotin-(aminohexanoic acid)₂ linker is implied. “+” of “positively charged” indicates basic functional groups, expected to be positively charged under the conditions employed in the examples.

TABLE 6
Characterization Information for Peptoids

| | Abbreviation of | | | | | | |
|---|---|---|---|---|---|---|---|
| | Sequence | | | | | | |
| SEQ ID NO | Characteristics | prep method | t_{R} (prep) | analytical method | t_{R} (analytical) | exact mass | mass observed |
| 21 | PAPAPA | A | 5.53 | positive, high mass | 2.14 | 1255.67 | 1256.4 |
| 15 | +++A+A | B | 4.52 | positive, high mass | 1.49 | 1275.79 | 1277.5 |
| 16 | ++++++ | B | 2.73 | positive, high mass | 1.27 | 1237.84 | 1238.5 |
| 17 | +++++++ | B | 2.8 | positive, high mass | 1.16 | 1365.94 | 1367.7 |
| 18 | ++AA++A | C | 4.09 | positive, high mass | 1.67 | 1422.86 | 1425.5 |
| 19 | +A+A+A+ | C | 4.03 | positive, high mass | 1.64 | 1422.86 | 1424.5 |
| 20 | +++AAA+ | C | 4.18 | positive, high mass | 1.72 | 1422.86 | 1425.5 |
| 22 | −−−−−−− | B | 4.37 | negative, high mass | 1.41 | 1372.57 | 1371.3 |
| 23 | −−AA−−A | C | 6.53 | negative, high mass | 1.97 | 1426.65 | 1425.3 |
| 24 | −A−A−A− | C | 6.52 | negative, high mass | 1.96 | 1426.65 | 1425.3 |
| 25 | −−−AAA− | C | 6.84 | negative, high mass | 2.02 | 1426.65 | 1425.3 |
| 26 | ++−−++− | B | 2.97 | positive, high mass | 1.30 | 1368.78 | 1369.5 |
| 27 | +−+−+−+ | B | 3.18 | positive, high mass | 1.32 | 1368.78 | 1371.4 |
| 28 | +++−−−+ | B | 3.12 | positive, high mass | 1.31 | 1368.78 | 1371.4 |
| 29 | −−−A−A | C | 5.59 | negative, high mass | 1.81 | 1279.58 | 1278.3 |

Analytical HPLC-MS: Agilent 1100, 0.8 ml/min flow rate, 5-95% MeCN/H₂O/TFA over 3.5 min, 100 × 2.1 mm 5 μm Hypersil ODS column, UV detection at 214 nm for 6 min, MS mode noted.
Preparatory HPLC: 30 ml/min flow rate, gradient noted below MeCN/H₂O/TFA over 16 min, 30 × 50 mm SF C18 column, Waters detector, UV detection at 214 nm for 16 min.

### Pulldown of Prion Aggregates

The three biotinylated peptoid analogs shown in FIG. 15a were conjugated onto streptavidin-derivatized magnetic beads. Two known bead conjugates, Dynal M270 beads directly coated with PSR1 (positive control) or glutathione (negative control), were also tested for comparison. The five bead conjugates were assayed using the Misfolded Protein Assay (FIG. 3). The five bead conjugates were added to wells of a 96 well plate. 10% brain homogenates (w/v) from prion infected hamster, known to be rich sources of the large aggregates of the misfolded form of the prion protein, PrP^{Sc}, were used as the sample. Brain homogenate was spiked into buffer, cerebrospinal fluid (CSF), and plasma each at three levels, 300, 100, and 0 mL/mL. The brain homogenate solutions were added to the beads and allowed to incubate for a period of time, typically 1 hr at 37° C., with rotation. A magnet was applied to the sample to allow separation of the bead bound material from the supernatant. After removal of the supernatant, an elution buffer was applied to denature the aggregates and dissociate the eluted material from the bead. The eluted material was then applied to a sandwich ELISA assay specific for the protein of interest.

The results of the Prp^{Sc} capture experiments are shown in FIG. 16. No signal was observed from the glutathione beads or the uncharged beads (PAPAPA). Signal was observed with the 100 or 300 ng/mL spike levels when using the PSR1 coated beads, the biotinylated analog of PSR1 (+++A+A), and the peptoid containing 6 positive charges (++++++). The signals were not significantly different between these three peptoid-bead conjugates for assays performed in buffer, but PSR1 coated beads and ++++++++ provided a moderate increase in signal in the other two matrices relative to the biotinylated analog of PSR1 (+++A+A). No signal was observed in the no-spike samples. Similar signal/noise levels for captured Prp^{Sc} were observed for the biotinylated PSR1 (+++A+A) and the previously described beads directly coated with PSR1, validating the library format shown in FIG. 14. A peptoid bearing 6 positive charges efficiently captured PrP^{Sc} as well.

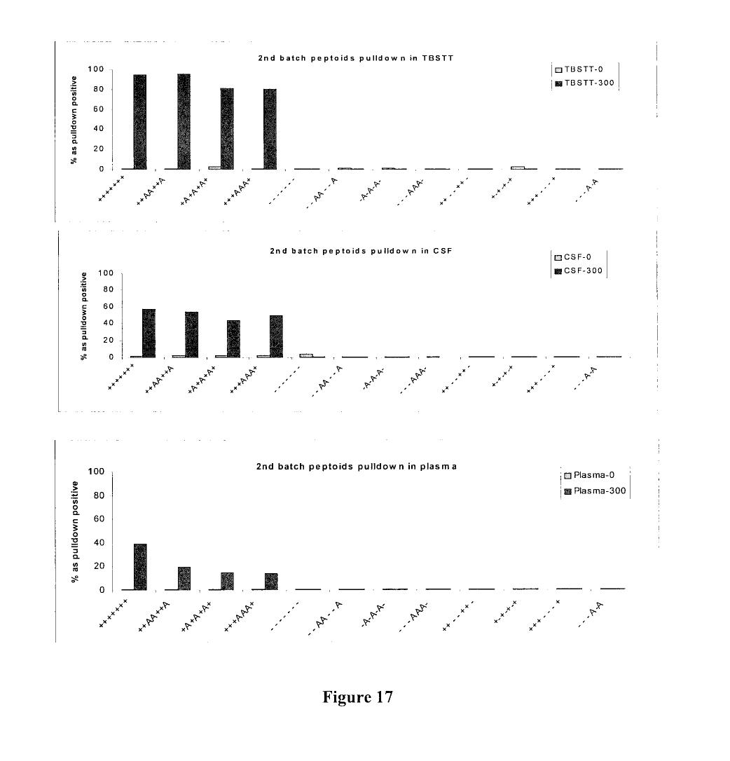

Biotinylated peptoid analogs shown in FIG. 15b were conjugated onto streptavidin-derivatized magnetic beads. Homogenates from prion infected hamster brains were tested as described above, except that only 0 and 300 ng/mL spike levels were explored. The results of these experiments are shown in FIG. 17 (data shown is in triplicate). The data is shown relative to beads coated with PSR1 (positive control, +++A+A) in buffer. The four peptoids with the highest overall positive charge provided a signal comparable with PSR1 in buffer, while negatively charged or charge neutral, zwitterionic peptoids showed significantly lower signals. Decreased signal was observed from samples spiked into CSF or plasma relative to the buffer assays, but the positively charged peptoids still showed significant capture. Peptoids bearing overall positive charges of +4 and +7 efficiently captured Prp^{Sc}. For peptoids bearing 3 aromatic and 4 positive submonomers, the order of the submonomers did not dramatically impact signal.

### Pulldown of Aβ Aggregates

A similar approach to the prion assay described above was used to assess Aβ aggregate capture by the peptoid-bead conjugates. 10% b rain homogenate (w/v) from an Alzheimer's Disease patient brain was used as a positive control, as these samples are known to be rich in large aggregates of Aβ(1-40), Aβ(1-42), and tau.

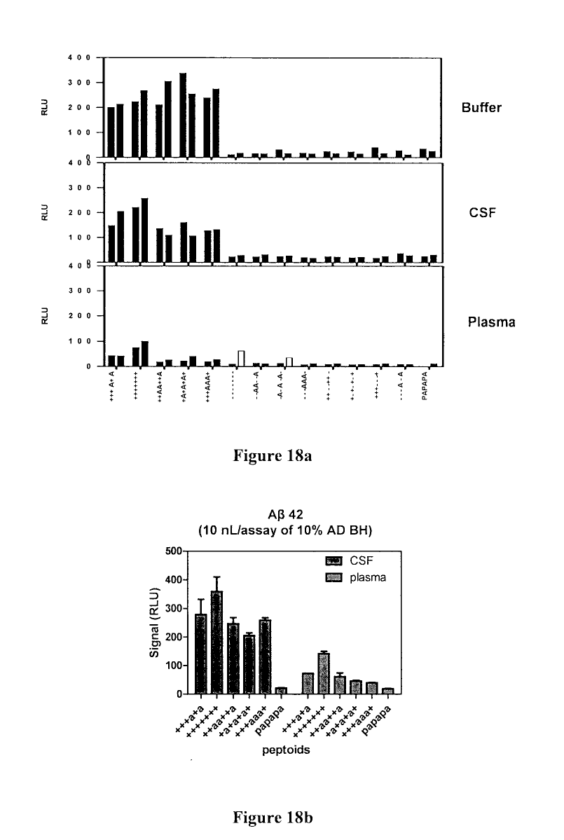

For Aβ(1-42), 10 nL of 10% brain homogenate was added to each well, and the signals from duplicate assays were detected (FIG. 18A). The overall trend from these experiments showed a similar trend to the prion capture experiments described above. Low signal was observed for peptoids with low overall positive charge, and capture efficiency was strongest in buffer. To further investigate the utility of positively charged peptoids for capturing Aβ(1-42), a second experiment focusing on the overall positively charged peptoids was performed (FIG. 18B). Little sequence specificity was observed between the peptoids containing various orders of 3 aromatic and 4 positive charges, however the peptoid with 7 positive charges provided increased signal over the ones with 4 positive charges in both plasma and CSF.

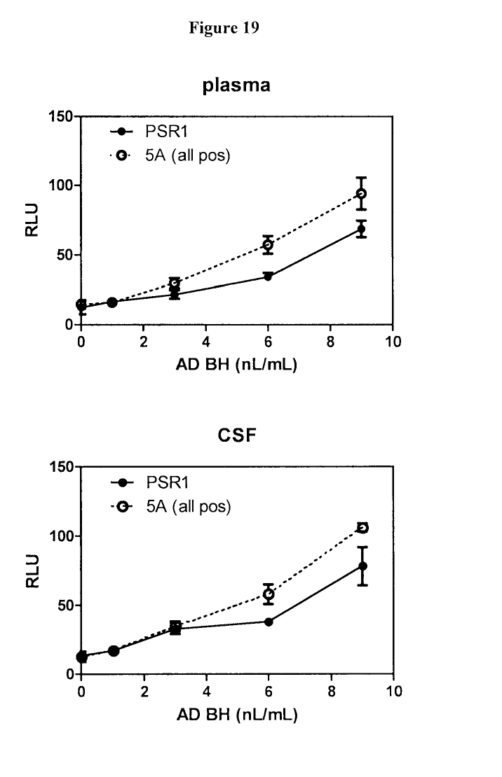

Similar limits of detection between +++A+A (biotinylated PSR1) and +++++++ for Aβ(1-42) capture in CSF and plasma were observed (FIG. 19) (1.3 vs. 1.6 mL/assay in CSF and 3.8 vs. 2.5 mL/assay in plasma).

Peptoids bearing overall positive charges of +4 to +7 efficiently captured Aβ(1-42) in buffer, and to a lesser extent, in CSF and plasma. For peptoids with 3 aromatic and 4 positive submonomers, the order of the submonomers did not dramatically impact signal. Similar limits of detection were found for +++A+A (biotinylated PSR1) and +++++++.

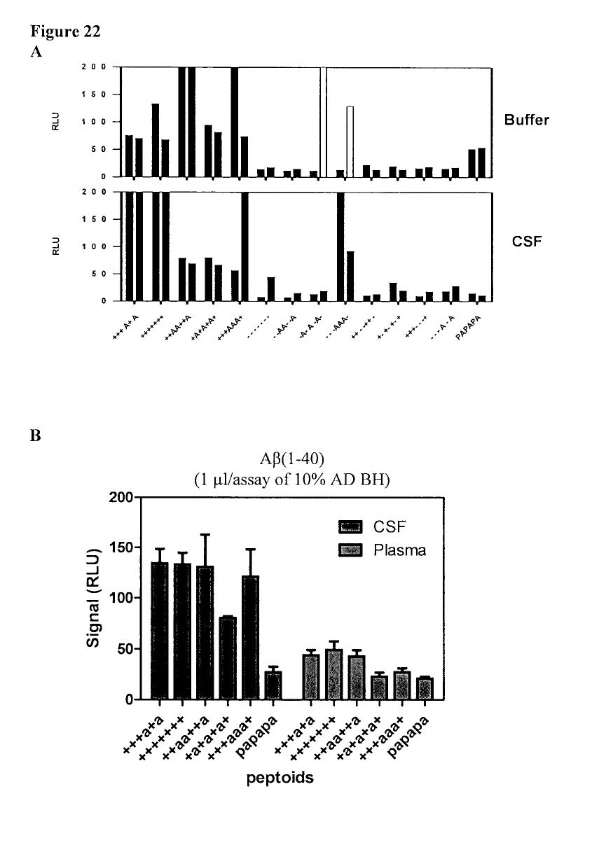

For Aβ(1-40), 1 μL of 10% brain homogenate was added to each well and the signal from duplicate assays detected (FIG. 22A). While larger variability in signal was observed in this data, the overall trend from these experiments showed a similar trend to the prion work. In general, higher signal was observed in the assays with higher positively charged peptoids for buffer and CSF.

The results for −A−A−A− and −−−AAA− were inconsistent with previous findings showing that positive charge is a requirement for preferential binding to aggregates. However, given the poor reproducibility within the replicates, this result is not conclusive and requires further confirmation.

To further investigate the utility of positively charged peptoids for capturing Aβ(1-40), a second experiment focusing on the overall positive charged peptoids was performed (FIG. 22B). The signal from the assays using the peptoid with 7 positive charges was as high or higher than those containing 4 positive charges. Peptoids bearing overall positive charges of +4 to +7 efficiently captured Aβ(1-40) in buffer, and, to a lesser extent, in CSF and plasma. For peptoids with 3 aromatic and 4 positive submonomers, the order of the submonomers did not dramatically impact signal.

### Pulldown of Tau Aggregates

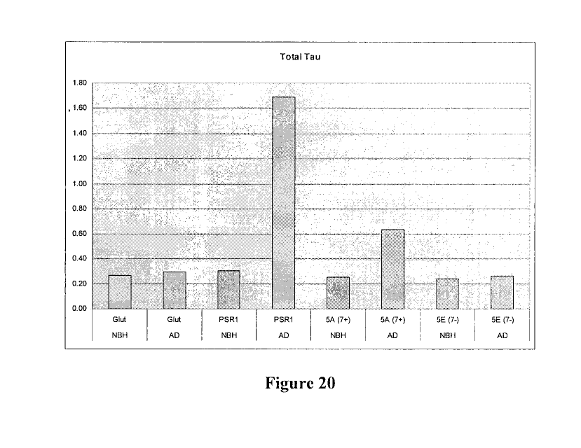

A similar approach to the prion assay described above was used to assess tau capture by the peptoid-bead conjugates. 160 mL of brain homogenate from Alzheimer's Disease (AD) patient brain was used as a positive control, as these samples are known to be rich in large aggregates of Aβ(1-40), Aβ(1-42), and tau. As a control, the results were compared to normal brain homogenate, which should have minimal tau aggregates. A comparison of the bead coated with PSR1 (+++A+A) and bead coated with glutathione to biotinylated +++++++ and −−−−−−− showed that both PSR1 (+++A+A) and (+++++++) had higher signals in the AD samples relative to the normal brain homogenates (NBH), whereas the glutathione control and the −−−−−−− peptoid had similar signals with both samples. PSR1 (+++A+A) had the highest signal (FIG. 20).

### Measuring Effect of PSR1Density on Bead in Binding Pathogenic Prion Aggregates

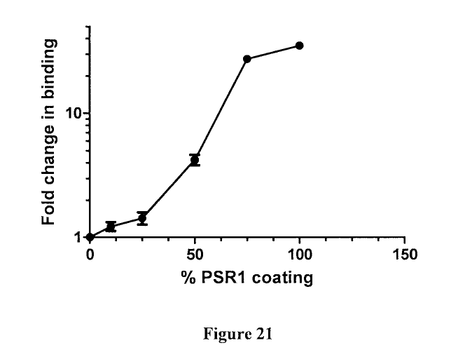

Since the analytes in the above assays are presumed to be protein aggregates, the capture efficiency of PSR1 as a function of density on the bead surface was investigated. Streptavidin magnetic beads were treated with solutions containing different ratios of biotinylated PSR1 (+++A+A) and charge neutral control peptoid (PAPAPA). After washing away unbound peptoids from the beads, the beads were mixed with human plasma spiked with Syrian hamster brain homogenate containing pathogenic prion aggregates. Excess proteins were washed away, and prion aggregates were eluted from beads and detected with ELISA specific for the prion protein (FIG. 3). As expected, beads conjugated with a charge neutral control peptoid (PAPAPA) led to minimal signal in the ELISA, whereas beads conjugated only with PSR1 (+++A+A) yielded a ˜35 fold higher signal. Consistent with the charge density requirements seen in the oligomer assays, a linear correlation was not observed between Bio-PSR1 (+++A+A) charge density and signal (Table 7 and FIG. 21). Increasing PSR1 conjugate concentration from 0 to 50% (˜60 nmol charge/m2) yielded a 4 fold S/N increase, while further increase by 25% (−30 nmol charge/m2) to 75% (−90 nmol charge/m2) yielded almost 27 fold increase in S/N. Further increasing the PSR1 (+++A+A) conjugate concentration to 100% (−120 nmol charge/m2) yielded moderate S/N increase over the 75% readout.

TABLE 7
Total prion signal as captured by Streptavidin magnetic beads conjugated
with increasing density of PSR1 (+++A+A)

| | charge density of Bio-PSR1 | | | | | |
|---|---|---|---|---|---|---|
| | (nmol charge/m²) | | | | | |
| | ~120 | ~90 | ~60 | ~30 | ~12 | 0 |
| Bio-PSR1 (+++A+A) | 100% | 75% | 50% | 25% | 10% | 0% |
| Charge Neutral (PAPAPA) | 0% | 25% | 50% | 75% | 90% | 100% |
| Read 1 | 53.5 | 39.5 | 6.6 | 2.2 | 1.7 | 1.3 |
| Read 2 | 59.4 | 43.5 | 6.3 | 2.8 | 1.9 | 1.8 |
| Read 3 | 54.4 | 47.7 | 7.2 | 1.8 | 2.3 | 1.6 |
| Average | 55.8 | 43.6 | 6.7 | 2.3 | 1.9 | 1.6 |
| SD | 3.2 | 4.1 | 0.4 | 0.5 | 0.3 | 0.2 |
| Signal/Noise | 34.9 | 27.2 | 4.2 | 1.4 | 1.2 | 1.0 |

### Example 4

### Detection of Aggregate Proteins in Patient Samples

This Example demonstrates that the peptoid capture reagent depicted in FIG. 1, PSR1, can distinguish between monomers and aggregates in several diseases associated with misfolded protein aggregates.

Experiments were carried out according to the method described in Example 1. 75 mL of 10% AD brain homogenate was spiked into 1×TBSTT and incubated with 3 ul PSR1 beads for 1 hr. PSR1 beads were subsequently washed and bound Aβ42 or tau aggregates were eluted and detected by Aβ42 and tau-specific sandwich ELISAs, respectively.

### Aggregate Capture in Brain Homogenates

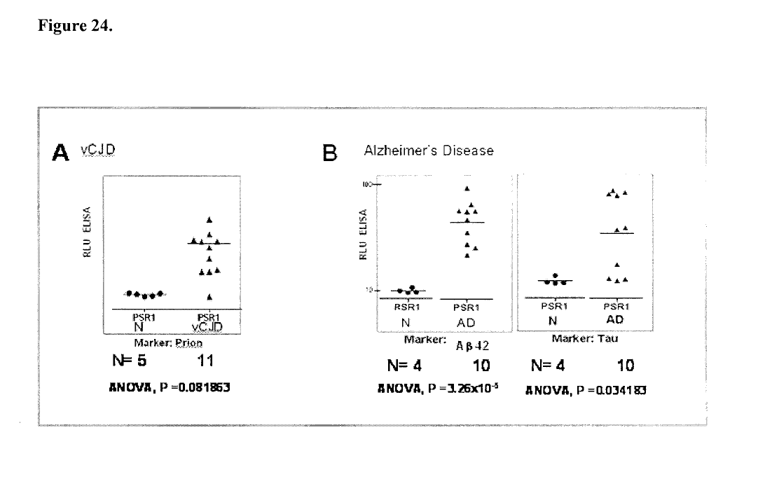

NMPAs were performed on brain homogenates from control, variant Creutzfeldt-Jakob Disease (vCJD), or Alzheimer's disease (AD) patients from Dr. Adriano Aguzzi at the University of Zurich Hospital. For vCJD, prion protein was detectεd. For AD, both Abeta (1-42) and Tau were detected. Results showed that PSR1 clearly distinguished between control and either vCJD or AD samples (FIG. 24).

### Example 5

### Determining the Role of E22 in Globulomer Capture

This Example demonstrates that charge, structure, and size of the aggregate contribute to its recognition by a peptoid aggregate-specific binding reagent attached to beads.

As described above, charge interactions between aggregate-specific binding reagents and aggregates are an important component of the binding mechanism. Example 2 demonstrated that positively charged reagents provided significant capture of oligomers. Thus, surface-exposed negatively charged residues on the oligomers are likely to be involved in binding to these reagents. Structural studies of the beta amyloid fibril and the N-Met preglobulomer suggested that the negatively charged E22 residue is surface-exposed (Luhrs et al, PNAS, 2005; Yu et al., Biochemistry, 2009). Therefore, studies were carried out to determine if exposed E22 is critical for capture of beta amyloid by PSR1, a positively charged capture reagent.

Three beta amyloid 1-42 peptides were generated to test the role of E22: a wild-type peptide, a mutant peptide containing Arctic mutation E22G with a neutral charge, and a mutant peptide containing Italian mutation E22K with a positive charge. Synthetic peptides were commercially available from Anaspec.

Mutant peptides were oligomerized according to the methods described in Example 3.

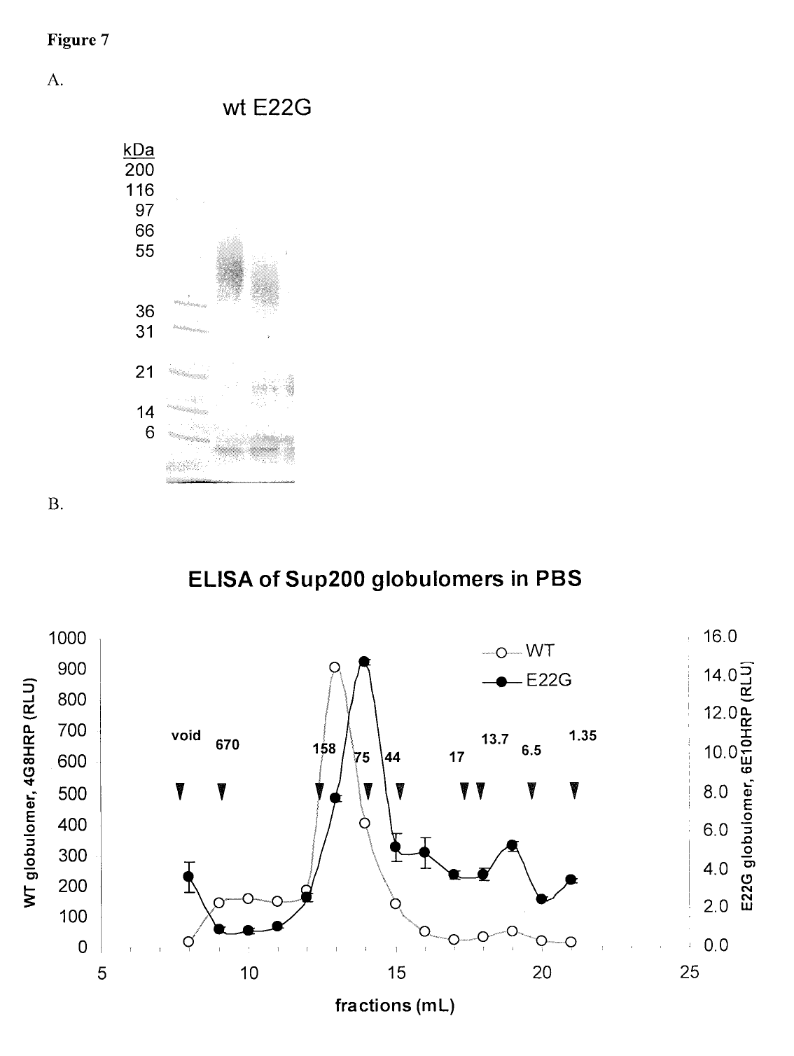

SDS-PAGE and size exclusion chromatography analyses of the E22G globulomer demonstrated that its structure is similar to that of a wild-type globulomer (FIG. 7). Oligomers were separated by 4-20% Tris-Glycine SDS-PAGE (Invitrogen) for 1.5-2 hr at 120V and gels were stained with Coomassie Blue. Oligomers were separated by SEC on a Superdex200 column in PBS, running at a flow rate of 1 mL/min. 1 mL fractions were collected and analyzed by an Aβ42-specific ELISA.

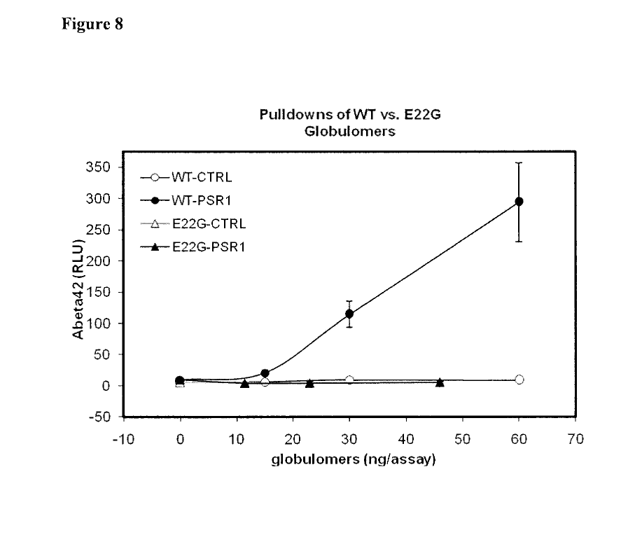

NMPA was used to evaluate the ability of PSR1 to capture the E22G globulomer. The methods used are described in Example 2. The neutrally charged E22G globulomer was not captured by PSR1 (FIG. 8), indicating that charge interactions are a key factor in the recognition of misfolded proteins.

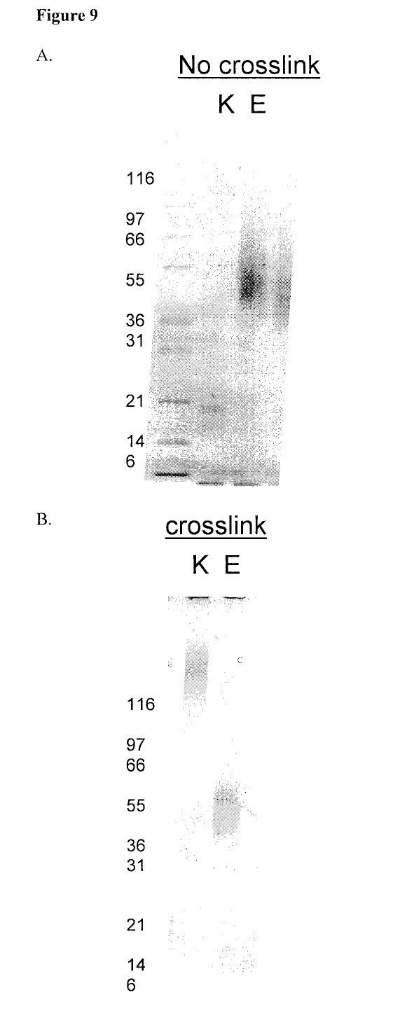

The E22K globulomer was evaluated and compared to wild-type by SDS-PAGE. The E22K globulomer formed an SDS-unstable oligomer as indicated by loss of the wild-type band at approximately 55 kilo Daltons (FIG. 9A). Crosslinking of the oligomers with glutaraldehyde showed that the E22K globulomer has a higher molecular weight than wild-type (FIG. 9B).

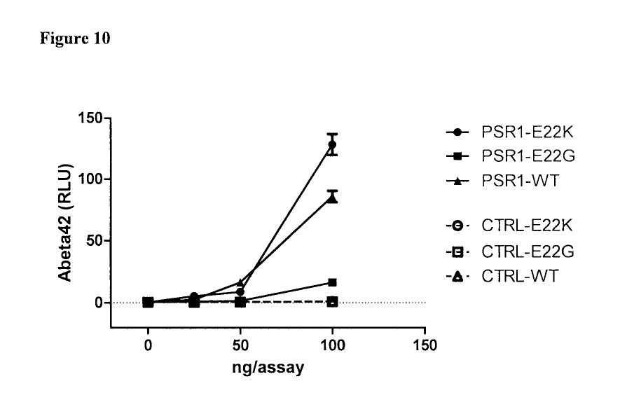

In contrast to the neutrally charged E22G mutant globulomer, the positively charged E22K globulomer was captured efficiently by PSR1 (FIG. 10). This result demonstrates that structure and size also contribute to PSR1 recognition of misfolded proteins, and that charged surfaces on protein structures contribute to PSR1 binding more than net charge.

### Example 6

### Evaluating Binding Ability of Additional Reagents

This Example shows the binding ability of additional species of charged and hydrophobic reagents and further demonstrates the effects of different capture reagent properties on their ability to bind preferentially to oligomers over monomers. Oligomer capture increases exponentially with increasing cationic residues and the capture is more dependent on charge distribution relative to the bead surface than on chirality and orientation, which together suggest that the binding is a multimodal interaction. Increasing the aromaticity/hydrophobicity of the reagents improves oligomer capture, but a balance between charge and hydrophobicity need to be maintained to maintain specificity.

### Materials and Methods

A series of new potential aggregate-specific binding reagents were designed as shown below and conjugated onto magnetic Dynal M270 beads as described in Example 2. Typically 7-12 nmol of ligand (each candidate aggregate-specific binding reagent) was coated onto 1 mg of beads.

Aliquots of the beads (typically 3 ul) were added to wells of a 96 well plate, followed by sample (with or without Abeta 1-42 globulomer (an Abeta42 oligomer model) spiked in 80:20 CSF:TBSTT, typically 125 ul). The plate was sealed and incubated for 1 hour at 37° C. with shaking. The plate was washed with aqueous solutions of detergent (typically polyethylene glycol sorbitan monolaurate and n-tetradecyl-N,N-dimethyl-3-ammonio-1-propanesulfonate) to remove ubound material and the residual buffer removed. A denaturing solution (typically 0.1-0.15 N NaOH) was added to each well and the plate heated to 80° C. for 30 min with shaking. After cooling the plate to room temperature, a neutralizing buffer (typically 0.12-0.18 M NaH₂PO₃ in 0.4% TWEEN20,) was added and the plate was shaken briefly at room temperature. The bead eluate was analyzed using either an Aβ42-specific ELISA or the MSD® 96-Well MULTI-SPOT® Human/Rodent [4G8] Abeta Triplex Ultra-Sensitive Assay from Meso Scale Discovery (Gaithersburg, Md.). For the Aβ42-specific assay, the samples were eluted from the beads and a detection antibody (4G8 HRP) was added to a plate bearing the Aβ42-specific antibody 12F4. The plate was incubated for 1 hour, washed, the substrate was added (SuperSignal West Femto Maximum Sensitivity Substrate from Thermo Fisher, Rockville, Md.), and the luminescence was measured. The MSD plate assay was performed in a similar fashion, according to the manufacturer's protocol.

### Results

### Impact of Positive Charge Number on Oligomer Capture

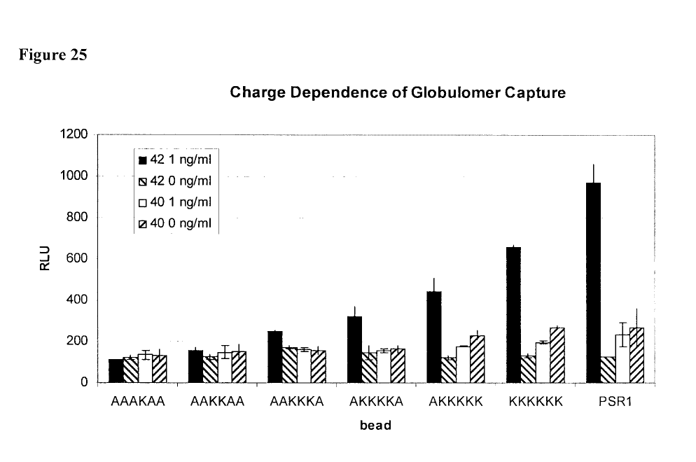

In this experiment the preferred number of charges within a given scaffold was identified. An Ala/Lys peptide framework was utilized, where the inclusion of each Lys residue increases the peptides' net charge by +1. Six hexapeptides with increasing charge (+1→+6), AAAKAA, AAKKAA, AAKKKA, AKKKKA, AKKKKK, and KKKKKK, were prepared and conjugated on the beads. The ability of these beads to capture Abeta 1-42 globulomer spiked at 1 ng/mL into CSF was tested, and the result is shown in FIG. 25. The globulomer capture level (“42 1 ng/ml” or the closed bars) was compared to the background signal of Abeta 1-42 detected in the unspiked CSF (“42 0 ng/ml”) and the background signal of Abeta 1-40 detected in unspiked CSF (“40 0 ng/ml”) or CSF spiked with AB42 oligomers “40 1 ng/ml”. The charge density, as conjugated to the magnetic beads, of each reagent evaluated in this experiment is shown below in Table 8.

TABLE 8
Charge densities of
postitively-charged peptide reagents

| peptide | umol charge/m2 |
|---|---|
| AAAKAA | 2.8 |
| AAKKAA | 2.6 |
| AAKKKA | 3.3 |
| AKKKKA | 4.3 |
| AKKKKK | 5.2 |
| KKKKKK | 6.5 |

The result of this experiment shows that globulomer capture increases with cationic residues (the closed bars), whereas background Abeta 1-42 or 1-40 signal coming from the CSF remains relatively low. These studies suggest that peptides in this framework need at least +2 charge to capture the oligomer, and a charge density of ˜2-3 μmol charge/m².

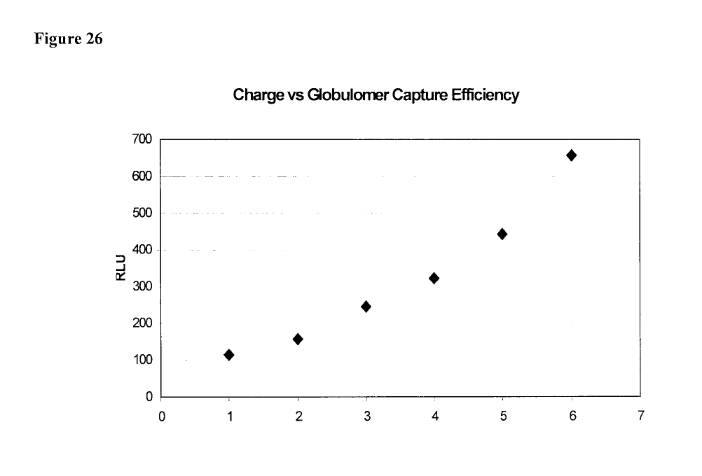

Based on this result, it can also be investigated how charge impacted capture. By plotting the theoretical peptide net charge at pH 7 vs. capture (as shown in FIG. 26), it appears see that there is an exponential relationship between charge and capture, suggesting that increasing the charge will dramatically improve capture. It also appears that while both the “signal” (Abeta1-42 globulomer capture level) and “noise” (Abeta 1-40 monomer capture level) increase with increasing charge, the ratio of signal:noise improves with increasing charge (see Table 9 below, the “42:40” column).

TABLE 9
Signal vs noise level captured by the postitively-
charged peptide reagents of the Ala/Lys scaffold

| | 1 ng/ml average reading | | |
|---|---|---|---|
| Reagent | 42 | 40 | 42:40 |
| AAAKAA | 113.5 | 134.5 | 0.8 |
| AAKKAA | 159.5 | 147 | 1.1 |
| AAKKKA | 247 | 159 | 1.6 |
| AKKKKA | 322.5 | 155.5 | 2.1 |
| AKKKKK | 442 | 176 | 2.5 |
| KKKKKK | 658 | 197 | 3.3 |
| PSR1 | 970.5 | 234.5 | 4.1 |

The numbers in the “42” column represent average RLUs obtained from Abeta 1-42 globulomer spiked CSF samples captured with each peptide reagent. The numbers in the “40” column represent average RLUs obtained from Abeta 1-40 monomer spiked CSF samples captured with each peptide reagent. The “42:40” column shows the ration of “42” to “40”.

Besides the Ala/Lys scaffold, another reagent that has a net charge of +2 but no aromatic residue that was studied is KIGVVR. A similar experiment to the above one on the Ala/Lys scaffold was carried out on this reagent side-by-side with PSR1. The result showed that KIGVVR captured Abeta 1-42 globulomer at a high level that's similar to PSR1's globulomer capture level, and that it had low levels of monomeric Abeta 1-40 noise similar to PSR1's in Abeta 1-40-spiked CSF and low background in non-spike samples.

### Impact of Chirality, Orientation of Charge Relative to Bead, and Orientation of Backbone on Oligomer Capture

From the above experiments, it appeared that the Ala/Lys peptides captured less globulomer than PSR1. PSR1 is also cationic and has 6 residues, but has two features that separate it from the Ala/Lys framework peptides: two aromatic residues, and a different backbone. To better understand which of these features played into the capture efficiency, we investigated each of these properties separately.

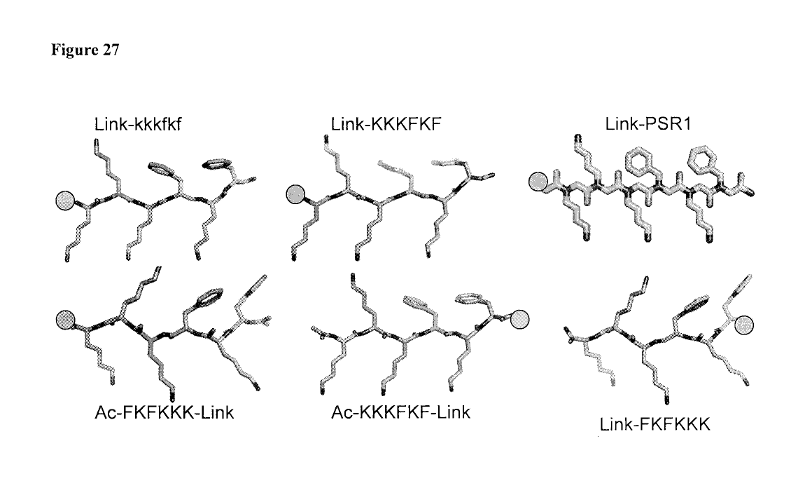

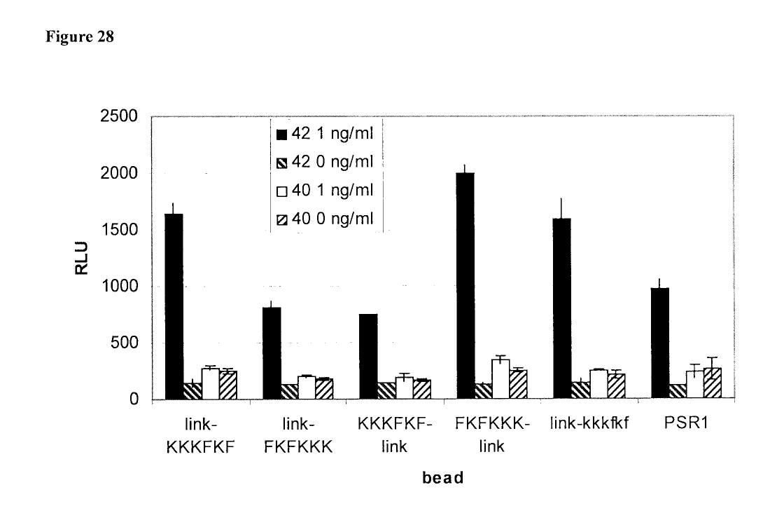

To identify the preferred scaffold, PSR1 and its peptide analog, KKKFKF were studied, and derivatives of KKKFKF were generated. Five different peptide reagents with the same overall charge pattern of KKKFKF were designed to study the impact of chirality, orientation of charge relative to bead, and orientation of backbone (FIG. 27). One peptide, kkkfkf, has D-isoform amino acids instead of the normal L-isforms. A globulomer capture assay was performed on these reagents in a similar way as described above for the reagents with the Ala/Lys scaffold. The peptides were conjugated to the magnetic beads at about 4-5 nmol/mg beads, or about 4.8-6 μmol/m² charge. PSR1 was conjugated at about 12 nmol/mg beads, or about 14 μmol/m² charge. The result of the assay is shown in FIG. 28, and the signal vs. noise comparison is shown below in Table 10.

TABLE 10
Signal vs noise level captured by the
postitively-charged peptide reagents in the
KKKFKF scaffold

| | 1 ng/ml average reading | | |
|---|---|---|---|
| Reagent | 42 | 40 | 42:40 |
| link-KKKFKF | 1646 | 279 | 5.9 |
| link-FKFKKK | 808 | 198 | 4.1 |
| KKKFKF-link | 748 | 191.5 | 3.9 |
| FKFKKK-link | 1995 | 348 | 5.7 |
| link-kkkfkf | 1600 | 253.5 | 6.3 |
| PSR1 | 970.5 | 234.5 | 4.1 |

The assay result shows that, while all of these reagents were able to capture globulomers, the reagents with the charge closest to the beads (link-KKKFKF, FKFKKK-link, and link-kkkfkf,) were significantly better than the remainder of the beads, KKKFKF-link and link-FKFKKK (FIG. 28). The improvement in capture was generally independent of chirality (compare link-kkkfkf vs. link-KKKFKF) and backbone orientation (compare link-KKKFKF and FKFKKK-link). Overall, this lack of dependence on orientation and chirality, but dependence on charge density relative to the bead suggests the globulomers are interacting with the reagent in a multimodal fashion, rather than a traditional small molecule-protein “lock and key” interaction.

### Impact of Hydrophobic/Aromatic Residues

Given that changes to the backbone had only moderate impact on capture, we next explored the utility of aromatic residues, first by comparing the +4 hexapeptide/hexapeptoid reagents of the two different scaffolds as shown above. The comparison of the RLU levels and signal:noise ratio of these reagents is shown below in Table 11.

TABLE 11
Comparison of the +4 hexapeptide/
hexapeptoid reagents of scaffolds with or
without hydrophobic/aromatic residues

| | 1 ng/ml average reading | | |
|---|---|---|---|
| Reagent | 42 | 40 | 42:40 |
| AKKKKA | 322.5 | 155.5 | 2.1 |
| link-KKKFKF | 1646 | 279 | 5.9 |
| link-FKFKKK | 808 | 198 | 4.1 |
| KKKFKF-link | 748 | 191.5 | 3.9 |
| FKFKKK-link | 1995 | 348 | 5.7 |
| link-kkkfkf | 1600 | 253.5 | 6.3 |
| PSR1 | 970.5 | 234.5 | 4.1 |

From this comparison, it appears that the +4 Ala/Lys peptide (AKKKKA) has a significantly lower globulomer capture efficiency and a lower signal:noise ratio than the other reagents which contain aromatic residues (Table 11). This suggests that aromatic and/or hydrophobic residues are beneficial for capture efficiency.

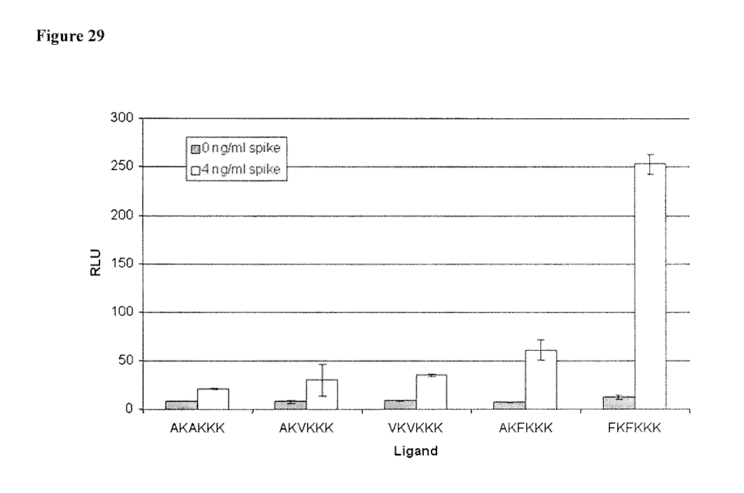

To compare the benefits of aromatic vs. nonaromatic residues for aggregate binding, additional peptides were designed and compared in a globulomer capture assay. The reagents and the assay result are shown in FIG. 29. The right bar for each reagent represents the Abeta 1-42 globulomer level captured and detected from a sample with 4 ng/mL globulomer spiked. The result shows that aromatic residues (represented by the Phe in AKFKKK and FKFKKK) yielded improved globulomer capture relative to nonaromatic residues, although reagents containing nonaromatic hydrophobic residues such as aliphatic residues also captured globulomers (FIG. 29). It is worth noting that even the presence of only one aromatic residue in the peptide, as demonstrated in AKFKKK, was able to significantly increase globulomer capture.

To further explore the requirement for hydrophobic/aromatic residues, we studied a series of peptides that featured fewer charged residues and higher hydrophobic content. The result is shown below in Table 12. Here it can be observed that the most hydrophobic and least charged peptides were efficient at capturing globulomer (FKFSLFSG, FKFNLFSG, and IRYVTHQYILWP), but that they captured significant amounts of background monomeric species as well, suggesting that the interaction was less specific than with a peptide with a more balanced charged/hydrophobic nature.

TABLE 12
Analysis of reagents with high hydrophobic/aromatic content

| | % hydrophobicity/ | 1 ng/mL average reading | | |
|---|---|---|---|---|
| Peptide | % charged | 42 | 40 | 42:40 |
| ANFFAHSS | 30.75/13% | 994.5 | 543 | 1.8 |
| FKFSLFSG | 44.75/13% | 3078.5 | 2011 | 1.5 |
| DFKLNFKF | 32.75/38% | 515.5 | 259.5 | 2 |
| FKFNLFSG | 41.88/13% | 3230.5 | 1327.5 | 2.4 |
| IRYVTHQYILWP | 45.67/17% | 3242 | 1011.5 | 3.2 |
| PSR1 | 33%/67 | 970.5 | 234.5 | 4.1 |

Overall, these results suggest that there is a binding mechanism between the bead-bound reagents and oligomers depends on avidity. Optimal capture efficiency is achieved with a conjugates that yield a charged core and hydrophobic exterior, but the exact sequence/structure of these reagent is less critical. Scaffold changes (e.g., peptides vs peptoid) and chirality are less critical for binding than the charge distribution, so scaffolds with D, L, natural amino acids, unnatural amino acids, peptidomimetics, or organic molecules with similar charged and hydrophobic features will likely show a similar ability to capture oligomers. Finally, increasing hydrophobic content increases capture efficiency, but reduces specificity for the oligomeric form, so maintaining a balance between charge and specificity is important for an effective oligomeric-selective reagent.

### Diverse Aromatic Residues Tested

A variety of alternative aromatic residues, natual or unnatual ones, were introduced into peptide scaffold to generate additional aggregate-binding reagents, and the new reagents were assayed for globulomer binding as described above.

The peptide scaffold used for this study is Ac-FKFKKK-Link (more specifically Ac-FKFKKK-Ahx-Ahx-Cys-NH₂) whose structure is shown below.

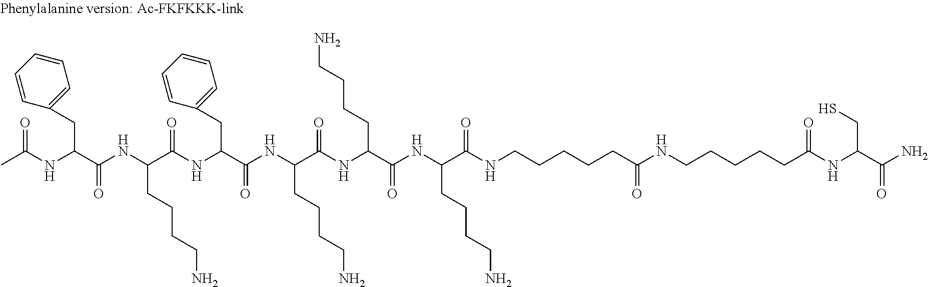 [SMILES: CC(=O)NC(Cc1ccccc1)C(=O)NC(CCCCN)C(=O)NC(Cc1ccccc1)C(=O)NC(CCCCN)C(=O)NC(CCCCN)C(=O)NC(CCCCN)C(=O)NCCCCCC(=O)NCCCCCC(=O)NC(CS)C(N)=O]

The phenylalanines were replaced with different types of aromatic residues in different reagents, represented by each of the following natual or unnatual residues.

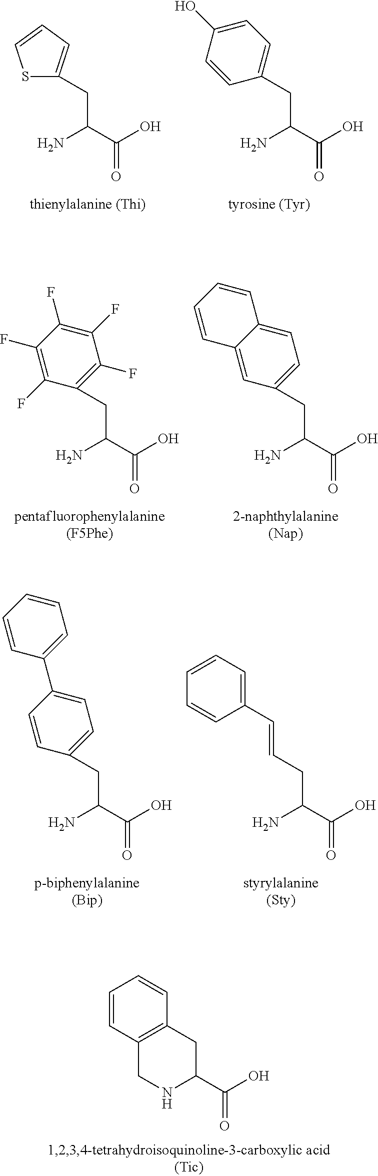

Also studied was the peptide scaffold Ac-KKKFKF-link (more specifically Ac-FKFKKK-Ahx-Ahx-Cys-NH2). The phenylalanines were replaced with different types of aromatic residues in different reagents, represented by each of the following unnatural residues.

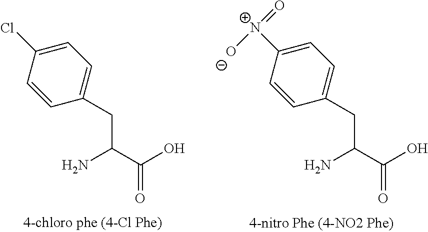 [SMILES: NC(Cc1ccc(Cl)cc1)C(=O)O ; NC(Cc1ccc([N+](=O)[O-])cc1)C(=O)O]

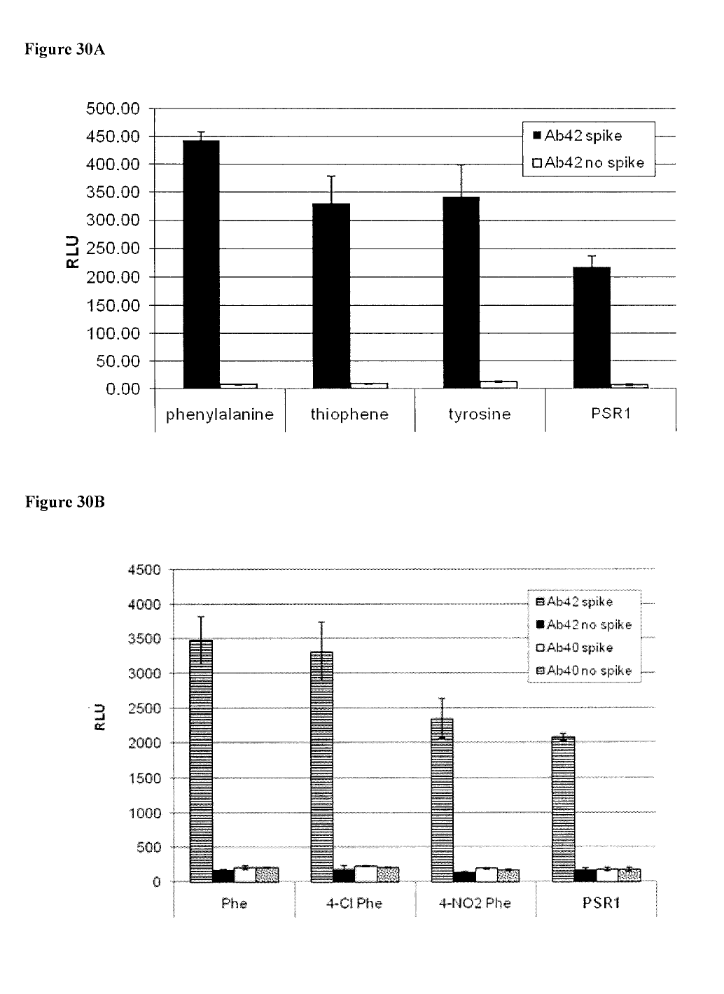

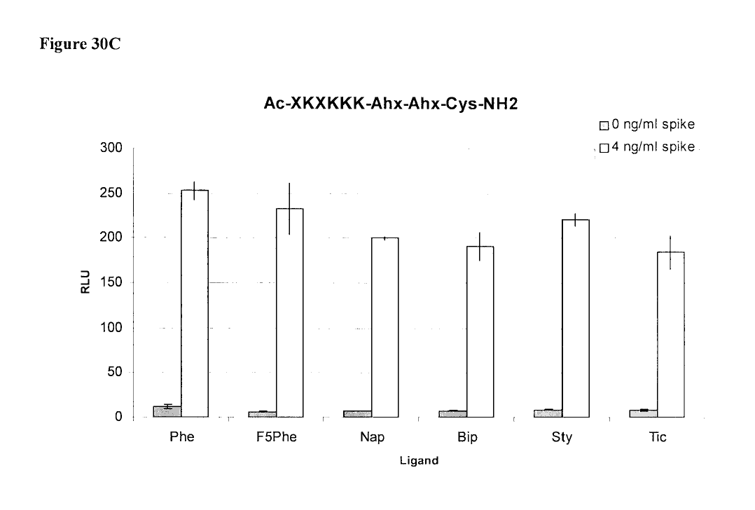

The results of the globulomer binding assays for the reagents with the substituted aromatic residues are shown in FIGS. 30A, 30B and 30C. The results show that all types of substituted aromatic residues worked for specific globulomer capture. The relatively flat Structure-Activity Relationship, as reflected in the similar range of the catpure and detection level with various reagents in this experiment, confirms that this peptide scaffold in general improves globulomer capture.

Another peptide reagent was designed to incorporate positive charge and aromatic features in the same residues—charged aromatics. The sequence of this unnatural peptide is Ala-AmF-AmF-Phe-AmF-Ala (AmF=4-methylaminophenylalanine, a charged aromatic residue). The structure of the peptide is shown below.

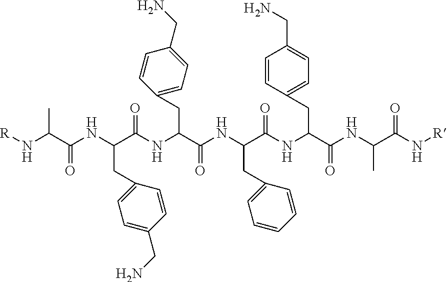

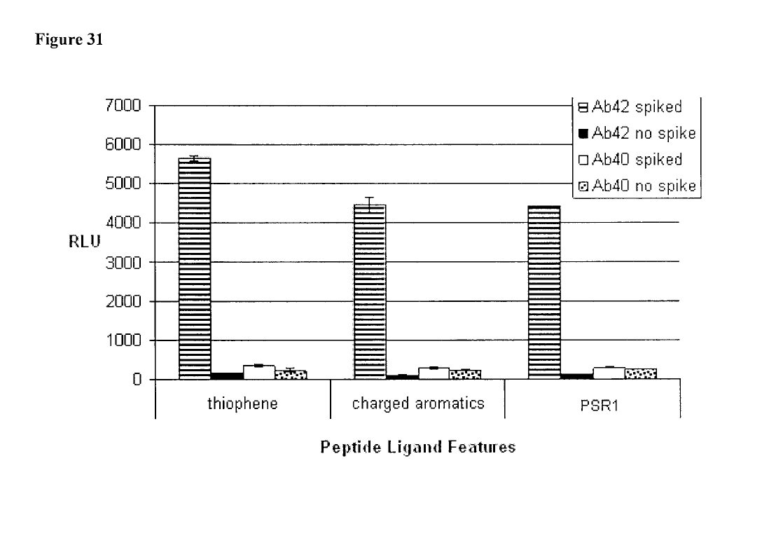

A globulomer binding assay was performed on this “charged aromatics” reagent as described above, and the result is shown in FIG. 31. This experiment again demonstrates the importance of positively-charged and aromatic residues and the flexibility of the structures of them.

### Impact of Spacing of Aromatics

A series of peptide reagents with different spacing of aromatics were generated and tested for aggregate-binding ability in a globulomer capture assay. The sequences of these reagents comprise KKKFKF, KKFKKF, KFKKKF, and FKFKKK, respectively. The result of similar levels of detection indicates that spacing of aromatics plays minimal role in aggregate capture (data not shown, but all these reagents captured globulomers specifically).

### Impact of Spacing of Primary Amines

Spacing of primary amines was also studied for its impact on aggregate capture. Two peptides, one comprising a shorter chain Lys analogue Fmoc-2,4-diaminobutanoic acid (“fdb”, which has an α primary amine), the other comprising 8 primary amines (the delta amino acid 2,5-diaminopentanoic acid, abbreviated as “o”) were generated. The peptides were conjugated to magnetic beads with the Ahx-Ahx-Cys-NH₂ linker and tested for aggregate-binding ability in a globulomer capture assay. The structures of the two peptides are shown below.

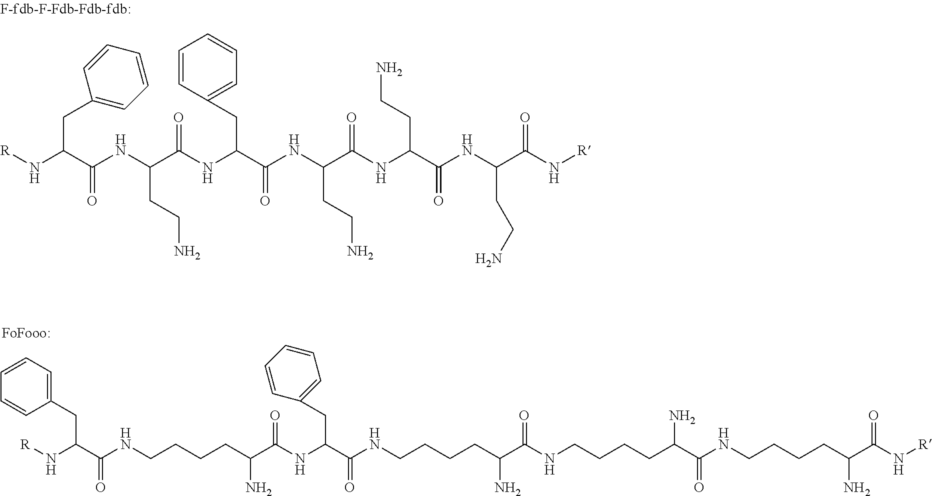

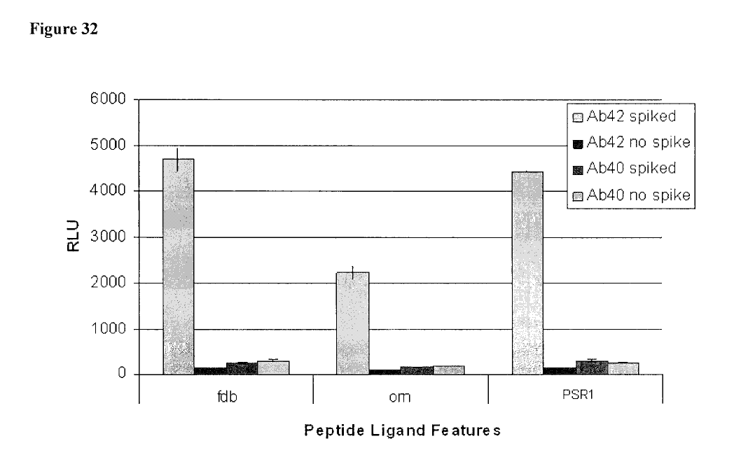

The result of the globulomer capture assay for the reagents with differently spaced primary amines is shown in FIG. 32. It appears from this result that the peptide with the shorter chain Lys analogue, thus more closely spaced primary amines, is more effective than the δ peptide, which has farther spaced primary amines, in capturing aggregates, although they both captured globulomers specifically.

Addition of Quarternary Amines

The addition of quarternary amines to the scaffold was studied with 4 peptides including Ac-KKKFKF and the three whose structures are shown below (a control with a secondary amine and two different quaternary amines). The peptides were conjugated to magnetic beads with the Ahx-Ahx-Cys-NH₂ linker and assayed for globulomer capture as described above. The result of similar levels of detection indicates that inclusion of a single quarternary amines plays minimal role in aggregate capture (data not shown, but all these reagents captured globulomer specifically).

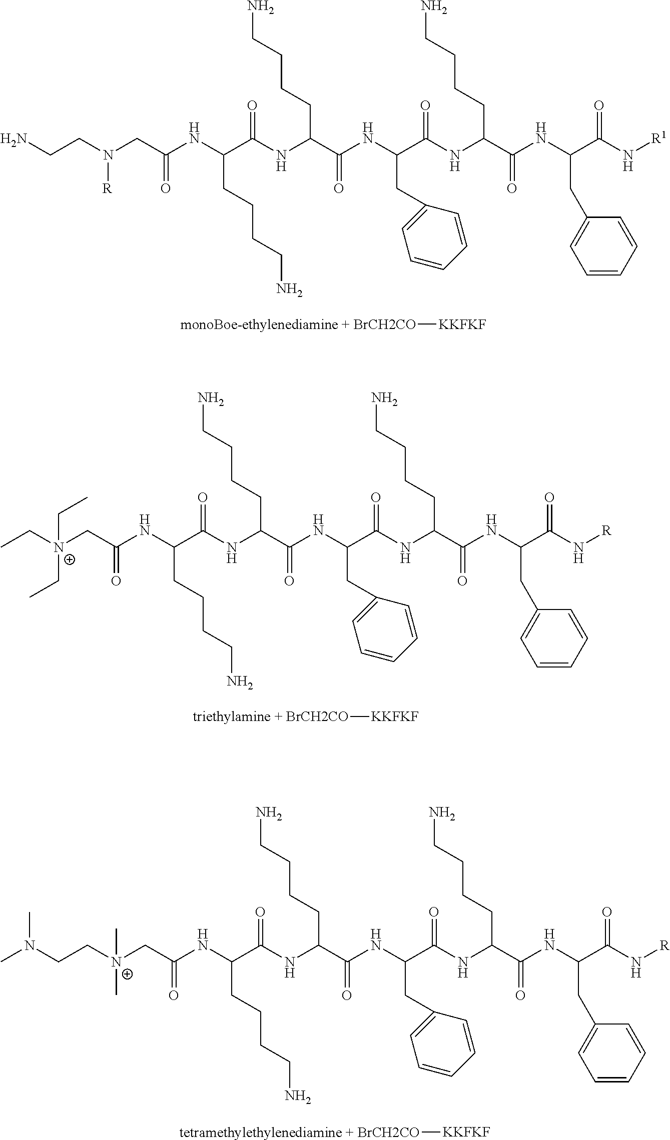

### Additional Aggregate Binding Reagents Tested

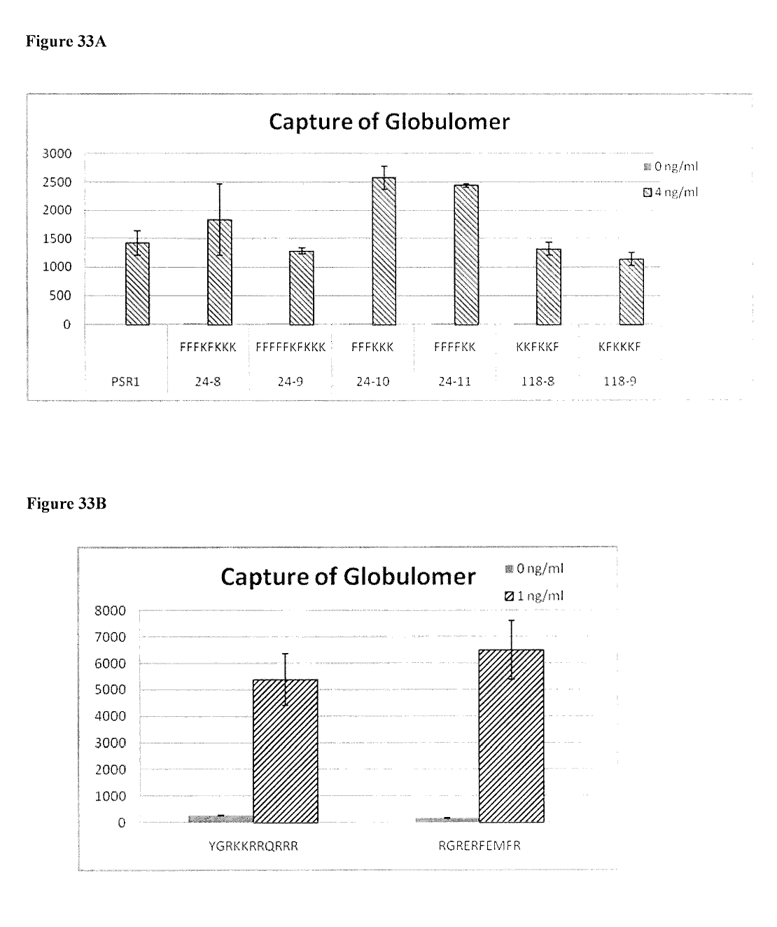

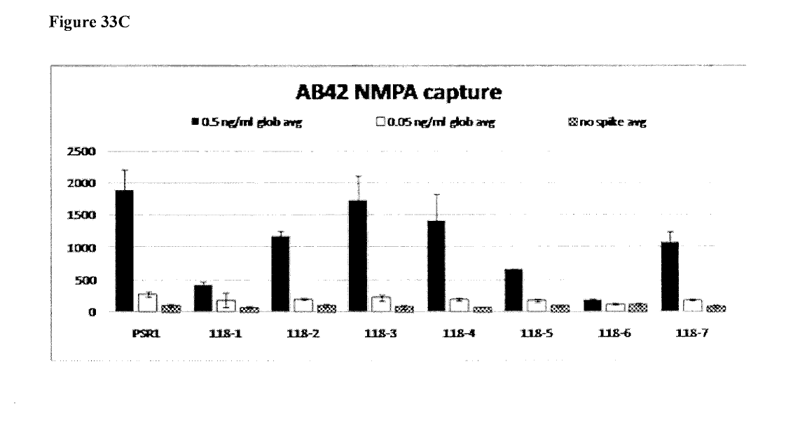

A few additional peptide reagents and peptoid reagents, as shown in Table 13 and Table 14, were generated and tested in a globulomer capture assay as describe above. With the exception of Nbn-Nhye-Ndpc-Ngab-Nthf-Ncpm (118-6), which is neutral, all other peptide and peptoid reagents listed in the two tables captured globulomer specifically (see FIGS. 33A, 33B, and 33C).

TABLE 13
Additional peptide and peptoid
sequences for making ASB reagents

| | SEQ |
|---|---|
| Peptide/ Peptoid Sequence | ID NO |
| FFFKFKKK | 49 |
| FFFFFKFKKK | 50 |
| FFFKKK | 51 |
| FFFFKK | 52 |
| YGRKKRRQRRR | 48 |
| RGRERFEMFR | 47 |
| Nea-Ndpc-Napp-Nffb-Nme-Nthf | 91 |
| Nall-Nhpe-Ncpm-Nchm-Ngab | 92 |
| Nmba-Nfur-Nbn-Nlys-Nea-Nbsa | 93 |
| Namp-Ncpm-Nhye-Nffb-Nlys-Nchm | 94 |
| Nglu-Nlys-Nhpe-Nbsa-Nme-Nea | 95 |
| (Nlys-Nspe-Nspe)4 | 96 |
| Nbn-Nhye-Ndpc-Ngab-Nthf-Ncpm | 97 |

TABLE 14
Structures and net charge of additional peptoid sequences for making ASB reagents

| SEQ ID | | |
|---|---|---|
| NO and | | |
| Reagent | | |
| Code as | | |
| Shown | | |
| in | | Net |
| FIG. 33C | Structure | Charge |
| SEQ ID NO: 91 (118-1) | 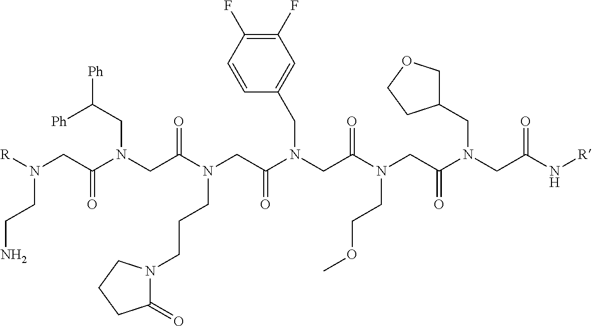 | +1 |
| SEQ ID NO: 92 (118-2) | 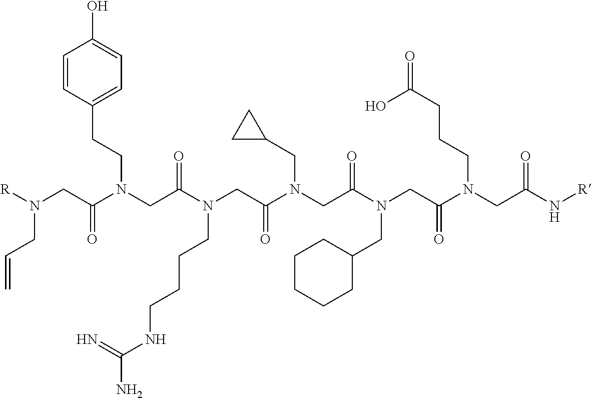 | +1 |
| SEQ ID NO: 93 (118-3) | 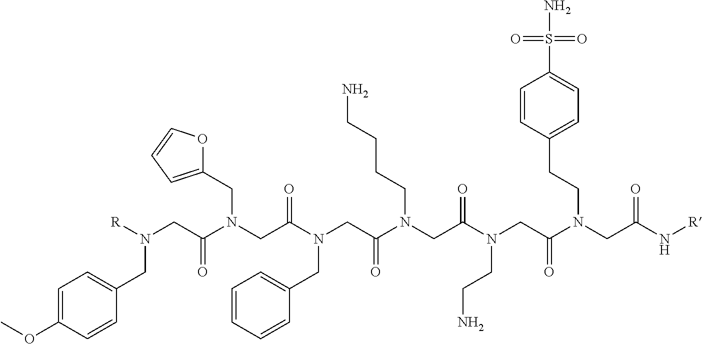 | +3 |
| SEQ ID NO: 94 (118-4) | 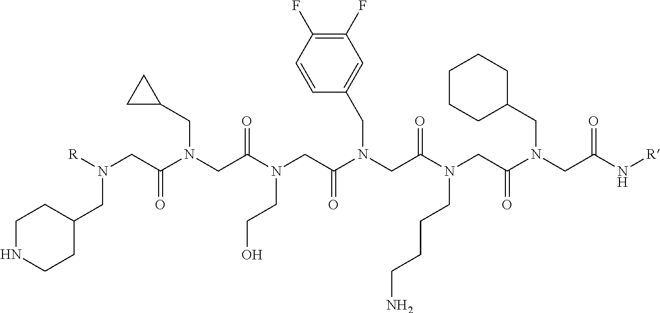 | +3 |
| SEQ ID NO: 95 (118-5) | 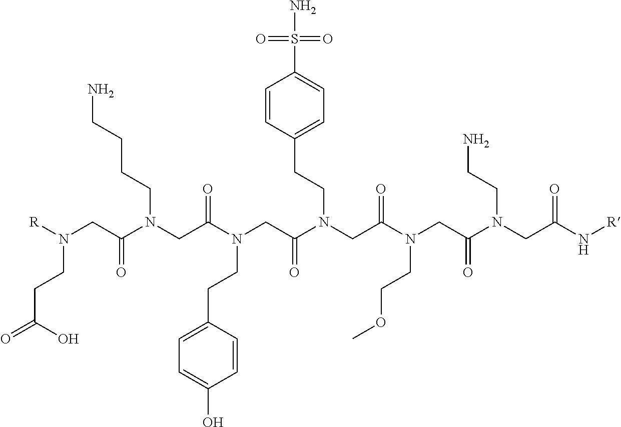 | +2 |
| SEQ ID NO: 96 (118-7) | 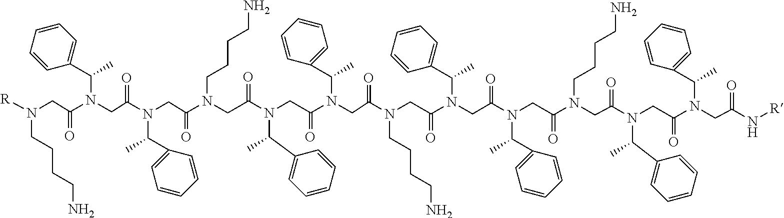 | +5 |
| SEQ ID NO: 97 (118-6) | 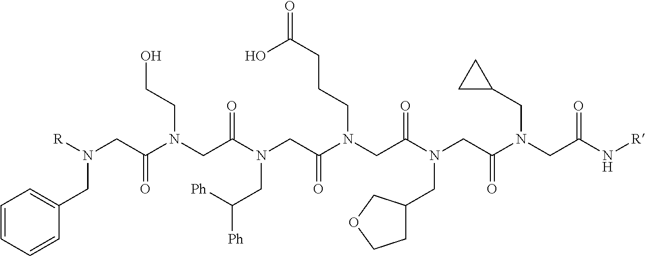 | 0 |

### Example 7

### Screening for New Potential Aggregate-Specific Binding Reagents on Membrane Arrays

In addition to designing peptide or peptoid sequences for candidate aggregate-specific binding reagents, random peptide sequences spotted on cellulose membranes were also tested for specific binding of aggregates over monomer. Many peptides were found to specifically bind aggregates in this study.

A cellulose membrane array prepared to display 1120 random 12mer peptides was purchased from the University of British Columbia's peptide center (Vancouver, Canada, whose service is currently available through http://www.kinexus.ca/). The loading density of the peptides on this membrane is not readily available. However, using a membrane array synthesis method that should be similar to what was described by the manufacturer, we measured the peptide loading density to be about 2-4 mmol ligand/m², which means that the peptides with the lowest number of positive charge, +1, were probably coated to the membrane array at the same density, 2-4 mmol net charge/m². The array synthesis method we used to coat random peptides is described as follows.

A cellulose membrane (Whatman 50) was immersed in 10:1:90 solution of epibromohydrin:perchloric acid:dioxane and allowed to incubate 1-3 hour at room temperature. After washing with methanol and drying, the membrane was aminated by incubation in neat trioxadecanediamine at 70 C for 1 h. After washing, the membrane was quenched (in 3M NaOMe), washed and dried again. Spots were demarcated by spotting 1 ul of a 0.4M solution of FmocGly preactivated with HOBT and DIC in NMP and incubating for 20 min. The coupling was repeated and the membrane capped with 2% acetic anhydride in DMF, followed by 2% acetic anhydride/2% DIEA in DMF. The membrane was washed with DMF, deprotected with 4% DBU in DMF (2×10-20 min), washed with DMF and methanol, and then dried. Subsequent amino acids could then be attached using standard solid phase synthesis methods, using a cycle of: 1) spotting activated Fmoc amino acid solutions to the membrane, 2) capping with acetic anhydride, 3) deprotection with DBU. The final membrane was capped, washed with DMF and methanol and dried before use.

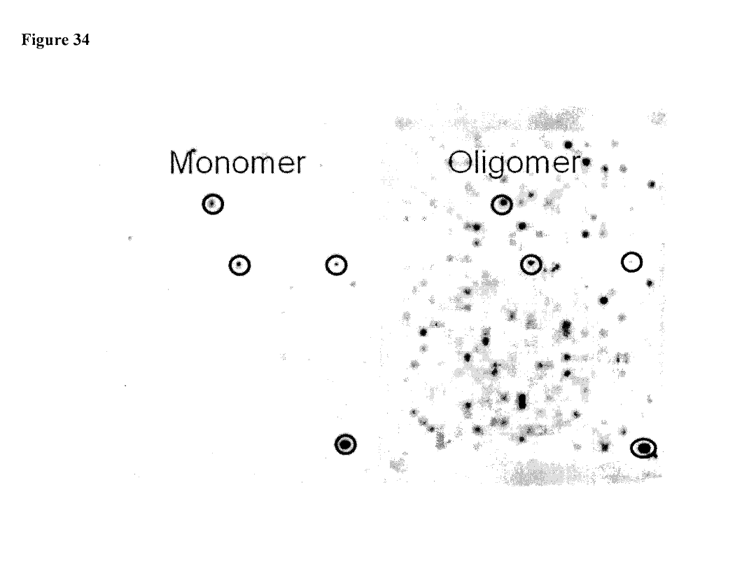

The membrane purchased from the University of British Columbia was incubated in a 1% milk solution for 60 minutes, washed 4 times for 10 minutes each, and then subjected to a solution of 3 ng/mL Abeta 1-42 globulomer (the “Oligomer” sample) or 3 ng/ml, Abeta 1-42 monomer (the “Monomer” sample, prepared as described above in Example 2) in TBST for 60 minutes. After washing, the membrane was incubated in a solution of anti Abeta antibody (6E10) diluted in 1% milk for 60 minutes. After washing, the membrane was subjected to a secondary antibody (goat-anti-mouse-HRP) diluted in 1% milk for 60 minutes. Following a wash step, a chemiluminescent substrate (DURA WEST from Thermo Fisher, Rockville, Md.) was added to the array and images were taken on a Kodak imager. The resultant image is shown in FIG. 34, and the positively charged peptides that specifically bound globulomers among the top specific binders on the membrane are shown below in Table 15. Several peptides that specifically bound globulomers on the membrane, although not among the top specific binders, were also included in Table 15 because they were later validated on magnetic beads.

TABLE 15
Positively charged peptides that
specifically bound Abeta 42
globulomers on cellulose membrane

| | Specifically bound | |
|---|---|---|
| | Aβ42 globulomers | SEQ |
| Peptide sequence | on beads? | ID NO |
| KFYLYAIDTHRM | Yes | 6 |
| KIIKWGIFWMQG | Yes | 7 |
| NFFKKFRFTFTM | NT (Not Tested) | 8 |
| MKFMKMHNKKRY | Yes | 67 |
| LTAVKKVKAPTR | Yes | 68 |
| LIPIRKKYFFKL | Yes | 69 |
| KLSLIWLHTHWH | Yes | 70 |
| IRYVTHQYILWP | Yes | 71 |
| YNKIGVVRLFSE | Yes | 72 |
| YRHRWEVMLWWP | Yes | 73 |
| WAVKLFTFFMFH | Yes | 74 |
| YQSWWFFYFKLA | Yes | 75 |
| WWYKLVATHLYG | NT | 76 |
| QTLSLHFQTRPP | NT | 77 |
| TRLAMQYVGYFW | NT | 78 |
| RYWYRHWSQHDN | NT | 79 |
| AQYIMFKVFYLS | NT | 80 |
| TGIRIYSWKMWL | NT | 81 |
| SRYLMYVNIIYI | NT | 82 |
| RYWMNAFYSPMW | NT | 83 |
| NFYTYKLAYMQM | NT | 84 |
| MGYSSGYWSRQV | NT | 85 |
| YFYMKLLWTKER | NT | 86 |
| RIMYLYHRLQHT | NT | 87 |
| RWRHSSFYPIWF | NT | 88 |
| QVRIFTNVEFKH | NT | 89 |
| RYLHWYAVAVKV | NT | 90 |

Selected peptides from Table 15 were conjugated to DYNAL beads using the linker Ac-Cys-Lys-Ahx-Ahx at the amino terminus of the peptides, following the same protocol as described above. The charge density of these reagents on beads was as low as about 4000 nmol/m², for the peptides with only one positively charged residue, and proportionally higher for the peptides with more than one positively charged residues. The peptide-conjugated beads were assayed for Abeta42 globulomer binding ability in CSF containing physiological levels of Abeta40 and 42. All of the sequences listed in table 15 that were tested on beads were validated to bind Abeta42 globulomer specifically when coated to beads (Table 15 and data not shown)

### Example 8

### Reducing Binding Background in CSF with Detergent Treatment

In this example, potential interference of aggregate detection from biological samples such as CSF was studied, and a solution to reduce such interference was found by testing various detergents in post-capture washing steps.

First, the limit of detection (LoD) of Abeta 42 globulomer spiked into normal CSF (pooled CSF samples from healthy people) was compared to globulomer spiked into buffer (TBSTT), using PSR1-conjugated beads. The result showed that the LoD of globulomer was about 10 pM when spiked into CSF or about 5 pM when spiked into buffer, indicating that CSF samples have high background of binding (data not shown).

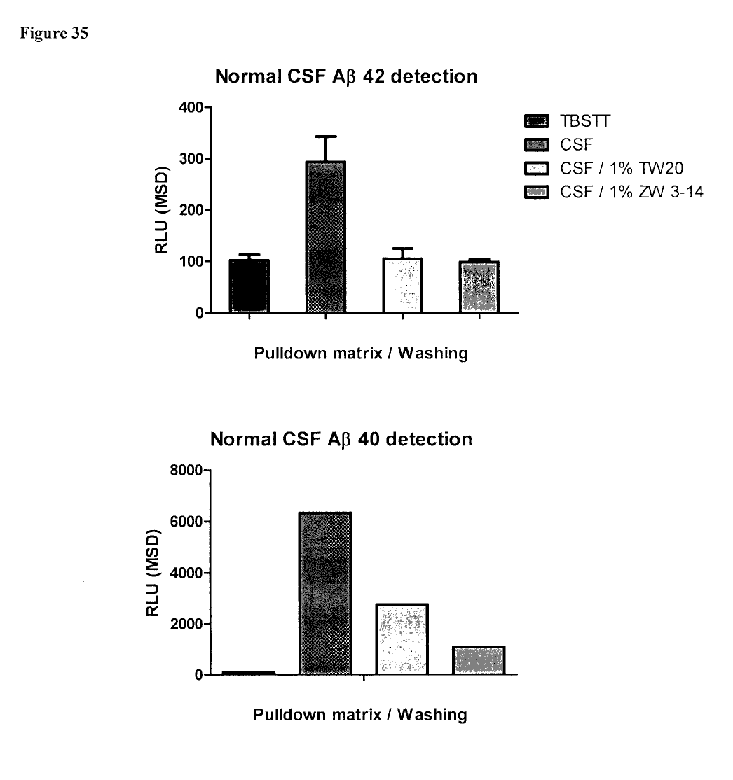

Second, two neutral detergents, polyethylene glycol sorbitan monolaurate (available as TWEEN 20 from Sigma-Aldrich, St. Louis, Mo.) and n-tetradecyl-N,N-dimethyl-3-ammonio-1-propanesulfonate (available as ZWITTERGENT 3-14 from EMI) Chemicals, Gibbstown, N.J.) were used to treat the globulomer-spiked CSF samples that had been contacted with PSR1 beads. For each assay, a mixture of 30 μl of PSR1 beads (coated at about 7-12 nmol PSR1 ligand/mg DYNAL beads, which were used in this example and in the following examples unless otherwise stated) and 70 μl of 1×TBSTT was immediately pipetted into each well on the pulldown plate. The liquid was removed on a magnetic separator. Fifty micro liters of 5×TBSTT was added to each well. The beads were suspended by briefly shaking at 750 rpm. Next 200 μA of TBSTT or CSF sample without globulomer was added to each well. The pulldown plate was sealed and incubated at 37° C. for 1 hour with shaking at 500 rpm. After incubation, the beads were washed 8 times with TBST on the plate washer. After the plate wash, residual TBST buffer was removed from the beads on the magnetic separator. The beads were then incubated with 100 μl of either TBS, 1% Tween20 or 1% Zwittergent 3-14 for 30 minutes at room temperature at 750 rpm, (followed by an additional 8 washes with TBST on the plate washer. After removing residual TBST on the magnetic separator, 20 μl of denaturing solution, typically 0.1-0.15 N NaOH, was added to the beads. The plate was covered with an aluminum foil plate sealer and incubated at 80° C. for 30 minutes with shaking at 750 rpm. After incubation, the plate was cooled to room temperature and twenty micro liters of neutralizing solution, typically 0.12-0.18 M NaH₂PO₄+0.4% Tween20) was added into each well and the plate was incubated at room temperature for 5 minutes with shaking at 750 rpm. After magnetically separating the beads from the eluate, the supernatant was transferred to a previously blocked MSD ELISA plate. MSD Abeta Triplex Assay was preformed according to the manufacturer's instructions. Background levels of Abeta42 and Abeta40 detected from CSF samples is shown in FIG. 35. The results show that washing with either TWEEN 20 or ZWITTERGENT 3-14 reduced the detection of normal CSF Aβ42 to the background levels observed when PSR1 is incubated with TBSTT buffer alone. They also reduced the detection of normal CSF Aβ40 significantly, with ZWITTERGENT 3-14 appearing to work better than TWEEN 20 for reducing Aβ 40 detection level normal CSF.

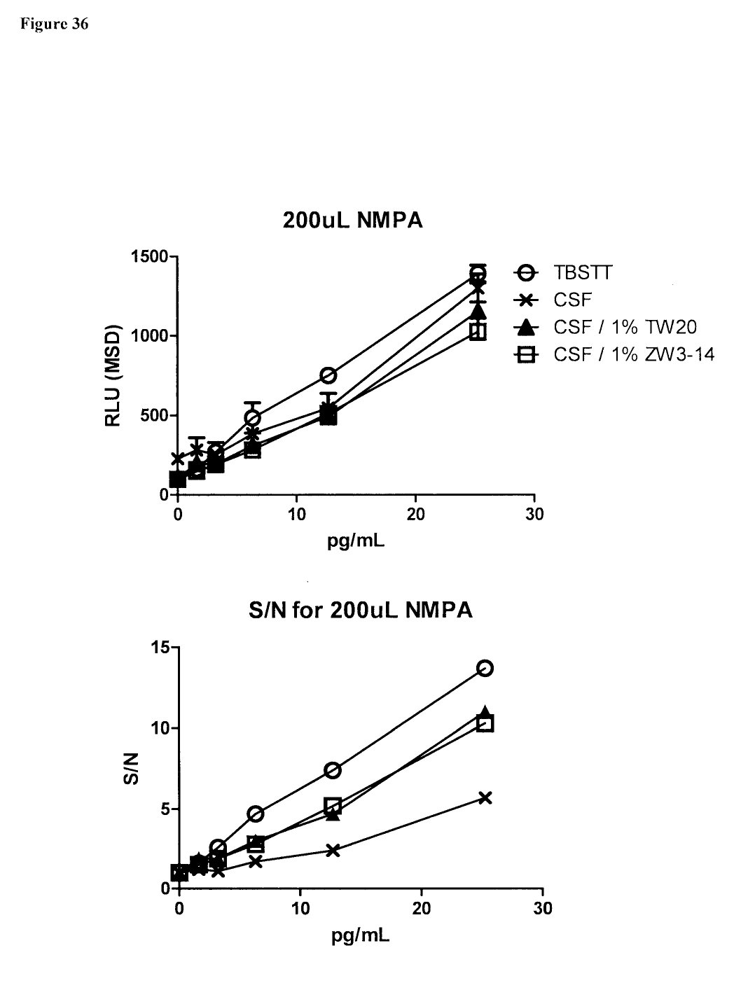

Next, the effect of detergent treatment was studied in samples spiked with globulomer. Various concentrations of Abeta 42 globulomer (from 0-25 pg/mL) were spiked into either 200 ul TBSTT or CSF. CSF samples were mixed with 50 ul 5×TBSTT before samples were contacted with 30 μl of PSR1 beads. The capture, washing, and detection steps were performed as described above. The result of Abeta42 detection levels of globulomer spiked into different matrices and treated with different washing buffers, as well as calculated signal/noise (where signal is the RLU of the sample and the noise is the signal from an equivalently treated sample that is not spiked with globulomer), are shown in FIG. 36. The result shows that treatment of the globulomer-spiked CSF samples with ZWITTERGENT 3-14 or TWEEN 20, after PSR1 pulldown, improved the signal/noise of globulomer detection.

The LoD's of MPA globulomer detection, based on a S/N=2, as well as signal/noise ratio at 25.3 pg/mL globulomer spike level of are calculated and shown below in Table 16. The calculated results indicate that ZWITTERGENT 3-14 and TWEEN 20 also reduced the LoD's of globulomer in CSF significantly, down to the LoD's of globulomer in buffer.

TABLE 16
Globulomer detection RLU's and calculated LoD's and S/N's of samples
treated with different washing buffers post capture

| Pulldown | TBSTT | CSF | | |
|---|---|---|---|---|
| Washing (added detergent) | None | None | 1% TW20 | 1% ZW 3-14 |

LoD of globulomer

| | | | | |
|---|---|---|---|---|
| RLU (S/N = 2) | 204 | 456 | 211 | 199 |
| pg/mL (Cal) | 2.34 | 6.16 | 2.52 | 2.33 |

S/N

| | | | | |
|---|---|---|---|---|
| 25.3 pg/mL | 13.7 | 5.7 | 11.0 | 10.3 |

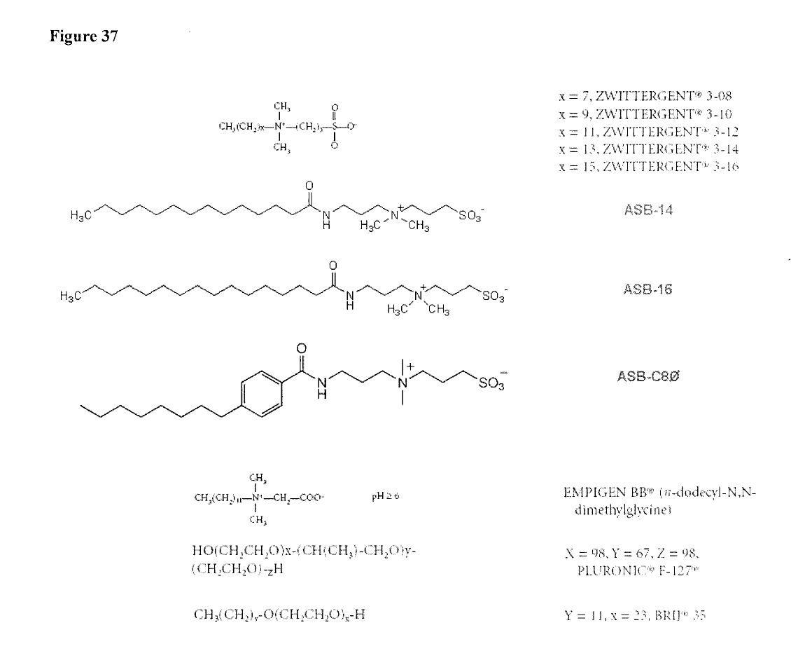

Finally, a range of detergents were tested according to the method described in this example, and some were found to reduce the background Abeta aggregate binding of CSF samples. The detergents tested and their structures are shown in FIG. 37. A summary of the assay results and calculated signal/noise is shown below in Table 17.

TABLE 17
Globulomer detection RLU's and calculated S/N's of samples treated with different detergents post capture

| Abeta 42 | | | | | ASB- | | | | Pluronic | Brij |
|---|---|---|---|---|---|---|---|---|---|---|
| globulomer | ZW3-14 | ZW3-08 | ZW3-12 | ZW3-16 | C8phn | ASB-14 | ASB-16 | Empigen | F-127 | 35 |
| | Abeta 42 RLU | | | | | | | | | |
| 0 ng/mL | 110.0 | 97.7 | 90.7 | 67.0 | 100.7 | 89.7 | 95.7 | 76.7 | 117.0 | 117.0 |
| Average | | | | | | | | | | |
| SD | 5.3 | 2.5 | 5.7 | 11.4 | 19.0 | 9.1 | 18.3 | 13.8 | 14.4 | 23.6 |
| CV % | 4.8 | 2.6 | 6.3 | 17.0 | 18.9 | 10.1 | 19.2 | 18.0 | 12.3 | 20.2 |
| 0.5 ng/mL | 842.0 | 668.7 | 642.0 | 730.7 | 936.3 | 779.3 | 788.7 | 670.3 | 739.3 | 741.0 |
| Average | | | | | | | | | | |
| SD | 42.6 | 149.0 | 70.2 | 142.7 | 263.9 | 82.0 | 36.9 | 29.7 | 65.2 | 53.3 |
| CV % | 5.1 | 22.3 | 10.9 | 19.5 | 28.2 | 10.5 | 4.7 | 4.4 | 8.8 | 7.2 |
| | S/N | | | | | | | | | |
| Abeta 42 | 7.7 | 6.8 | 7.1 | 10.9 | 9.3 | 8.7 | 8.2 | 8.7 | 6.3 | 6.3 |
| globulomer- | | | | | | | | | | |
| spiked/unspiked ^{a} | | | | | | | | | | |
| Abeta 42 | 2.2 | 0.9 | 1.4 | 3.7 | 1.4 | 2.0 | 2.8 | 2.6 | 0.9 | 1.0 |
| globulomer- | | | | | | | | | | |
| spiked/Abeta 40 ^{b} | | | | | | | | | | |

^{a} S/N of Abeta 42 globulomer-spiked/unspiked = (Abeta 42 RLU of sample with 0.5 ng/mL globulomer)/(Abeta 42 RLU of sample with 0 ng/mL globulomer)
^{b} S/N of Abeta 42 globulomer-spiked/Abeta 40 = (Abeta 42 RLU of 0.5 ng/mL globulomer)/(average Abeta 40 RLU of sample with 0 ng/mL globulomer and sample with 0.5 ng/mL globulomer)

The result shows that ZWITTERGENT detergents with longer carbon chains (ZWITTERGENT 3-14 and 3-16) improved the signal/noise ratio for Abeta 42 globulomer detection more significantly. In another experiment, ZWITTERGENT detergents with longer carbon chains, especially ZWITTERGENT 3-16, improved Abeta 40 aggregates capture S/N even more significantly in AD CSF (data not shown).

The detergents that reduced the background Abeta aggregate binding of CSF samples and that resulted in a S/N of Abeta 42 globulomer-spiked/Abeta 40 that's greater than 1.0 are the following ones.

TWEEN 20 (Polyethylene glycol sorbitan monolaurate), ZWITTERGENT 3-14 (n-Tetradecyl-N,N-dimethyl-3-ammonio-1-propanesulfonate), ZWITTERGENT 3-16 (n-Hexadecyl-N,N-dimethyl-3-ammonio-1-propanesulfonate), ZWITTERGENT 3-12 (n-Dodecyl-N,N-dimethyl-3-ammonio-1-propanesulfonate), ASB-14 (Amidosulfobetaine-14, 3-[N,N-Dimethyl(3-myristoylaminopropyl)ammonio]propanesulfonate), ASB-16 (Amidosulfobetain-16, 3-[N,N-Dimethyl-N-(3-palmitamidopropyl)ammonio]propane-1-sulfonate), ASB-C8 phenol (4-n-Octylbenzoylamido-propyl-dimethylammonio Sulfobetaine), and EMPIGEN BB (N,N-Dimethyl-N-dodecylglycine betaine). They are all available from Sigma-Aldrich and/or EMD Chemicals.

### Example 9

### Conformational Specificity of Aggregate-specific Binding Reagents

This Example characterizes the conformational specificity of PSR1 and PSR1's peptide analog-Ac-FKFKKK.

### Materials and Methods

The Ab42 aggregates were prepared as previously described: fibrils were prepared per Stine et al (JBC 2003, 278, p11612), globulomers were prepared per Barghorn et al (J Neurology 2005 95 p834), ADDLs were prepared per Lambert et al (PNAS 1998, 95 p6448), and ASPDs were prepared per Noguchi et al (JBC 2009 284 p32895). Normal CSF was pooled from clinically characterized non-demented patient CSF samples. AD CSF was pooled from clinically-characterized AD patient samples. Alzheimer's Disease Brain Homogenate (ADBH) was prepared by sonication of clinically diagnosed AD patient brain samples in 0.2M.sucrose (1:10 w/v).

For the native gel, each sample was loaded onto a 4-20% gradient gel and run under native conditions for 5 h and treated with Coomassie stain (simply Blue Safe Stain).

For the capture assay (Misfolded Protein Assay), aliquots of the beads (30 ul) were added to wells of a 96 well plate, followed by 125 ul of sample in 80:20 CSF:TBSTT. The plate was sealed and incubated for 1 h at 37 C with shaking. The plate was washed with TBST and the residual buffer removed. A denaturing solution (0.1 N NaOH) was added to each well and the plate heated to 80 C. for 30 min with shaking. After cooling the plate to RT, a neutralizing buffer (0.12 M NaH2PO₃-0.4% TW20,) was added and the plate shaken briefly at RT. The assay was analyzed using the triplex Mesoscale Discovery ELISA kit for Aβ. The Aβ in the samples eluted from the beads were detected per the manufacturer's protocol.

For the Limit of Detection (LoD) studies, serial dilutions of each aggregate were spiked into CSF and were assayed per the protocol described above. The LoD was defined as 2× over background levels.

For discriminating between AD and normal CSF, pooled normal CSF or pooled Alzheimer's Disease patient CSF, were assayed per the protocol described above.

### Results

Seven Aβ species of different sizes and shapes were selected for binding studies

TABLE 18
Aβ Species

| Model | Components | Size | Shape | Reference |
|---|---|---|---|---|
| Monomer | Aβ42 | ~5 KDa | Unstructured? | N/A |
| Globulomer | Aβ42, DMSO, | ~60 KDa | Globular | Barghorn et al |
| | SDS | | | |
| ADDL | Aβ42, media? | KDa-MDa | Micellar, | Klein et al |
| | | | fibrillar | |
| ASPD | Aβ42, media? | KDa-MDa | Micellar, | Hoshi et al |
| | | | fibrillar | |
| Fibrils | Aβ42 | MDa | Fibrillar | multiple |
| ADBH | Aβ40, Aβ42, +? | MDa | Fibrillar? | N/A |

### Native Gel Analysis

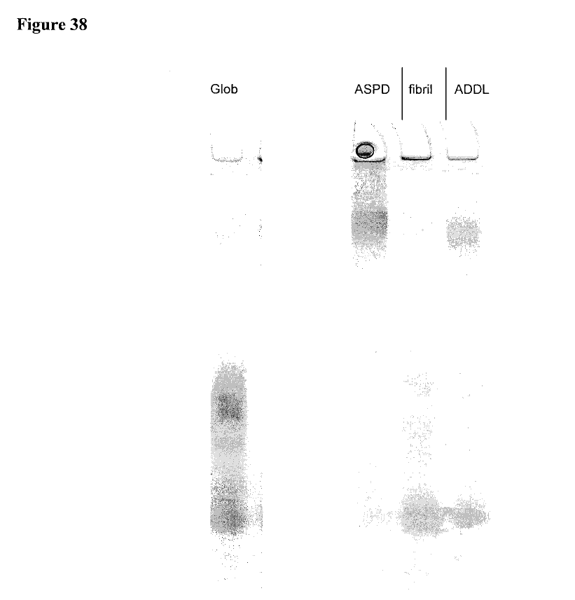

Native gel analysis was conducted in order characterize the different Aβ species (FIG. 38). All of the tested aggregates had moderate homogeneity. All contained some amount of monomer. All but globulomer have large material that does not pass into gel. The globulomer appears smallest of models tested. The ASPD and ADDL showed similar properties.

### Capture Profile of Reagents

Capture studies using the Misfolded Protein Assay showed that PSR1 is capable of capturing diverse aggregate conformations and sizes. All of the aggregate species were captured by PSR1 at sub-fmol levels.

TABLE 19
LoD of PSR1 for Aggregates

| | | | | LoD | LoD |
|---|---|---|---|---|---|
| Model | Components | Size | Shape | (pg/mL) | (amol) |
| Monomer | Aβ42 | ~5 | Unstructured? | ~1000 | ~2.2 × |
| | | KDa | | | 10⁴ |
| Globulomer | Aβ42, DMSO, | ~60 | Globular | ~75 | ~140 |
| | SDS | KDa | | | |
| ADDL | Aβ42, media? | KDa- | Micellar, | ~200 | ~20 |
| | | MDa | fibrillar | | |
| ASPD | Aβ42, media? | KDa- | Micellar, | ~200 | ~20 |
| | | MDa | fibrillar | | |
| Fibrils | Aβ42 | MDa | Fibrillar | ~50 | <2 |
| ADBH | Aβ40, Aβ42, +? | MDa | Fibrillar? | ~0.6-4 | <0.2 |

ASR1 and Ac-FKFKKK universally bind all the different aggregate species tested with similar binding preferences. The exact numbers for LoD are dependent on CSF and aggregate lot.

TABLE 20
LoD of Reagents for Aggregates

| ADBH | ADDL | ASPD | Fibril | Globulomer | |
|---|---|---|---|---|---|
| (pg/ml) | (pg/ml) | (pg/ml) | (pg/ml) | (pg/ml) | |
| <5 | 200 | 200 | 50 | 75 | PSR1 |
| <10 | 300 | 300 | 100 | 75-120 | Ac-FKFKKK |

The LoDs demonstrate a preference of capturing larger fibrillar material>smaller oligomeric species>>monomers. This pattern minors the capture selectivity observed when these species are tested with 3 ul of PSR1 beads and 1 ng/mL of aggregate.

TABLE 21
LoD of Reagents for Aggregates

| | | | Globul- | No | Denatured | |
|---|---|---|---|---|---|---|
| ADDL | ASPD | Fibril | omer | spike | globulomer | |
| 1219 | 1096 | 3636 | 1267 | [symbol] | [symbol] | PSR1 |
| (±106) | (±74) | (±658) | (±145) | | | |
| 1241 | 1321 | 4138 | 3409 | [symbol] | [symbol] | Ac- |
| (±55) | (±72) | (±143) | (±148) | | | FKFKKK |

[symbol]

indicates data missing or illegible when filed

### Discrimination Between AD and Normal CSF

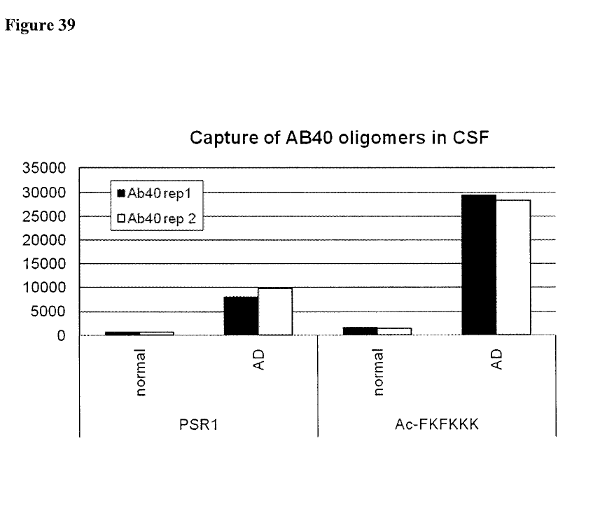

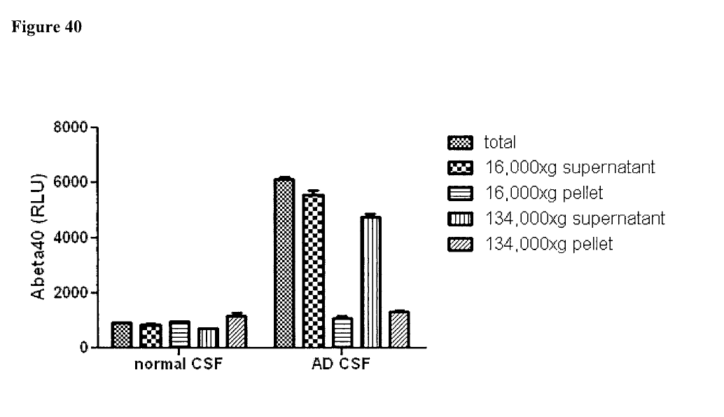

Reagents were tested to identify ones useful for discriminating between AD and normal CSF. See FIG. 39. Both PSR1 and Ac-FKFKKK were shown to have higher Aβ40 signal in positive pooled AD CSF relative to unmatched normal pool, suggesting that the reagent is capturing in vivo Aβ40 aggregates present in the AD CSF. Ac-FKFKKK provides the largest change in signal.

### Example 10

### Sizing Aβ40 Oligomers from AD CSF by Differential Centrifugation

This Example characterizes the physical properties of the aggregates captured from Alzheimer's Disease CSF by PSR1.

### Materials and Methods

AD CSF or normal pooled CSF spiked with nothing, 5 ng/mL globulomer or 200 mL/mL ADBH (with or without sonication) were centrifuged at 16,000×g for 10 min or 134,000×g for 1 hour at 4 C. Supernatant and pellet fractions were taken to separate tubes (pellets were reconstituted in CSF with the same volume as the original sample) and subjected to the Misfolded Protein Assay (MPA).

Misfolded Protein Assay: 100 ul sample was incubated with 25 ul 5×TBSTT buffer (250 mM Tris, 750 mM NaCl, 5% Tween20, 5% TritonX-100 pH 7.5) and 30 ul PSR1 beads for 1 hour at 37 C. Beads were washed 6× with TBST followed by a 30 minute incubation with 1% Zwittergent 3-14 and another TBST wash. Abeta peptide was eluted with 0.15 M NaOH for 30 minutes at room temperature, followed by neutralization of the eluate with 0.18 M NaH2PO4+0.5% Tween20 and detection by Mesoscale's triplex Aβ immunoassay according to manufacturer's instructions.

### Results

The behavior of the various aggregates of known sizes was determined to provide molecular weight references for the aggregates found in Alzheimer's CSF. Although the solubility of aggregates does not necessarily have a linear relationship with molecular weight (and is subject to variability depending on the conformation of the aggregates), these studies provide some frame of reference for aggregate size. Abeta fibrils from an unsonicated Alzheimer's Disease Brain Homogenate (ADBH) pelleted at both 16,000 g and 134,000 g. These aggregates eluted near the void volume of a TSK4000 column and are likely to be greater than 1 MDa. Some proportion of Abeta aggregates from a sonicated ADBH was soluble at 16,000 g but pelleted at 134,000 g. Size exclusion chromatography estimated these aggregates to be about 0.5-1 MDa. Globulomers (estimated to be approximately 54 KDa) were soluble at both 16,000 g and 134,000 g.

Endogenous Aβ40 oligomers in AD CSF stay in solution after a 1 hour centrifugation at 134,000 g, suggesting that they may be smaller than “intermediate-sized” aggregates of 0.5 to 1 MDa found in a sonicated ADBH sample. These data indicate that the oligomers found in AD CSF have different behavior with respect to solubility when compared to aggregates deposited in tissues (ADBH) and is suggestive that they are smaller in size.

### Example 11

### Detection of AA Protein in Mice with Spleen AA

This Example demonstrates that PSR1 binds preferentially to serum amyloid A aggregates which develop in AA amyloidoses and certain cases of chronic inflammation.

### Materials and Methods

### Animals

Inbreed 8-10 weeks old C57BL/6J mice were used. All mice were maintained under specific pathogen-free conditions. Housing and experimental protocols were in accordance with Swiss Animal Welfare Law and in compliance with the regulations of the Cantonal Veterinary Office, Zürich.

### Induction of Amyloidosis

Amyloid enhancing factor (AEF) was extracted from amyloid-laden liver as described earlier [1], and used for amyloid induction in four different groups of mice. Each mouse received 20 ug of protein extract as an intravenous injection in the tail vein and systemic inflammation was stimulated by concomitant subcutaneous injection of 0.2 ml 1% silver nitrate (AgNO3). Further inflammatory stimuli were given once a week on day 7, 14 and 21. Mice were sacrificed in several time points on day 5, 9, 16 and 23. Control mice received only single silver nitrate injection and were sacrificed 16 hrs later.

### Histology

Spleen was fixed in 10% neutral buffered formalin and embedded in paraffin. The presence of amyloid was investigated in 5 um thick sections after Congo red staining [2] and the amyloid amount was quantified according to the following scale: 0; absent; 1+ trace of amyloid; 2+ small amyloid deposits; 3+ moderate amyloid deposits; 4+ extensive amounts of amyloid [1].

### Tissue Preparation

10% spleen homogenates were prepared in PBS, p117.4, using an ultra-sound tissue homogenizator. Homogenates were centrifuged at 200×g for 1 min and the supernatants were used for the PSR1 bead-based capture assay. For immunoblotting analysis the PBS-insoluble tissue pellet was solubilised in 8M urea for 24 hrs on a wheel at room temperature.

PSR-1 Bead-Based Capture of AA Species from Spleen Homogenates

PSR1-conjugated beads were washed two times with 1 ml of TBS-TT (TBS, 1% TritonX, 1% Tween20) before incubation with 10% spleen homogenate in a total volume of 100 μl TBS-TT for 1 hr at 37° C. and under shaking at 750 rpm. Unbound material was removed from the beads by washing five times with 1 ml TBS-T (TBS, 0.05% Tween20). Subsequently, beads were resuspended in 50 ul TBS-T and the captured proteins were eluted with 75 ul denaturation buffer (1M NaOH pH 12.3) for 10 min at 37° C. or 80° C. under shaking at 750 rpm or 1200 rpm, respectively. Thereafter, samples were neutralized with 30 ul 1 M NaH₂PO₄, pH 4.3 for 10 min at 37° C. or 80° C. under shaking at 750 rpm or 1200 rpm, respectively. 150 ul of eluate was aspirated from the beads and the presence of SAA/AA proteins were analysed using the mouse SAA ELISA from Tridelta Ltd. or by immunoblotting. 3, 6 and 9 ul of PSR-1 beads and 1, 4, and 8 ul of 10% spleen homogenates and ratios thereof have been tested.

### Immunoblotting

Samples were heated to 95° C. for 5 minutes prior to electrophoresis through a 10-20% Tris-Tricine precast gel (Invitrogen), followed by transfer to a nitrocellulose membrane by wet blotting. To detect the mouse SAA/AA proteins, two different primary antibodies: anti-mouseSAA antibody (1:1000; Tridelta Ltd.) and a polyclonal anti-mouse SAA/AA antibody (1:1000) that were kindly provided by Prof. Gunilla Westermark (Uppsala University, Sweden) were used. The secondary antibodies were goat-anti-rat-HRP (1:8000) and goat-anti-rabbit-1110 (1:10000), respectively. Protein bands were visualized with the SuperSignal West Pico Chemiluminiscent substrate (Pierce) and exposing the blot in Stella detector (Raytest).

### Results:

### PSR1-Coated Beads can Capture AA-Related Moieties:

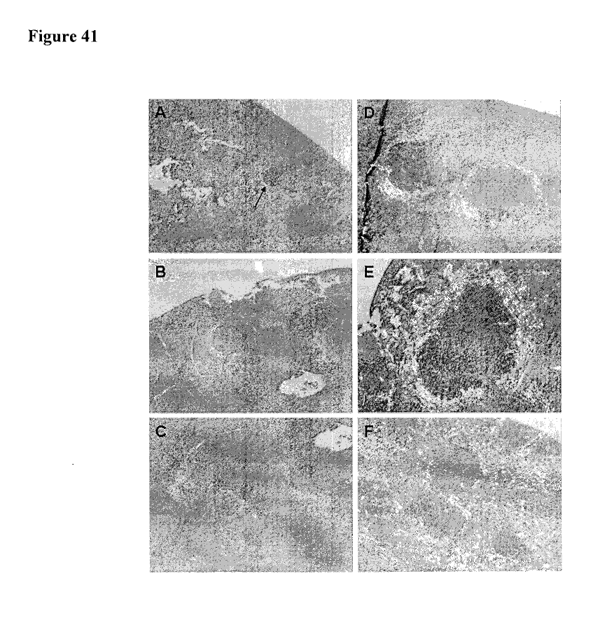

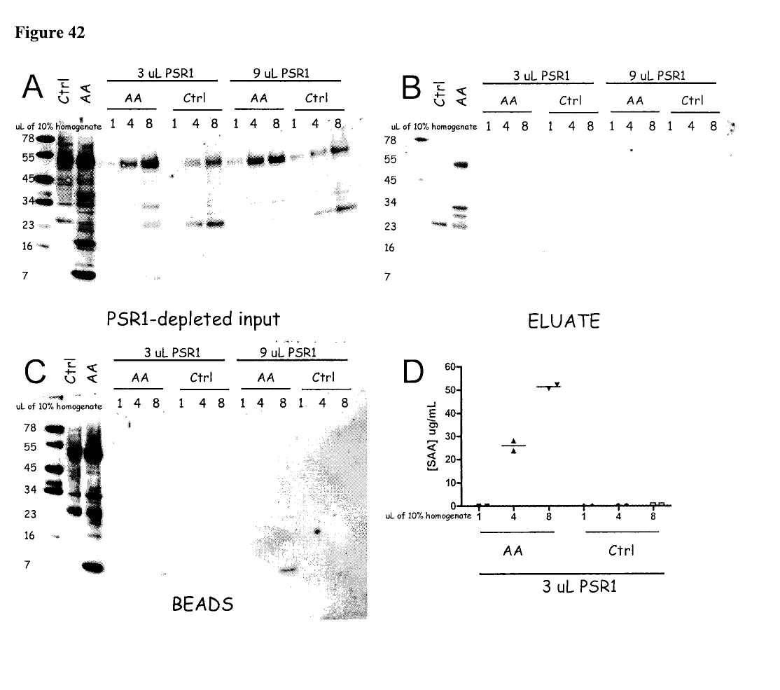

To test whether PSR1-coated beads can capture AA-related moieties, the MPA was performed with 3 or 9 uL of PSR1-coated beads (30 mg/mL) using 1, 4 or 8 uL of 10% w/v spleen homogenate from a mouse with splenic AA (34 score as assessed histologically by Congo red staining, FIG. 41) and a control untreated mouse as inputs. Western blot analysis on beads, eluate and PSR1-depleted input fractions using an anti-mouse SAA antibody revealed the presence of a short fragment, with an electrophoretic mobility similar to the one of the 7 kDa component of the molecular weight marker, in the eluate and in the beads fractions, only in the AA-containing sample (FIG. 42 a-c). Eluate fractions of the MPA performed using 3 uL of PSR 1 beads were tested also by a mouse SAA sandwich ELISA. SAA could be detected only in the eluates from the AA-containing sample (FIG. 42 d).

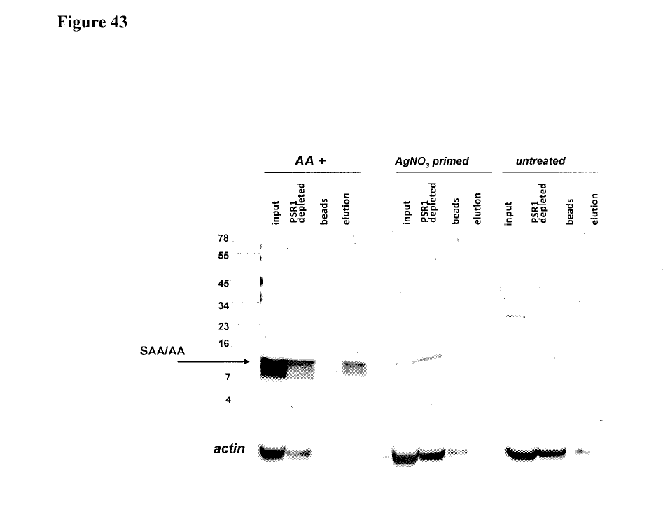

In some test tubes the elution of captured AA moieties was not optimal and signal could be detected on immunoblots in the bead fractions as well (FIG. 42c). Therefore, more stringent conditions for the elution (80° C. and 1200 rpm) were tested. These conditions resulted in the complete elution of AA captured moieties (FIG. 43). When spleen homogenates from amyloid negative but AgNO₃ primed animals, that have very high levels of full-length SAA in circulation, or spleen homogenates from untreated animals are subjected to the MPA assay, no signal was detected in the eluates (FIG. 43). Importantly, for this immunoblot another anti-mouse SAA antibody was used to remove several shorter AA fragments in the eluates from amyloid positive samples.

These experiments indicate that PSR1-coated beads can capture AA-related moieties.

Denaturation of AA aggregates prevents the detection of AA-related moieties.

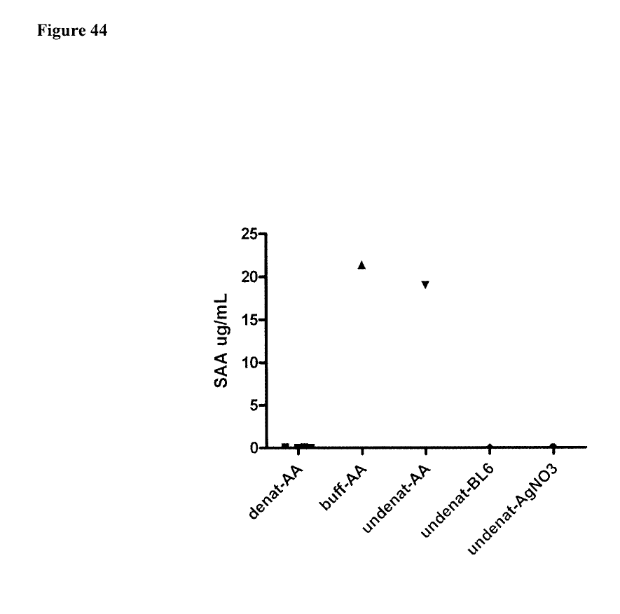

To test whether PSR1-capturing of AA-related moieties is restricted to aggregates, the MPA was performed on denatured, buffered and undenatured AA-containing samples, as well as on spleen homogenates from a control AgNO₃-treated mouse and a control untreated mouse. Eluate fractions were tested by ELISA. SAA could be detected only in the eluates from undenatured or buffered AA-containing samples (FIG. 44). These data indicate that denaturation of the input material interferes with the MPA by preventing capturing and/or elution of AA-related moieties by/from PSR1-coated beads, that capturing of such moieties by the PSR1 beads is aggregate specific under the tested conditions.

### REFERENCES

1. Lundmark K, Westermark G T, Nystrom S, Murphy C L, Solomon A, et al. (2002) Transmissibility of systemic amyloidosis by a prion-like mechanism. Proc Natl Acad Sci USA 99: 6979-6984.
2. Puchtler H, Sweat F (1965) Congo red as a stain for fluorescence microscopy of amyloid. J Histochem Cytochem 13: 693-694.

### Example 12

### Detection of Amylin Aggregates

This Example demonstrates that PSR1 binds preferentially to amylin aggregates which develop in Type II diabetes. More specifically, this Example describes how PSR1 binds preferentially to amylin fibrils over monomers, whether the amylin was generated in vitro or extracted from pancreatic tissue.

### Materials and Methods

In vitro amylin fibrils were generated by reconstituting monomeric amylin peptide in 10 mM Tris buffer (pH7.5) at 100 uM, and incubating at RT for more than three days. 10% pancreatic tissue homogenate was made in sucrose solution. The samples were denatured by combining 1 volume of sample with 9 volumes of 6M guanidine thiocyanate, and incubating at RT for at least 30 minutes.

The monomeric amylin in the samples was detected by Linco human amylin (total) ELISA kit (Millipore Cat# EZHAT-51K), and the aggregated amylin was detected by MPA (Misfolded Protein Assay) using PSR1 beads. To run the MPA, native or denatured samples (in vitro model or tissue) are spiked into buffer or normal human plasma, and subjected to PSR1 or a negative control beads. After incubation, the beads were washed, and the aggregated amylin bound on beads were eluted and denatured by 6M guanidine thiocyanate. The eluate was then diluted into sample buffer and detected by ELISA using the Linco human amylin (total) ELISA kit described above.

### Results

### In Vitro Synthesized Amylin

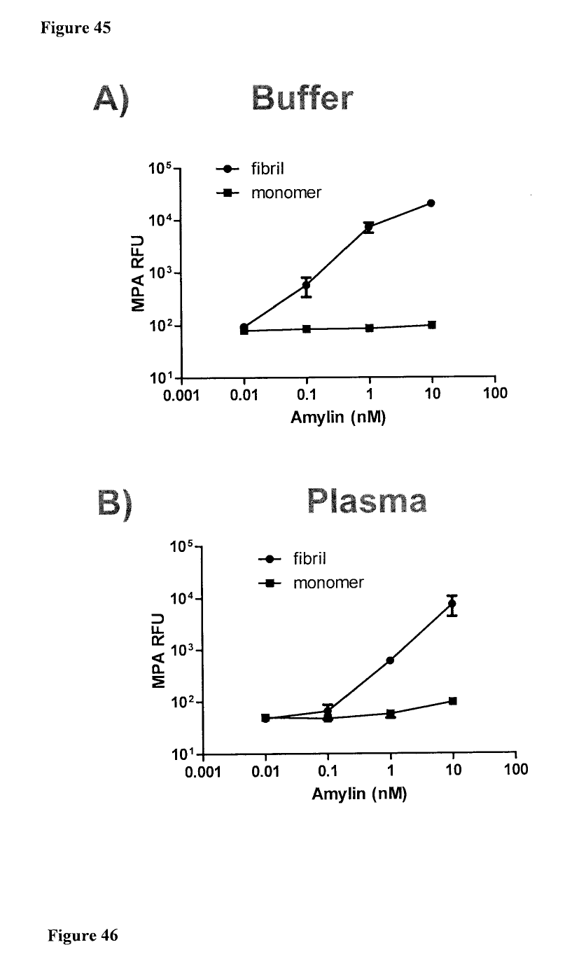

FIGS. 45A and B demonstrates that MPA detects amylin in vitro fibrils but not monomers in both buffer and plasma.

Endogenous Amylin from Pancreatic Tissue

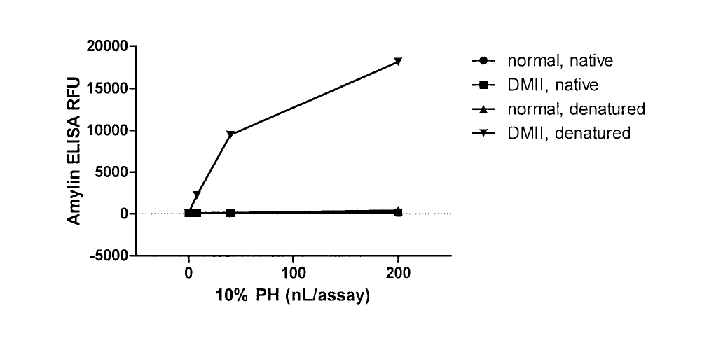

Pancreatic tissue from the Type II diabetes patients contains high concentration of aggregated amylin as compared to normal pancreatic tissue. However, this aggregated amylin cannot be detected by ELISA directly unless the sample is denatured to monomeric form (see FIG. 46).

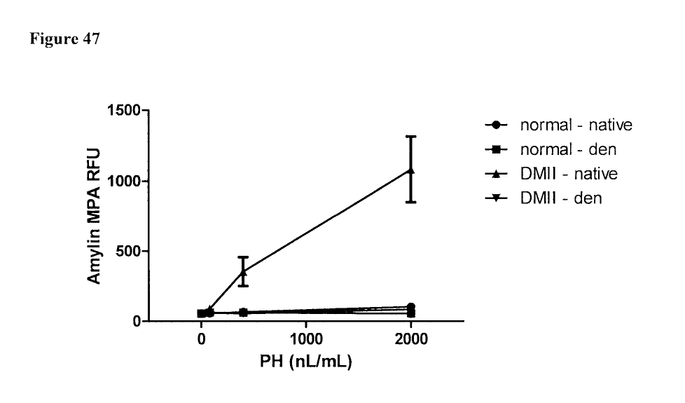

When the MPA assay was run, it detected the aggregated amylin in pancreatic tissue from Type II diabetes patient spiked into human plasma. Denaturation of the sample, which converts the aggregated amylin to monomers, abolished detection by MPA. (see FIG. 47).

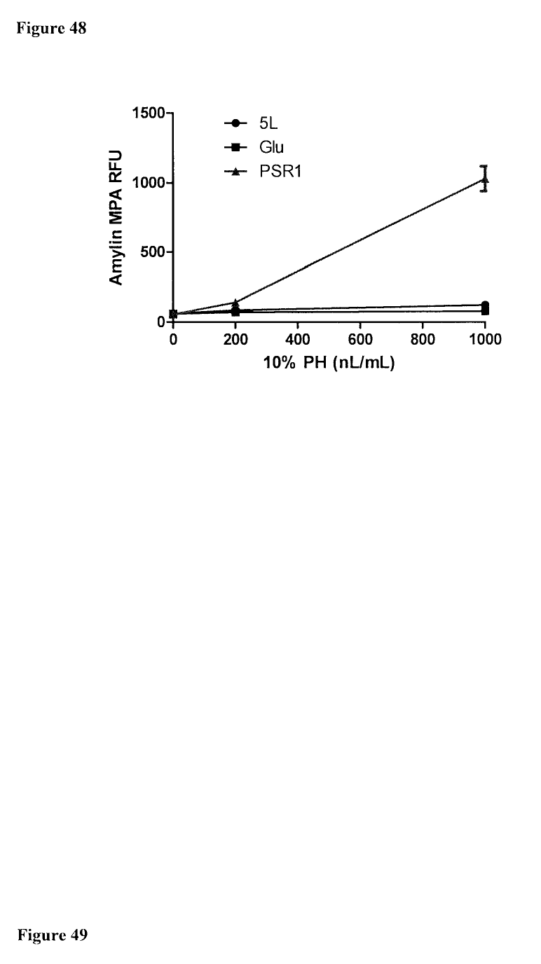

The detection of aggregated amylin in pancreatic tissue from Type II diabetes patients is due to PSR1 specific binding to amylin fibrils. FIG. 48 shows the same type II diabetes pancreatic tissue spiked into plasma bound to PSR1, but not to control beads with either neutral glutathione or a peptoid (5 L) which is a negatively charged version of PSR1.

### Example 13

### Detection of Alpha-Synuclein

This Example demonstrates that PSR1 binds preferentially to alpha-synuclein aggregates which develop in Parkinson's disease, as well as other synucleinopathies such as Gaucher's disease, multisystem atrophy, and Lewy body dementia.

### Materials and Methods

### ELISA

Amylin fibrils were prepared as reported in J. Biological Chem. (1999) 274, No. 28, pp 19509-19512. To denature the fibrils, samples were treated with 5.4 M guanidine thiocyanate for 30 minutes at room temperature. The samples were then diluted to the indicated concentrations and alpha synuclein was detected by a sandwich ELISA (Invitrogen; catalog #KHB0061) according to the manufacturer's instructions.

### Misfolded Protein Assay

The specificity of PSR1 beads for aggregated alpha synuclein was tested by incubating 3 ul PSR1 beads with fibrillar alpha-synuclein with or without a pretreatment with chemical denaturant (5.4 M guanidine thiocyanate for 30 minutes at room temperature). Alpha synuclein was diluted into 125 ul 80% CSF or plasma in TBSTT (50 mM Tris, 150 mM NaCl, 1% TritonX-100, 1% Tween20) at the indicated concentrations. Samples were incubated for 1 hour at 37 C and 550 rpm before washing with TBS 0.05% Tween20 buffer. Bound alpha synuclein was eluted from the beads with 4 ul 6 M guanidine thiocyanate (30 minutes at room temperature), diluted with 246 ul ELISA diluent buffer and then detected by sandwich ELISA (Invitrogen; catalog #KHB0061). Nonspecific binding of alpha synuclein fibrils to the beads was also tested with a control bead (glutathione conjugated Dynal beads).

### Results

### PSR1Beads Bind to Alpha Synuclein Fibrils

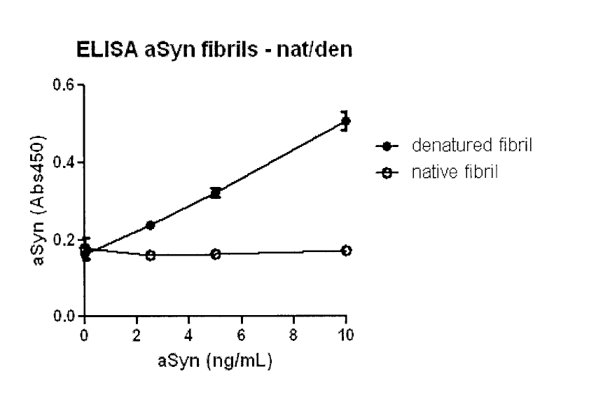

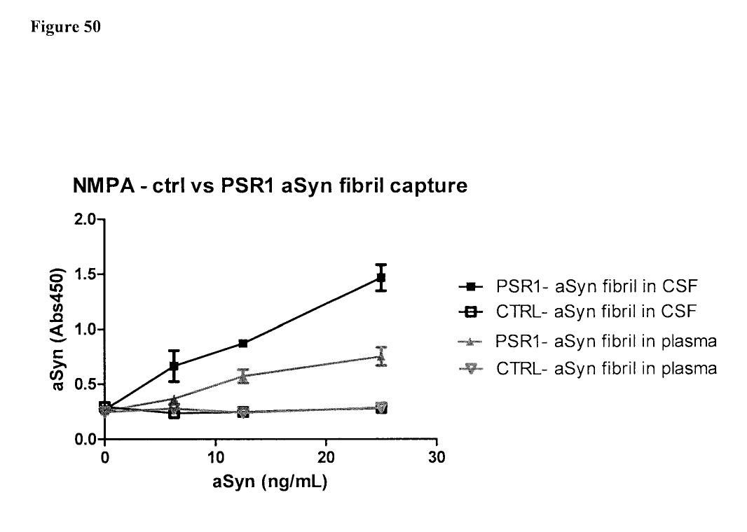

Alpha synuclein (aSyn) fibrils were not detected by sandwich ELISA (Invitrogen Catalog #KHB0061) unless they were pretreated with a denaturant that exposes antibody epitopes that were masked within the fibril. Only guanidine-treated alpha synuclein fibrils could be detected, suggesting that denaturation is necessary for optimal detection of the aggregate's constituent monomers. Because denaturation of large volumes sample containing low concentrations of aggregates is difficult, PSR1 is a useful tool to capture and enrich these aggregates. See FIG. 49.

PSR1 beads used in the Misfolded Protein Assay (MPA) specifically bound to alpha synuclein (aSyn) fibrils diluted into CSF or plasma as compared to a control bead (CTRL) conjugated with glutathione molecules. See FIG. 50.

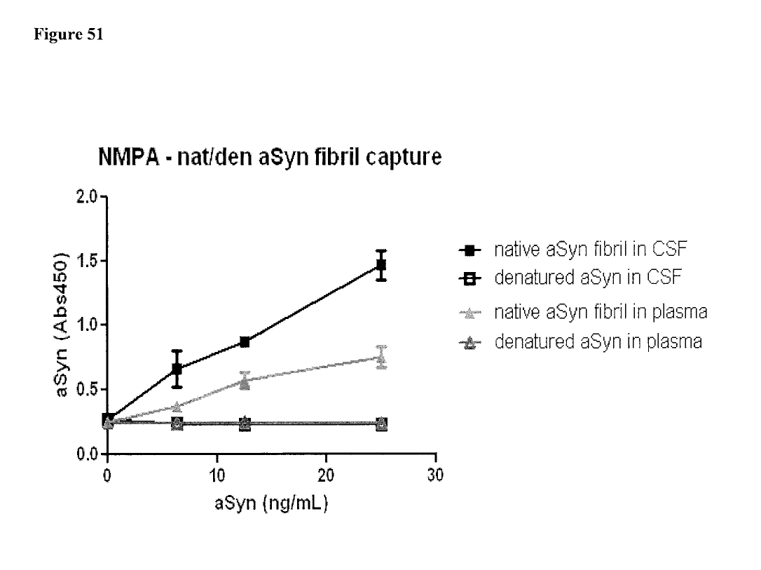

PSR1 beads used in the Misfolded Protein Assay bound preferentially to alpha synuclein fibrils (native aSyn fibril) diluted into CSF or plasma but not to alpha synuclein monomers generated by pretreating fibrils with a chemical denaturant (denatured aSyn). PSR1 was able to capture and enrich low levels of aSyn fibrils from biological matrices containing an excess of aSyn monomeric proteins, demonstrating significant selectivity for aggregated aSyn. See FIG. 51.

### Optimization of MPA Assay Elution Conditions for Alpha-Synuclein Fibrils

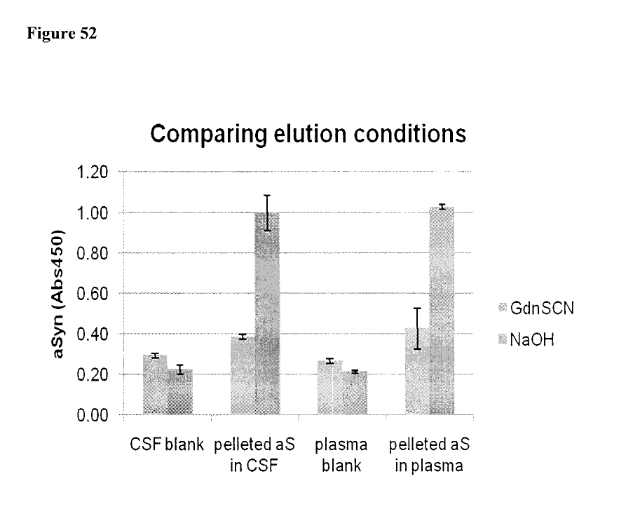

In order to optimize conditions for use of the PSR1 beads for detection of alpha-synuclein fibrils in the MPA assay, different elution conditions were tested 1) 6 M GdnSCN for 30 min versus 2) 0.10 N NaOH for 10 min. 0.1 N NaOH elution for 10 minutes performed better than the guanidine thiocyanate elution. See FIG. 52.

### Example 14

### Mouse Infectivity of PSR1—Prion

This Example demonstrates that PSR1 binds preferentially to the infectious form of the prion protein.

### Material and Methods:

### Preparation of Hamster Plasma

Golden Syrian hamsters at a one month weanling stage were inoculated intraperitoneally with 100 μl of 263K-infected 1% hamster brain homogenate (w/v) estimated at 10⁷ LD₅₀ infectious units (Kimberlin & Walker, 1986) or with 100 μl of a 1% uninfected hamster brain homogenate. Thereafter, the hamsters were sacrificed and blood was harvested in the presence of EDTA-anticoagulant by cardiac puncture at 0, 30, 50, and 80 days post-inoculation. Animals were also sacrificed and samples taken at the symptomatic stage when clinical signs of ataxia, poor grooming, and loss of appetite appeared. Individual blood samples were centrifuged at 950×g for 10 minutes and the plasma in the supernatant fraction was transferred to another tube and frozen at −80° C.

### Bead-Based Capture of PrP^{Sc}

For the sensitivity assay, 30 μl of serial ten-fold dilutions of a 263 K-infected 10% brain homogenate in PBS were intracerebrally inoculated into Tg(SHaPrP) mice (groups=4-8) (Scott et al, 1989).

For the PSR1 capture assay, the plasma from 11 symptomatic hamsters that were scarified at 143 dpi and 154 dpi were combined for pool 1, from 14 symptomatic hamsters scarified between 104-106 dpi for pool 2, from 20 pre-symptomatic hamsters at 50 dpi for pool 3 and from 15 symptomatic hamsters at 117-118 dpi for pool 4. 21 μl of PSR1-conjugated beads ((Lau et al, 2007); Gao, et al. 2010 manuscript in submission) were washed five times in 1 ml PBS (8 mM Na₂HPO₄, 1.5 mM KH₂PO₄, 137 mM NaCl, 2.7 mM KCl, pH7.4) before incubation with 500 μl of pooled hamster plasma overnight at 4° C. on a shaker.

Unbound material was removed from the beads by washing five times with 1 ml of PBS or TBSTT. The beads were resuspended in 60 μl or 120 μl in PBS and 30 μl, respectively, of the resuspended beads were intracerebrally inoculated into Tg(SHaPrP) mice with groups of at least 4 mice.

Mice were monitored every second day, and TSE (transmissible spongifirm encephalitis) was diagnosed according to clinical criteria including ataxia, wobbling, and hind leg paresis. At the onset of terminal disease Tg(SHaPrP) mice were sacrificed. Mice were maintained under conventional conditions, and all experiments were performed in accordance with the animal welfare guidelines of the Kanton of Zürich.

### Histopathology and Immunohistochemical Stains

Two-μm thick sections were cut onto positively charged silanized glass slides and stained with hematoxylin and eosin, or immunostained using antibodies for PrP (SAF84), for astrocytes (GFAP). For PrP staining, sections were deparaffinized and incubated for 6 min in 98% formic acid, then washed in distilled water for 5 min.

Sections were heated to 100° C. in a pressure cooker in citrate buffer (pH 6.0), cooled for 3 min to room temperature, and washed in distilled water for 5 min. Immunohistochemical stains were performed on an automated NEXES immunohistochemistry staining apparatus (Ventana Medical Systems, Switzerland) using an IVIEW DAB Detection Kit (Ventana). After incubation with protease 1 (Ventana) for 16 min, sections were incubated with anti-PrP SAF-84 (SPI bio; 1:200) for 32 min. Sections were counterstained with hematoxylin. GFAP immunohistochemistry for astrocytes (rabbit anti-mouse GFAP polyclonal antibody 1:1000 for 24 min; DAKO) was similarly performed, however with antigen retrieval by heating to 100° C. in EDTA buffer (pH=8.0).

Histoblot analysis was performed by using a modified standard protocol according to Taraboulos et al., 1992. 10 μm thick cryosections were mounted on glass slides and immediately pressed to a Nitrocellulose membrane (Protran, Schleicher & Schuell), soaked with lysis buffer (10 mM Tris, 100 mM NaCl, 0.05% Tween 20, pH 7.8) and air dried. After protein transfer, sections were rehydrated in TBST for 1 hour previous to Proteinase K digestion with 20, 50 and 100 μg/mL in 10 mM Tris-HCl pH 7.8 containing 100 mM NaCl and 0.1% Brij35), for 4 hours at 37° C. After washing the membrane 3 times in TBST, a denaturation step with 3 M Guanidinium thiocyanate in 10 mM Tris-HCl, pH 7.8, was performed for 10 min at room temperature. The membrane was washed and blocked with 5% non-fat milk (in TBST) and incubated with anti-PrP antibody POM-1 (epitope in the globular domain, aa 121-231), 1:10000, over night at 4° C. (Polymenidou et al, 2008). The blots were washed again and an alkaline-phosphate-conjugated goat anti mouse antibody was added (DAKO, 1:2000). Another washing step with TBST and B3 buffer (100 mM Tris, 100 mM NaCl, 100 mM MgCl₂, pH 9.5) was followed by the visualisation step with BCIP/NBT (Roche) for 45 minutes. The colour development step was stopped with distilled water. Blots were air dried and pictures were taken with an Olympus SZX12 Binocular and Olympus Camera.

### Western Blots

10% brain homogenates were prepared in 0.32 M sucrose using a Precellys24 (Bertin). Extracts of 50-90 μg protein were digested with 50 μg/mL proteinase-K in DOC/NP-40 0.5% for 45 minutes at 37° C. The reaction was stopped by adding 3 μl complete protease inhibitor cocktail and 8 μl of a lauryl dodecyl sulfate (LDS)-based sample buffer. The samples were heated to 95° C. for 5 minutes prior to electrophoresis through a 12% Bis-Tris precast gel (Invitrogen), followed by transfer to a nitrocellulose membrane by wet blotting. Proteins were detected by incubating with anti-PrP POM1 antibody (1:10000) overnight at 4° C. For secondary detection an HRP-conjugated anti-mouse IgG antibody (Zymed, Invitrogen) was used. Signals were visualized with the ECL detection kit (Pierce).

### Results

### Sensitivity Assay to Determine the Titre of the 263K Hamster Strain in Tg(SHaPrP)

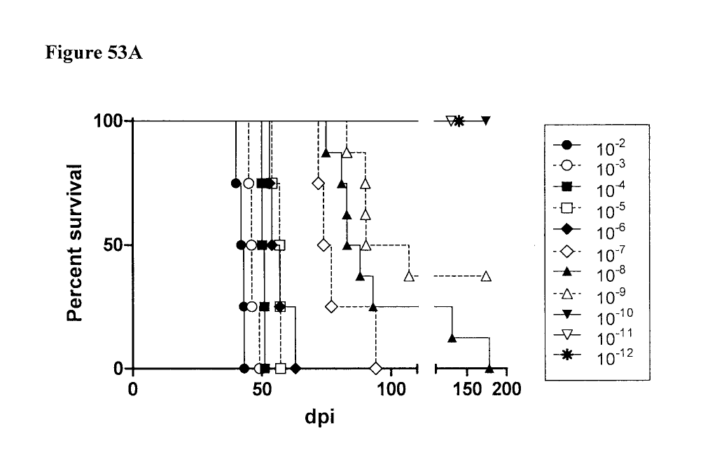

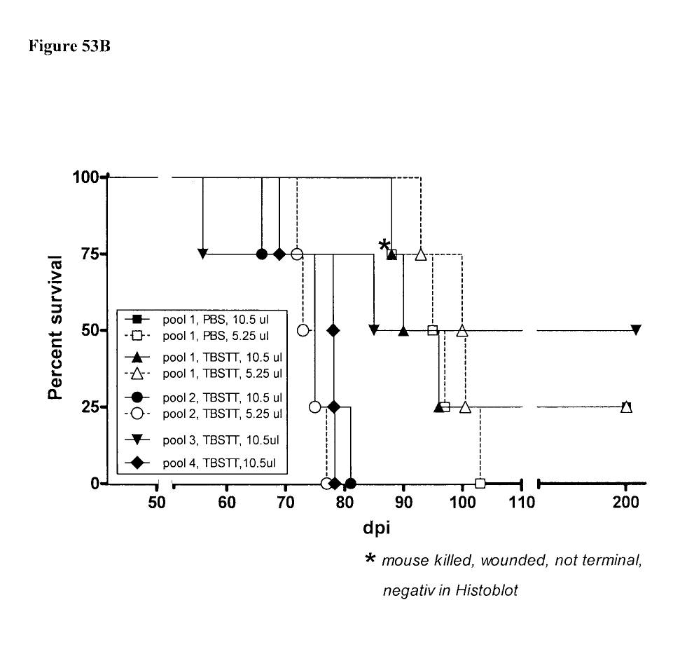

To generate a standard curve for the prion infectivity captured by the PSR1 beads from plasma we intracerebrally inoculated 10 fold serial dilutions obtained from a 10 (wt/vol) % 263K hamster brain homogenate in the end point format into Tg(SHaPrP) mice that 32-fold overexpress the hamster prion protein (Scott et al, 1989) (FIG. 53, Table 22). Mice inoculated with dilutions ranging from 10⁻² to 10⁻⁸ developed clinical signs after mean incubation times of 40 to 98 days. Since the end point is not reached yet, further dilutions will be performed to obtain a complete standard curve.

TABLE 22
Summary of end-point titrations of the 263 K inoculums in Tg(SHaPrP)

| Dilution of brain | (Clinical TSE/total | Mean incubation period |
|---|---|---|
| homogenate^{a} | inoculated) | (days) |
| 10⁻² | 4/4 | 42 ± 1 |
| 10⁻³ | 4/4 | 47 ± 1.4 |
| 10⁻⁴ | 4/4 | 51 ± 0.5 |
| 10⁻⁵ | 4/4 | 56 ± 1.2 |
| 10⁻⁶ | 4/4 | 57 ± 3.3 |
| 10⁻⁷ | 4/4 | 79 ± 7.4 |
| 10⁻⁸ | 8/8 | 100 ± 34.4 |
| 10⁻⁹ | 5/8 | 83, 90, 90, 90, 107, >174, |
| | | >174, >174; ongoing |
| 10⁻¹⁰ | | Ongoing |
| 10⁻¹¹ | | Ongoing |
| 10⁻¹² | | ongoing |

^{a}Dilutions were started from a 10% brain homogenate.

Bioassay with Plasma Coated PSR1 Beads from Prion Infected Hamster in Tg(SHaPrP) Mice

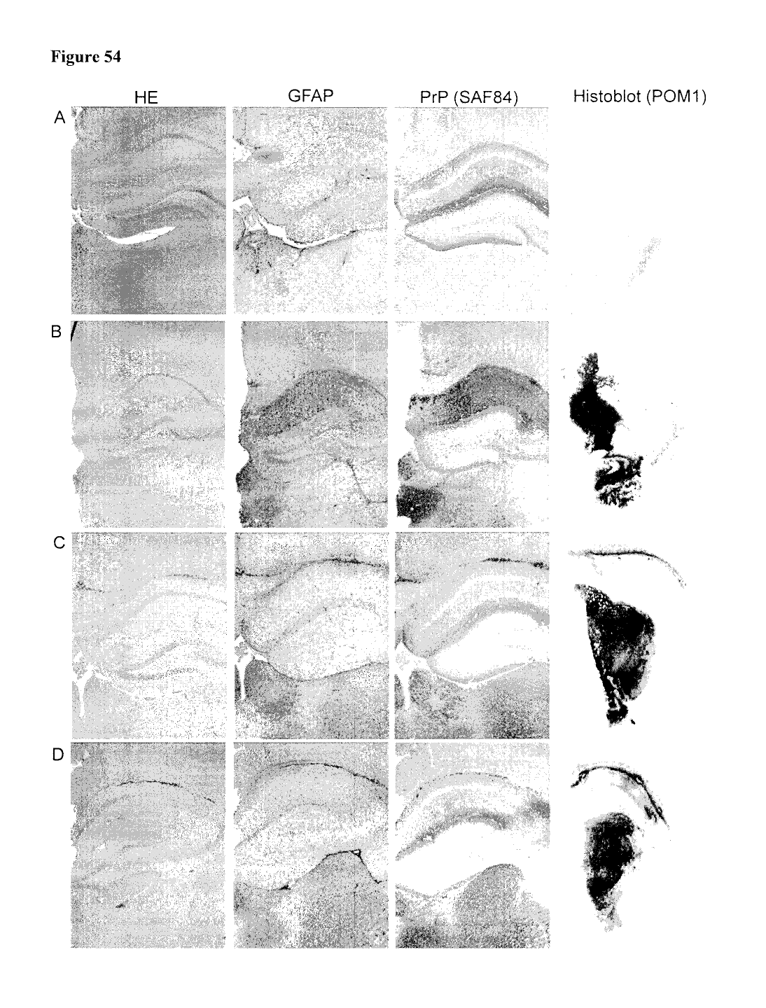

PSR1 beads were incubated with plasma samples that were either pooled from presymptomatic or symptomatic groups of 263K prion-infected hamster (Table 23) and i.e. inoculated into Tg(SHaPrP) mice. Mice inoculated with beads of plasma pools from symptomatic hamster developed disease after mean incubation times of 74-94 days post inoculation (FIG. 54, Table 23). Mice inoculated with beads obtained from presymptomatic hamster developed disease after 56 and 85 days post inoculation (FIG. 54, Table 23). The observed incubation times correlate to infectious dilutions of 10⁻⁷-10⁻⁸ of 30 μl 263K hamster brain homogenate.

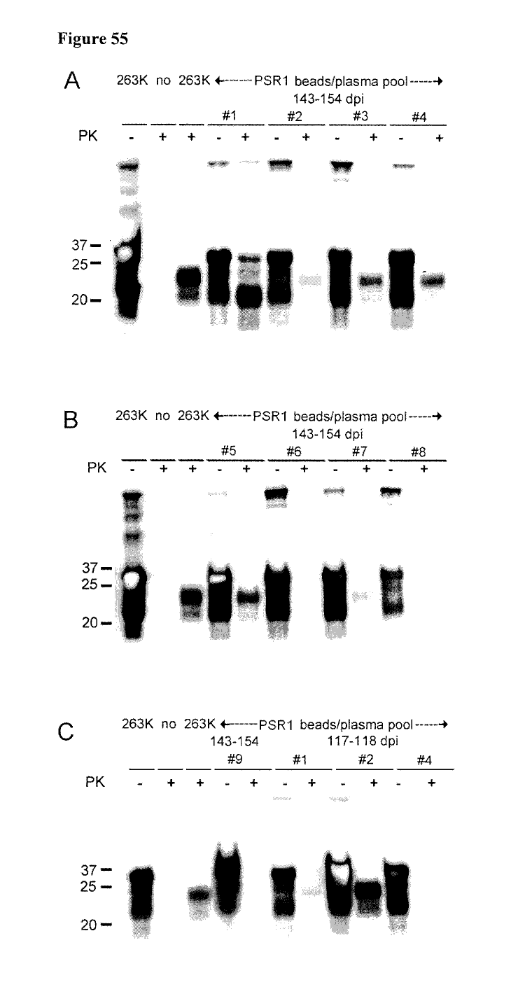

The occurrence of a prion disease in clinically diseased mice was manifested by histopathological and immunohistochemical analysis (FIG. 54) and by the detection of proteinase K resistant material in Histoblot and Western blot analysis (FIG. 55).

These data show that PSR1 beads capture prion infectivity from prion-infected blood samples and transmit it with high efficiency to Tg(SHaPrP) mice.

TABLE 23
Summary of the bioassay of Tg(SHaPrP) mice that were inoculated with
plasma coated PSR1 beads

| | Attack rate | | |
|---|---|---|---|
| | (Clinical | Mean | |
| | TSE/total | incubation | |
| Plasma Pools | inoculated) | period (days) | |
| Pool 1: | 81% (13/16) | 94 ± 5 dpi | 125 μl pooled plasma |
| Symptomatic | | (plus 3 | corresponds to 30 μl of a |
| hamster, 11 | | survivor | 10⁻⁸ dilution of 263 K |
| animals, 143 dpi | | stopped at | hamster brain homogenate |
| and 154 dpi | | 200 dpi) | (0.000001%) |
| Pool 2: | 100% (8/8) | 74 ± 3 dpi | 125 μl pooled plasma |
| symptomatic | | | corresponds to 30 μl of a |
| hamster, 14 | | | 10⁻⁷ dilution of 263 K |
| animals, 104-106 | | | hamster brain homogenate |
| dpi | | | (0.00001%) |
| Pool 3: pre- | 50% (2/4) | 56, 85 | |
| symptomatic | | (plus 2 | |
| hamster, 20 | | survivor | |
| animals, 50 dpi | | stopped at | |
| | | 200 dpi) | |
| Pool 4: | 100% (4/4) | 76 ± 5 dpi | 125 μl pooled plasma |
| symptomatic | | | corresponds to 30 μl of a |
| hamster, 15 | | | 10⁻⁷ dilution of 263 K |
| animals, 117-118 | | | hamster brain homogenate |
| dpi | | | (0.00001%) |

### REFERENCES

Kimberlin R H, Walker C A (1986) Pathogenesis of scrapie (strain 263K) in hamsters infected intracerebrally, intraperitoneally or intraocularly. J Gen Virol 67: 255-263
Lau A L, Yam A Y, Michelitsch M M, Wang X, Gao C, Goodson R J, Shimizu R, Timoteo G, Hall J, Medina-Selby A, Coit D, McCoin C, Phelps B, Wu P, Hu C, Chien D, Peretz D (2007) Characterization of prion protein (PrP)-derived peptides that discriminate full-length PrPSc from PrPC. Proc Natl Acad Sci USA 104: 11551-11556
Polymenidou M, Moos R, Scott M, Sigurdson C, Shi Y Z, Yajima B, Hafner-Bratkovic I, Jerala R, Hornemann S, Wuthrich K, Bellon A, Vey M, Garen G, James M N, Kay N, Aguzzi A (2008) The POM monoclonals: a comprehensive set of antibodies to non-overlapping prion protein epitopes. PLoS One 3: 805-814
Scott M, Foster D, Mirenda C, Serban D, Coufal F, Walchli M, Torchia M, Groth D, Carlson G, DeArmond S J, Westaway D, Prusiner S B (1989) Transgenic mice expressing hamster prion protein produce species-specific scrapie infectivity and amyloid plaques. Cell 59: 847-857
Taraboulos A, Jendroska K, Serban D, Yang S L, DeArmond S J, Prusiner S B (1992) Regional mapping of prion proteins in the brain. Proc Nail Acad Sci USA 89: 7620-7624.

1.-134. (canceled)

135. A method for detecting the presence of oligomer in a sample comprising the steps of:
(a) providing a sample suspected of containing oligomer, wherein said sample lacks aggregates other than oligomers; or providing a sample suspected of containing oligomer and removing aggregate other than oligomer from said sample;
(b) contacting said sample with an aggregate-specific binding reagent under conditions that allow binding of said reagent to said oligomer, if present, to form a complex; and
(c) detecting the presence of oligomer, if any, in said sample by its binding to said aggregate-specific binding reagent;
wherein said aggregate-specific binding reagent has a net charge of at least about positive one at the pH at which said sample is contacted with said aggregate-specific binding reagent,
is attached to a solid support at a charge density of at least about 2000 nmol net charge per square meter, and
binds preferentially to aggregate over monomer when attached to said solid support.

136. The method of claim 135, wherein said method comprises the steps of:
contacting a sample suspected of containing oligomer with an aggregate-specific binding reagent under conditions that allow binding of said reagent to said oligomer, if present, to form a complex;
contacting said complex with a second reagent, wherein said reagent binds preferentially to either oligomer or aggregates other than oligomer;
detecting the presence of oligomer, if any, in said sample by its binding or lack of binding to said second reagent.

137. The method of claim 135, wherein said aggregate other than oligomer comprises fibrils.

138. A method for detecting the presence of aggregate in a sample comprising the steps of:
(a) contacting a sample suspected of containing aggregate with an aggregate-specific binding reagent under conditions that allow binding of said reagent to said aggregate, if present, to form a complex; and
(b) detecting the presence of aggregate, if any, in said sample by its binding to said aggregate-specific binding reagent;
wherein said aggregate-specific binding reagent has a net charge of at least about positive one at the pH at which said sample is contacted with said aggregate-specific binding reagent,
is attached to a solid support at a charge density of at least about 60 nmol net charge per square meter, and
binds preferentially to aggregate over monomer when attached to said solid support.

139. The method of claim 138, wherein said method comprises the following additional steps after step (a):
(i) removing unbound sample;
(ii) dissociating said aggregate from said complex thereby providing dissociated aggregate; and
(iii) contacting said dissociated aggregate with a first conformational protein-specific binding reagent under conditions that allow binding to form a second complex; and
wherein said detecting the presence of aggregate in said sample is performed by detecting the formation of said second complex.

140. The method of claim 138, wherein said aggregate comprises Aβ protein and said conformational protein-specific binding reagent is an anti-Aβ antibody.

141. The method of claim 138, wherein step (a) comprises:
contacting a sample suspected of containing aggregate with a conformational protein-specific binding reagent under conditions that allow binding of said conformational protein-specific binding reagent to said aggregate, if present, to form a complex;
removing unbound sample; and
contacting said complex with an aggregate-specific binding reagent under conditions that allow the binding of said aggregate-specific binding reagent to said aggregate, wherein said aggregate-specific binding reagent comprises a detectable label.

142. The method of claim 138, wherein said method comprises the following additional steps preceding step (a):
providing a solid support comprising an aggregate-specific binding reagent;
combining said solid support with a detectably labeled ligand, wherein said aggregate-specific binding reagent's binding avidity to said detectably labeled ligand is weaker than said reagent's binding avidity to said aggregate;
wherein step (a) comprises combining a sample suspected of containing aggregate with said solid support under conditions which allow said aggregate, when present in said sample, to bind to said reagent and replace said ligand; and
wherein step (b) comprises detecting complexes formed between said aggregate and said aggregate-specific binding reagent.

143. A method for reducing the amount of aggregate in a polypeptide sample comprising the steps of:
contacting a polypeptide sample suspected of containing aggregate with the aggregate-specific binding reagent under conditions that allow binding of said reagent to said aggregate, if present, to form a complex; and
recovering unbound polypeptide sample,
wherein said aggregate-specific binding reagent has a net charge of at least about positive one at the pH at which said sample is contacted with said aggregate-specific binding reagent,
is attached to a solid support at a charge density of at least about 60 nmol net charge per square meter, and
binds preferentially to aggregate over monomer when attached to said solid support.

144. The method of claim 135, 138 or 143, wherein said aggregate-specific binding reagent is attached to a solid support at a charge density of at least about:
90 nmol net charge per square meter;
120 nmol net charge per square meter;
500 nmol net charge per square meter,
1000 nmol net charge per square meter, or
2000 nmol net charge per square meter.

145. The method of claim 135, 138 or 143, wherein said oligomer or aggregate is soluble.

146. The method of claim 135, 138 or 143, wherein said sample is a biological sample comprising bodily tissues or fluid.

147. The method of claim 135, 138 or 143, wherein biological sample comprises whole blood, blood fractions, blood components, plasma, platelets, serum, cerebrospinal fluid (CSF), bone marrow, urine, tears, milk, lymph fluid, organ tissue, brain tissue, nervous system tissue, muscle tissue, non-nervous system tissue, biopsy, necropsy, fat biopsy, cells, feces, placenta, spleen tissue, lymph tissue, pancreatic tissue, bronchoalveolar lavage, or synovial fluid.

148. The method of claim 135, 138 or 143, wherein said aggregate-specific binding reagent has a net charge of at least about positive two, at least about positive three, at least about positive four, at least about positive five, at least about positive six, or at least about positive seven at the pH at which the sample is contacted with said aggregate-specific binding reagent.

149. The method of claim 135, 138 or 143, wherein said aggregate-specific binding reagent:
has a binding affinity and/or avidity for aggregate that is at least about two times higher than the binding affinity and/or avidity for monomer.

150. The method of claim 135, 138 or 143, wherein said aggregate-specific binding reagent:
comprises at least one positively charged functional group having a pKa at least about 1 pH unit higher than the pH at which the sample is contacted with said aggregate-specific binding reagent.

151. The method of claim 150, wherein said at least one positively charged functional group is closest to said solid support among all functional groups of said aggregate-specific binding reagent.

152. The method of claim 135, 138 or 143, wherein said aggregate-specific binding reagent comprises a hydrophobic functional group.

153. The method of claim 135, 138 or 143, wherein said aggregate-specific binding reagent comprises:
(a) only one positively charged functional group and at least one hydrophobic functional group;
(b) at least one positively charged functional group and only one hydrophobic functional group; or
(c) only one positively charged functional group and only one hydrophobic functional group.

154. The method of claim 135, 138 or 143, wherein said oligomer or aggregate is pathogenic.

155. The method of claim 135, 138 or 143, wherein said oligomer or aggregate is associated with preeclampsia, tauopathy, TDP-43 proteinopathy, or serpinopathy or an amyloid disease.

156. The method of claim 135, 138 or 143, wherein said oligomer or aggregate is selected from the group consisting of amyloid-beta (Aβ) protein, tau protein, amylin, Amyloid A protein, anti-trypsin and alpha-synuclein.

157. The method of claim 156, wherein said oligomer or aggregate is associated with amyloid disease and said amyloid disease is selected from the group consisting of systemic amyloidosis, AA amyloidosis, synucleinopathy, Alzheimer's disease, ALS, immunoglobulin-related diseases, serum amyloid A-related diseases, Huntington's disease, Parkinson's disease, diabetes type II, dialysis amyloidosis, and cerebral amyloid angiopathy.

158. The method of claim 135, 138 or 143, wherein said aggregate-specific binding reagent comprises: at least one amino acid that is an L-isomer or is a D-isomer; and/or wherein said aggregate-specific binding reagent comprises a natural amino acid selected from the group consisting of lysine and arginine; and/or an unnatural amino acid selected from the group consisting of ornithine, methyllysine, diaminobutyric acid, homoarginine, and 4-aminomethylphenylalanine.

159. The method of claim 135, 138 or 143, wherein said aggregate-specific binding reagent comprises:
(a) a peptide selected from the group consisting of KKKFKF (SEQ ID NO: 1), KKKWKW (SEQ ID NO: 2), KKKLKL (SEQ ID NO: 3), KKKKKK (SEQ ID NO: 4), KKKKKKKKKKKK (SEQ ID NO: 5), AAKKAA (SEQ ID NO: 32), AAKKKA (SEQ ID NO: 33), AKKKKA (SEQ ID NO: 34), AKKKKK (SEQ ID NO: 35), FKFKKK (SEQ ID NO: 36), kkkfkf (SEQ ID NO: 37), FKFSLFSG (SEQ ID NO: 38), DFKLNFKF (SEQ ID NO: 39), FKFNLFSG (SEQ ID NO: 40), YKYKKK (SEQ ID NO: 41), KKFKKF (SEQ ID NO: 42), KFKKKF (SEQ ID NO: 43), KIGVVR (SEQ ID NO: 44), AKVKKK (SEQ ID NO: 45), AKFKKK (SEQ ID NO: 46), RGRERFEMFR (SEQ ID NO: 47), YGRKKRRQRRR (SEQ ID NO: 48), FFFKFKKK (SEQ ID NO: 49), FFFFFKFKKK (SEQ ID NO: 50), FFFKKK (SEQ ID NO: 51), and FFFFKK (SEQ ID NO: 52);
(b) a peptide selected from the group consisting of F-fdb-F-fdb-fdb-fdb (SEQ ID NO: 53), FoF000 (SEQ ID NO: 54), monoBoc-ethylenediamine+BrCH2CO-KKFKF (SEQ ID NO: 55), triethylamine+BrCH2CO-KKFKF (SEQ ID NO: 56), tetramethylethylenediamine+BrCH2CO-KKFKF (SEQ ID NO: 57) and SEQ ID NOs: 58-66;
(c) a peptide selected from the group consisting of KFYLYAIDTHRM (SEQ ID NO: 6), KIIKWGIFWMQG (SEQ ID NO: 7), NFFKKFRFTFTM (SEQ ID NO: 8), MKFMKMHNKKRY (SEQ ID NO: 67), LTAVKKVKAPTR (SEQ ID NO: 68), LIPIRKKYFFKL (SEQ ID NO: 69), KLSLIWLHTHWH (SEQ ID NO: 70), IRYVTHQYILWP (SEQ ID NO: 71), YNKIGVVRLFSE (SEQ ID NO: 72), YRHRWEVMLWWP (SEQ ID NO: 73), WAVKLFTFFMFH (SEQ ID NO: 74), YQSWWFFYFKLA (SEQ ID NO: 75), WWYKLVATHLYG (SEQ ID NO: 76), QTLSLHFQTRPP (SEQ ID NO: 77), TRLAMQYVGYFW (SEQ ID NO: 78), RYWYRHWSQHDN (SEQ ID NO: 79), AQYIMFKVFYLS (SEQ ID NO: 80), TGIRIYSWKMWL (SEQ ID NO: 81), SRYLMYVNIIYI (SEQ ID NO: 82), RYWMNAFYSPMW (SEQ ID NO: 83), NFYTYKLAYMQM (SEQ ID NO: 84), MGYSSGYWSRQV (SEQ ID NO: 85), YFYMKLLWTKER (SEQ ID NO: 86), RIMYLYHRLQHT (SEQ ID NO: 87), RWRHSSFYPIWF (SEQ ID NO: 88), QVRIFTNVEFKH (SEQ ID NO: 89), and RYLHWYAVAVKV (SEQ ID NO: 90); or
(d) a peptoid selected from the group consisting of: 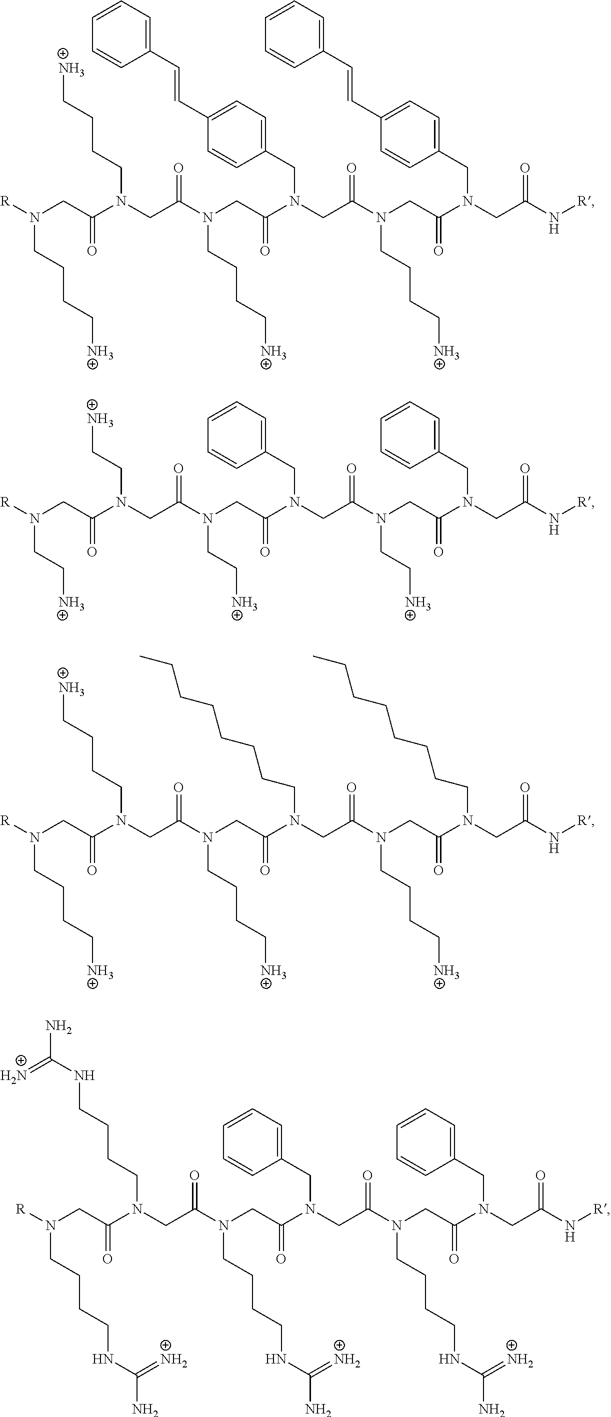 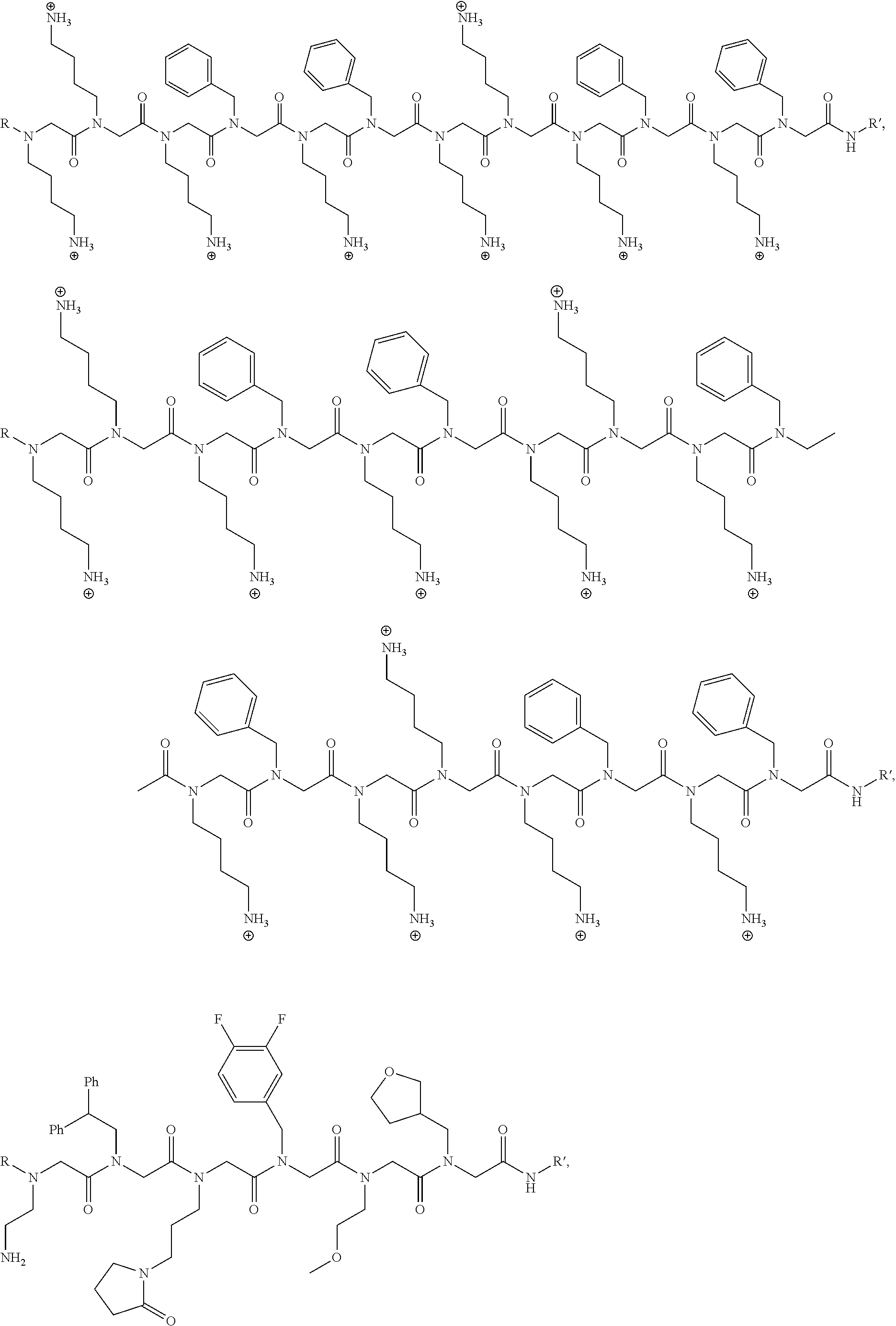 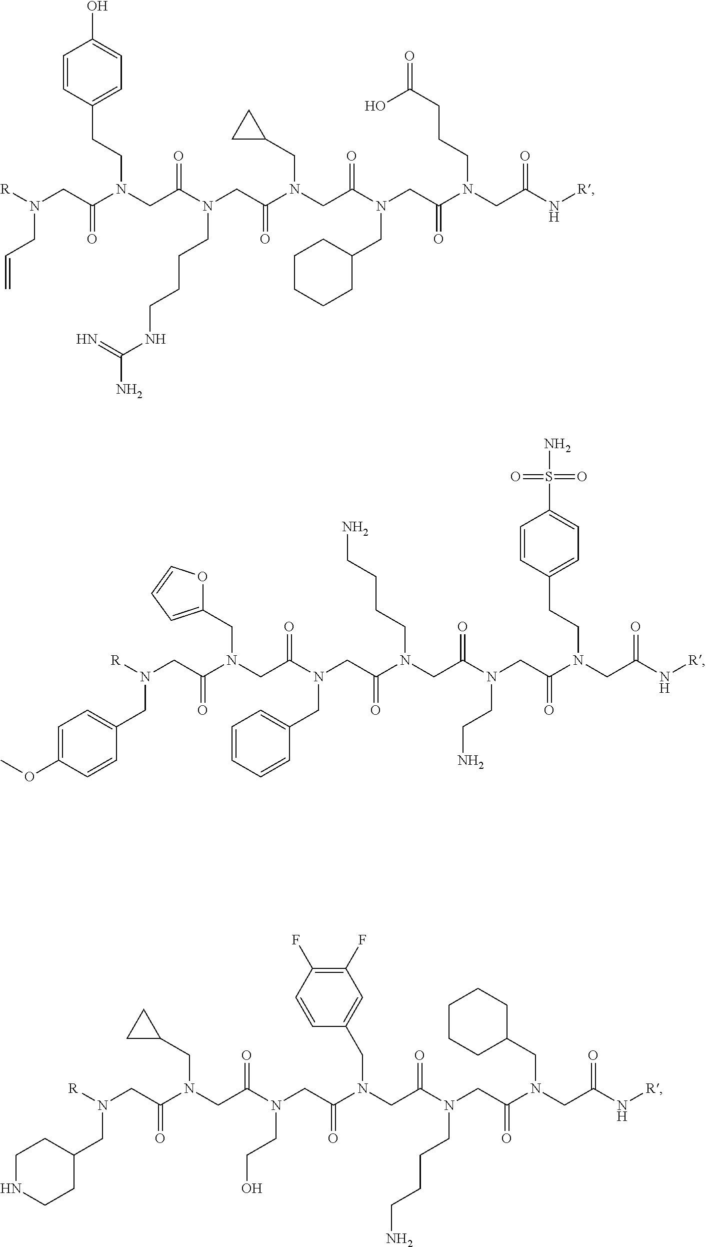 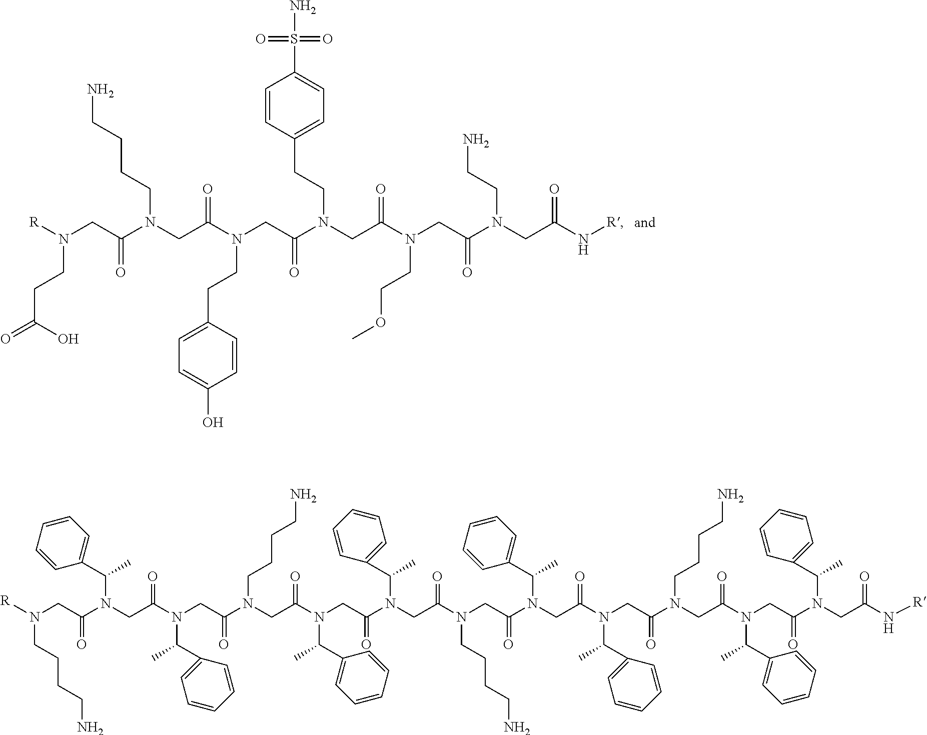
wherein R and R′ are any group;
(e) a peptoid selected from the group consisting of SEQ ID NOs: 9-14 and 91-96; or 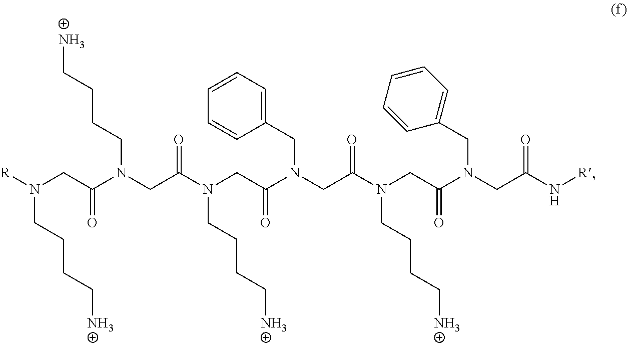
wherein R and R′ are any group.

160. The method of claim 135, 138 or 143, wherein said aggregate-specific binding reagent comprises the Dendron

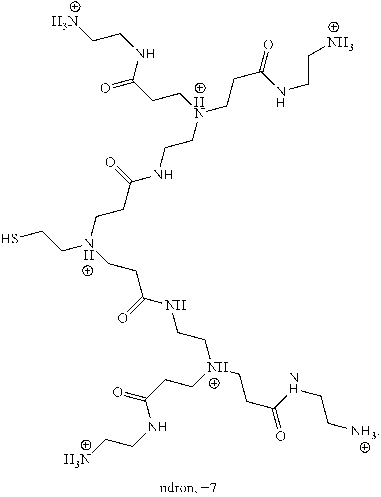

161. The method of claim 135, 138 or 143, wherein said aggregate-specific binding reagent comprises:
repeating motifs; and/or
positively charged groups with the same spacing as that of the negatively charged groups of the aggregate.

162. The method of claim 135, 138 or 143, wherein said aggregate-specific binding reagent comprises SEQ ID NO:1.

163. The method of claim 135, 138 or 143, wherein said aggregate-specific binding reagent comprises SEQ ID NO:15.

164. The method of claim 135, 138 or 143, wherein:
(a) said oligomer or aggregate comprises amylin, wherein said aggregate-specific binding reagent comprises: SEQ ID NO: 15, and wherein said aggregate-specific binding reagent is attached to a solid support at a charge density of at least about 8000 nmol to about 15000 nmol net charge per square meter;
(b) said oligomer or aggregate comprises alpha-synuclein, wherein said aggregate-specific binding reagent comprises SEQ ID NO: 15, and wherein said aggregate-specific binding reagent is attached to a solid support at a charge density of at least about 8000 nmol to about 15000 nmol net charge per square meter; or
(c) said oligomer or aggregate comprises Amyloid A protein, wherein said aggregate-specific binding reagent comprises SEQ ID NO: 15, and wherein said aggregate-specific binding reagent is attached to a solid support at a charge density of at least about 8000 nmol to about 15000 nmol net charge per square meter.

165. The method of claim 139, wherein said aggregate comprises Aβ and said first conformational protein-specific binding reagent is a first anti-Aβ antibody coupled to a solid support and said detecting the formation of said second complex uses a detectably labeled second anti-Aβ antibody.

166. The method of claim 135, 138 or 143, wherein said aggregate-specific binding reagent comprises:
(a) a peptoid selected from the group consisting of: 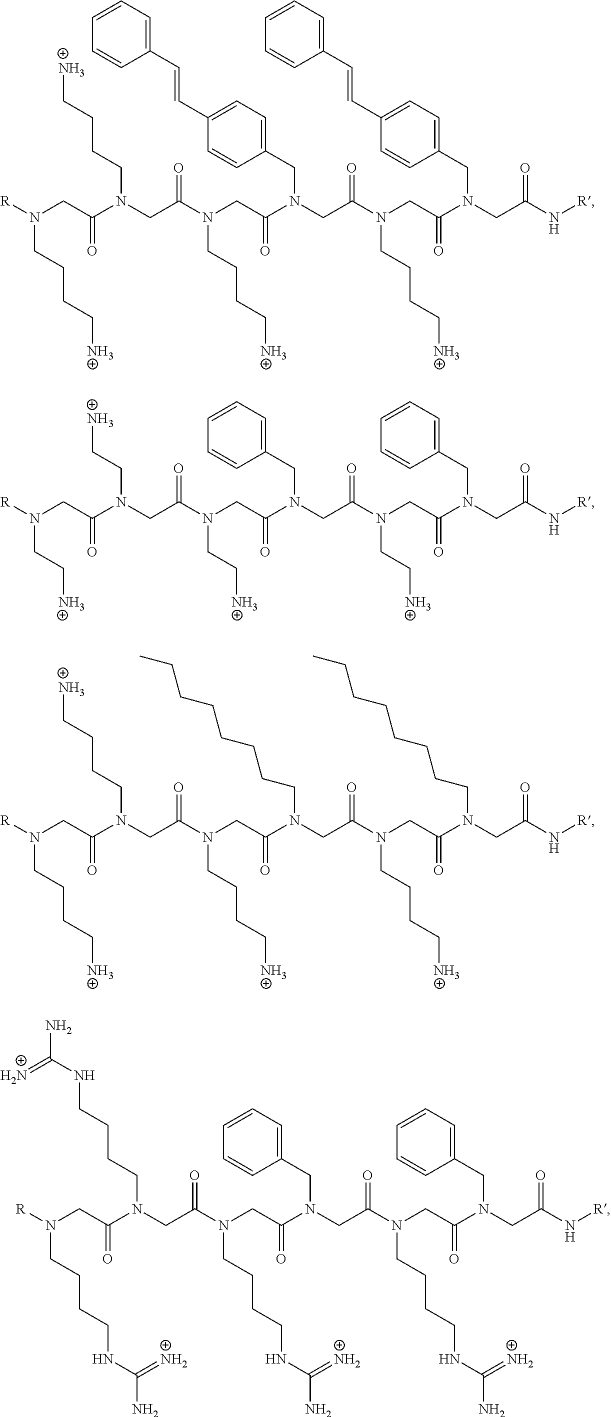 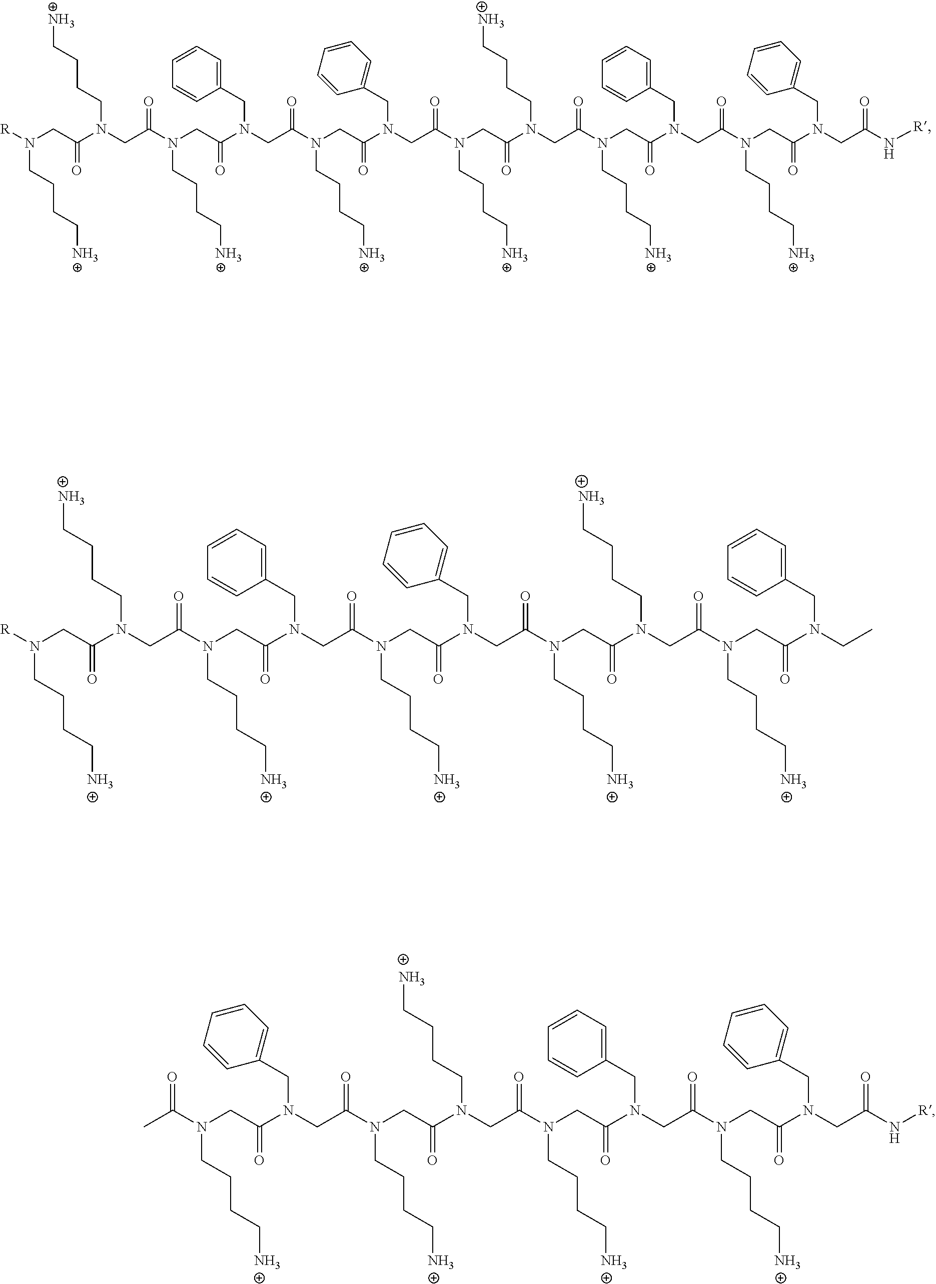 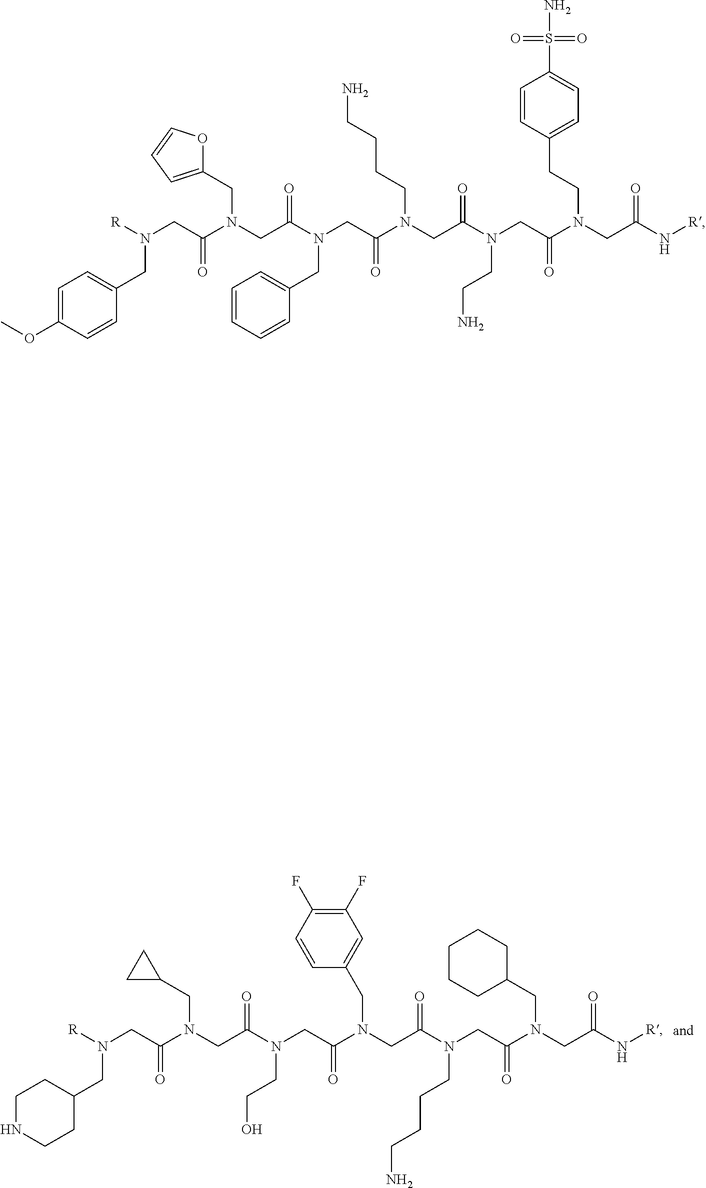 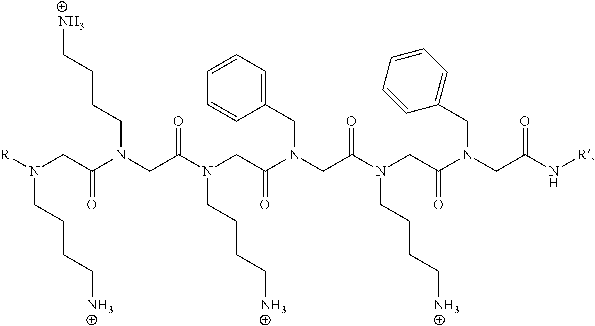
wherein R and R′ are any group; or
(b) a peptide selected from the group consisting of: KKKFKF (SEQ ID NO: 1), KKKWKW (SEQ ID NO: 2), KKKLKL (SEQ ID NO: 3), FKFKKK (SEQ ID NO: 36), FFFKFKKK (SEQ ID NO: 49), FFFFFKFKKK (SEQ ID NO: 50), FFFKKK (SEQ ID NO: 51), FFFFKK (SEQ ID NO: 52), KKFKKF (SEQ ID NO: 42), KFKKKF (SEQ ID NO: 43), kkkfkf (SEQ ID NO: 37), KIGVVR (SEQ ID NO: 44), MKFMKMHNKKRY (SEQ ID NO: 67), LIPIRKKYFFKL (SEQ ID NO: 69), RGRERFEMFR (SEQ ID NO: 47), and SEQ ID NOs 53, 55, 56 and 58-66.

167. The method of claim 135, 138 or 143, further comprising a step of treating said complex formed between said aggregate-specific binding reagent and said aggregate or oligomer with a neutral detergent, wherein said detergent comprises: (a) both positive and negative charges; or (b) a long carbon chain.
